(19) United States
(12) Patent Application Publication
(10) Pub. No.: US 2011/0183889 A1
(43) Pub. Date: Jul. 28, 2011

(54) SALICYLANILIDE MODIFIED PEPTIDES FOR USE AS ORAL THERAPEUTICS

(75) Inventors: Alan M. Fogelman, Beverly Hills, CA (US); Mohamad Navab, Los Angeles, CA (US)

(73) Assignee: The Regents of the University of California, Oakland, CA (US)

(21) Appl. No.: 12/674,394

(22) PCT Filed: Aug. 28, 2008
(86) PCT No.: PCT/US08/74624

§ 371 Date: Mar. 3, 2011

Related U.S. Application Data
(60) Provisional application No. 60/968,815, filed on Aug. 29, 2007.

(51) Int. Cl. A61K 38/10; C07K 1/113; C07K 1/00; A61K 38/02; A61K 38/16; A61K 38/07; A61K 38/08; A61P 9/10; A61P 29/00; A61P 19/10; A61P 25/28; A61P 11/06; A61P 3/10; A61P 13/12; A61P 31/12; A61P 11/00

(52) U.S. Cl. 514/1.7; 530/345; 530/409; 530/359; 530/326; 530/327; 530/324; 530/330; 530/328; 530/329; 530/333; 514/1.1; 514/21.4; 514/21.3; 514/21.5; 514/21.9; 514/21.6; 514/21.7; 514/21.8; 514/1.9; 514/16.6; 514/16.9; 514/17.8; 514/17.9; 514/6.9; 514/15.4

(57) ABSTRACT

This invention pertains to the surprising discovery that salicylanilides, e.g., niclosamide and/or niclosamide analogues can be reacted with a therapeutically active peptide to produce a modified peptide complex that shows increased resistance to proteolysis and that shows higher bioactivity when orally administered than the unmodified peptide.

### CROSS-REFERENCE TO RELATED APPLICATIONS

This application claims priority to and benefit of U.S. Ser. No. 60/968,815, filed Aug. 29, 2007, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported, in part, by USPHS Grant 2 P01 HL-030568. The government of the United States of America may possess certain rights in this invention.

### FIELD OF THE INVENTION

The present invention relates to oral peptide pharmaceuticals where the active compounds include a plurality of amino acids and at least one peptide bond in their molecular structures, and to methods of enhancing bioavailability of such peptide compounds when administered orally.

### BACKGROUND OF THE INVENTION

Numerous human hormones, neurotransmitters, or therapeutic antibodies are peptides or comprise peptides as a substantial part of their molecular structures. Therapeutically effective amounts of such biologically relevant peptides may be administered to patients in a variety of ways. Oral delivery of pharmacologically active agents is generally the delivery route of choice since it is convenient, self administration is relatively easy and generally painless, resulting in greater patient compliance as compared to other modes of delivery.

Biological, chemical and physical barriers such as varying pH in the gastrointestinal tract, powerful digestive enzymes in the stomach and intestine, and active agent impermeable gastrointestinal membranes, however, often makes the effective delivery of peptide pharmaceuticals problematic. For example, the oral delivery of calcitonins, has proven difficult due, at least in part, to the insufficient stability of calcitonin in the gastrointestinal tract as well as the inability of calcitonin to be readily transported through the intestinal walls into the blood stream.

Consequently peptide pharmaceuticals used in the prior art frequently have been administered by injection or by nasal administration. Insulin is one example of a peptide pharmaceutical frequently administered by injection. Injection and nasal administration, however, are significantly less convenient than, and involve more patient discomfort than, oral administration. Often this inconvenience or discomfort results in substantial patient noncompliance with a treatment regimen. Thus, there is a need in the art for more effective and reproducible oral administration of peptide pharmaceuticals like insulin, calcitonin and others discussed in more detail herein.

### SUMMARY OF THE INVENTION

This invention pertains to the surprising discovery that salicylanilides, e.g., niclosamide and/or niclosamide analogues when orally administered in conjunction with a peptide pharmaceutical (e.g., a class A amphipathic helical peptide as described herein) or when reacted with a therapeutic peptide to produce a modified peptide (e.g., peptide-salicylanilide complex) significantly increase the bioavailability of that peptide. Methods of peptide delivery using such “delivery agents” and pharmaceutical formulations are provided.

Thus, in certain embodiments, this invention provides a method of enhancing the in vivo activity of a therapeutic peptide orally administered to a mammal, the method comprising reacting the peptide with a salicylanilide and/or with the parent acid or amine of the salicylanilide and/or with acetyl salicylic acid or a derivative of acetyl salicylic to form a complex with the peptide whereby the peptide-salicylanilide complex shows enhanced in vivo activity as compared to the untreated peptide. In certain embodiments the reacting is under acidic conditions (e.g. ranging from about pH 0.5, 1, 1.5 2, 2.5, 3, or 3.5 to about pH 4, 4.5, 5, 5.5, 6, 6.5, 6.8, or 6.9). In certain embodiments the reacting is at a temperature ranging from 20° C., 25° C., 30° C., 35° C., or 37° C. to about 50° C., 55° C., 60° C., 65° C., or 70° C. In various embodiments the reaction will be under sterile conditions. In certain embodiments the reaction can simply be run overnight at room temperature or at about 37° C. Typically, the reaction will be run for a period ranging from about 0.5, 1, 2, 3, 4, 5, 6, 7, 8, 9, 10, 11, or 12 hours to about 13, 14, 15, 16, 17, 18, 19, 20, 21, 22, 23, or 24 or more hours depending on temperature and pH. In various embodiments the modified peptide is purified by HPLC, e.g., as shown in FIGS. 36-40. In various embodiments the salicylanilide is niclosamide or a niclosamide analogue. In certain embodiments the niclosamide or niclosamide analogue is 2′5-dichloro-4′-nitrosalicylanilide, 5-chloro-salicyl-(2-chloro-4-nitro) anilide 2-aminoethanol salt, 5-chloro-salicyl-(2-chloro-4-nitro) anilide piperazine salt, and 5-chloro-salicyl-(2-chloro-4-nitro) anilide monohydrate. In certain embodiments the niclosamide analogue is a compound in FIG. 2, 3, 4, 5, 6, 7, and/or Table 1. In various embodiments the parent acid and/or the parent amine is an acid or amine in Table 1. In various embodiments the peptide ranges in length from 3, 5, 10, 15, or 18 amino acids to about 30, 36, 50, 100, 150, 200, 250, or 300 amino acids. In certain embodiments, the peptide ranges in length from about 5, 10, 15, 18, 20, 25, or 30 amino acids to about 50, 70, 90, 100, 150, 200, 250, or 300 amino acids. In certain embodiments the peptide comprises an amphipathic helix (e.g. a class A amphipathic helix). In certain embodiments the peptide is ApoJ, ApoA-I, ApoA-I milano, or 18A. In certain embodiments the peptide is ApoAI, an Apo A-1 derivatives and/or agonists (see, e.g., therapeutic peptides described in U.S. Patent Publications 20050004082, 20040224011, 20040198662, 20040181034, 20040122091, 20040082548, 20040029807, 20030149094, 20030125559, 20030109442, 20030065195, 20030008827, and 20020071862, and U.S. Pat. Nos. 6,831,105, 6,790,953, 6,773,719, 6,713,507, 6,703,422, 6,699,910, 6,680,203, 6,673,780, 6,646,170, 6,617,134, 6,559,284, 6,506,879, 6,506,799, 6,459,003, 6,423,830, 6,410,802, 6,376,464, 6,367,479, 6,329,341, 6,287,590, 6,090,921, 5,990,081, and the like which are incorporated herein by reference in their entirety for all purposes. In various embodiments the peptide consists of all “L” or all “D” amino acids, or at least one “D” amino acid. In certain embodiments the peptide is a D or L peptide whose sequence is shown in any of Tables 2-11 and/or SEQ ID Nos:1-995. In certain embodiments the peptide consists of all L amino acids. In various embodiments the peptide comprises a protecting group at the amino or carboxyl terminus (e.g., a first protecting group coupled to the amino terminus and a second protecting group coupled to the carboxyl terminus). In various embodiments the protecting group(s) are independently selected from the group consisting of acetyl, amide, and 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Born), t-butoxycarbonyl (Boc), cyclohexyloxy (cHxO), t-butoxymethyl (Bum), t-butoxy (tBuO), t-Butyl (tBu), Acetyl (Ac), and Trifluoroacetyl (TFA). In certain embodiments the first protecting group is a protecting group selected from the group consisting of acetyl, propeonyl, and a 3 to 20 carbon alkyl. In certain embodiments the second protecting group is an amide. In certain embodiments the peptide is a D or L peptide comprising the amino acid sequence DWFKAFYDKVAEKFKEAF (SEQ ID NO:5) or the amino acid sequence FAEKFKEAVKDYFAKFWD (SEQ ID NO:104). The peptide can comprise amino and/or carboxyl terminal protecting groups, e.g., as described above. In certain embodiments this peptide comprises a carboxyl terminal protecting group and an amino terminal protecting group the carboxyl terminal protecting group is an amide; and the amino terminal protecting group is an acetyl.

Also provided is a method of preparing an orally deliverable therapeutic peptide. The method involves synthesizing the peptide with one or more amino acids that are acetylated at the epsilon position of the amino acid with a salicylanilide and/or with the parent acid or amine of the salicylanilide (e.g., as shown in Table 1) and/or with acetyl salicylic acid or a derivative of acetyl salicylic to form an adduct with the peptide whereby the peptide adduct shows enhanced in vivo activity as compared to the untreated peptide. In certain embodiments the peptide is acylated at one or more lysines.

In certain embodiments this invention provides a composition comprising a modified peptide having the structure of a complex formed by reacting a therapeutically active peptide with a salicylanilide and/or with the parent acid or amine of the salicylanilide and/or with acetyl salicylic acid or a derivative of acetyl salicylic to form a complex with the peptide whereby the modified peptide shows enhanced in vivo activity as compared to the untreated peptide, e.g., as described above. In various embodiments the peptide can be any therapeutic peptide, e.g., as described above. In certain embodiments the peptide is a D or L peptide comprising the amino acid sequence DWFKAFYDKVAEKFKEAF (SEQ ID NO:5) or the amino acid sequence FAEKFKEAVKDYFAKFWD (SEQ ID NO:104). In various embodiments the peptide comprises one or more lysines acetylated. The peptide can be optionally protected at the carboxyl and/or amino terminus, e.g., as described above.

Also provided is an orally deliverable therapeutic peptide the peptide comprising a therapeutic peptide comprising one or more amino acids that are acetylated at the epsilon position of the amino acid with the a salicylanilide and/or with the parent acid or amine of the salicylanilide and/or with acetyl salicylic acid (e.g., as shown in Table 1) or a derivative of acetyl salicylic to form a modified peptide (peptide-salicylanilide complex) whereby the peptide-salicylanilide complex shows enhanced resistance to proteolysis and/or increased in vivo activity as compared to the untreated peptide. The peptide can be any therapeutic peptide, e.g., as described above. In certain embodiments the peptide is a D or L peptide comprising the amino acid sequence DWFKAFYDKVAEKFKEAF (SEQ ID NO:5) or the amino acid sequence FAEKFKEAVKDYFAKFWD (SEQ ID NO:104). In various embodiments the peptide comprises one or more lysines acetylated. The peptide can be optionally protected at the carboxyl and/or amino terminus, e.g., as described above. In various embodiments the peptide ranges in length from 3 amino acids to 300 amino acids. In certain embodiments the peptide comprises an amphipathic helix. In certain embodiments the peptide is ApoJ, ApoA-I, ApoA-I milano, or 18A.

Methods are also provided for mitigating one or more symptoms of a pathology characterized by an inflammatory response in a mammal (e.g., a human, or a non-human mammal). The methods typically involve orally administering to the mammal a modified amphipathic helical peptide that mitigates one or more symptoms of atherosclerosis or other pathology characterized by an inflammatory response in conjunction with niclosamide or a niclosamide analogue, whereby the oral delivery provides in vivo activity of the peptide to mitigate one or more symptoms of the pathology, and where the modified peptide has the structure of a peptide-salicylanilide complex formed by reacting a therapeutically active peptide with a salicylanilide and/or with the parent acid or amine of the salicylanilide and/or with acetyl salicylic acid or a derivative of acetyl salicylic to form an adduct with the peptide whereby the peptide adduct shows enhanced in vivo activity as compared to the untreated peptide. The modified peptide (peptide-salicylanilide complex) can include any one or more peptides having the structure of a peptide modified as described herein. In certain embodiments the peptide is combined with a pharmaceutically acceptable excipient. In certain embodiments the peptide is administered by a route selected from the group consisting of oral administration, nasal administration, rectal administration, intraperitoneal injection, intravascular injection, subcutaneous injection, transcutaneous administration, inhalation administration, intraocular administration, and intramuscular injection. In certain embodiments peptide is formulated as a unit dosage formulation. In certain embodiments the pathology is selected from the group consisting of atherosclerosis, rheumatoid arthritis, lupus erythematous, polyarteritis nodosa, osteoporosis, Alzheimer's disease, multiple sclerosis, chronic obstructive pulmonary disease, asthma, diabetes, chronic renal disease, and a viral illness.

In certain embodiments niclosamide analogs used in the methods and compositions described herein include, but are not limited to those defined by Formula I, where substituents R¹, R², R⁵, R⁶, R⁷, R⁸, R⁹, R¹⁰, R¹¹ and R¹² are as described herein. In certain embodiments these substituents do not comprise one or more of the following moieties: carboxylic acid, and/or alkyl carboxylates, and/or hydroxamic acid and/or alkyl hydroxamates, and/or sulfonic acid and/or alkyl sulfones, and/or phosphoric acid and/or alkyl phosphates, and/or tetrazole.

### DEFINITIONS

The phrase “enhancing the in vivo activity” or “enhancing the apparent activity” when referring to the agents described herein indicates that the agents, when administered in conjunction with an orally delivered pharmaceutical produce a greater biological response in the organism than the same dosage orally administered without the agent. Without being bound to a particular theory, the in vivo activity can be enhanced by any of a number of mechanisms including, but not limited to increased absorption, decreased degradation, a combination of increased absorption and decreased degradation, enhanced active transport, and the like.

The terms “coadministration” or “administration in conjunction with” when used in reference to the use of a delivery agent (e.g., niclosamide, niclosamide analogue or other delivery agent described herein) in conjunction with an orally administered pharmaceutical (e.g., a therapeutic peptide such as L-4F) indicates that the delivery agent and the orally administered pharmaceutical are administered so that there is at least some chronological overlap in the activity of the delivery agent and administration of the pharmaceutical such that the delivery agent enhances in vivo activity (e.g., via increased uptake and/or bioavailability) of the pharmaceutical. In sequential administration there may even be some substantial delay (e.g., minutes or even hours) between administration of the delivery agent and the pharmaceutical as long as the delivery agent is present in a manner that enhances in vivo activity of the pharmaceutical.

The term mammal includes essentially any mammal including, but not limited to dogs, cats, sheep, cattle, horses, goats, mice, rabbits, hamsters, pigs, monkeys and other non-human primates, and humans. Thus, veterinary as well as medical applications of this invention are contemplated.

The term “oral bioavailability” refers to the bioavailability (e.g., plasma concentration) of an active agent when administered orally (e.g., in an oral formulation).

The term “L form peptide” refers to a peptide comprising all L form amino acids.

The term “D form peptide” refers to a peptide comprising at least one D amino acid. In certain embodiments at least half, and preferably all of the amino acids are D amino acids.

The term “treat” when used with reference to treating, e.g., a pathology or disease refers to the mitigation and/or elimination of one or more symptoms of that pathology or disease, and/or a reduction in the rate of onset or severity of one or more symptoms of that pathology or disease, and/or the prevention of that pathology or disease.

The terms “isolated”, “purified”, or “biologically pure” when referring to an isolated polypeptide refer to material that is substantially or essentially free from components that normally accompany it as found in its native state. With respect to nucleic acids and/or polypeptides the term can refer to nucleic acids or polypeptides that are no longer flanked by the sequences typically flanking them in nature. Chemically synthesized polypeptides are “isolated” because they are not found in a native state (e.g., in blood, serum, etc.). In certain embodiments, the term “isolated” indicates that the polypeptide is not found in nature.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Where the amino acid sequence of a peptide is provided the description of that peptide includes L peptides, D peptides, inverse peptides, retro peptides, and retroinverse peptides. Peptides can also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an α-ester, a β-ester, a thioamide, phosphonamide, carbomate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “residue”” as used herein refers to natural, synthetic, or modified amino acids. Various amino acid analogues include, but are not limited to 2-aminoadipic acid, 3-aminoadipic acid, beta-alanine (beta-aminopropionic acid), 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4diaminobutyric acid, desmosine, 2,2′-diaminopimelic acid, 2,3-diaminopropionic acid, n-ethylglycine, n-ethylasparagine, hydroxylysine, allo-hydroxylysine, 3-hydroxyproline, 4-hydroxyproline, isodesmosine, allo-isoleucine, n-methylglycine, sarcosine, n-methylisoleucine, 6-n-methyllysine, n-methylvaline, norvaline, norleucine, ornithine, and the like. These modified amino acids are illustrative and not intended to be limiting.

The term “an amphipathic helical peptide” refers to a peptide comprising at least one amphipathic helix (amphipathic helical domain). Certain amphipathic helical peptides of this invention can comprise two or more (e.g., 3, 4, 5, etc.) amphipathic helices.

The term “class A amphipathic helix” refers to a protein structure that forms an α-helix producing a segregation of a polar and nonpolar faces with the positively charged residues residing at the polar-nonpolar interface and the negatively charged residues residing at the center of the polar face (see, e.g., Segrest et al. (1990) Proteins: Structure, Function, and Genetics 8: 103-117).

“Apolipoprotein J” (apo J) is known by a variety of names including clusterin, TRPM2, GP80, and SP 40 (see, e.g., Fritz (1995) Pp 112 In: Clusterin: Role in Vertebrate Development, Function, and Adaptation (Harmony JAK Ed.), R. G. Landes, Georgetown, Tex.). It was first described as a heterodimeric glycoprotein and a component of the secreted proteins of cultured rat Sertoli cells (see, e.g., Kissinger et al. (1982) Biol. Reprod.; 27: 233240). The translated product is a single-chain precursor protein that undergoes intracellular cleavage into a disulfide-linked 34 kDa α subunit and a 47 kDa β subunit (see, e.g., Collard and Griswold (1987) Biochem., 26: 3297-3303). It has been associated with cellular injury, lipid transport, apoptosis and it may be involved in clearance of cellular debris caused by cell injury or death. Clusterin has been shown to bind to a variety of molecules with high affinity including lipids, peptides, and proteins and the hydrophobic probe 1-anilino-8-naphthalenesulfonate (Bailey et al. (2001) Biochem., 40: 11828-11840).

The class G amphipathic helix is found in globular proteins, and thus, the name class G. The feature of this class of amphipathic helix is that it possesses a random distribution of positively charged and negatively charged residues on the polar face with a narrow nonpolar face. Because of the narrow nonpolar face this class does not readily associate with phospholipid (see, e.g., Segrest et al. (1990) Proteins: Structure, Function, and Genetics. 8: 103-117; Erratum (1991) Proteins: Structure, Function and Genetics, 9: 79). Several exchangeable apolipoproteins possess similar but not identical characteristics to the G amphipathic helix. Similar to the class G amphipathic helix, this other class possesses a random distribution of positively and negatively charged residues on the polar face. However, in contrast to the class G amphipathic helix which has a narrow nonpolar face, this class has a wide nonpolar face that allows this class to readily bind phospholipid and the class is termed G* to differentiate it from the G class of amphipathic helix (see, e.g., Segrest et al. (1992) J. Lipid Res., 33: 141-166; Anantharamaiah et al. (1993) Pp. 109-142 In: The Amphipathic Helix, Epand, R. M. Ed CRC Press, Boca Raton, Fla.). Computer programs to identify and classify amphipathic helical domains have been described by Jones et al. (1992) J. Lipid Res. 33: 287-296) and include, but are not limited to the helical wheel program (WHEEL or WHEEL/SNORKEL), helical net program (HELNET, HELNET/SNORKEL, HELNET/Angle), program for addition of helical wheels (COMBO or COMBO/SNORKEL), program for addition of helical nets (COMNET, COMNET/SNORKEL, COMBO/SELECT, COMBO/NET), consensus wheel program (CONSENSUS, CONSENSUS/SNORKEL), and the like.

The term “ameliorating” when used with respect to “ameliorating one or more symptoms of atherosclerosis” refers to a reduction, prevention, or elimination of one or more symptoms characteristic of atherosclerosis and/or associated pathologies. Such a reduction includes, but is not limited to a reduction or elimination of oxidized phospholipids, a reduction in atherosclerotic plaque formation and rupture, a reduction in clinical events such as heart attack, angina, or stroke, a decrease in hypertension, a decrease in inflammatory protein biosynthesis, reduction in plasma cholesterol, and the like.

The term “enantiomeric amino acids” refers to amino acids that can exist in at least two forms that are nonsuperimposable mirror images of each other. Most amino acids (except glycine) are enantiomeric and exist in a so-called L-form (L amino acid) or D-form (D amino acid). Most naturally occurring amino acids are “L” amino acids. The terms “D amino acid” and “L amino acid” are used to refer to absolute configuration of the amino acid, rather than a particular direction of rotation of plane-polarized light. The usage herein is consistent with standard usage by those of skill in the art. Amino acids are designated herein using standard 1-letter or three-letter codes, e.g., as designated in Standard ST.25 in the Handbook on Industrial Property Information and Documentation.

The term “protecting group” refers to a chemical group that, when attached to a functional group in an amino acid (e.g., a side chain, an alpha amino group, an alpha carboxyl group, etc.) blocks or masks the properties of that functional group. In certain embodiments amino-terminal protecting groups include, but are not limited to acetyl, or amino groups. Other amino-terminal protecting groups include, but are not limited to alkyl chains as in fatty acids, propeonyl, formyl and others. In certain embodiments, preferred carboxyl terminal protecting groups include, but are not limited to, groups that form amides or esters.

The phrase “protect a phospholipid from oxidation by an oxidizing agent” refers to the ability of a compound to reduce the rate of oxidation of a phospholipid (or the amount of oxidized phospholipid produced) when that phospholipid is contacted with an oxidizing agent (e.g.; hydrogen peroxide, 13-(S)-HPODE, 15-(S)-HPETE, HPODE, HPETE, HODE, HETE, etc.).

The terms “low density lipoprotein” or “LDL” is defined in accordance with common usage of those of skill in the art. Generally, LDL refers to the lipid-protein complex which when isolated by ultracentrifugation is found in the density range d=1.019 to d=1.063.

The terms “high density lipoprotein” or “HDL” is defined in accordance with common usage of those of skill in the art. Generally “HDL” refers to a lipid-protein complex which when isolated by ultracentrifugation is found in the density range of d=1.063 to d=1.21.

The term “Group I HDL” refers to a high density lipoprotein or components thereof (e.g., apo A-I, paraoxonase, platelet activating factor acetylhydrolase, etc.) that reduce oxidized lipids (e.g., in low density lipoproteins) or that protect oxidized lipids from oxidation by oxidizing agents.

The term “Group II HDL” refers to an HDL that offers reduced activity or no activity in protecting lipids from oxidation or in repairing (e.g., reducing) oxidized lipids.

The term “HDL component” refers to a component (e.g., molecules) that comprises a high density lipoprotein (HDL). Assays for HDL that protect lipids from oxidation or that repair (e.g., reduce oxidized lipids) also include assays for components of HDL (e.g., apo A-I, paraoxonase, platelet activating factor acetylhydrolase, etc.) that display such activity.

The terms “human apo A-I peptide” or “human apo A-I protein” can refer to a full-length human apo A-I peptide or to a fragment or domain thereof comprising a class A amphipathic helix.

A “monocytic reaction” as used herein refers to monocyte activity characteristic of the “inflammatory response” associated with atherosclerotic plaque formation. The monocytic reaction is characterized by monocyte adhesion to cells of the vascular wall (e.g., cells of the vascular endothelium), and/or chemotaxis into the subendothelial space, and/or differentiation of monocytes into macrophages.

The following abbreviations may be used herein: PAPC: L-α-1-palmitoyl-2-arachidonoyl-sn-glycero-3-phosphocholine; POVPC: 1-palmitoyl-2-(5-oxovaleryl)-sn-glycero-3-phosphocholine; PGPC: 1-palmitoyl-2-glutaryl-sn-glycero-3-phosphocholine; PEIPC: 1-palmitoyl-2-(5,6-epoxyisoprostane E₂)-sn-glycero-3-phosphocholine; ChC18:2: cholesteryl linoleate; ChC18:2-OOH: cholesteryl linoleate hydroperoxide; DMPC: 1,2-ditetradecanoyl-rac-glycerol-3-phosphocholine; PON: paraoxonase; HPF: Standardized high power field; PAPC: L-α-1-palmitoyl-2-arachidonoyl-sn-glycero-3-phosphocholine; BL/6: C57BL/6J; C3H:C3H/HeJ.

The term “conservative substitution” is used in reference to proteins or peptides to reflect amino acid substitutions that do not substantially alter the activity (specificity (e.g., for lipoproteins)) or binding affinity (e.g., for lipids or lipoproteins)) of the molecule. Typically conservative amino acid substitutions involve substitution one amino acid for another amino acid with similar chemical properties (e.g., charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. With respect to the peptides of this invention sequence identity is determined over the full length of the peptide.

One example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The phrases “adjacent to each other in a helical wheel diagram of a peptide” or “contiguous in a helical wheel diagram of a peptide” when referring to residues in a helical peptide indicates that in the helical wheel representation the residues appear adjacent or contiguous even though they may not be adjacent or contiguous in the linear peptide.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Illustrative cyclic groups include cyclopropyl, cyclobutyl, cyclopentyl, and cyclohexyl groups. The C₁₋₁₀ alkyl group can be substituted or unsubstituted. Illustrative substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₁₀ alkyls include, but are not limited to, methyl, ethyl, n-propyl, isopropyl, cyclopropyl, cyclopropylmethyl, cyclopropylethyl, n-butyl, iso-butyl, sec-butyl, tert-butyl, cyclobutyl, cyclobutylmethyl, cyclobutylethyl, n-pentyl, cyclopentyl, cyclopentylmethyl, cyclopentylethyl, 1-methylbutyl, 2-methylbutyl, 3-methylbutyl, 2,2-dimethylpropyl, 1-ethylpropyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-timethylpropyl, 1-ethyl-1-methylpropyl, 1-ethyl-2-methylpropyl, cyclohexyl, and the like.

A “C₂₋₁₀ alkenyl” refers to a branched or unbranched hydrocarbon group containing one or more double bonds and having from 2 to 10 carbon atoms. A C₂₋₁₀ alkenyl can optionally include monocyclic or polycyclic rings, in which each ring has from three to six members. The C₂₋₁₀ alkenyl group can be substituted or unsubstituted. Illustrative substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₁₀ alkenyls include, but are not limited to, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl, 2,2-dimethyl-3-butenyl, and the like.

A “C₂₋₁₀ alkynyl” refers to a branched or unbranched hydrocarbon group containing one or more triple bonds and having from 2 to 10 carbon atoms. A C₂₋₁₀ alkynyl can optionally include monocyclic, bicyclic, or tricyclic rings, in which each ring has five or six members. The C₂₋₁₀ alkynyl group can be substituted or unsubstituted. Illustrative substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₁₀ alkynyls include, but are not limited to, ethynyl, 1-propynyl, 2-propynyl, 1-butynyl, 2-butynyl, 3-butenyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butenyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl, 3-methyl-4-pentynyl, and the like.

A “C₇₋₆ heterocyclyl” refers to a stable 5- to 7-membered monocyclic or 7- to 14-membered bicyclic heterocyclic ring that is saturated, partially unsaturated or unsaturated (aromatic), and that consists of 2 to 6 carbon atoms and 1, 2, 3 or 4 heteroatoms independently selected from the group consisting of N, 0, and S and including any bicyclic group in which any of the above-defined heterocyclic rings is fused to a benzene ring. The heterocyclyl group can be substituted or unsubstituted. Illustrative substituents include, but are not limited to alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms can optionally be oxidized. The heterocyclic ring can be covalently attached via any heteroatom or carbon atom that results in a stable structure, e.g., an imidazolinyl ring can be linked at either of the ring-carbon atom positions or at the nitrogen atom. A nitrogen atom in the heterocycle can optionally be quaternized. In certain embodiments, when the total number of S and O atoms in the heterocycle exceeds 1, then these heteroatoms are not adjacent to one another. Heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidole, pyridothiazole, pyridinyl, pyridyl, pyrimidinyl, pyrrolidinyl, pyrrolinyl, pyrrolyl, quinolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. In certain embodiments, 5 to 6 membered heterocycles include, but are not limited to, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, tetrazolyl, and the like.

A “C₆₋₁₂ aryl” refers to an aromatic group having a ring system comprised of carbon atoms with conjugated electrons (e.g., phenyl). The aryl group typically has from 6 to 12 carbon atoms. Aryl groups can optionally include monocyclic, bicyclic, or tricyclic rings, in which each ring has five or six members. The aryl group can be substituted or unsubstituted. Illustrative substituents include, but are not limited to, alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, quaternary amino groups, and the like.

A “C₇₋₁₄ alkaryl” refers to an alkyl substituted by an aryl group (e.g., benzyl, phenethyl, or 3,4-dichlorophenethyl) having from 7 to 14 carbon atoms.

A “C₃₋₁₀ alkheterocyclyl” refers to an alkyl substituted heterocyclic group having from 3 to 10 carbon atoms in addition to one or more heteroatoms (e.g., 3-furanylmethyl, 2-furanylmethyl, 3-tetrahydrofuranylmethyl, 2-tetrahydrofuranylmethyl, and the like).

A “C₁₋₁₀ heteroalkyl” refers to a branched or unbranched alkyl, alkenyl, or alkynyl group having from 1 to 10 carbon atoms in addition to one or more heteroatoms, where one or more methylenes (CH₂) or methines (CH) are replaced by nitrogen, oxygen, sulfur, carbonyl, thiocarbonyl, phosphoryl, or sulfonyl. Heteroalkyls include, but are not limited to, tertiary amines, secondary amines, ethers, thioethers, amides, thioamides, carbamates, thiocarbamates, phosphoramidates, sulfonamides, and disulfides. A heteroalkyl can optionally include monocyclic, bicyclic, or tricyclic rings, in which each ring has three to six members. The heteroalkyl group can be substituted or unsubstituted. Illustrative substituents include, but are not limited to alkoxy, aryloxy, sulfhydryl, allylthio, arylthio, halide, hydroxyl, fluoroalkyl, perfluoralkyl, amino, amino alkyl, disubstituted amino, quaternary amino, hydroxyalkyl, hydroxyalkyl, carboxyalkyl, and carboxyl groups.

The term “acyl” refers to a chemical moiety with the formula R—C(O)—, where R is selected from C₁₋₁₁₀ alkyl, C₁₋₁₀ alkenyl, C₁₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, and the like.

A “halide” refers to meant bromine, chlorine, iodine, or fluorine.

### BRIEF DESCRIPTION OF THE DRAWINGS

FIG. 1, panels A-D, shows various forms of niclosamide. A: 2′5-dichloro-4′-nitrosalicylanilide; B: 5-chloro-salicyl-(2-chloro-4-nitro) anilide 2-aminoethanol salt; C: 5-chloro-salicyl-(2-chloro-4-nitro) anilide piperazine salt; and D: 5-chloro-salicyl-(2-chloro-4-nitro)anilide monohydrate.

FIG. 2 illustrates various niclosamide analogues. A: Oxyclozanide (3,3′,5,5′,6-pentachloro-2′-hydroxy salicylanilide; 2,3,5-trichloro-N-(3,5-dichloro-2-hydroxyphenyl)-6-hydroxybenzamide); B: Closantel (5′-Chloro-alpha-4-(p-chlorophenyl)-alpha-4-cyano-3,5-diiodo-2′,4′-salicyloxylidide; N-[5-Choloro-4-[(4-Chlorophenyl) Cyanomethyl]-2-Methylphenyl-2-Hydroxy-3-5-Diiodobenzamide); C: Rafoxanide (also known as Disalan; Flukanide; N-(3-chloro-4-(4-chlorophenoxy)phenyl)-2-hydroxy-3,5-diiodobenzamide; 3′-Chloro-4′-(p-chlorophenoxy)-3,5-diiodosalicylanilide); D: Flusalan (3,5-Dibromo-2-hydroxy-N-(3-trifluoromethyl-phenyl)-benzamide); E: Tribromsalan (3,5-Dibromo-N-(4-bromo-phenyl)-2-hydroxy-benzamide); F: Resorantel (N-(4-Bromo-phenyl)-2,6-dihydroxy-benzamide); G: Clioxanide (Acetic acid 2-(4-chloro-phenylcarbamoyl)-4,6-diiodo-phenyl ester).

FIG. 3 illustrates various niclosamide analogues and salts thereof.

FIG. 4 illustrates niclosamide analogues in which one halogen group is relocated within the same ring (see, e.g., compounds A-D) or both halogen groups are relocated within the same ring (see, e.g., compounds E-G).

FIG. 5 illustrates niclosamides in which the nitro group is relocated within the same ring (see, e.g., compounds A-C) and niclosamide analogues where the hydroxyl group is relocated within the same ring (see, e.g., compounds D-F).

FIG. 6 illustrates niclosamide analogues where both halogen and hydroxy and/or nitro groups are relocated while keeping the substituents within the aromatic ring (see, e.g., compounds A-F) and niclosamide analogues having a nitro- and a hydroxyl group relocation (see, e.g., compounds G-I).

FIG. 7 illustrates niclosamide analogues comprising a single halogen exchange (see, e.g., compounds A-D), niclosamide analogues comprising a double halogen exchange (see, e.g., compounds E-F), niclosamide analogues comprising an exchange of Cl— to Br— (see, e.g., compound G), and niclosamide analogs comprising an exchange of Cl— to F— (see, e.g., compound H).

FIG. 8 shows HDL inflammatory index for apoE null mice fed chow containing or not containing additions. C: Mice were given chow alone; D: Mice given chow supplemented with 8.0 micrograms of niclosamide; E: Mice given chow supplemented with 2.0 micrograms of L-4F; F: Mice given chow supplemented with 8.0 micrograms of Niclosamide together with 2.0 micrograms of L-4F (free base) per gram of chow. The mouse HDL (C-J) was also compared to a standard human HDL (B) that was added at the same concentrations as the mouse HDL. The resulting monocyte chemotactic activity was normalized to the standard control LDL added alone (A). The results are plotted as the HDL-inflammatory index, which is the result of dividing the monocyte chemotactic activity measured for each condition by the monocyte chemotactic activity obtained by the standard control LDL added alone, which was normalized to 1.0. G-I: A second experiment. G: Chow alone; H: chow supplemented with 100 micrograms of Niclosamide per gram of chow; I: Chow supplemented with 10 micrograms of L-4F (free base) per gram of mouse chow; J: Chow supplemented with 10 micrograms of L-4F (free base) together with 100 micrograms of Niclosamide per gram of chow. The data shown are the Mean±S.D.

FIG. 9 shows that administration of niclosamide as an oral bolus by gastric gavage (stomach tube) immediately followed by administration of L-4F as an oral bolus by stomach tube rendered apoE null mouse HDL anti-inflammatory. The HDL-containing fractions were tested for their ability to inhibit the induction of monocyte chemotactic activity by a standard control human LDL, which was added to cultures of human aortic endothelial cells. The values obtained after the addition of the standard control HDL or the mouse HDL were compared to the values obtained by the standard control LDL alone to give the HDL Inflammatory Index. The values shown are the Mean±S.D.

FIG. 10 shows that administration of Niclosamide as an oral bolus by stomach tube immediately followed by administration of L-4F as an oral bolus by stomach tube significantly reduced the ability of apoE null mouse LDL to induce monocyte chemotactic activity in cultures of human aortic endothelial cells. The LDL fractions from the mice described in FIG. 9 were tested for their ability to induce monocyte chemotactic activity in cultures of human aortic endothelial cells and compared to a standard control human LDL whose values were normalized to 1.0 for the LDL-inflammatory index. The data shown are the Mean±S.D.

FIG. 11 shows that oral administration of niclosamide (5.0 mg/kg body weight) immediately followed by oral administration of L-4F (0.5 mg/kg/body weight) renders monkey HDL anti-inflammatory. The data shown are the Mean±S.D. for the HDL

FIG. 12 shows that oral administration of niclosamide (5.0 mg/kg body weight) immediately followed by oral administration of L-4F (0.5 mg/kg/body weight) significantly reduced the ability of monkey LDL to induce monocyte chemotactic activity in cultures of human aortic endothelial cells. The LDL fractions from the monkey plasma described in FIG. 11 were tested as described in FIG. 10. The data shown are the Mean±S.D.

FIG. 13 shows that an amphipathic helical peptide (L-4F) increases the solubility of niclosamide in an aqueous system. Niclosamide at 10 mg per mL was added to water or to water containing 1.0 mg/mL L-4F (free base) and was homogenized in a glass-glass homogenizer. The solutions were stored at 4° C. for ten days and photographed

FIG. 14 shows the HDL inflammatory index for female apoE null mice that were given by gastric gavage (stomach tube) 100 μL water alone or 100 μL water containing niclosamide or containing niclosamide in combination with L-4F at the doses shown on the X-axis. The solutions of niclosamide with or without L-4F shown in FIG. 13 were serially diluted and given by gastric gavage (stomach tube) to fasting seven month old female apoE null mice in a volume of 100 microliters per mouse (n=8 per group). Blood was collected 6 hours following treatment while the mice were still fasting and the plasma was separated by FPLC and the HDL fractions were tested as described in FIG. 8. The data shown are the Mean±S.D, h=human, m=mouse.

FIG. 15 LDL from the mice described in FIG. 14 was tested for its ability to induce human aortic endothelial cells to produce monocyte chemotactic activity. The data are plotted as the LDL-inflammatory index as described for FIG. 10. The values shown are the Mean±S.D.

FIG. 16 shows the HDL from mice that were given niclosamide in mouse chow at 250 μg per day per mouse with or without L-4F (free base). Seven month old female apoE null mice (n=8 per treatment group) were given niclosamide in mouse chow at 250 micrograms per day per mouse with or without L-4F (free base) at 25 micrograms per day per mouse in the drinking water or in mouse chow (food) with the niclosamide. After three days the mice were bled, their plasma was fractionated by FPLC and the ability of the mouse HDL (m) to inhibit LDL-induced monocyte chemotactic activity was determined in cultures of human aortic endothelial cells and calculated as the HDL-inflammatory index as described in FIG. 8. Normal anti-inflammatory human (h) HDL was included in the assays as a positive control. The values shown are the mean±standard deviation (S.D.).

FIG. 17 shows the results of LDL from the mice (m) in FIG. 16 tested for its ability to induce monocyte chemotactic activity in cultures of human aortic endothelial cells. The data is expressed as the LDL-inflammatory index by comparing the results to the monocyte chemotactic activity induced by a standard control human (h) LDL alone, which was normalized to 1.0. The values shown are the Mean±S.D; h=human, m=mouse.

FIG. 18 shows pre-beta HDL formation in mice administered niclosamide with L-4F compared to D-4F.

FIG. 19 shows the HDL-inflammatory index after oral administration of D-4F or L-4F. Niclosamide was homogenized with or without D-4F or L-4F (both as the free-base) in a ratio of 10:1 (niclosamide:peptide; wt:wt) in ABCT buffer pH 7.0 and incubated at 37° C. for 1 hour. The buffer without peptide or with the peptides at 2.5, 5.0, or 10 μg was administered to 3 month old fasting female apoE null mice (n=8 per group) in 100 μL by stomach tube. Six hours later the mice were bled and their plasma separated by FPLC and the HDL fractions from the mice were tested in cultures of human aortic endothelial cells exposed to normal human LDL to determine the HDL-inflammatory index as described in FIG. 8. In the absence of added HDL (0) the monocyte chemotactic activity obtained after addition of the normal control LDL was normalized to 1.0. The monocyte chemotactic activity after addition of the human LDL plus a normal control human HDL (h) or mouse HDL (m) was divided by the monocyte chemotactic activity obtained following addition of the human LDL without HDL to give the HDL-inflammatory index. The data shown are the Mean±S.D; h=human, m=mouse.

FIG. 20 shows the results of a cell-free assay of HDL taken from mice receiving oral D-4F or L-4F. The HDL from the mice described in FIG. 19 was tested in the cell-free assay. The data shown are the Mean±S.D.

FIG. 21 shows plasma paraoxonase activity from the mice described in FIG. 19. The data shown are the Mean±S.D.

FIG. 22 shows that co-administration of niclosamide with L-4F renders apoE null mouse HDL anti-inflammatory to a degree that is similar to normal human HDL. Free base D-4F or L-4F were homogenized with or without niclosamide in a ratio of 10:1 (niclosamide:peptide; wt:wt) in ABCT buffer adjusted to pH 8.0 using 0.1 NaOH. The buffer without the peptide or with the peptides at 10 μg in 100 μL was administered to 4-month-old fasting apoE null female mice (n=8 per group) by stomach tube. Seven hours later the mice were bled and their plasma separated by FPLC and the HDL fractions from the mice were tested in cultures of human aortic endothelial cells exposed to normal human LDL to determine the HDL-inflammatory index as described in FIG. 8. The data shown are the Mean±S.D; h=human, m=mouse.

FIG. 23 the LDL-inflammatory index from the mice described in FIG. 22. The data shown are the Mean±S.D; h=human, m=mouse.

FIG. 24 shows that new salicylanilides (BP-1001 and BP-1012) are more potent than niclosamide in improving the HDL-inflammatory index. Niclosamide (BP-124) or BP-1001, or BP-1012 were homogenized with or without D-4F or L-4F (both as the free base) in a ratio of 10:1 (wt:wt) in ABCT buffer. The buffer without peptide or with peptide at 5 μg in 100 μL was administered to 4-month-old fasting apoE null mice (n=4 per group) by stomach tube. Six hours later the mice were bled and their plasma separated by FPLC and the HDL fractions from the mice were tested in cultures of human aortic endothelial cells exposed to normal human LDL to determine the HDL-inflammatory index as described in FIG. 8. The data shown are the Mean±S.D; h=human, m=mouse.

FIG. 25 shows the LDL-inflammatory index for LDL taken from the mice described in FIG. 24 determined as described in FIG. 10. The data shown are the Mean±S.D; h=human, m=mouse.

FIG. 26 shows a comparison of niclosamide (BP-124) with other salicylanilides. Niclosamide (BP-124) or the salicylanilides whose numbers (BP#) are shown on the X-axis were homogenized with L-4F (as the free base) in a ratio of 10:1 (salicylanilide:L-4F; wt:wt) in ABCT buffer which was adjusted to pH 8.0 with 0.1N NaOH. The buffer without peptide or salicylanilide or with salicylanilide at 100 μg together with L-4F at 10 μg in 100 μL was administered to 5-month-old fasting male apoE null mice (n=4 per group) by stomach tube. Eight hours later the mice were bled and their plasma separated by HPLC and the HDL fractions from the mice were tested in cultures of human aortic endothelial cells exposed to normal human LDL to determine the HDL-inflammatory index as described in FIG. 8. The data shown are the Mean±S.D; h=human, m=mouse.

FIG. 27 shows that niclosamide increases L-4F absorption in apoE null mice. Fasted apoE null mice 6-months of age (n=4 per group) were administered by stomach tube ¹⁴C-L-4F (21,000 dpm containing 10 micrograms of L-4F per mouse) with or without 100 micrograms of niclosamide in 200 microliters. Fasting was continued and the mice were bled at the time points shown on the X-axis and the dpm per mL plasma determined.

FIG. 28 demonstrates that the ¹⁴C-L-4F used in FIG. 27 was biologically active. The HDL inflammatory index was determined as described in FIG. 8 after administration of the compounds shown in FIG. 27.

FIG. 29 shows aortic sinus lesion score in apoE null mice receiving oral doses of niclosamide, L-4F, or niclosamide together with L-4F. Seventeen week old female apoE null mice who were on chow were divided into three groups and the following additions were made to the chow for each group: Group I: Niclosamide at 250 micrograms/mouse/day; Group II: L-4F at 25 micrograms/modse/day; Group III: L-4F at 25 micrograms/mouse/day plus Niclosamide at 250 micrograms/mouse/day. All groups received 50 micrograms/mouse/day of pravastatin in their drinking water. After 14 weeks the mice were sacrificed and aortic sinus lesion area was determined as described previously (Navab et al. (2005) Arterioscler. Thromb. Vasc. Biol., 25: 1426-1432).

FIG. 30 shows the percent aortic surface area determined by en face analysis for the mice described in FIG. 29.

FIG. 31 shows the percent macrophage lesion area for the mice described in FIG. 29.

FIG. 32 shows that oral administration of L-4F together with niclosamide causes lesion regression in old apoE null mice. Ninety-five female apoE null mice age 9.5 months from the UCLA breeding colony were identified. Twenty-three were sacrificed at time Zero (Group I) to establish lesion area at the start of the experiment. The remaining mice were divided into three groups of 24 mice each and the following additions were made to the chow for each group: Group II: Niclosamide at 2,000 micrograms/mouse/day; Group III: L-4F at 200 micrograms/mouse/day; Group IV: L-4F at 200 micrograms/mouse/day plus Niclosamide at 2,000 micrograms/mouse/day. All groups received 50 micrograms/mouse/day of pravastatin in their drinking water. At the veterinarian's request because of fighting and/or ulcerative dermatitis mice were euthanized prior to the end of the experiment as follows: 6 mice from Group II; 5 mice from Group III; 4 mice from Group IV. After six months the remaining mice were sacrificed and aortic sinus lesion area was determined as described previously (Id.).

FIG. 33 shows the percent aortic surface lesion area determined by en face analysis for the mice described in FIG. 32.

FIG. 34 shows the percent macrophage lesion area for the mice described in FIG. 32.

FIG. 35 shows the HDL-inflammatory index determined for apoE-null mice administered L[113-122]apoJ or L-4F with and without niclosamide. Ten month old apoE null mice (n=4 per group) were administered by stomach tube 2 mg of niclosamide or 200 micrograms of L-[113-122]apoJ or 2 mg of niclosamide plus 200 micrograms of L-[113-122]apoJ. Eight hours later the mice were bled, their plasma separated by FPLC and the HDL-inflammatory index determined as described in FIG. 8. The data shown are Mean±S.D.

FIG. 36, panels A-C show an experiment in which 50 mg of L-4F alone, in 40 mL of 0.01 N HCl was incubated at 37° C. or was incubated together with 500 mg niclosamide. As a control 500 mg of niclosamide alone was also incubated under the same conditions. After 48 hours the solutions were centrifuged at 1500×g for 5 minutes and the supernatants were removed and centrifuged at 1800×g for 5 minutes. The supernatants from the 1800×g spin were removed and allowed to sit at room temperature overnight at which time they were centrifuged at 12 000×g for 15 min at room temperature. When niclosamide alone was subjected to this protocol as shown in the HPLC chromatogram (C-18 column, 0-100% acetonitrile gradient run over 100 min) (FIG. 36, panel A) there was no precipitate and the supernatant from the 12 000×g spin did not contain any niclosamide indicating that the free niclosamide had been completely removed by the low speed spins. When L-4F alone was subjected to this protocol, L-4F was found in the 12 000×g supernatant and no pellet was formed (FIG. 36, panel B). However, when L-4F and niclosamide were incubated together in this protocol, the 12 000×g spin yielded a pellet. When this pellet was dissolved in 100 μL of TFA and injected into the HPLC system both L-4F and niclosamide were identified indicating that a complex had formed (FIG. 36, panel C). The 12 000×g supernatant after incubation of L-4F and niclosamide contained only non-complexed L-4F but no niclosamide (data not shown).

FIG. 37 shows the results of administering the various fractions from FIG. 36 to fasting 6 month old female apoE null mice (n=4 per group). Vehicle alone (ABCT), 200 μL, or 200 μL ABCT containing 10 μg of niclosamide alone (Niclos. Alone), or 10 μg of L-4F contained in the 1,800×g supernatant after incubation of L-4F+niclosamide (1800S), or 10 μg of L-4F contained in the 12,000×g pellet after incubation of L-4F+niclosamide (12KP), or 10 μg of L-4F contained in the 12,000×g supernatant after incubation of L-4F+niclosamide (12KS) were administered to the mice by stomach tube. Six hours later the mice were bled and their lipoproteins fractionated by HPLC and the HDL-inflammatory index was determined. The data shown are the Mean±S.D. The data demonstrate that only the L-4F-niclosamide complex was orally bioactive (i.e. the complex contained in the 1800×g supernatant or in the 12,000×g pellet), neither niclosamide alone nor L-4F alone (12KS) significantly improved the HDL-inflammatory index.

FIG. 38 shows an HPLC chromatogram of L-4F after treatment of 225 μg of L-4F alone (i.e., supernatant) or L-4F complexed with niclosamide (i.e. pellet) with 10 μg of trypsin for one hour at 37° C. The number 47.540 is the time of the peak in minutes in this HPLC system. L-4F not subjected to any treatment was detected at 47.254 minutes (data not shown) and niclosamide was detected at 59.358 minutes (data not shown) in this HPLC system. The peak at 47.540 minutes was confirmed to be L-4F by mass spectrometry (data not shown). The data show that the L-4F-niclosamide complex was much more resistant to trypsin digestion than L-4F alone.

FIG. 39 shows that L-4F (molecular weight 2310 daltons) complexed to niclosamide (molecular weight 327 daltons) in an aqueous environment alters the self-association of L-4F. In the absence of niclosamide L-4F self-associates in an aqueous environment to produce micelles that have a molecular weight of >100 kDa. When complexed to niclosamide L-4F forms micelles with a much smaller molecular weight as demonstrated by the non-denaturing gel (4-20% stained with coomassie blue) shown in FIG. 39 where L-4F alone is shown in lane 2 and L-4F+niclosamide is shown in lane 3. Lane 1 contains molecular weight markers (HMW). The data demonstrate that the complex of L-4F and niclosamide alters the self-association of L-4F in an aqueous environment resulting in smaller micelles.

FIG. 40 shows Fourier Transform Infrared Spectroscopy (FTIR)-Attenuated Total Reflectance measured in ethanol or deuterium (heavy water; D₂O) and confirms that niclosamide decreases L-4F self-association. Infrared spectra were recorded at 25° C. using a Brucker Vector™ FTIR spectrometer with a DTGS dector, averaged over 256 scans at a gain of 4 and resolution of 2 cm⁻¹. Peptide samples were prepared by spreading the material onto a 50×20×2 mm 45 degree ATR crystal fitted for the Brucker (Pike Technologies) spectrometer. The dry sample was then hydrated by passing deuterium saturated nitrogen gas through the sample chamber for one hour prior to measurement. For determination of the infrared spectrum of L-4F in ethanol, the sample was air-dried from a solution of the solvent onto the ATR crystal surface. The sample was then carefully covered with ethanol to saturate the peptide with this solvent. The spectrum of L-4F in the L-4F-niclosamide complex was obtained by digital subtraction of peptide-free niclosamide in deuterium (heavy water, D₂O). L-4F was freely soluble in ethanol. The FTIR spectra for L-4F in ethanol had a major amide I band centered at 1655 cm⁻¹, indicating a predominant α-helical conformation with only minor contributions from turn and disordered conformations. When L-4F was hydrated with deuterium vapor to simulate the peptide in water there was a decrease in helical conformations and an enhanced anti-parallel beta sheet population indicated by a signature amide I band at 1630 cm⁻¹ and minor band at 1690 cm⁻¹. Since beta sheets require the formation of intermolecular hydrogen bonds, the occurrence of a sizeable beta sheet population suggests that at the concentrations used in this study there was self-association of the peptide in aqueous environments. When L-4F was co-solvated with niclosamide and hydrated with deuterium, the helical amide band shifted from 1655 cm⁻¹ to 1650 cm⁻¹ indicating that the dominant helical conformation was slightly less ordered. There was also a greater representation of random conformations compare with the peptide in ethanol. The data in FIG. 40 show that co-solvating L-4F with niclosamide conserved the peptide's helical structure and minimized the formation of beta sheet aggregates.

### DETAILED DESCRIPTION

This invention pertains to the surprising discovery that salicylanilides, including, but not limited to niclosamide and/or niclosamide analogues, when orally administered in conjunction with a pharmaceutical (e.g., a peptide pharmaceutical such as a helical peptide (e.g., a class A amphipathic helical peptide, a G* helical peptide, etc.) as described herein) significantly decreases the susceptibility to proteolysis and/or increases the bioavailability and/or apparent in vivo activity of that peptide. Moreover, the increase in bioavailability or apparent activity is sufficient so that peptide pharmaceuticals previously formulated as “D” amino acid isomers and protected at both termini to permit oral administration can readily be formulated utilizing all L form amino acids with optionally protected termini for oral administration. This significantly reduces the cost to manufacture such peptides and increases the predictability of the peptide's behavior in mammalian systems since the biological activity of L peptides is generally better characterized and understood.

Moreover, it was a surprising discovery that when salicylanilides, including, but not limited to niclosamide and/or niclosamide analogues, are combined (e.g., under acidic conditions) with peptide or protein therapeutics (e.g., amphipathic helical peptides, e.g., apolipoprotein A-1 [apoA-1] or portions of apoA-I, or ApoJ, etc.) the salicylanilide and the peptide form a complex that enhances resistance of the peptide/protein to proteolysis and/or increases the apparent solubility of peptide/protein and/or the bioavailability of the peptide/protein. It is believed the salicylanilide can be combined with the peptide at essentially any pH (e.g., about pH 2 to about pH8, pH 9, or pH 10), however, complex formation appears to be enhanced at an acidic pH.

The oral administration of peptides and proteins synthesized from all L-amino acids has proven challenging because of the degradation of these peptides and proteins in the digestive tract. It was a fortuitous and surprising discovery that administration of salicylanilides before, with, or after oral administration of L-4F resulted in significant bioactivity including converting pro-inflammatory HDL to anti-inflammatory and causing lesion regression in mouse models of atherosclerosis.

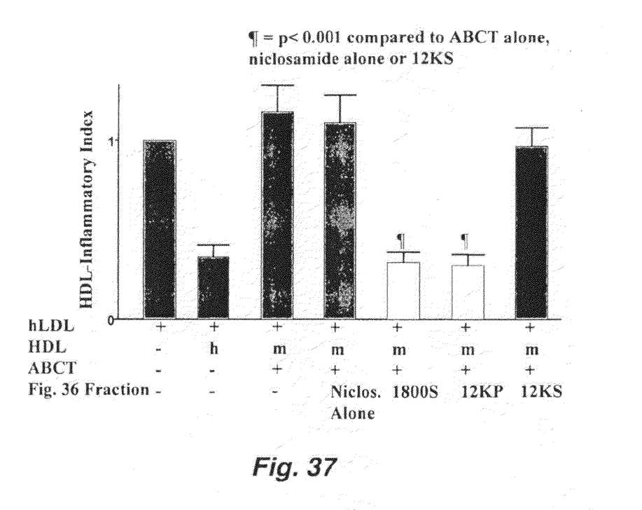

This discovery led to the realization that salicylanilides such as niclosamide could form complexes with L-4F (and other peptides) resulting in a peptide synthesized from all L-amino acids that was bioactive (see, e.g., copending application U.S. Ser. No. 11/835,338, filed on 7 Aug. 2007, and PCT/US2007/017551 for illustrative peptides, which are incorporated herein by reference in their entirety). We discovered that incubating L-4F (or other peptides) with salicylanilides (e.g., niclosamides or niclosamides derivatives, etc.) in vitro prior to oral administration results in a new “modified peptide” that is significantly more potent than the unmodified peptide given orally (see, e.g., FIG. 37).

These unexpected findings have led us to discover a new method for preparing peptides from L-amino acids suitable for oral delivery. In various embodiments the methods entail reacting the peptide with a salicylanilide (e.g., niclosamides, niclosamides analogue, etc.) and/or with the parent acid or amine of the salicylanilide and/or with acetyl salicylic acid and/or a derivative of acetyl salicylic acid at an appropriate pH for an appropriate period of time to produce a modified (orally available) peptide.

In certain embodiments, the peptide(s) can be synthesized with amino acids such as lysine which have been acetylated at the epsilon position of the amino acid with the appropriate reagent (e.g., a salicylanilide (e.g., niclosamides, niclosamides analogue, etc.) and/or with the parent acid or amine of the salicylanilide and/or with acetyl salicylic acid and/or a derivative of acetyl salicylic acid) prior to the synthesis of the peptide.

Thus, in certain embodiments, this invention contemplates methods of enhancing the uptake and in vivo activity of a peptide orally delivered by producing a modified peptide as described herein (e.g., by reacting the polypeptide with a salicylanilide or synthesizing the peptide with modified residues).

In certain other embodiments, this invention contemplates methods of enhancing the uptake and in vivo activity of a peptide orally administered to a mammal by orally administering the peptide in conjunction with an amount of niclosamide or a niclosamide analogue sufficient to enhance in vivo activity (e.g., via enhanced uptake and/or bioavailability) of the peptide. To facilitate such methods, in certain embodiments, pharmaceutical formulations are contemplated that comprise both the peptide pharmaceutical(s) along with niclosamide and/or a niclosamide analogue. In certain embodiments the result of the reaction between the salicylanilide (e.g., niclosamide or niclosamides analogue) with the peptide or protein will be achieved by chemical synthesis prior to administration of the peptide/protein comprising the salicylanilide-derived adduct.

It was also a surprising discovery that the amphipathic helical peptides described herein can increase the solubility of niclosamide and/or niclosamide analogues in aqueous systems thereby enhancing/facilitating the incorporation of niclosamide in a pharmaceutical formulation. Thus, in certain embodiments, this invention contemplates pharmaceutical formulations comprising a combination of a therapeutic amphipathic helical peptide (e.g., D-4F, L-4F, L-5F, etc.) and niclosamide or a niclosamide analogue, wherein said niclosamide in the formulation shows substantially greater solubility in an aqueous solution than niclosamide in an aqueous solution absent the amphipathic helical peptide.

In certain embodiments, this invention also pertains to the surprising discovery that agents such as N-(5-chlorosalicyloyl)-8-aminocaprylic acid (5-CNAC), N-(10-[2-hydroxybenzoyl]aminodecanoic acid (SNAD), and N-(8-[2-hydroxybenzoyl]amino)caprylic acid (SNAC), and the like, can increase the oral bioavailability and/or apparent activity of L form peptides to therapeutically relevant levels. This permits the use of such L form peptides as orally delivered therapeutics where previously D form peptides were preferred. In certain preferred embodiments the L form peptides are the amphipathic helical peptides described herein (e.g., L-4F, L-5F, etc.).

In certain embodiments, the peptides derivatized with salicylanilides as described herein, or when administered in conjunction niclosamide and/or niclosamide analogues as described herein (including, but not necessarily limited to those shown in Formula I and/or Table 1), L-form peptides, e.g., as described herein, do not even require amino or carboxyl terminal blocking/protecting groups. Peptides lacking such blocking groups can easily be synthesized using recombinant expression systems rather than chemical peptide synthesis methods. Bioreactors can thus readily be used to prepare such unprotected peptides at very low cost (as compared to chemically synthesized peptides).

In various embodiments formulations comprising one or more therapeutic peptides in combination with niclosamide and/or niclosamide analogues as described herein, are contemplated. The formulations are typically suitable for oral administration. In certain embodiments the formulations can provide for release of niclosamide and/or niclosamide analogues and/or permeability enhancer(s) before the peptide.

While niclosamide and niclosamide analogues and/or other “permeability” enhancers described herein are particularly useful for enhancing the oral bioavailability of L peptides as described herein, the uses of these agents is not so limited. Thus, in certain embodiments the use of such agents with protected L peptides and or protected or unprotected peptides comprising one or more D amino acid residues is also contemplated.

### I. Chemically Modifying Peptides for Oral Administration.

In various embodiments, this invention pertains to the discovery that modification of peptides by reaction with salicylanilides or de novo synthesis of such peptides using similarly derivatized residues can produce modified peptides that show improved bioactivity when orally administered.

Accordingly, in various embodiments, this invention provides modified therapeutic peptides that show improved in vivo bioactivity and/or bioavailability. In certain embodiments, the peptides are modified by reacting the peptide with a salicylanilide such as niclosamides or niclosamides analog (e.g., as illustrated in Table 1), or with the parent acid or amine of the salicylanilide (e.g., as illustrated in Table 1) or with acetyl salicylic acid or a derivative of acetyl salicylic acid at an appropriate pH for an appropriate period of time.

In various typical embodiments, the peptide can be reacted at an acidic pH. In certain embodiments the pH ranges from about pH 1 to about pH 7. In certain embodiments the pH ranges from about pH 1, 1.5, 2, 2.5, 3, or 3.5 to about 4, 4.5, 5, 5.5, 6, 6.5, 6.8, or 6.9. The reaction proceeds readily at room temperature. In various embodiments, however, the reaction can be conducted at a temperature ranging from about 20° C., 25° C., 30° C., 35° C., or 37° C. to about 50° C., 55° C., 60° C., 65° C., or 70° C. In various embodiments the reaction will be under sterile conditions. In certain embodiments the reaction can simply be run overnight. Typically, the reaction will be run for a period ranging from about 0.5, 1, 2, 3, 4, 5, 6, 7, 8, 9, 10, 11, or 12 hours to about 13, 14, 15, 16, 17, 18, 19, 20, 21, 22, 23, or 24 or more hours depending on temperature and pH.

In various embodiments this invention provides for modified peptides having the structure of a peptide modified as described above (e.g., a modified peptide character of the HPLC shown in FIG. 37), regardless as to the method of preparation. Thus, in certain embodiments, the peptide can be synthesized with amino acids such as lysine that have been acetylated, e.g., at the epsilon position of the amino acid with the appropriate reagent prior to the synthesis of the peptide.

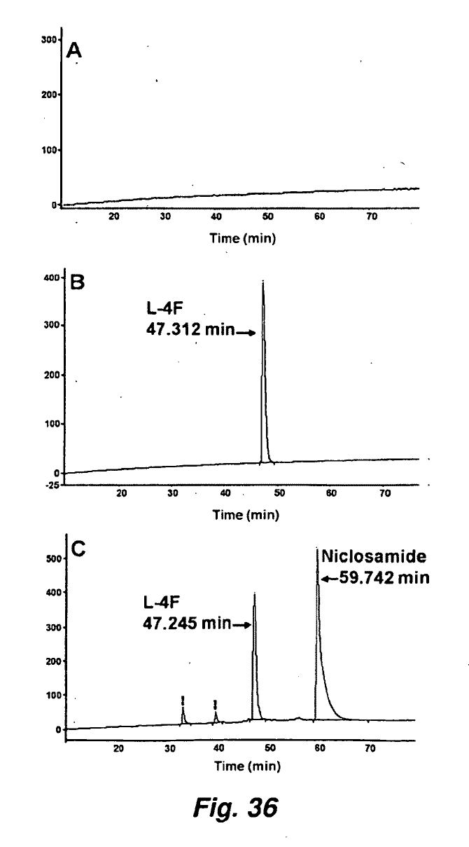

As illustrated in FIGS. 36-38 the reaction described above clearly produces a modified peptide, and the resulting peptide is significantly more bioactive after oral administration compared to oral administration of the native peptide.

### II. Salicylanilides to Enhance Pharmaceutical In Vivo Activity.

As indicated above, it is a surprising discovery that various salicylanilides including, but not limited to niclosamide and niclosamide analogues are effective to substantially increase the in vivo activity (e.g., bioavailability, bioactivity, etc.) of a pharmaceutical (e.g., a therapeutic peptide) orally administered to a mammal when they are reacted with the peptide or administered in conjunction with the peptide. Moreover, it was particularly surprising that the salicylanilides can be reacted with the peptide to form a peptide-salicylanilde complex that shows greater resistance to proteolysis than the peptide alone, but that retains, or even increases, the peptide activity in vivo.

A) Niclosamide and Niclosamide Analogues

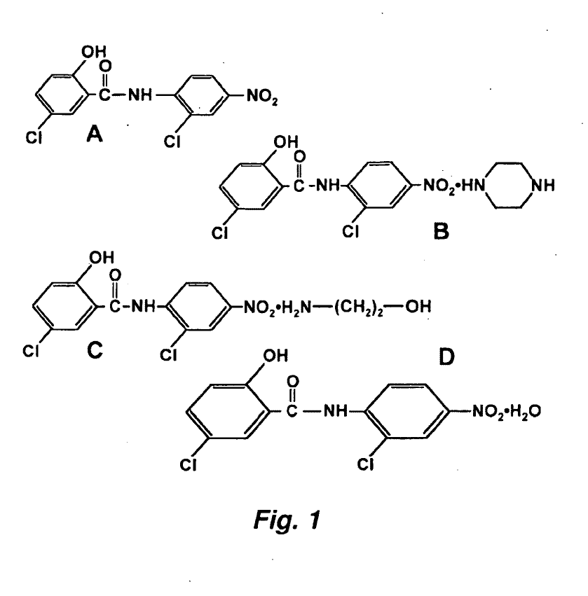

Niclosamide is a chloronitrophenol derivative (see compound A in FIG. 1) principally used against aquatic snails but also as an antiparasitic drug in human and veterinary medicine. Niclosamide is known by the IUPAC designation: 2′5-dichloro-4′-nitrosalicylanilide and by the CAS designation: CAS: 5-chloro-N-(2-chloro-4-nitrophenyl)-2-hydroxybenzamide.

Niclosamide is not very water soluble, 5-8 mg/L at 20° C., sparingly soluble in ether, ethanol and chloroform, and soluble in acetone; the ethanolamine salt dissolves in distilled water 180-280 mg/L at 20° C. It was a surprising discovery, however, that the inclusion of an amphipathic helical peptide, e.g., as described herein, significantly increases the solubility of niclosamide and facilitates the preparation of pharmaceutical formulations.

In tablets niclosamide undergoes a biodegradation in moist environments but niclosamide itself is stable in an aqueous solution for several months. The ethanolamine salt is stable to heat, hydrolyzed by concentrated acid or alkali, and stable in aquatic environments.

Niclosamide is readily available in a number of formulations. These include, but are not limited to, the ethanolamine salt (see compound C in FIG. 1) known by the IUPAC designation 5-chloro-salicyl-(2-chloro-4-nitro) anilide 2-aminoethanol salt or the CAS designation 5-chloro-N-(2-chloro-4-nitrophenyl)-2-hydroxybenzamide with 2-aminoethanol (1:1), the piperazine salt (see compound B in FIG. 1) known by the IUPAC designation 5-chloro-salicyl-(2-chloro-4-nitro) anilide piperazine salt or the CAS designation 5-chloro-N-(2-chloro-4-nitrophenyl)-2-hydroxybenzamide with piperazine (2:1), and niclosamide monohydrate (see compound D in FIG. 1) known by the IUPAC designation 5-chloro-salicyl-(2-chloro-4-nitro) anilide monohydrate or the CAS designation 5-chloro-N-(2-chloro-4-nitrophenyl)-2-hydroxybenzamide with monohydrate (1:1).

Niclosamide is commercially available in a number of formulations including, but not limited to BAYER 73®, BAYER 2353®, BAYER 25 6480, BAYLUSCID®, BAYLUSCIDE®, CESTOCID®, CLONITRALID, DICHLOSALE®, FENASAL®, HL 2447®, IOMESAN@, IOMEZAN®, LINTEX®, MANOSIL®, NASEMO®, NICLOSAMID®, PHENASAL®, TREDEMINE®, SULQUI®, VERMITID®, VERMITIN®, YOMESAN®, and the like.

In certain embodiments, this invention also contemplates the use of various niclosamide analogues to enhance the in vivo of orally administered pharmaceuticals (e.g., therapeutic peptides). Such analogues include, but are not limited to, compounds according to Formula I:

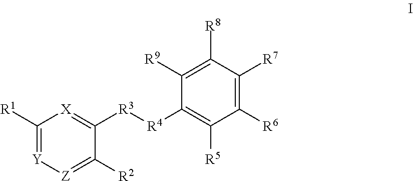

where X is N or CR¹⁰; Y is N or CR¹¹; Z is N or CR¹²; and each of R¹, R², R⁵, R⁶, R⁷, R⁸, R⁹, R¹⁰, R¹¹ and R¹² is independently selected from H, halide (F, Cl, Br, or I), NO₂, OH, OR¹³, SR¹⁴, NR¹⁵R¹⁶, CN, CF₃, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, or is described by one of the Formulas II-XIV:

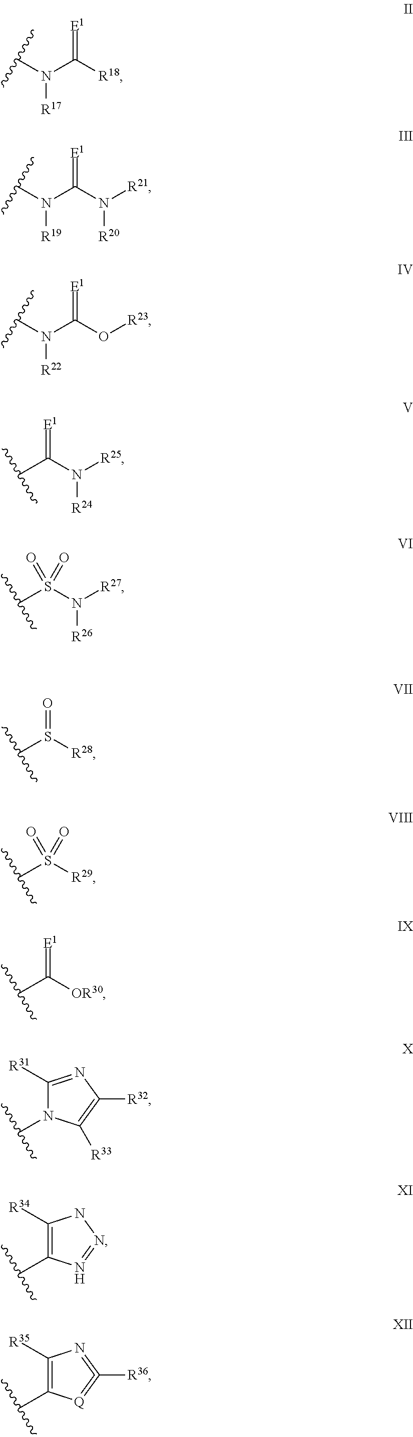

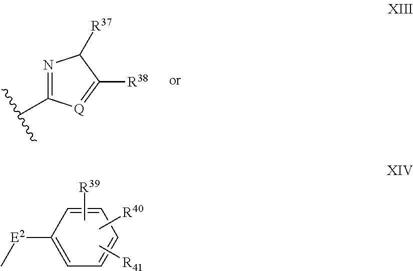

In compounds of formula I, R³ and R⁴ are independently selected from the group consisting of C═O, C═S, C═NR⁴², NH, NR⁴³, CHOR⁴⁴, CH₂, and the like. Groups R² and R⁴; X and R⁴; R⁵ and R³; R⁹ and R³ may combine to form a six-membered ring, using connections described by one of the groups:

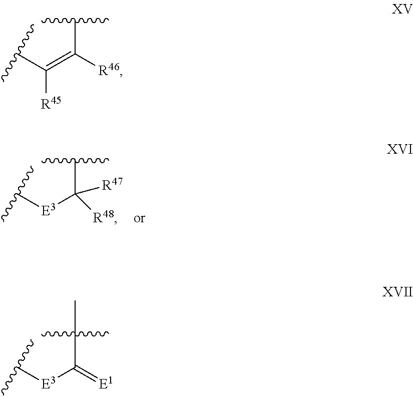

For compounds of formula I, each E¹ is independently O, S, or NR⁴²; each E² is independently CR⁴⁹R⁵⁰, O or S; each E³ is independently CR⁵¹R⁵², O, S, or NR⁵³; each Q is, independently, O, S, or NR⁵⁴. R¹³ and R¹⁴ are each independently, acyl, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl; R¹⁸, R²³, R²⁸, R²⁹, R³⁰, R⁴², R⁵⁴ are each, independently, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C_{1-10 heteroalkyl; R}¹⁵, R¹⁶, R¹⁷, R¹⁹, R²⁰, R²¹, R²², R²⁴, R²⁵, R²⁶, R²⁷, R⁴³, R⁴⁴, R⁴⁵, R⁴⁶, R⁴⁷, R⁴⁸, R⁵¹, R⁵², and R⁵³ are each, independently, H, C₁₋₁₀ alkyl, C₂₋₁₀ alkenyl, C₂₋₁₀ alkynyl, C_{2-6 heterocyclyl, C}₆₋₁₂ aryl, C₇₋₁₄ alkaryl, C₃₋₁₀ alkheterocyclyl, C₁₋₁₀ heteroalkyl; R³¹, R³², R³³, R³⁴, R³⁵, R³⁶, R³⁷, R³⁸, R³⁹, R⁴⁰, R⁴¹, R⁴⁹, and R⁵⁰ are each, independently, H, halide, NO₂, CN, CF₃, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In certain embodiments, compounds of formula I are further described by any of formulas XVIII-XXI:

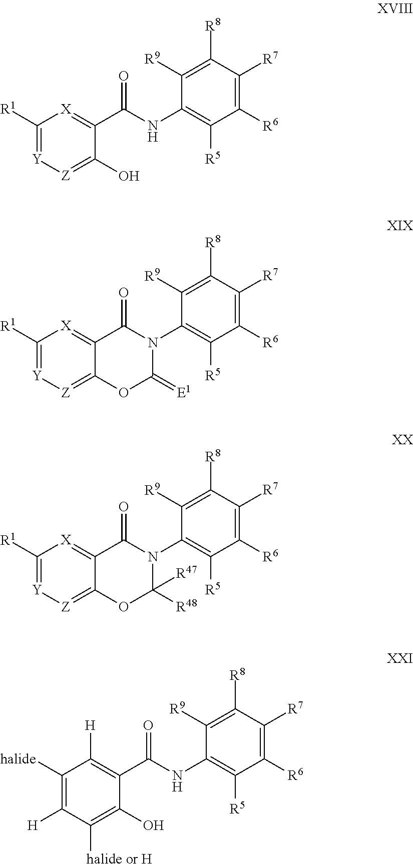

where X, Y, Z, E¹, R¹, R⁵, R⁶, R⁷, R⁸, R⁹, R⁴⁷, and R⁴⁸ are as defined above.

In certain embodiments compounds include compounds described by Formula XXII:

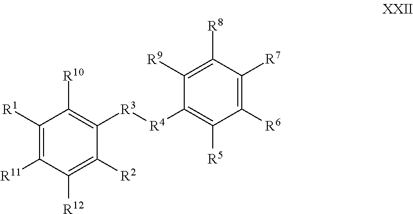

where R¹, R², R⁵, R⁶, R⁷, R⁸, R⁹, R¹⁰, R¹¹ and R¹² are independently selected from the group consisting of H, halide, NO₂, CF₃, OH, acyl, CN, C₁-C₁₀ alkyl (preferably C₁-C₃ alkyl), C₁-C₁₀ heteroalkyl (preferably C₁-C₃ heteroalkyl); and wherein R³ and R⁴ are as defined above. In certain embodiments, R³ is C═O, while R⁴ is NH or R³ is NH while R⁴ is C═O. In these and certain other embodiments, only two of R¹, R², R¹⁰, R¹¹, and R¹² are present, and one is H or OH, while the other is halogen (e.g., Cl, Br, or F).

In these and certain other embodiments, only two of R⁵, R⁶, R⁷, R⁸, and R⁹ are present and these are NO₂ and halogen (e.g., Cl, Br, or F).

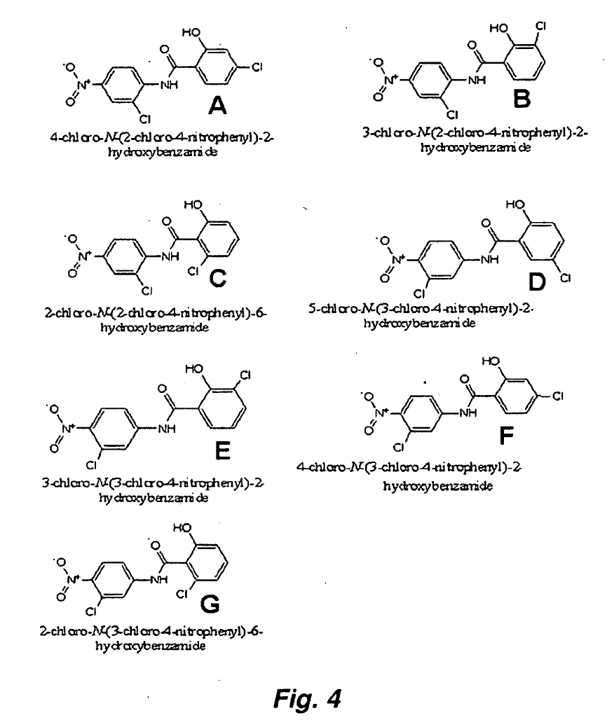

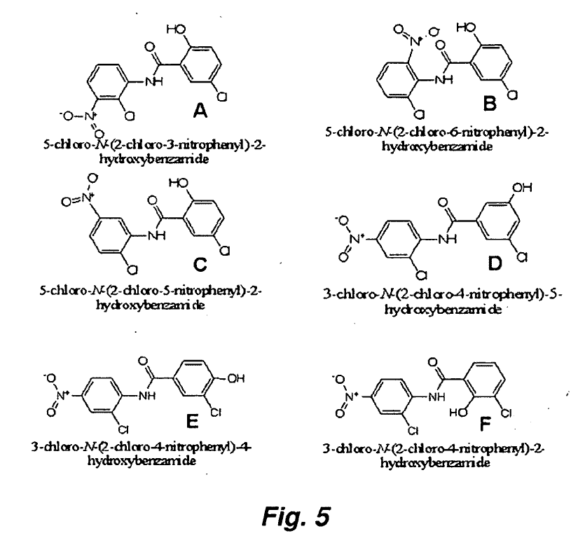

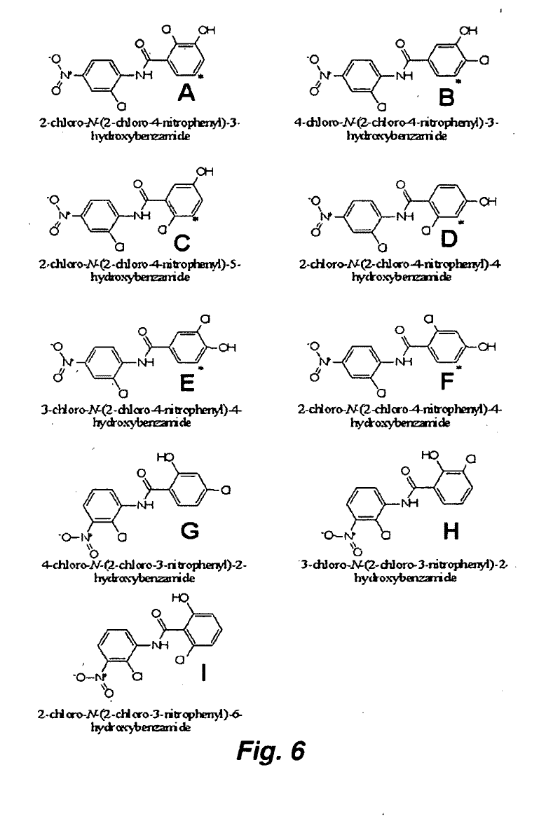

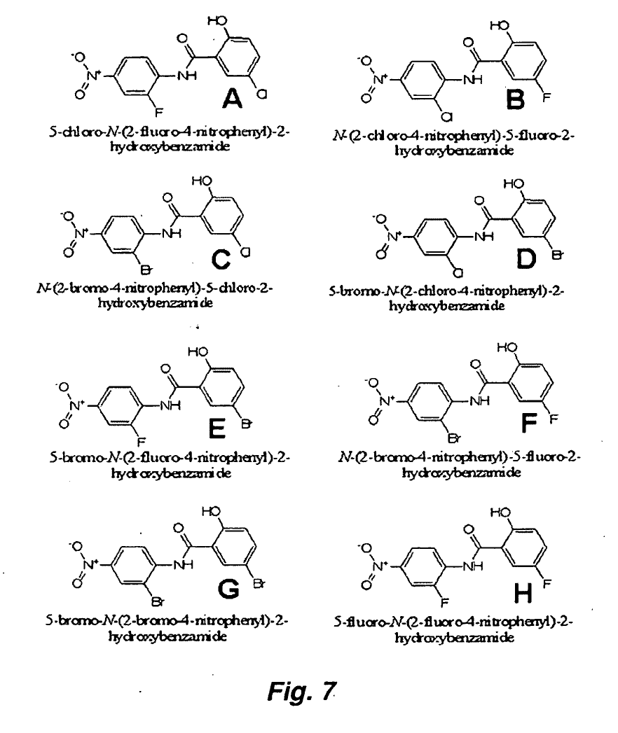

In certain embodiments niclosamide analogues include, but are not limited to niclosamide analogues in which one halogen group is relocated within the same ring (see, e.g., compounds A-D in FIG. 4) or both halogen groups are relocated within the same ring (see, e.g., compounds E-G in FIG. 4), niclosamides in which the nitro group is relocated within the same ring (see, e.g., compounds A-C in FIG. 5), niclosamide analogues where the hydroxyl group is relocated within the same ring (see, e.g., compounds D-F in FIG. 5), niclosamide analogues where both halogen and hydroxy and/or nitro groups are relocated while keeping the substituents within the aromatic ring (see, e.g., compounds A-F in FIG. 6), compounds like A-F in FIG. 6, except having except (3-chloro-4-nitrophenyl) in place of (2-chloro-4-nitrophenyl), niclosamide analogues having a nitro- and a hydroxyl group relocation (see, e.g., compounds G-I in FIG. 6), niclosamide analogues comprising a single halogen exchange (see, e.g., compounds A-D in FIG. 7), niclosamide analogues comprising a double halogen exchange (see, e.g., compounds E-F in FIG. 7), niclosamide analogs comprising an exchange of Cl— to Br— (see, e.g., compound G in FIG. 7), niclosamide analogs comprising an exchange of Cl— to F— (see, e.g., compound H in FIG. 7), and the like.

In certain embodiments the niclosamide analogues include, but are not limited to compounds according to Formula XXIII:

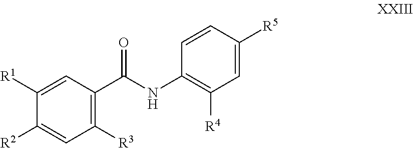

where R¹, R², R³, R⁴, and R⁵, are independently present or absent, and when present are independently selected from the group consisting of Cl, Br, alkyl, methyl, hydroxyalkyl, and the like. These analogues are meant to be illustrative and not limiting. Using the teaching provided herein, other suitable niclosamide analogs will be recognized by one of skill in the art.

In certain embodiments the salicylanilides include, but are not limited to salicylanilides shown in Table 1.

TABLE 1
Illustrative salicylanilides.

| Cmpd | Salicylanilide | Parent Acid | Parent Amine |
|---|---|---|---|
| BP 1001 | 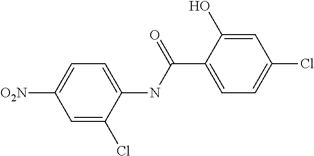 | 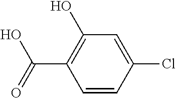 | 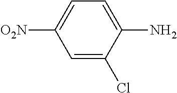 |
| BP 1002 | 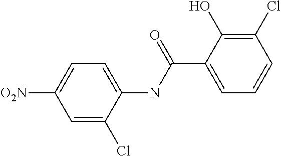 | 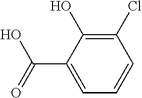 | 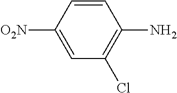 |
| BP 1003 | 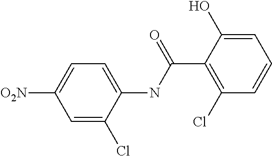 | 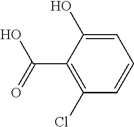 | 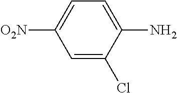 |
| BP 1004 | 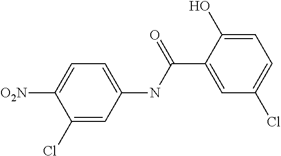 | 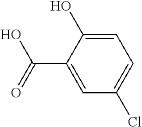 | 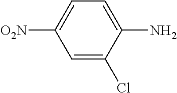 |
| BP 1005 | 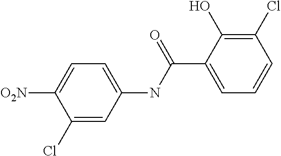 | 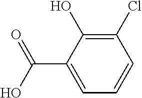 | 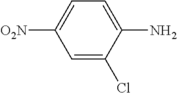 |
| BP 1006 | 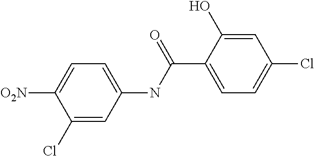 | 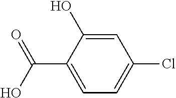 | 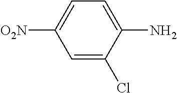 |
| BP 1007 | 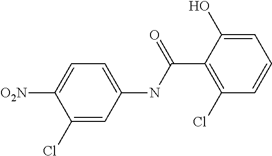 | 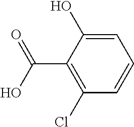 | 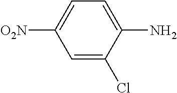 |
| BP 1008 | 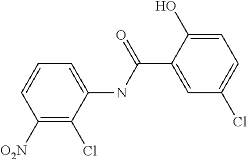 | 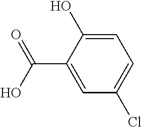 | 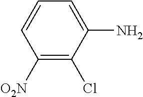 |
| BP 1009 | 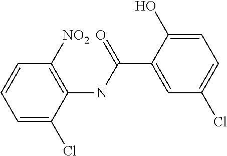 | 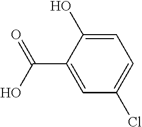 | 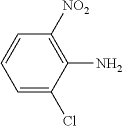 |
| BP 1010 | 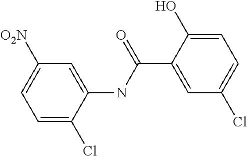 | 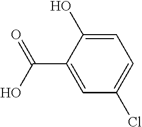 | 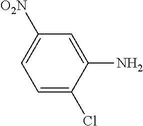 |
| BP 1011 | 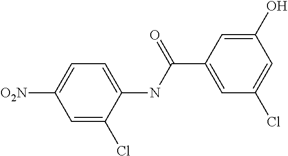 | 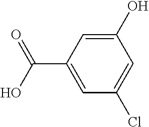 | 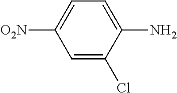 |
| BP 1012 | 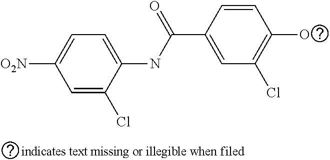 | 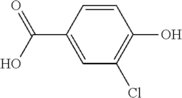 | 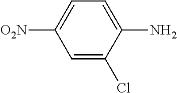 |
| BP 1013 | 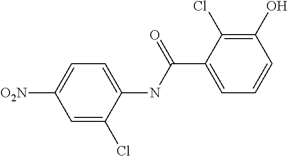 | 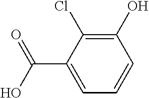 | 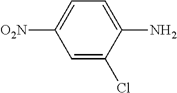 |
| BP 1014 | 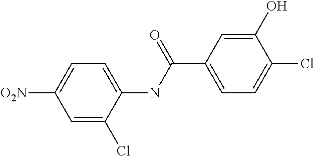 | 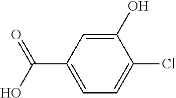 | 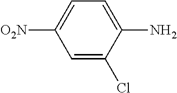 |
| BP 1015 | 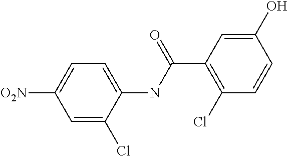 | 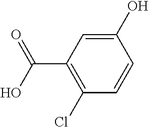 | 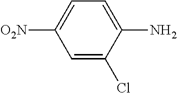 |
| BP 1016 | 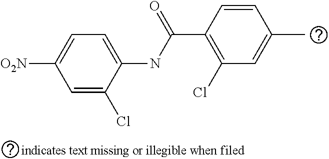 | 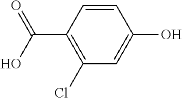 | 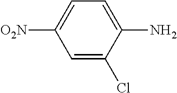 |
| BP 1017 | 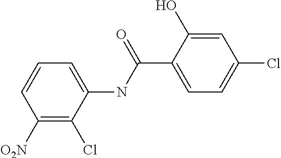 | 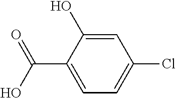 | 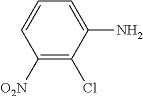 |
| BP 1018 | 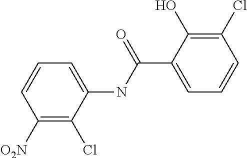 | 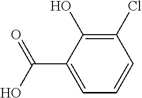 | 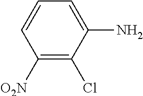 |
| BP 1019 | 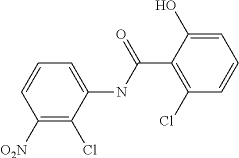 | 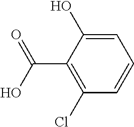 | 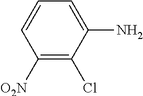 |
| BP 1020 | 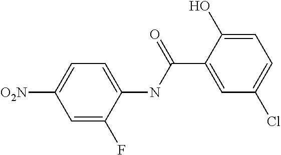 | 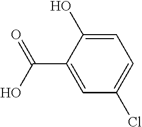 | 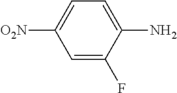 |
| BP 1021 | 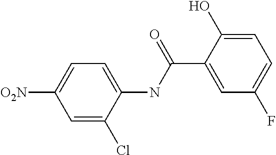 | 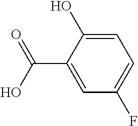 | 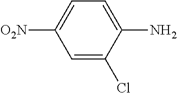 |
| BP 1022 | 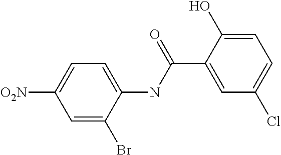 | 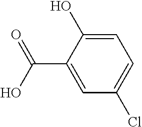 | 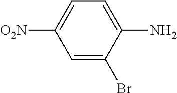 |
| BP 1023 | 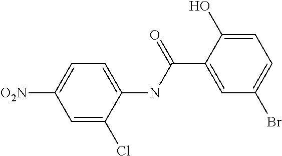 | 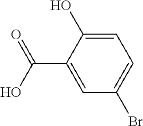 | 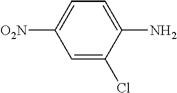 |
| BP 1024 | 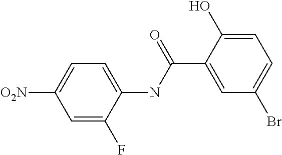 | 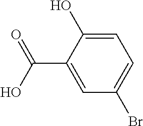 | 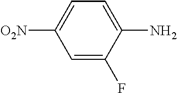 |
| BP 1025 | 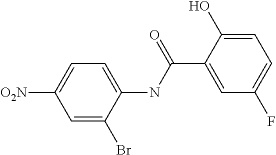 | 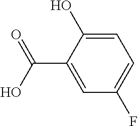 | 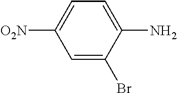 |
| BP 1026 | 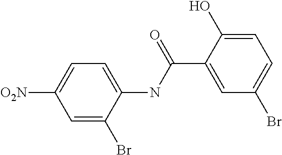 | 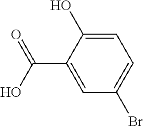 | 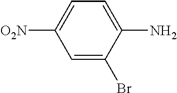 |
| BP 1027 | 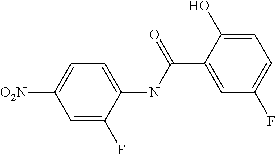 | 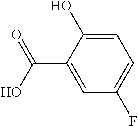 | 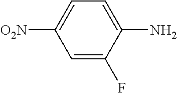 |
| BP 1028 | 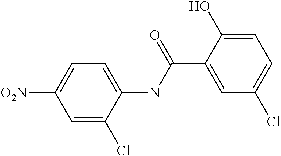 | 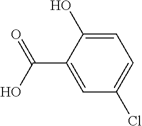 | 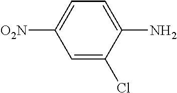 |
| BP 1029 | 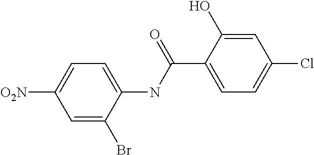 | 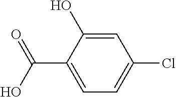 | 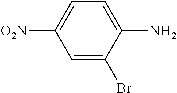 |
| BP 1030 | 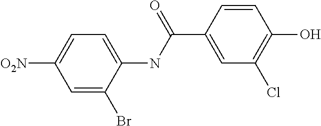 | 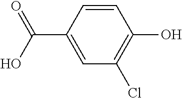 | 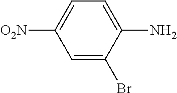 |
| BP 1031 | 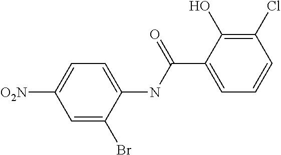 | 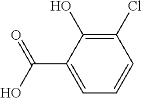 | 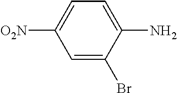 |
| BP 1032 | 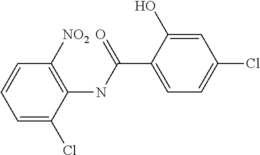 | 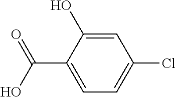 | 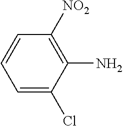 |
| BP 1033 | 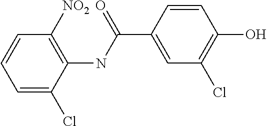 | 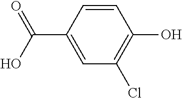 | 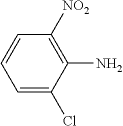 |
| BP 1034 | 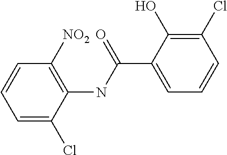 | 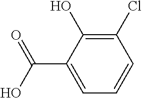 | 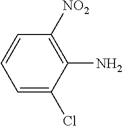 |
| BP 1035 | 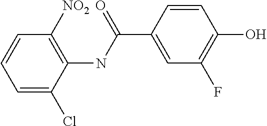 | 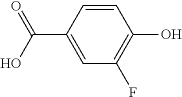 | 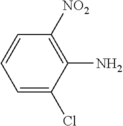 |
| BP 1036 | 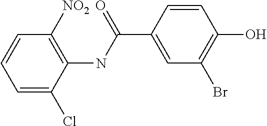 | 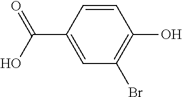 | 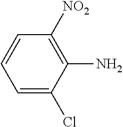 |
| BP 1037 | 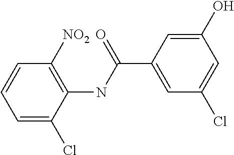 | 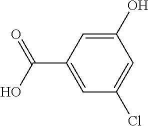 | 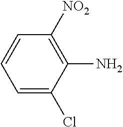 |
| BP 1038 | 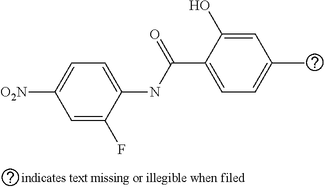 | 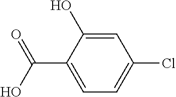 | 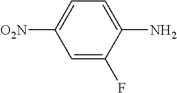 |
| BP 1039 | 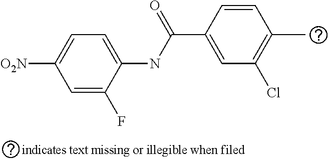 | 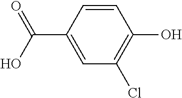 | 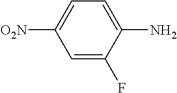 |
| BP 1040 | 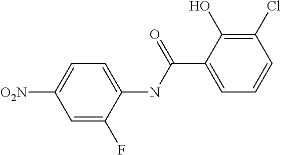 | 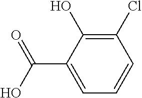 | 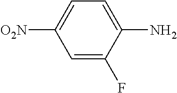 |
| BP 1041 | 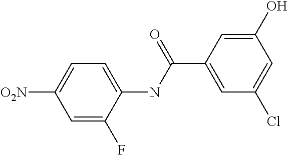 | 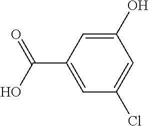 | 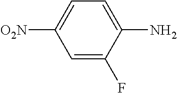 |
| BP 1042 | 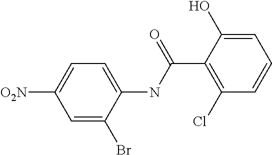 | 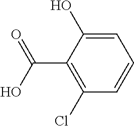 | 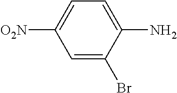 |
| BP 1043 | 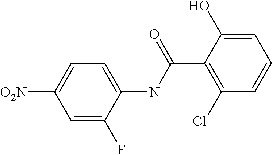 | 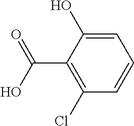 | 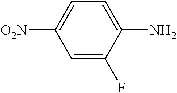 |
| BP 1044 | 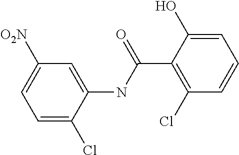 | 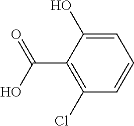 | 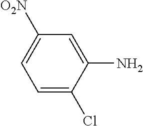 |
| BP 1045 | 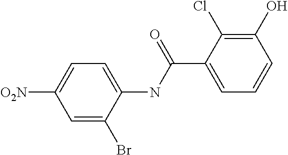 | 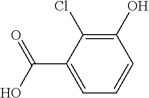 | 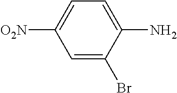 |
| BP 1046 | 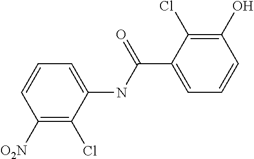 | 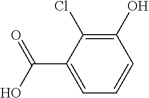 | 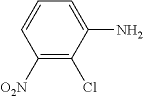 |
| BP 1047 | 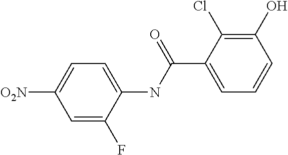 | 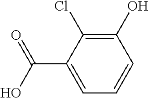 | 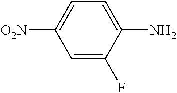 |
| BP 1048 | 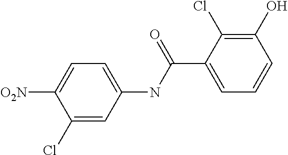 | 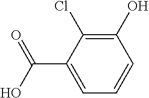 | 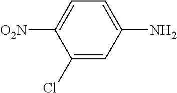 |
| BP 1049 | 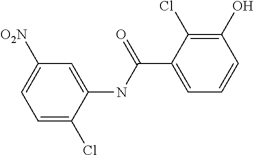 | 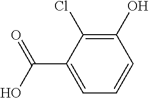 | 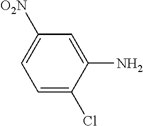 |
| BP 1050 | 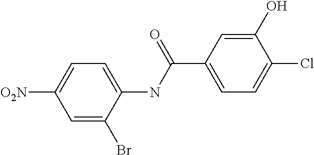 | 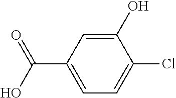 | 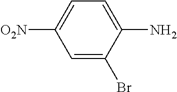 |
| BP 1051 | 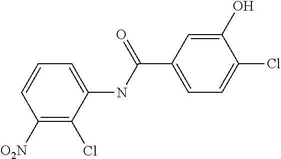 | 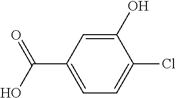 | 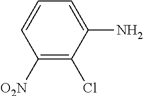 |
| BP 1052 | 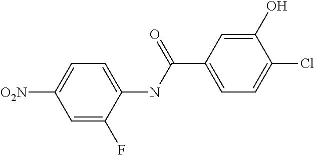 | 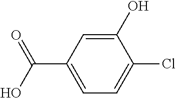 | 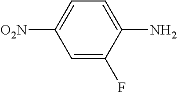 |
| BP 1053 | 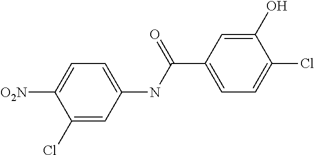 | 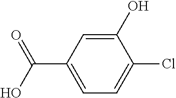 | 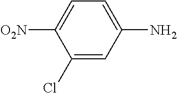 |
| BP 1055 | 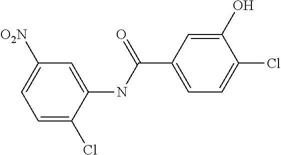 | 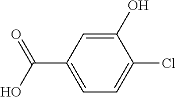 | 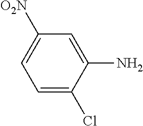 |
| BP 1056 | 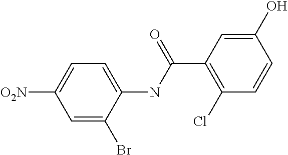 | 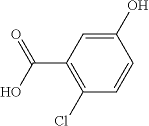 | 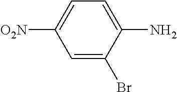 |
| BP 1057 | 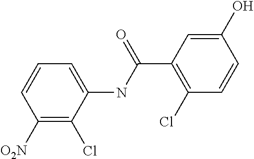 | 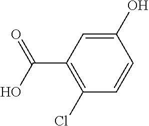 | 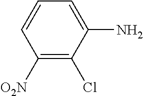 |
| BP 1058 | 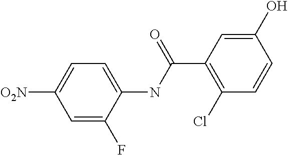 | 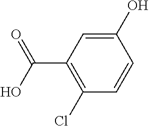 | 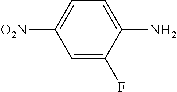 |
| BP 1059 | 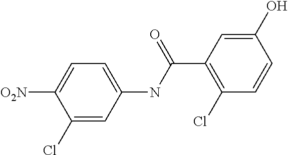 | 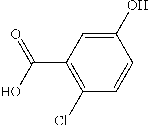 | 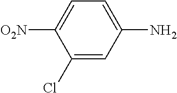 |
| BP 1061 | 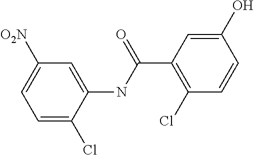 | 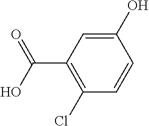 | 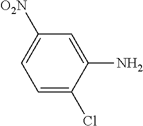 |
| BP 1063 | 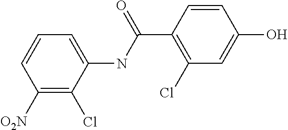 | 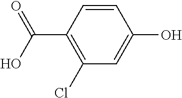 | 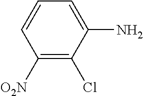 |
| BP 1064 | 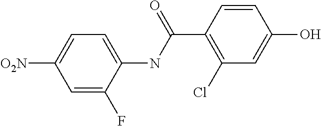 | 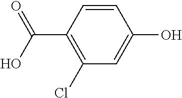 | 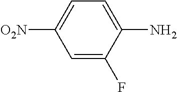 |
| BP 1065 | 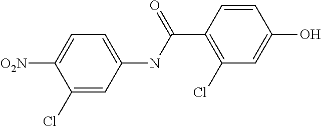 | 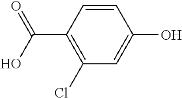 | 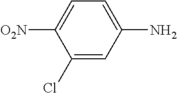 |
| BP 1067 | 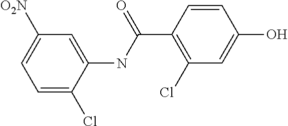 | 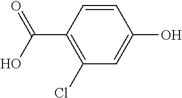 | 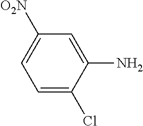 |
| BP 1068 | 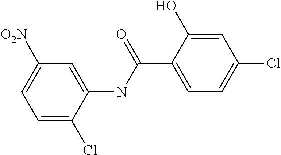 | 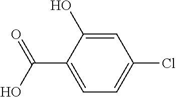 | 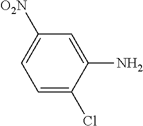 |
| BP 1069 | 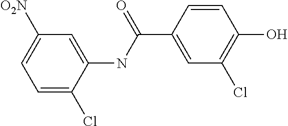 | 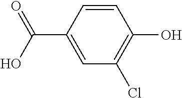 | 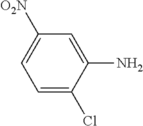 |
| BP 1070 | 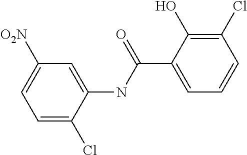 | 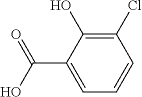 | 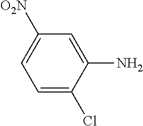 |
| BP 1071 | 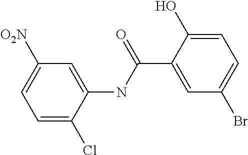 | 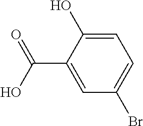 | 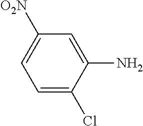 |
| BP 1072 | 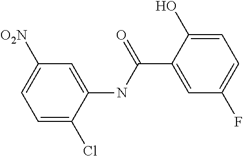 | 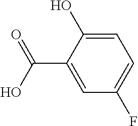 | 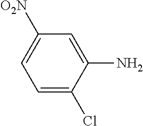 |
| BP 1073 | 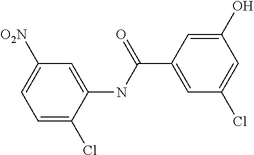 | 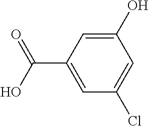 | 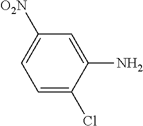 |

B) Other Salicylanilides

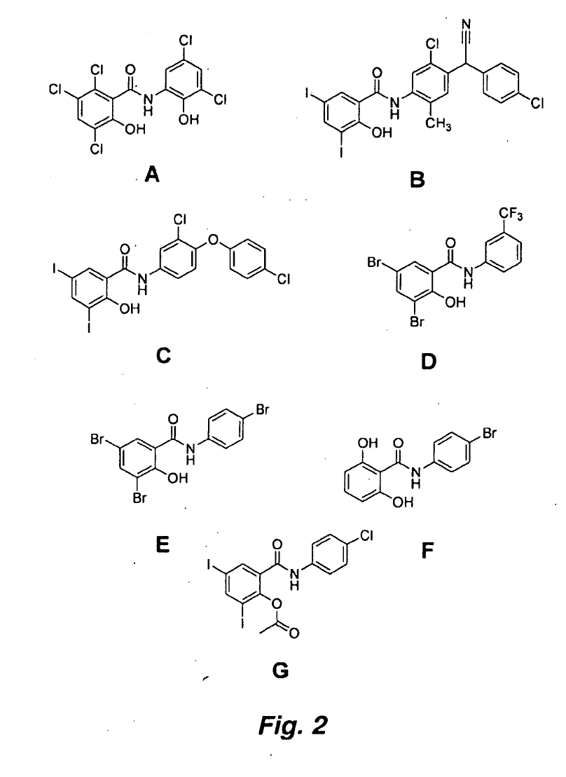

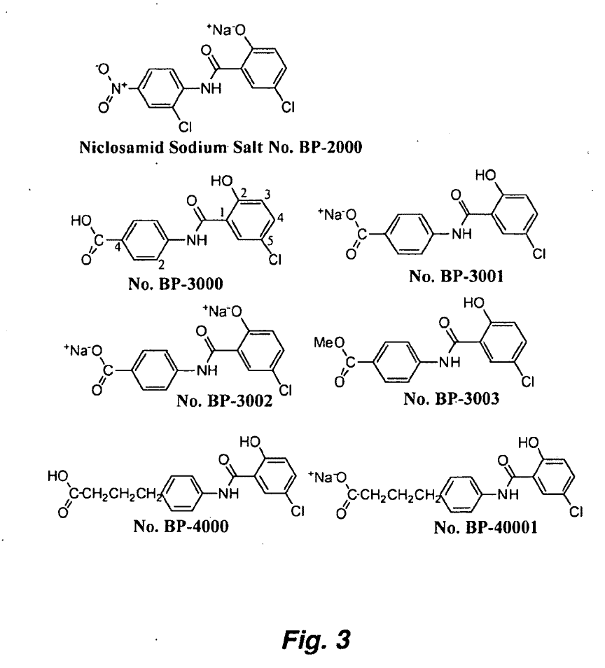

Without being bound by a particular theory, it is believed that a number of other salicylanilides can act in a manner similar to niclosamide to enhance in vivo activity of orally administered pharmaceuticals (e.g., therapeutic peptides). Illustrative salicylanilides include, but are not limited to Closantel (CAS #: 57808-65-8, see, e.g., FIG. 2, compound A), Oxyclozanide (CAS #: 2277-92-1, see, e.g., FIG. 2, compound B), Rafoxanide (CAS #: 22662-39-1, see, e.g., FIG. 2, compound C), Flusalan (CAS #: 4776-06-1, see, e.g., FIG. 2, compound D), Tribromsalan (CAS #: 87-10-5, see, e.g., FIG. 2, compound E), Resorantel (CAS #: 20788-07-2, see, e.g., FIG. 2, compound F), Clioxanide (CAS #: 14437-41-3, see, e.g., FIG. 2, compound G) Other suitable salicylanilides include Brotianide (CAS #: 23233-88-7), 4′-chloro-3-nitrosalicylanilide, 4′-chloro-5-nitrosalicylanilide, 2′-chloro-5′-methoxy-3-nitrosalicylanilide, 2′-methoxy-3,4′-dinitrosalicylanilide, 2′,4′-dimethyl-3-nitrosalicylanilide, 4′,5-dibromo-3-nitrosalicylanilide, 2′-chloro-3,4′-dinitrosalicylanilide, 2′-ethyl-3-nitrosalicylanilide, 2′-bromo-3-nitrosalicylanilide, and the like. In certain embodiments the salicylanilides include one or more of the compounds shown in FIG. 3.

It is noted that these salicylanilides are intended to be illustrative and not limiting. Methods of making salicylanilides are well known to those of skill in the art (see, e.g., PCT/US2003/022026 (WO 2004/006906) which is herein incorporated by reference for all purposes).

C) Identifying Effective Salicylanilides.

Using the teaching provided herein, other suitable salicylanilides can readily be identified using only routine experimentation. Various salicylanilides can be purchased from commercial vendors (e.g., Sigma Chemical, Aldrich, etc.) and then screened for their ability to enhance the apparent in vivo activity of an orally administered pharmaceutical (e.g., a peptide such as L-4F). Such screening methods can include for example, administering the salicylanilide in question in conjunction with L-4F (SEQ ID NO:5) to an apoE null mouse with appropriate controls and evaluating HDL-containing blood fractions for their ability to inhibit monocyte chemotactic activity induced by a standard control human LDL in cultures of human aortic endothelial cells. Salicylanilides that, when administered with L-4F produce more protective HDL than L-4F alone are compounds that enhance the in vivo activity (apparent activity) of that peptide. Such assays are illustrated herein in Example 1.

### III. Other Delivery Agents.

Without being bound to a particular theory, in view of the niclosamide data presented herein, it is also believed that number of other delivery agents are also capable of enhancing the in vivo activity (apparent activity) of therapeutic orally administered pharmaceuticals, including, but not limited to amphipathic helical peptides (e.g., ApoA-I, ApoA-I milano, 4F, D18A, etc.) such that the L form of the peptide achieves therapeutically relevant levels of bioavailability when administered with the delivery agent(s).

Such delivery agents include, but are not limited to agents such N-(5-chlorosalicyloyl)-8-aminocaprylic acid (5-CNAC), N-(10-[2-hydroxybenzoyl]aminodecanoic acid (SNAD), and N-(8-[2-hydroxybenzoyl]amino)caprylic acid (SNAC) and various salts (e.g., disodium salts) thereof. In certain embodiments such delivery agents include any one or more of the modified amino acids disclosed in aforementioned U.S. Pat. No. 5,866,536 or any one of the modified amino acids described in U.S. Pat. No. 5,773,647, which are incorporated herein by reference. Also included are various salts of such agents including, but not limited to the disodium salts described in WO 00/059863 which is incorporated herein by reference.

In certain embodiments the delivery agents comprise a compound selected from the group consisting of 4-{4-{N-(4-bromobenzoyl)aminophenyl]}butyric acid, 4-{4-N-(2-iodobenzoyl)aminophenyl]}butyric acid, 3-(4-(2,5-dimethoxybenzoyl)aminophenyl)propionic acid, 4-{n-[4-(3-iodobenzoyl)aminophenyl]}butyric acid, 4-(o-anisoyl)aminophenylacetic acid, 3-[4-(2,4-dimethoxybenzoyl)aminophenyl]propionic acid, 4-{4-[N-(4-iodobenzoyl)]aminophenyl}butyric acid, 3-4-(2,3-dimethoxybenzoyl)aminophenyl]propionic acid, 4-{N-2[N-2-bromobenzoyl)]aminophenyl}butyric acid, 4-{N-2[N-3-bromobenzoyl]aminophenyl}butyric acid, 4-{-[N-(4-bromobenzoyl)aminophenyl]}butyric acid, 4-{N-[4-(2-methoxy-4-nitrobenzoyl)aminophenyl]}butyric acid, 4-(4-(2,3-dimethoxybenzoyl)aminophenyl)butyric acid, 4-[4-N-(4-methoxy-3-nitrobenzoyl)aminophenyl]butyric acid, and the like.

### IV. Therapeutic Peptides.

In various embodiments, this invention pertains to the use of salicylanilides (e.g., niclosamide) as well as other delivery agents to facilitate/permit the oral delivery of therapeutic peptides even when the peptides are L-form peptides and/or are unprotected. A therapeutic peptide is a peptide that is used to mitigate one or more symptoms of a disease or pathology.

A wide variety of therapeutic peptides are known to those of skill in the art and can be used in the formulations and methods of this invention. Such peptides include, for example, growth hormone (e.g., isolated and/or human, porcine, or bovine growth hormones), natural, synthetic, or recombinant growth hormone releasing hormones (GHRH), interferons (e.g., alpha, beta, and gamma interferon), interleukins (e.g., interleukin-1, interleukin, 2, etc.), natural, synthetic or recombinant insulin (e.g., porcine, bovine, human insulins), insulin-like growth factor-1 (IGF-1), insulin-like growth factor-2 (IGF2, somatostatin), heparin, heparinoids, dermatans, chondroitins, calcitonin (e.g., natural, synthetic, or recombinant salmon, procine, eel, chicken, and human calcitonin), antigens (e.g., influenza antigen', hepatitis A, B, C antigen, HPV antigen, etc), antibodies (polyclonal and monoclonal) (e.g., HERCEPTIN®, RITUXAN®, AVASTIN®, ERBITUX®, etc.), oxytocin, leutinizing-hormone-releasing hormone (LHRH), follicle stimulating hormone (FSH); glucocerebrosidase, thrombopoietin; filgrastim; prostaglandins; vasopressin; cromolyn sodium (e.g., sodium or disodium chromoglycate), vancomycin, desferrioxamine (DFO); parathyroid hormone (PTH) including its fragments, antimicrobials (e.g., anti-bacterial agents, including anti-fungal agents, etc.), and the like. In addition, the therapeutic peptides include analogs, fragments, mimetics or modified derivatives of these compounds (e.g., polyethylene glycol (PEG)-modified derivatives, glycosylated derivatives, etc.), or any combination thereof.

In certain preferred embodiments, the therapeutic peptides are peptides that ameliorate one or more symptoms of a pathology associated with an inflammatory response (e.g., atherosclerosis). Such peptides include, but are not limited to ApoA-I (natural, synthetic, recombinant), ApoA-I milano, (natural, synthetic, recombinant), apolipoprotein M, 18A, and related peptides (see, e.g., U.S. Pat. No. 4,643,988, U.S. Pat. No. 6,037,323, and PCT Publication WO 97/36927 all of which are incorporated herein by reference).

In certain particularly preferred embodiments, the therapeutic peptides used in the methods and formulations described herein include one or more of the peptides described below.

A) Class A Amphipathic Helical Peptides.

In certain embodiments, the peptides for use in the method of this invention include class A amphipathic helical peptides, e.g., as described in U.S. Pat. No. 6,664,230, and PCT Publications WO 02/15923 and WO 2004/034977. It was discovered that peptides comprising a class A amphipathic helix (“class A peptides”), in addition to being capable of mitigating one or more symptoms of atherosclerosis are also useful in the treatment of one or more of the other indications described herein.

Class A peptides are characterized by formation of an α-helix that produces a segregation of polar and non-polar residues thereby forming a polar and a nonpolar face with the positively charged residues residing at the polar-nonpolar interface and the negatively charged residues residing at the center of the polar face (see, e.g., Anantharamaiah (1986) Meth. Enzymol., 128: 626-668). It is noted that the fourth exon of apo A-I, when folded into 3.667 residues/turn produces a class A amphipathic helical structure.

One class A peptide, designated 18A (see, e.g., Anantharamaiah (1986) Meth. Enzymol., 128: 626-668) was modified as described herein to produce peptides orally administrable and highly effective at inhibiting or preventing one or more symptoms of atherosclerosis and/or other indications described herein. Without being bound by a particular theory, it is believed that the peptides of this invention may act in vivo by picking up/sequestering seeding molecule(s) that mitigate oxidation of LDL.

We determined that increasing the number of Phe residues on the hydrophobic face of 18A would theoretically increase lipid affinity as determined by the computation described by Palgunachari et al. (1996) Arteriosclerosis, Thrombosis, & Vascular Biol. 16: 328-338. Theoretically, a systematic substitution of residues in the nonpolar face of 18A with Phe could yield six peptides. Peptides with an additional 2, 3 and 4 Phe would have theoretical lipid affinity (X) values of 13, 14 and 15 units, respectively. However, the λ values jumped four units if the additional Phe were increased from 4 to 5 (to 19λ units). Increasing to 6 or 7 Phe would produce a less dramatic increase (to 20 and 21λ units, respectively).

A number of these class A peptides were made including, the peptide designated 4F (L-4F), D-4F, 5F (L-5F), and D-5F, and the like. Various class A peptides inhibited lesion development in atherosclerosis-susceptible mice. In addition, the peptides show varying, but significant degrees of efficacy in mitigating one or more symptoms of the various pathologies described herein. A number of such peptides are illustrated in Table 2.

TABLE 2
Illustrative class A amphipathic helical peptides
for use in this invention.

| Peptide | | |
|---|---|---|
| Name | Amino Acid Sequence | SEQ ID NO. |
| 18A | D-W-L-K-A-F-Y-D-K-V-A-E-K-L-K-E-A-F | 1 |
| 2F | Ac-D-W-L-K-A-F-Y-D-K-V-A-E-K-L-K-E-A-F-NH₂ | 2 |
| 3F | Ac-D-W-F-K-A-F-Y-D-K-V-A-E-K-L-K-E-A-F-NH₂ | 3 |
| 3F14 | Ac-D-W-L-K-A-F-Y-D-K-V-A-E-K-F-K-E-A-F-NH₂ | 4 |
| 4F | Ac-D-W-F-K-A-F-Y-D-K-V-A-E-K-F-K-E-A-F-NH₂ | 5 |
| 5F | Ac-D-W-L-K-A-F-Y-D-K-V-F-E-K-F-K-E-F-F-NH₂ | 6 |
| 6F | Ac-D-W-L-K-A-F-Y-D-K-F-F-E-K-F-K-E-F-F-NH₂ | 7 |
| 7F | Ac-D-W-F-K-A-F-Y-D-K-F-F-E-K-F-K-E-F-F-NH₂ | 8 |
| | Ac-D-W-L-K-A-F-Y-D-K-V-A-E-K-L-K-E-F-F-NH₂ | 9 |
| | Ac-D-W-L-K-A-F-Y-D-K-V-F-E-K-F-K-E-A-F-NH₂ | 10 |
| | Ac-D-W-L-K-A-F-Y-D-K-V-F-E-K-L-K-E-F-F-NH₂ | 11 |
| | Ac-D-W-L-K-A-F-Y-D-K-V-A-E-K-F-K-E-F-F-NH₂ | 12 |
| | Ac-D-W-L-K-A-F-Y-D-K-V-F-E-K-F-K-E-F-F-NH₂ | 13 |
| | Ac-E-W-L-K-L-F-Y-E-K-V-L-E-K-F-K-E-A-F-NH₂ | 14 |
| | Ac-E-W-L-K-A-F-Y-D-K-V-A-E-K-F-K-E-A-F-NH₂ | 15 |
| | Ac-E-W-L-K-A-F-Y-D-K-V-A-E-K-L-K-E-F-F-NH₂ | 16 |
| | Ac-E-W-L-K-A-F-Y-D-K-V-F-E-K-F-K-E-A-F-NH₂ | 17 |
| | Ac-E-W-L-K-A-F-Y-D-K-V-F-E-K-L-K-E-F-F-NH₂ | 18 |
| | Ac-E-W-L-K-A-F-Y-D-K-V-A-E-K-F-K-E-F-F-NH₂ | 19 |
| | Ac-E-W-L-K-A-F-Y-D-K-V-F-E-K-F-K-E-F-F-NH₂ | 20 |
| | AC-A-F-Y-D-K-V-A-E-K-L-K-E-A-F-NH₂ | 21 |
| | Ac-A-F-Y-D-K-V-A-E-K-F-K-E-A-F-NH₂ | 22 |
| | Ac-A-F-Y-D-K-V-A-E-K-F-K-E-A-F-NH₂ | 23 |
| | Ac-A-F-Y-D-K-F-F-E-K-F-K-E-F-F-NH₂ | 24 |
| | Ac-A-F-Y-D-K-F-F-E-K-F-K-E-F-F-NH₂ | 25 |
| | Ac-A-F-Y-D-K-V-A-E-K-F-K-E-A-F-NH₂ | 26 |
| | Ac-A-F-Y-D-K-V-A-E-K-L-K-E-F-F-NH₂ | 27 |
| | Ac-A-F-Y-D-K-V-F-E-K-F-K-E-A-F-NH₂ | 28 |
| | Ac-A-F-Y-D-K-V-F-E-K-L-K-E-F-F-NH₂ | 29 |
| | Ac-A-F-Y-D-K-V-A-E-K-F-K-E-F-F-NH₂ | 30 |
| | Ac-K-A-F-Y-D-K-V-F-E-K-F-K-E-F-NH₂ | 31 |
| | Ac-L-F-Y-E-K-V-L-E-K-F-K-E-A-F-NH₂ | 32 |
| | Ac-A-F-Y-D-K-V-A-E-K-F-K-E-A-F-NH₂ | 33 |
| | Ac-A-F-Y-D-K-V-A-E-K-L-K-E-F-F-NH₂ | 34 |
| | Ac-A-F-Y-D-K-V-F-E-K-F-K-E-A-F-NH₂ | 35 |
| | Ac-A-F-Y-D-K-V-F-E-K-L-K-E-F-F-NH₂ | 36 |
| | Ac-A-F-Y-D-K-V-A-E-K-F-K-E-F-F-NH₂ | 37 |
| | Ac-A-F-Y-D-K-V-F-E-K-F-K-E-F-F-NH₂ | 38 |
| | Ac-D-W-L-K-A-L-Y-D-K-V-A-E-K-L-K-E-A-L-NH₂ | 39 |
| | Ac-D-W-F-K-A-F-Y-E-K-V-A-E-K-L-K-E-F-F-NH₂ | 40 |
| | Ac-D-W-F-K-A-F-Y-E-K-F-F-E-K-F-K-E-F-F-NH₂ | 41 |
| | Ac-E-W-L-K-A-L-Y-E-K-V-A-E-K-L-K-E-A-L-NH₂ | 42 |
| | Ac-E-W-L-K-A-F-Y-E-K-V-A-E-K-L-K-E-A-F-NH₂ | 43 |
| | Ac-E-W-F-K-A-F-Y-E-K-V-A-E-K-L-K-E-F-F-NH₂ | 44 |
| | Ac-E-W-L-K-A-F-Y-E-K-V-F-E-K-F-K-E-F-F-NH₂ | 45 |
| | Ac-E-W-L-K-A-F-Y-E-K-F-F-E-K-F-K-E-F-F-NH₂ | 46 |
| | Ac-E-W-F-K-A-F-Y-E-K-F-F-E-K-F-K-E-F-F-NH₂ | 47 |
| | Ac-D-F-L-K-A-W-Y-D-K-V-A-E-K-L-K-E-A-W-NH₂ | 48 |
| | Ac-E-F-L-K-A-W-Y-E-K-V-A-E-K-L-K-E-A-W-NH₂ | 49 |
| | Ac-D-F-W-K-A-W-Y-D-K-V-A-E-K-L-K-E-W-W-NH₂ | 50 |
| | Ac-E-F-W-K-A-W-Y-E-K-V-A-E-K-L-K-E-W-W-NH₂ | 51 |
| | Ac-D-K-L-K-A-F-Y-D-K-V-F-E-W-A-K-E-A-F-NH₂ | 52 |
| | Ac-D-K-W-K-A-V-Y-D-K-F-A-E-A-F-K-E-F-L-NH₂ | 53 |
| | Ac-E-K-L-K-A-F-Y-E-K-V-F-E-W-A-K-E-A-F-NH₂ | 54 |
| | Ac-E-K-W-K-A-V-Y-E-K-F-A-E-A-F-K-E-F-L-NH₂ | 55 |
| | Ac-D-W-L-K-A-F-V-D-K-F-A-E-K-F-K-E-A-Y-NH₂ | 56 |
| | Ac-E-K-W-K-A-V-Y-E-K-F-A-E-A-F-K-E-F-L-NH₂ | 57 |
| | Ac-D-W-L-K-A-F-V-Y-D-K-V-F-K-L-K-E-F-F-NH₂ | 58 |
| | Ac-E-W-L-K-A-F-V-Y-E-K-V-F-K-L-K-E-F-F-NH₂ | 59 |
| | Ac-D-W-L-R-A-F-Y-D-K-V-A-E-K-L-K-E-A-F-NH₂ | 60 |
| | Ac-E-W-L-R-A-F-Y-E-K-V-A-E-K-L-K-E-A-F-NH₂ | 61 |
| | Ac-D-W-L-K-A-F-Y-D-R-V-A-E-K-L-K-E-A-F-NH₂ | 62 |
| | Ac-E-W-L-K-A-F-Y-E-R-V-A-E-K-L-K-E-A-F-NH₂ | 63 |
| | Ac-D-W-L-K-A-F-Y-D-K-V-A-E-R-L-K-E-A-F-NH₂ | 64 |
| | Ac-E-W-L-K-A-F-Y-E-K-V-A-E-R-L-K-E-A-F-NH₂ | 65 |
| | Ac-D-W-L-K-A-F-Y-D-K-V-A-E-K-L-R-E-A-F-NH₂ | 66 |
| | Ac-E-W-L-K-A-F-Y-E-K-V-A-E-K-L-R-E-A-F-NH₂ | 67 |
| | Ac-D-W-L-K-A-F-Y-D-R-V-A-E-R-L-K-E-A-F-NH₂ | 68 |
| | Ac-E-W-L-K-A-F-Y-E-R-V-A-E-R-L-K-E-A-F-NH₂ | 69 |
| | Ac-D-W-L-R-A-F-Y-D-K-V-A-E-K-L-R-E-A-F-NH₂ | 70 |
| | Ac-E-W-L-R-A-F-Y-E-K-V-A-E-K-L-R-E-A-F-NH₂ | 71 |
| | Ac-D-W-L-R-A-F-Y-D-R-V-A-E-K-L-K-E-A-F-NH₂ | 72 |
| | Ac-E-W-L-R-A-F-Y-E-R-V-A-E-K-L-K-E-A-F-NH₂ | 73 |
| | Ac-D-W-L-K-A-F-Y-D-K-V-A-E-R-L-R-E-A-F-NH₂ | 74 |
| | Ac-E-W-L-K-A-F-Y-E-K-V-A-E-R-L-R-E-A-F-NH₂ | 75 |
| | Ac-D-W-L-R-A-F-Y-D-K-V-A-E-R-L-K-E-A-F-NH₂ | 76 |
| | Ac-E-W-L-R-A-F-Y-E-K-V-A-E-R-L-K-E-A-F-NH2 | 77 |
| | D-W-L-K-A-F-Y-D-K-V-A-E-K-L-K-E-A-F-P-D-W- | 78 |
| | L-K-A-F-Y-D-K-V-A-E-K-L-K-E-A-F | |
| | D-W-L-K-A-F-Y-D-K-V-A-E-K-L-K-E-F-F-P-D-W- | 79 |
| | L-K-A-F-Y-D-K-V-A-E-K-L-K-E-F-F | |
| | D-W-F-K-A-F-Y-D-K-V-A-E-K-L-K-E-A-F-P-D-W- | 80 |
| | F-K-A-F-Y-D-K-V-A-E-K-L-K-E-A-F | |
| | D-K-L-K-A-F-Y-D-K-V-F-E-W-A-K-E-A-F-P-D-K- | 81 |
| | L-K-A-F-Y-D-K-V-F-E-W-L-K-E-A-F | |
| | D-K-W-K-A-V-Y-D-K-F-A-E-A-F-K-E-F-L-P-D-K- | 82 |
| | W-K-A-V-Y-D-K-F-A-E-A-F-K-E-F-L | |
| | D-W-F-K-A-F-Y-D-K-V-A-E-K-F-K-E-A-F-P-D-W- | 83 |
| | F-K-A-F-Y-D-K-V-A-E-K-F-K-E-A-F | |
| | D-W-L-K-A-F-V-Y-D-K-V-F-K-L-K-E-F-F-P-D-W- | 84 |
| | L-K-A-F-V-Y-D-K-V-F-K-L-K-E-F-F | |
| | D-W-L-K-A-F-Y-D-K-F-A-E-K-F-K-E-F-F-P-D-W- | 85 |
| | L-K-A-F-Y-D-K-F-A-E-K-F-K-E-F-F | |
| | Ac-E-W-F-K-A-F-Y-E-K-V-A-E-K-F-K-E-A-F-NH₂ | 86 |
| | Ac-D-W-F-K-A-F-Y-D-K-V-A-E-K-F-NH₂ | 87 |
| | Ac-F-K-A-F-Y-D-K-V-A-E-K-F-K-E-NH₂ | 88 |
| | Ac-F-K-A-F-Y-E-K-V-A-E-K-F-K-E-NH₂ | 89 |
| | NMA-F-K-A-F-Y-D-K-V-A-E-K-F-K-E-NH₂ | 90 |
| | NMA-F-K-A-F-Y-E-K-V-A-E-K-F-K-E-NH₂ | 91 |
| | NMA-D-W-F-K-A-F-Y-D-K-V-A-E-K-F-K-E-A-F-NH₂ | 92 |
| | NMA-E-W-F-K-A-F-Y-E-K-V-A-E-K-F-K-E-A-F-NH₂ | 93 |
| | NMA-A-F-Y-D-K-V-A-E-K-F-K-E-A-F-NH₂ | 94 |
| | NMA-D-W-F-K-A-F-Y-D-K-V-A-E-K-F-NH₂ | 95 |
| | Ac-D-W-L-K-A-F-Y-D-K-V-F-E-K-F-K-E-F-F-NH₂ | 96 |
| | NMA-D-W-L-K-A-F-Y-D-K-V-F-E-K-F-K-E-F-F-NH₂ | |
| | Ac-E-W-L-K-A-F-Y-E-K-V-F-E-K-F-K-E-F-F-NH₂ | 97 |
| | NMA-E-W-L-K-A-F-Y-E-K-V-F-E-K-F-K-E-F-F-NH₂ | |
| | Ac-A-F-Y-D-K-V-F-E-K-F-K-E-F-F-NH₂ | 98 |
| | NMA-A-F-Y-D-K-V-F-E-K-F-K-E-F-F-NH₂ | |
| | Ac-A-F-Y-E-K-V-F-E-K-F-K-E-F-F-NH₂ | 99 |
| | NMA-A-F-Y-E-K-V-F-E-K-F-K-E-F-F-NH₂ | |
| | Ac-D-W-L-K-A-F-Y-D-K-V-F-E-K-F-NH₂ | 100 |
| | NMA-D-W-L-K-A-F-Y-D-K-V-F-E-K-F-NH₂ | |
| | Ac-E-W-L-K-A-F-Y-E-K-V-F-E-K-F-NH₂ | 101 |
| | NMA-E-W-L-K-A-F-Y-E-K-V-F-E-K-F-NH₂ | |
| | Ac-L-K-A-F-Y-D-K-V-F-E-K-F-K-E-NH₂ | 102 |
| | NMA-L-K-A-F-Y-D-K-V-F-E-K-F-K-E-NH₂ | |
| | Ac-L-K-A-F-Y-E-K-V-F-E-K-F-K-E-NH₂ | 103 |
| | NMA-L-K-A-F-Y-E-K-V-F-E-K-F-K-E-NH₂ | |

¹Linkers are underlined.
NMA is N-Methyl Anthranilyl.

In certain preferred embodiments, the peptides include variations of 4F ((SEQ ID NO:5 in Table 2), also known as L-4F, where all residues are L form amino acids) or D-4F where one or more residues are D form amino acids). In any of the peptides described herein, the C-terminus, and/or N-terminus, and/or internal residues can be blocked with one or more blocking groups as described herein. Also, with respect to any of the peptides disclosed herein this invention contemplates L-form peptides as well as D form peptides, retro-sequences, inverse-sequences, and retro-inverse sequences.

In addition, while various peptides of Table 2, are illustrated with an acetyl group or an N-methylanthranilyl group protecting the amino terminus and an amide group protecting the carboxyl terminus, any of these protecting groups may be eliminated and/or substituted with another protecting group as described herein. In particularly preferred embodiments, the peptides comprise one or more D-form amino acids as described herein. In certain embodiments, every amino acid (e.g., every enantiomeric amino acid) of the peptides of Table 2 is a D-form amino acid.

It is also noted that Table 2 is not fully inclusive. Using the teachings provided herein, other suitable class A amphipathic helical peptides can routinely be produced (e.g., by conservative or semi-conservative substitutions (e.g., D replaced by E), extensions, deletions, and the like). Thus, for example, one embodiment utilizes truncations of any one or more of peptides shown herein (e.g., peptides identified by SEQ ID Nos:2-20 and 39—in Table 2). Thus, for example, SEQ ID NO:21 illustrates a peptide comprising 14 amino acids from the C-terminus of 18A comprising one or more D amino acids, while SEQ ID NOS:22-38 illustrate other truncations.

Longer peptides are also suitable. Such longer peptides may entirely form a class A amphipathic helix, or the class A amphipathic helix (helices) can form one or more domains of the peptide. In addition, this invention contemplates multimeric versions of the peptides (e.g., concatamers). Thus, for example, the peptides illustrated herein can be coupled together (directly or through a linker (e.g., a carbon linker, or one or more amino acids) with one or more intervening amino acids). Illustrative polymeric peptides include 18A-Pro-18A and the peptides of SEQ ID NOs:78-85, in certain embodiments comprising one or more D amino acids, more preferably with every amino acid a D amino acid as described herein and/or having one or both termini protected.

It will also be appreciated in addition to the D-form and L-form peptide sequences expressly illustrated herein, this invention also contemplates retro and retro-inverso forms of each of these peptides. In retro forms, the direction of the sequence is reversed. In inverse forms, the chirality of the constituent amino acids is reversed (i.e., L form amino acids become D form amino acids and D form amino acids become L form amino acids). In the retro-inverso form, both the order and the chirality of the amino acids is reversed. Thus, for example, a retro form of the 4F peptide (DWFKAFYDKVAEKFKEAF, SEQ ID NO:5), where the amino terminus is at the aspartate (D) and the carboxyl terminus is at the phenylalanine (F), has the same sequence, but the amino terminus is at the phenylalanine and the carboxy terminus is at the aspartate (i.e., FAEKFKEAVKDYFAKFWD, SEQ ID NO:104). Where the 4F peptide comprises all L amino acids, the retro-inverso form will have the sequence shown above (SEQ ID NO:104) and comprise all D form amino acids. As illustrated in the helical wheel diagrams shown in related application U.S. Ser. No. 11/407,390 and PCT/US2006/014389, which are incorporated herein by reference, 4F and retroinverso (Rev-4F) are mirror images of each other with identical segregation of the polar and nonpolar faces with the positively charged residues residing at the polar-nonpolar interface and the negatively charged residues residing at the center of the polar face. These mirror images of the same polymer of amino acids are identical in terms of the segregation of the polar and nonpolar faces with the positively charged residues residing at the polar-nonpolar interface and the negatively charged residues residing at the center of the polar face. Thus, 4F and Rev-4F are enantiomers of each other. For a discussion of retro- and retro-inverso peptides see, e.g., Chorev and Goodman, (1995) TibTech, 13: 439-445.

Where reference is made to a sequence and orientation is not expressly indicated, the sequence can be viewed as representing the amino acid sequence in the amino to carboxyl orientation, the retro form (i.e., the amino acid sequence in the carboxyl to amino orientation), the retro form where L amino acids are replaced with D amino acids or D amino acids are replaced with L amino acids, and the retro-inverso form where both the order is reversed and the amino acid chirality is reversed.

B) Class A Amphipathic Helical Peptide Mimetics of Apoa-I Having Aromatic or Aliphatic Residues in the Non-Polar Face.

In certain embodiments, this invention also provides modified class A amphipathic helix peptides. Certain preferred peptides incorporate one or more aromatic residues at the center of the nonpolar face, e.g., 3F^{Cπ}, (as present in 4F), or with one or more aliphatic residues at the center of the nonpolar face, e.g., 3F^{Iπ}, see, e.g., Table 3. Without being bound to a particular theory, we believe the central aromatic residues on the nonpolar face of the peptide 3F^{Cπ}, due to the presence of it electrons at the center of the nonpolar face, allow water molecules to penetrate near the hydrophobic lipid alkyl chains of the peptide-lipid complex, which in turn would enable the entry of reactive oxygen species (such as lipid hydroperoxides) shielding them from the cell surface. Similarly, we also believe the peptides with aliphatic residues at the center of the nonpolar face, e.g., 3F^{Iπ}, will act similarly but not quite as effectively as 3F^{Cπ}.

Preferred peptides will convert pro-inflammatory HDL to anti-inflammatory HDL or make anti-inflammatory HDL more anti-inflammatory, and/or decrease LDL-induced monocyte chemotactic activity generated by artery wall cells equal to or greater than D-4F or other peptides shown in Table 2.

TABLE 3
Examples of certain preferred
peptides.

| Name | Sequence | SEQ ID NO |
|---|---|---|
| (3F^{Cπ}) | Ac-DKWKAVYDKFAEAFKEFL-NH₂ | 105 |
| (3F^{Iπ}) | Ac-DKLKAFYDKVFEWAKEAF-NH₂ | 106 |

C) Other Class A and Some Class Y Amphipathic Helical Peptides.

In certain embodiments this invention also contemplates class a amphipathic helical peptides that have an amino acid composition identical to one or more of the class a amphipathic helical peptides described above. Thus, for example, in certain embodiments this invention contemplates peptides having an amino acid composition identical to 4F. Thus, in certain embodiments, this invention includes peptides that comprise 18 amino acids, where the 18 amino acids consist of 3 alanines (A), 2 aspartates (D), 2 glutamates (E), 4 phenylalanines (F), 4 lysines (K), 1 valine (V), 1 tryptophan (W), and 1 tyrosine (Y); and where the peptide forms a class A amphipathic helix; and protects a phospholipid against oxidation by an oxidizing agent. In various embodiments, the peptides comprise least one “D” amino acid residue; and in certain embodiments, the peptides comprise all “D: form amino acid residues. A variety of such peptides are illustrated in Table 4. Reverse (retro-), inverse, retro-inverso-, and circularly permuted forms of these peptides are also contemplated.

TABLE 4
Illustrative 18 amino acid length class A amphipathic helical
peptides with the amino acid composition 3 alanines (A),
2 aspartates (D), 2 glutamates (E), 4 phenylalanines (F),
4 lysines (K), 1 valine (V), 1 tryptophan (W), and 1
tyrosine (Y).

| | | SEQ ID |
|---|---|---|
| Name | Sequence | NO |
| [Switch D-E]-4F analogs | | |
| [Switch D-E]-1-4F | Ac-EWFKAFYEKVADKFKDAF-NH2 | 107 |
| [Switch D-E]-2-4F | Ac-EWFKAFYDKVADKFKEAF-NH2 | 108 |
| [Switch D-E]-3-4F | Ac-DWFKAFYEKVADKFKEAF-NH2 | 109 |
| [Switch D-E]-4-4F | Ac-DWFKAFYEKVAEKFKDAF-NH2 | 110 |
| [W-2,F-3 positions reversed] | | |
| 4F-2 | Ac-DFWKAFYDKVAEKFKEAF-NH₂ | 111 |
| [Switch D-E]-1-4F-2 | Ac-EFWKAFYEKVADKFKDAF-NH2 | 112 |
| [Switch D-E]-2-4F-2 | Ac-EFWKAFYDKVADKFKEAF-NH2 | 113 |
| [Switch D-E]-3-4F-2 | Ac-DFWKAFYEKVADKFKEAF-NH2 | 114 |
| [Switch D-E]-4-4F-2 | Ac-DFWKAFYEKVAEKFKDAF-NH2 | 115 |
| [F-6 and Y-7 positions | | |
| switched] | | |
| 4F-3 | Ac-DWFKAYFDKVAEKFKEAF-NH₂ | 116 |
| [Switch D-E]-1-4F-5 | Ac-EWFKAYFEKVADKFKDAF-NH2 | 117 |
| [Switch D-E]-2-4F-5 | Ac-EWFKAYFDKVADKFKEAF-NH2 | 118 |
| [Switch D-E]-3-4F-5 | Ac-DWFKAYFEKVADKFKEAF-NH2 | 119 |
| [Switch D-E]-4-4F-5 | Ac-DWFKAYFEKVAEKFKDAF-NH2 | 120 |
| [Y-7 and 10V positions | | |
| switched] | | |
| 4F-4 | Ac-DWFKAFVDKYAEKFKEAF-NH₂ | 121 |
| [Switch D-E]-1-4F-4 | Ac-EWFKAFVEKYADKFKDAF-NH2 | 122 |
| [Switch D-E]-2-4F-4 | Ac-EWFKAFVDKYADKFKEAF-NH2 | 123 |
| [Switch D-E]-3-4F-4 | Ac-DWFKAFVEKYADKFKEAF-NH2 | 124 |
| [Switch D-E]-4-4F | Ac-DWFKAFVEKYAEKFKDAF-NH2 | 125 |
| [V-10 and A-11 switched] | | |
| 4-F-5 | Ac-DWFKAFYDKAVEKFKEAF-NH₂ | 126 |
| [Switch D-E]-1-4F-5 | Ac-EWFKAFYEKAVDKFKDAF-NH2 | 127 |
| [Switch D-E]-2-4F-5 | Ac-EWFKAFYDKAVDKFKEAF-NH2 | 128 |
| [Switch D-E]-3-4F-5 | Ac-DWFKAFYEKAVDKFKEAF-NH2 | 129 |
| [Switch D-E]-4-4F-5 | Ac-DWFKAFYEKAVEKFKDAF-NH2 | 130 |
| [A-11 and F-14 switched] | | |
| 4F-6 | Ac-DWFKAFYDKVFEKAKEAF-NH₂ | 131 |
| [Switch D-E]-1-4F-6 | Ac-EWFKAFYEKVFDKAKDAF-NH2 | 132 |
| [Switch D-E]-2-4F-6 | Ac-EWFKAFYDKVFDKAKEAF-NH2 | 133 |
| [Switch D-E]-3-4F-6 | Ac-DWFKAFYEKVFDKAKEAF-NH2 | 134 |
| [Switch D-E]-4-4F-6 | Ac-DWFKAFYEKVFEKAKDAF-NH2 | 135 |
| [F-14 and A-17 switched] | | |
| 4F-7 | Ac-DWFKAFYDKVAEKAKEFF-NH₂ | 136 |
| [Switch D-E]-1-4F-7 | Ac-EWFKAFYEKVADKAKDFF-NH2 | 137 |
| [Switch D-E]-2-4F-7 | Ac-EWFKAFYDKVADKAKEFF-NH2 | 138 |
| [Switch D-E]-3-4F-7 | Ac-DWFKAFYEKVADKAKEFF-NH2 | 139 |
| [Switch D-E]-4-4F-7 | Ac-DWFKAFYEKVAEKAKDFF-NH2 | 140 |
| [A-17 and F-18 switched] | | |
| 4F-8 | Ac-DWFKAFYDKVAEKFKEFA-NH₂ | 141 |
| [Switch D-E]-1-4F-8 | Ac-EWFKAFYEKVADKFKDFA-NH2 | 142 |
| [Switch D-E]-2-4F-8 | Ac-EWFKAFYDKVADKFKEFA-NH2 | 143 |
| [Switch D-E]-3-4F-8 | Ac-DWFKAFYEKVADKFKEFA-NH2 | 144 |
| [Switch D-E]-4-4F-8 | Ac-DWFKAFYEKVAEKFKDFA-NH2 | 145 |
| [W-2 and A-17 switched] | | |
| 4F-9 | Ac-DAFKAFYDKVAEKFKEWF-NH₂ | 146 |
| [Switch D-E]-1-4F-9 | Ac-EAFKAFYEKVADKFKDWF-NH2 | 147 |
| [Switch D-E]-2-4F-9 | Ac-EAFKAFYDKVADKFKEWF-NH2 | 148 |
| [Switch D-E]-3-4F-9 | Ac-DAFKAFYEKVADKFKEWF-NH2 | 149 |
| [Switch D-E]-4-4F-9 | Ac-DAFKAFYEKVAEKFKDWF-NH2 | 150 |
| [W-2 and A-11 switched] | | |
| 4F-10 | Ac-DAFKAFYDKVWEKFKEAF-NH₂ | 151 |
| [Switch D-E]-1-4F-10 | Ac-EAFKAFYEKVWDKFKDAF-NH2 | 152 |
| [Switch D-E]-2-4F-10 | Ac-EAFKAFYDKVWDKFKEAF-NH2 | 153 |
| [Switch D-E]-3-4F-10 | Ac-DAFKAFYEKVWDKFKEAF-NH2 | 154 |
| [Switch D-E]-4-4F-10 | Ac-DAFKAFYEKVWEKFKDAF-NH2 | 155 |
| [W-2 and Y-7 switched] | | |
| 4F-11 | Ac-DYFKAFWDKVAEKFKEAF-NH₂ | 156 |
| [Switch D-E]-1-4F-11 | Ac-EYFKAFWEKVADKFKDAF-NH2 | 157 |
| [Switch D-E]-2-4F-11 | Ac-EYFKAFWDKVADKFKEAF-NH2 | 158 |
| [Switch D-E]-3-4F-11 | Ac-DYFKAFWEKVADKFKEAF-NH2 | 159 |
| [Switch D-E]-4-4F-11 | Ac-DYFKAFWEKVAEKFKDAF-NH2 | 160 |
| [F-3 and A-17 switched] | | |
| 4F-12 | Ac-DWAKAFYDKVAEKFKEFF-NH₂ | 161 |
| [Switch D-E]-1-4F-12 | Ac-EWAKAFYEKVADKFKDFF-NH2 | 162 |
| [Switch D-E]-2-4F-12 | Ac-EWAKAFYDKVADKFKEFF-NH2 | 163 |
| [Switch D-E]-3-4F-12 | Ac-DWAKAFYEKVADKFKEFF-NH2 | 164 |
| [Switch D-E]-4-4F-12 | Ac-DWAKAFYEKVAEKFKDFF-NH2 | 165 |
| [F-6 and A-17 switched] | | |
| 4F-13 | Ac-DWFKAAYDKVAEKFKEFF-NH₂ | 166 |
| [Switch D-E]-1-4F-13 | Ac-EWFKAAYEKVADKFKDFF-NH2 | 167 |
| [Switch D-E]-2-4F-13 | Ac-EWFKAAYDKVADKFKEFF-NH2 | 168 |
| [Switch D-E]-3-4F-13 | Ac-DWFKAAYEKVADKFKEFF-NH2 | 169 |
| [Switch D-E]-4-4F-13 | Ac-DWFKAAYEKVAEKFKDFF-NH2 | 170 |
| [Y-7 and A-17 switched | | |
| 4F-14 | Ac-DWFKAFADKVAEKFKEYF-NH₂ | 171 |
| [Switch D-E]-1-4F-14 | Ac-EWFKAFAEKVADKFKDYF-NH2 | 172 |
| [Switch D-E]-2-4F-14 | Ac-EWFKAFADKVADKFKEYF-NH2 | 173 |
| [Switch D-E]-3-4F-14 | Ac-DWFKAFAEKVADKFKEYF-NH2 | 174 |
| [Switch D-E]-4-4F | Ac-DWFKAFAEKVAEKFKDYF-NH2 | 175 |
| [V-10 and A-17 switched] | | |
| 4F-15 | Ac-DWFKAFYDKAAEKFKEVF-NH₂ | 176 |
| [Switch D-E]-1-4F-15 | Ac-EWFKAFYEKAADKFKDVF-NH2 | 177 |
| [Switch D-E]-2-4F-15 | Ac-EWFKAFYDKAADKFKEVF-NH2 | 178 |
| [Switch D-E]-3-4F-15 | Ac-DWFKAFYEKAADKFKEVF-NH2 | 179 |
| [Switch D-E]-4-4F-15 | Ac-DWFKAFYEKAAEKFKDVF-NH2 | 180 |
| [F3 and Y-7 switched] | | |
| 4F-16 | Ac-DWYKAFFDKVAEKFKEAF-NH₂ | 181 |
| [Switch D-E]-1-4F-16 | Ac-EWYKAFFEKVADKFKDAF-NH2 | 182 |
| [Switch D-E]-2-4F-16 | Ac-EWYKAFFDKVADKFKEAF-NH2 | 183 |
| [Switch D-E]-3-4F-16 | Ac-DWYKAFFEKVADKFKEAF-NH2 | 184 |
| [Switch D-E]-4-4F-16 | Ac-DWYKAFFEKVAEKFKDAF-NH2 | 185 |
| [F-3 and V-10 switched] | | |
| 4F-17 | Ac-DWVKAFYDKFAEKFKEAF-NH₂ | 186 |
| [Switch D-E]-1-4F-17 | Ac-EWVKAFYEKFADKFKDAF-NH2 | 187 |
| [Switch D-E]-2-4F-17 | Ac-EWVKAFYDKFADKFKEAF-NH2 | 188 |
| [Switch D-E]-3-4F-17 | Ac-DWVKAFYEKFADKFKEAF-NH2 | 189 |
| [Switch D-E]-4-4F-17 | Ac-DWVKAFYEKFAEKFKDAF-NH2 | 190 |
| [Y-7 and F-14 switched] | | |
| 4F-18 | Ac-DWFKAFFDKVAEKYKEAF-NH₂ | 191 |
| [Switch D-E]-1-4F-18 | Ac-EWFKAFFEKVADKYKDAF-NH2 | 192 |
| [Switch D-E]-2-4F-18 | Ac-EWFKAFFDKVADKYKEAF-NH2 | 193 |
| [Switch D-E]-3-4F-18 | Ac-DWFKAFFEKVADKYKEAF-NH2 | 194 |
| [Switch D-E]-3-4F-18 | Ac-DWFKAFFEKVADKYKEAF-NH2 | 195 |
| [Y-7 and F-18 switched] | | |
| 4F-19 | Ac-DWFKAFFDKVAEKFKEAY-NH₂ | 196 |
| [Switch D-E]-1-4F-19 | Ac-EWFKAFFEKVADKFKDAY-NH2 | 197 |
| [Switch D-E]-2-4F-19 | Ac-EWFKAFFDKVADKFKEAY-NH2 | 198 |
| [Switch D-E]-3-4F-19 | Ac-DWFKAFFEKVADKFKEAY-NH2 | 199 |
| [Switch D-E]-4-4F-19 | Ac-DWFKAFFEKVAEKFKDAY-NH2 | 200 |
| [V-10 and F-18 switched | | |
| 4F-20 | Ac-DWFKAFYDKFAEKFKEAV-NH₂ | 201 |
| [Switch D-E]-1-4F-20 | Ac-EWFKAFYEKFADKFKDAV-NH2 | 202 |
| [Switch D-E]-2-4F-20 | Ac-EWFKAFYDKFADKFKEAV-NH2 | 203 |
| [Switch D-E]-3-4F-20 | Ac-DWFKAFYEKFADKFKEAV-NH2 | 204 |
| [Switch D-E]-4-4F-20 | Ac-DWFKAFYEKFAEKFKDAV-NH2 | 205 |
| [W-2 and K13 switched] | | |
| 4F-21 | Ac-DKFKAFYDKVAEKFWEAF-NH₂ | 206 |
| [Switch D-E]-1-4F-21 | Ac-EKFKAFYEKVADKFWDAF-NH2 | 207 |
| [Switch D-E]-2-4F-21 | Ac-EKFKAFYDKVADKFWEAF-NH2 | 208 |
| [Switch D-E]-3-4F-21 | Ac-DKFKAFYEKVADKFWEAF-NH2 | 209 |
| [Switch D-E]-4-4F-21 | Ac-DKFKAFYEKVAEKFWDAF-NH2 | 210 |
| [W-3, F-13 and K-2 4F] | | |
| 4F-22 | Ac-DKWKAFYDKVAEKFFEAF-NH₂ | 211 |
| [Switch D-E]-1-4F-22 | Ac-EKWKAFYEKVADKFFDAF-NH2 | 212 |
| [Switch D-E]-2-4F-22 | Ac-EKWKAFYDKVADKFFEAF-NH2 | 213 |
| [Switch D-E]-3-4F-22 | Ac-DKWKAFYEKVADKFFEAF-NH2 | 214 |
| [Switch D-E]-4-4F-22 | Ac-DKWKAFYEKVAEKFFDAF-NH2 | 215 |
| [K-2, W10, V-13] | | |
| 4F-23 | Ac-DKFKAFYDKWAEVFKEAF-NH₂ | 216 |
| [Switch D-E]-4F analogs | | |
| [Switch D-E]-1-4F-23 | Ac-EKFKAFYEKWADVFKDAF-NH2 | 217 |
| [Switch D-E]-2-4F-23 | Ac-EKFKAFYDKWADVFKEAF-NH2 | 218 |
| [Switch D-E]-3-4F-23 | Ac-DKFKAFYEKWADVFKEAF-NH2 | 219 |
| [Switch D-E]-4-4F-23 | Ac-DKFKAFYEKWAEVFKDAF-NH2 | 220 |
| [K-2, F-13, W-14 4F] | | |
| 4F-24 | Ac-DKFKAFYDKVAEFWKEAF-NH₂ | 221 |
| [Switch D-E]-4F analogs | | |
| [Switch D-E]-1-4F-24 | Ac-EKFKAFYEKVADFWKDAF-NH2 | 222 |
| [Switch D-E]-2-4F-24 | Ac-EKFKAFYDKVADFWKEAF-NH2 | 223 |
| [Switch D-E]-3-4F-24 | Ac-DKFKAFYEKVADFWKEAF-NH2 | 224 |
| [Switch D-E]-4-4F-24 | Ac-DKFKAFYEKVAEFWKDAF-NH2 | 225 |
| Reverse 4F analogs | | |
| Rev-4F | Ac-FAEKFKEAVKDYFAKFWD-NH2 | 226 |
| [Switch D-E]-1-Rev-4F | Ac-FADKFKDAVKEYFAKFWE-NH2 | 227 |
| [Switch D-E]-2-Rev-4F | Ac-FADKFKEAVKDYFAKFWE-NH2 | 228 |
| [Switch D-E]-3-Rev-4F | Ac-FAEKFKDAVKEYFAKFWD-NH2 | 229 |
| [Switch D-E]-4-Rev-4F | Ac-FAEKFKDAVKDYFAKFWE-NH2 | 230 |
| [A-2 and W-17 switched] | | |
| Rev-4F-1 | Ac-FWEKFKEAVKDYFAKFAD-NH2 | 231 |
| [Switch D-E]-1-Rev-4F-1 | Ac-FWDKFKDAVKEYFAKFAE-NH2 | 232 |
| [Switch D-E]-2-Rev-4F-1 | Ac-FADKFKEAVKDYFAKFWE-NH2 | 233 |
| [Switch D-E]-3-Rev-4F-1 | Ac-FAEKFKDAVKEYFAKFWD-NH2 | 234 |
| [Switch D-E]-4-Rev-4F-1 | Ac-FAEKFKDAVKDYFAKFWE-NH2 | 235 |
| [Switch A-2 and F-16] | | |
| Rev-4F-2 | Ac-FFEKFKEAVKDYFAKAWD-NH2 | 236 |
| [Switch D-E]-1-Rev-4F-2 | Ac-FFDKFKDAVKEYFAKAWE-NH2 | 237 |
| [Switch D-E]-2-Rev-4F-2 | Ac-FFDKFKEAVKDYFAKAWE-NH2 | 238 |
| [Switch D-E]-3-Rev-4F-2 | Ac-FFEKFKDAVKEYFAKAWD-NH2 | 239 |
| [Switch D-E]-4-Rev-4F-2 | Ac-FFEKFKDAVKDYFAKAWE-NH2 | 240 |
| [switch F-5 and A-8] | | |
| Rev-4F-3 | Ac-FAEKAKEFVKDYFAKFWD-NH2 | 241 |
| [Switch D-E]-1-Rev-4F-3 | Ac-FADKAKDFVKEYFAKFWE-NH2 | 242 |
| [Switch D-E]-2-Rev-4F-3 | Ac-FADKAKEFVKDYFAKFWE-NH2 | 243 |
| [Switch D-E]-3-Rev-4F-3 | Ac-FAEKAKDFVKEYFAKFWD-NH2 | 244 |
| [Switch D-E]-4-Rev-4F-3 | Ac-FAEKAKDFVKDYFAKFWE-NH2 | 245 |
| [Switch A-8 and V9] | | |
| Rev-4F-4 | Ac-FAEKFKEVAKDYFAKFWD-NH2 | 246 |
| [Switch D-E]-1-Rev-4F-4 | Ac-FADKFKDVAKEYFAKFWE-NH2 | 247 |
| [Switch D-E]-2-Rev-4F-4 | Ac-FADKFKEVAKDYFAKFWE-NH2 | 248 |
| [Switch D-E]-3-Rev-4F-4 | Ac-FAEKFKDVAKEYFAKFWD-NH2 | 249 |
| [Switch D-E]-4-Rev-4F-4 | Ac-FAEKFKDVAKDYFAKFWE-NH2 | 250 |
| [Switch V-9 to Y-12] | | |
| Rev-4F-5 | Ac-FAEKFKEAYKDVFAKFWD-NH2 | 251 |
| [Switch D-E]-1-Rev-4F-5 | Ac-FADKFKDAYKEVFAKFWE-NH2 | 252 |
| [Switch D-E]-2-Rev-4F-5 | Ac-FADKFKEAYKDVFAKFWE-NH2 | 253 |
| [Switch D-E]-3-Rev-4F-5 | Ac-FAEKFKDAYKEVFAKFWD-NH2 | 254 |
| [Switch D-E]-4-Rev-4F-5 | Ac-FAEKFKDAYKDVFAKFWE-NH2 | 255 |
| [Switch Y-12 and F-13] | | |
| Rev-4F-6 | Ac-FAEKFKEAVKDFYAKFWD-NH2 | 256 |
| [Switch D-E]-1-Rev-4F-6 | Ac-FADKFKDAVKEFYAKFWE-NH2 | 257 |
| [Switch D-E]-2-Rev-4F-6 | Ac-FADKFKEAVKDFYAKFWE-NH2 | 258 |
| [Switch D-E]-3-Rev-4F-6 | Ac-FAEKFKDAVKEFYAKFWD-NH2 | 259 |
| [Switch D-E]-4-Rev-4F-6 | Ac-FAEKFKDAVKDFYAKFWE-NH2 | 260 |
| [Switch K-6 and W-17] | | |
| Rev-4F-7 | Ac-FAEKFWEAVKDYFAKFKD-NH2 | 261 |
| [Switch D-E]-1-Rev-4F-7 | Ac-FADKFWDAVKEYFAKFKE-NH2 | 262 |
| [Switch D-E]-2-Rev-4F-7 | Ac-FADKFWEAVKDYFAKFKE-NH2 | 263 |
| [Switch D-E]-3-Rev-4F-7 | Ac-FAEKFWDAVKEYFAKFKD-NH2 | 264 |
| [Switch D-E]-4-Rev-4F-7 | Ac-FAEKFWDAVKDYFAKFKE-NH2 | 265 |
| [Switch F-1 and A-2] | | |
| Rev-4F-8 | Ac-AFEKFKEAVKDYFAKFWD-NH2 | 266 |
| [Switch D-E]-1-Rev-4F-8 | Ac-AFDKFKDAVKEYFAKFWE-NH2 | 267 |
| [Switch D-E]-2-Rev-4F-8 | Ac-AFDKFKEAVKDYFAKFWE-NH2 | 268 |
| [Switch D-E]-3-Rev-4F-8 | Ac-AFEKFKDAVKEYFAKFWD-NH2 | 269 |
| [Switch D-E]-4-Rev-4F-8 | Ac-AFEKFKDAVKDYFAKFWE-NH2 | 270 |
| [F-1 and V-9 are switched] | | |
| Rev-F-9 | Ac-VAEKFKEAFKDYFAKFWD-NH2 | 271 |
| [Switch D-E]-1-Rev-4F-9 | Ac-VADKFKDAFKEYFAKFWE-NH2 | 272 |
| [Switch D-E]-2-Rev-4F-9 | Ac-VADKFKEAFKDYFAKFWE-NH2 | 273 |
| [Switch D-E]-3-Rev-4F-9 | Ac-VAEKFKDAFKEYFAKFWD-NH2 | 274 |
| [Switch D-E]-4-Rev4F-9 | Ac-VAEKFKDAFKDYFAKFWE-NH2 | 275 |
| [F-1 and Y-12 are switched] | | |
| Rev-4F-10 | Ac-YAEKFKEAVKDFFAKFWD-NH2 | 276 |
| [Switch D-E]-1-Rev-4F-10 | Ac-YADKFKDAVKEFFAKFWE-NH2 | 277 |
| [Switch D-E]-2-Rev-4F-10 | Ac-YADKFKEAVKDFFAKFWE-NH2 | 278 |
| [Switch D-E]-3-Rev-4F-10 | Ac-YAEKFKDAVKEFFAKFWD-NH2 | 279 |
| [Switch D-E]-4-Rev-4F-10 | Ac-YAEKFKDAVKDFFAKFWE-NH2 | 280 |
| [F-1 and A-8 are switched] | | |
| Rev-4F-11 | Ac-AAEKFKEFVKDYFAKFWD-NH2 | 281 |
| [Switch D-E]-1-Rev-4F-11 | Ac-AADKFKDFVKEYFAKFWE-NH2 | 282 |
| [Switch D-E]-2-Rev-4F-11 | Ac-AADKFKEFVKDYFAKFWE-NH2 | 283 |
| [Switch D-E]-3-Rev-4F-11 | Ac-AAEKFKDFVKEYFAKFWD-NH2 | 284 |
| Switch D-E]-4-Rev-4F-11 | Ac-AAEKFKDFVKDYFAKFWE-NH2 | 285 |
| [A-2 and F-5 are switched] | | |
| Rev-4F-12 | Ac-FFEKAKEAVKDYFAKFWD-NH2 | 286 |
| [Switch D-E]-1-Rev-4F-12 | Ac-FFDKAKDAVKEYFAKFWE-NH2 | 287 |
| [Switch D-E]-2-Rev-4F-12 | Ac-FFDKAKEAVKDYFAKFWE-NH2 | 288 |
| [Switch D-E]-3-Rev-4F-12 | Ac-FFEKAKDAVKEYFAKFWD-NH2 | 289 |
| [Switch D-E]-4-Rev-4F-12 | Ac-FFEKAKDAVKDYFAKFWE-NH2 | 290 |
| [A-2 and Y12 are switched | | |
| Rev-4F-13 | Ac-FYEKFKEAVKDAFAKFWD-NH2 | 291 |
| [Switch D-E]-1-Rev-4F-13 | Ac-FYDKFKDAVKEAFAKFWE-NH2 | 292 |
| [Switch D-E]-2-Rev-4F-13 | Ac-FYDKFKEAVKDAFAKFWE-NH2 | 293 |
| [Switch D-E]-3-Rev-4F-13 | Ac-FYEKFKDAVKEAFAKFWD-NH2 | 294 |
| [Switch D-E]-4-Rev-4F-13 | Ac-FYEKFKDAVKDAFAKFWE-NH2 | 295 |
| [A-2 and V-9 are switched] | | |
| Rev-4F-14 | Ac-FVEKFKEAAKDYFAKFWD-NH2 | 296 |
| [Switch D-E]-1-Rev-4F-14 | Ac-FVDKFKDAAKEYFAKFWE-NH2 | 297 |
| [Switch D-E]-2-Rev-4F-14 | Ac-FVDKFKEAAKDYFAKFWE-NH2 | 298 |
| [Switch D-E]-3-Rev-4F-14 | Ac-FVEKFKDAAKEYFAKFWD-NH2 | 299 |
| [Switch D-E]-4-Rev-4F-14 | Ac-FVEKFKDAAKDYFAKFWE-NH2 | 300 |
| [F-5 and Y-12 are switched] | | |
| Rev-4F-15 | Ac-FAEKYKEAVKDFFAKFWD-NH2 | 301 |
| [Switch D-E]-1-Rev-4F-15 | Ac-FADKYKDAVKEFFAKFWE-NH2 | 302 |
| [Switch D-E]-2-Rev-4F-15 | Ac-FADKYKEAVKDFFAKFWE-NH2 | 303 |
| [Switch D-E]-3-Rev-4F-15 | Ac-FAEKYKDAVKEFFAKFWD-NH2 | 304 |
| [Switch D-E]-4-Rev-4F-15 | Ac-FAEKYKDAVKDFFAKFWE-NH2 | 305 |
| [F-5 and V-9 are switched] | | |
| Rev-4F-16 | Ac-FAEKVKEAFKDYFAKFWD-NH2 | 306 |
| [Switch D-E]-1-Rev-4F-16 | Ac-FADKVKDAFKEYFAKFWE-NH2 | 307 |
| [Switch D-E]-2-Rev-4F-16 | Ac-FADKVKEAFKDYFAKFWE-NH2 | 308 |
| [Switch D-E]-3-Rev-4F-16 | Ac-FAEKVKDAFKEYFAKFWD-NH2 | 309 |
| [Switch D-E]-4-Rev-4F-16 | Ac-FAEKVKDAFKDYFAKFWE-NH2 | 310 |
| [A-8 and Y-12 switched] | | |
| Rev-4F-17 | Ac-FAEKFKEYVKDAFAKFWD-NH2 | 311 |
| [Switch D-E]-1-Rev-4F-17 | Ac-FADKFKDYVKEAFAKFWE-NH2 | 312 |
| [Switch D-E]-2-Rev-4F-17 | Ac-FADKFKEYVKDAFAKFWE-NH2 | 313 |
| [Switch D-E]-3-Rev-4F-17 | Ac-FAEKFKDYVKEAFAKFWD-NH2 | 314 |
| [Switch D-E]-4-Rev-4F-17 | Ac-FAEKFKDYVKDAFAKFWE-NH2 | 315 |
| [V-9 and F-13 are switched] | | |
| Rev-4F-18 | Ac-FAEKFKEAFKDYVAKFWD-NH2 | 316 |
| [Switch D-E]-1-Rev-4F-18 | Ac-FADKFKDAFKEYVAKFWE-NH2 | 317 |
| [Switch D-E]-2-Rev-4F-18 | Ac-FADKFKEAFKDYVAKFWE-NH2 | 318 |
| [Switch D-E]-3-Rev-4F-18 | Ac-FAEKFKDAFKEYVAKFWD-NH2 | 319 |
| [Switch D-E]-4-Rev-4F-18 | Ac-FAEKFKDAFKDYVAKFWE-NH2 | 320 |
| [V-9 and F-16 switched] | | |
| Rev-4F-19 | Ac-FAEKFKEAFKDYFAKVWD-NH2 | 321 |
| [Switch D-E]-1-Rev-4F-19 | Ac-FADKFKDAFKEYFAKVWE-NH2 | 322 |
| [Switch D-E]-2-Rev-4F-19 | Ac-FADKFKEAFKDYFAKVWE-NH2 | 323 |
| [Switch D-E]-3-Rev-4F-19 | Ac-FAEKFKDAFKEYFAKVWD-NH2 | 324 |
| Switch D-E]-4-Rev-4F-19 | Ac-FAEKFKDAFKDYFAKVWE-NH2 | 325 |
| [Y-12 and F-16 are switched | | |
| Rev-4F-20 | Ac-FAEKFKEAVKDFFAKYWD-NH2 | 326 |
| [Switch D-E]-1-Rev-4F-20 | Ac-FADKFKDAVKEFFAKYWE-NH2 | 327 |
| [Switch D-E]-2-Rev-4F-20 | Ac-FADKFKEAVKDFFAKYWE-NH2 | 328 |
| [Switch D-E]-3-Rev-4F-20 | Ac-FAEKFKDAVKEFFAKYWD-NH2 | 329 |
| [Switch D-E]-4-Rev-4F-20 | Ac-FAEKFKDAVKDFFAKYWE-NH2 | 330 |
| [W-1, F-6 and K-17 Rev 4F] | | |
| Rev-4F-21 | Ac-WAEKFFEAVKDYFAKFKD-NH2 | 331 |
| [Switch D-E]-1-Rev-4F-7 | Ac-WADKFFDAVKEYFAKFKE-NH2 | 332 |
| [Switch D-E]-2-Rev-4F-7 | Ac-WADKFFEAVKDYFAKFKE-NH2 | 333 |
| [Switch D-E]-3-Rev-4F-7 | Ac-WAEKFFDAVKEYFAKFKD-NH2 | 334 |
| Switch D-E]-4-Rev-4F-7 | Ac-WAEKFFDAVKDYFAKFKE-NH2 | 335 |
| [W-5, F-6 and K-17 Rev-4F] | | |
| Rev-4F-22 | Ac-FAEKWFEAVKDYFAKFKD-NH2 | 336 |
| [Switch D-E]-1-Rev-4F-22 | Ac-FADKWFDAVKEYFAKFKE-NH2 | 337 |
| [Switch D-E]-2-Rev-4F-22 | Ac-FADKWFEAVKDYFAKFKE-NH2 | 338 |
| [Switch D-E]-3-Rev-4F-22 | Ac-FAEKWFDAVKEYFAKFKD-NH2 | 339 |
| [Switch D-E]-4-Rev-4F-22 | Ac-FAEKWFDAVKDYFAKFKE-NH2 | 340 |
| [V-6, W-9, K-17 Rev-4F] | | |
| Rev-4F-23 | Ac-FAEKFVEAWKDYFAKFKD-NH2 | 341 |
| [Switch D-E]-1-Rev-4F-23 | Ac-FADKFVDAWKEYFAKFKE-NH2 | 342 |
| [Switch D-E]-2-Rev-4F-23 | Ac-FADKFVEAWKDYFAKFKE-NH2 | 343 |
| [Switch D-E]-3-Rev-4F-23 | Ac-FAEKFVDAWKEYFAKFKD-NH2 | 344 |
| [Switch D-E]-4-Rev-4F-23 | Ac-FAEKFVDAWKDYFAKFKE-NH2 | 345 |
| [Y-2, A-4, W-12, K-17 Rev- | | |
| 4F] | | |
| Rev-4F-24 | Ac-FYEKFAEAVKDWFAKFKD-NH2 | 346 |
| [Switch D-E]-1-Rev-4F-24 | Ac-FYDKFADAVKEWFAKFKE-NH2 | 347 |
| [Switch D-E]-2-Rev-4F-24 | Ac-FYDKFAEAVKDWFAKFKE-NH2 | 348 |
| [Switch D-E]-3-Rev-4F-24 | Ac-FYEKFADAVKEWFAKFKD-NH2 | 349 |
| [Switch D-E]-4-Rev-4F-24 | Ac-FYEKFADAVKDWFAKFKE-NH2 | 350 |

Based on helical wheel diagrams, it is possible to readily identify biologically active and useful peptides. Thus, for example, the following peptides have been accurately identified as active: 3F1; 3F2; 4F the inverse forms thereof, the reverse (retro) forms thereof and the retro-inverso forms thereof. Thus, in certain embodiments, this invention contemplates active agents comprising a peptide that is 18 amino acids in length and forms a class A amphipathic helix where the peptide has the amino acid composition 2 aspartates, 2 glutamates, 4 lysines, 1 tryptophan, 1 tyrosine, no more than one leucine, no more than 1 valine, no less than 1 and no more than 3 alanines, and with 3 to 6 amino acids from the group: phenylalanine, alpha-naphthalanine, beta-naphthalanine, histidine, and contains either 9 or 10 amino acids on the polar face in a helical wheel representation of the class A amphipathic helix including 4 amino acids with positive charge at neutral pH with two of the positively charged residues residing at the interface between the polar and non-polar faces and with two of the four positively charged residues on the polar face that are contiguous and on the non-polar face two of the amino acid residues from the group: phenylalanine, alpha-naphthalanine, beta-naphthalanine, histidine are also contiguous and if there are 4 or more amino acids from this group on the non-polar face there are also at least 2 residues from this group that are not contiguous.

In certain embodiments, this invention also contemplates certain class Y as well as class A amphipathic helical peptides. Class Y amphipathic helical peptides are known to those of skill in the art (see, e.g., Segrest et al. (1992) J. Lipid Res. 33: 141-166; Oram and Heinecke (2005) Physiol Rev. 85: 1343-1372, and the like). In various embodiments these peptides include, but are not limited to an 18 amino acid peptide that forms a class A amphipathic helix or a class Y amphipathic helix described by Formula XXIV (SEQ ID NO:351):

| | |
|---|---|
| D X X K Y X X D K X Y D K X K D Y X | XXIV |

where the D's are independently Asp or Glu; the Ks are independently Lys or Arg; the Xs are independently Leu, norLeu, Val, Ile, Trp, Phe, Tyr, β-NaI, or α-NaI and all X residues are on the non-polar face (e.g., when viewed in a helical wheel diagram) except for one that can be on the polar face between two K residues; the Y's are independently Ala, His, Ser, Gln, Asn, or Thr non-polar face (e.g., when viewed in a helical wheel diagram) and the Y's are independently one Ala on the polar face, one His, one Ser, one Gln one Asn, or one Thr on the polar face (e.g., when viewed in a helical wheel diagram), where no more than two K are be contiguous (e.g., when viewed in a helical wheel diagram); and where no more than 3 D's are contiguous (e.g., when viewed in a helical wheel diagram) and the fourth D is be separated from the other D's by a Y. Illustrative peptides of this kind which include peptides with histidine, and/or alpha- and/or beta-napthalanine are shown in Table 5. Reverse (retro-), inverse, retro-inverso-, and circularly permuted forms of these peptides are also contemplated.

TABLE 5
Illustrates various class A and/or class Y peptide analogs with His
incorporated into the sequence.

| | | SEQ ID |
|---|---|---|
| Short name | Peptide sequence | NO |
| [A-5 > H] 4F | Ac-DWFKHFYDKVAEKFKEAF-NH₂ | 352 |
| [A-5 > H, D-E switched]4F | Ac-EWFKHFYEKVADKFKDAF-NH₂ | 353 |
| [A-5 > H, D-1 > E]4F | Ac-EWFKHFYDKVAEKFKEAF-NH₂ | 354 |
| [A-5 > H, D-8 > E]4-F | Ac-DWFKHFYEKVAEKFKEAF-NH₂ | 355 |
| [A-5 > H, E-12 > D]4F | Ac-DWFKHFYDKVADKFKEAF-NH₂ | 356 |
| [A-5 > H, E-16 > D]4F | Ac-DWFKHFYDKVAEKFKDAF-NH₂ | 357 |
| [F-3 > H, A-5 > F]-4F | Ac-DWHKFFYDKVAEKFKEAF-NH₂ | 358 |
| [F-3 > H, A-5 > F, D-E switched]-4F | Ac-EWHKFFYEKVADKFKDAF-NH₂ | 359 |
| [F-3 > H, A-5 > F, D-1 > E]-4F | Ac-EWHKFFYDKVAEKFKEAF-NH₂ | 360 |
| [F-3 > H, A-5 > F, D-8 > E]-4F | Ac-DWHKFFYEKVAEKFKEAF-NH₂ | 361 |
| [F-3 > H, A-5 > F, E-12 > D]-4F | Ac-DWHKFFYDKVADKFKEAF-NH₂ | 362 |
| [F-3 > H, A-5 > F, E-16 > D]-4F | Ac-DWHKFFYDKVAEKFKDAF-NH₂ | 363 |
| [A-5 > F, F-6 > H]4F | Ac-DWFKFHYDKVAEKFKEAF-NH₂ | 364 |
| [A-5 > F, F-6 > H, D-E switched]4F | Ac-EWFKFHYEKVADKFKDAF-NH₂ | 365 |
| [[A-5 > F, F-6 > H, D-1 > E]4F | Ac-EWFKFHYDKVAEKFKEAF-NH₂ | 366 |
| [A-5 > F, F-6 > H, D-8 > E]4F | Ac-DWFKFHYEKVAEKFKEAF-NH₂ | 367 |
| [A-5 > F, F-6 > H, E-12 > D]4F | Ac-DWFKFHYDKVADKFKEAF-NH₂ | 368 |
| [A-5 > F, F-6 > H, E-16 > D]4F | Ac-DWFKFHYDKVAEKFKDAF-NH₂ | 369 |
| [A-5 > V, V-10 > H]4F | Ac-DWFKVFYDKHAEKFKEAF-NH₂ | 370 |
| [A-5 > V, V-10 > H, D-E switched]4F | Ac-EWFKVFYEKHADKFKDAF-NH₂ | 371 |
| [A-5 > V, V-10 > H, D-1 > E]4F | Ac-EWFKVFYDKHAEKFKEAF-NH₂ | 372 |
| [A-5 > V, V-10 > H, D-8 > E]4F | Ac-DWFKVFYEKHAEKFKEAF-NH₂ | 373 |
| [A-5 > V, V-10 > H, E-12 > D]4F | Ac-DWFKVFYDKHADKFKEAF-NH₂ | 374 |
| [A-5 > V, V-10 > H, E16 > D]4F | Ac-DWFKVFYDKHAEKFKDAF-NH₂ | 375 |
| [[A-17 > H]4F | Ac-DWFKAFYDKVAEKFKEHF-NH₂ | 376 |
| [A-17 > H, D-E switched]4F | Ac-EWFKAFYEKVADKFKDHF-NH₂ | 377 |
| [[A-17 > H,D-1 > E]4F | Ac-EWFKAFYDKVAEKFKEHF-NH₂ | 378 |
| [[A-17 > H, D-8 > E]4F | Ac-DWFKAFYEKVAEKFKEHF-NH₂ | 379 |
| [[A-17 > H, E-12 > D]4F | Ac-DWFKAFYDKVADKFKEHF-NH₂ | 380 |
| [[A-17 > H, E16 > D]4F | Ac-DWFKAFYDKVAEKFKDHF-NH₂ | 381 |
| [A-17 > F, F-18 > H]4F | Ac-DWFKAFYDKVAEKFKEFH-NH₂ | 382 |
| [A-17 > F, F-18 > H, D-E switched]4F | Ac-EWFKAFYEKVADKFKDFH-NH₂ | 383 |
| [A-17 > F, F-18 > H, D-1 > E]-4F | Ac-EWFKAFYDKVAEKFKEFH-NH₂ | 384 |
| [A-17 > F, F-18 > H]4F | Ac-DWFKAFYDKVAEKFKEFH-NH₂ | 385 |
| [A-17 > F, F-18 > H, D-8 > E]-4F | Ac-DWFKAFYEKVAEKFKEFH-NH₂ | 386 |
| [A-17 > F, F-18 > H, E-12 > D]4F | Ac-DWFKAFYDKVAEKFKEFH-NH₂ | 387 |
| [A-17 > F, F-18 > H], E-16 > D]-4F | Ac-DWFKAFYDKVAEKFKDFH-NH₂ | 388 |
| Rev-4F | Ac-FAEKFKEAVKDYFAKFWD-NH₂ | 389 |
| [A-2 > H]Rev4F | Ac-FHEKFKEAVKDYFAKFWD-NH₂ | 390 |
| Rev-[A-2 > H, D > E]-4F | Ac-FHEKFKEAVKEYFAKFWE-NH₂ | 391 |
| Rev-[A-2 > H, E > D]4F | Ac-FHDKFKDAVKDYFAKFWD-NH₂ | 392 |
| [A-2 > H, D-E switched]Rev-4F | Ac-FHDKFKDAVKEYFAKFWE-NH₂ | 393 |
| [A-2 > H, E-3 > D]Rev-4F | Ac-FHDKFKEAVKDYFAKFWD-NH₂ | 394 |
| [A-2 > H, E-7 > D]Rev-4F | Ac-FHEKFKDAVKDYFAKFWD-NH₂ | 395 |
| [A-2 > H, D-11 > E]Rev-4F | Ac-FHEKFKEAVKEYFAKFWD-NH₂ | 396 |
| [A-2 > H, D-18 > E]Rev-4F | Ac-FHEKFKEAVKDYFAKFWE-NH₂ | 397 |
| [F-1 > H, A-2 > F]Rev-4F | Ac-HFEKFKEAVKDYFAKFWD-NH₂ | 398 |
| [F-1 > H, A-2 > F, D-E switched]Rev- | Ac-HFDKFKDAVKEYFAKFWE-NH₂ | 399 |
| 4F | | |
| [F-1 > H, A-2 > F, D > E]Rev-4F | Ac-HFEKFKEAVKEYFAKFWE-NH₂ | 400 |
| [F-1 > H, A-2 > F, E-3 > D]Rev-4F | Ac-HFDKFKEAVKDYFAKFWD-NH₂ | 401 |
| [F-1 > H, A-2 > F, E-7 > D]Rev-4F | Ac-HFEKFKDAVKDYFAKFWD-NH₂ | 402 |
| [F-1 > H, A-2 > F, D-11 > E]Rev-4F | Ac-HFEKFKEAVKEYFAKFWD-NH₂ | 403 |
| [F-1 > H, A-2 > F, D-18 > E]Rev-4F | Ac-HFEKFKEAVKDYFAKFWE-NH₂ | 404 |
| [A-2 > F, F-5 > H]Rev D-4F | Ac-FFEKHKEAVKDYFAKFWD-NH₂ | 405 |
| [A-2 > F, F-5 > H, D-E switched]Rev | Ac-FFDKHKDAVKEYFAKFWE-NH₂ | 406 |
| D-4F | | |
| [A-2 > F, F-5 > H, D > E]Rev D-4F | Ac-FFEKHKEAVKEYFAKFWE-NH₂ | 407 |
| [A-2 > F, F-5 > H, E > D]Rev D-4F | Ac-FFDKHKDAVKDYFAKFWD-NH₂ | 408 |
| [A-2 > F, F-5 > H, E-3 > D]Rev D-4F | Ac-FFDKHKEAVKDYFAKFWD-NH₂ | 409 |
| [A-2 > F, F-5 > H, D-11 > E]Rev D-4F | Ac-FFEKHKEAVKEYFAKFWD-NH₂ | 410 |
| [A-2 > F, F-5 > H, D-18 > E]Rev D-4F | Ac-FFEKHKEAVKDYFAKFWE-NH₂ | 411 |
| [A-2 > V, V-9 > H]Rev D-4F | Ac-FVEKFKEAHKDYFAKFWD-NH₂ | 412 |
| [A-2 > V, V-9 > H, D-E switched]Rev | Ac-FVDKFKDAHKEYFAKFWE-NH₂ | 413 |
| D-4F | | |
| [A-2 > V, V-9 > H, D > E]Rev D-4F | Ac-FVEKFKEAHKEYFAKFWE-NH₂ | 414 |
| [A-2 > V, V-9 > H, E > D]Rev D-4F | Ac-FVDKFKDAHKDYFAKFWD-NH₂ | 415 |
| [A-2 > V, V-9 > H, E-3 > D]Rev D-4F | Ac-FVDKFKEAHKDYFAKFWD-NH₂ | 416 |
| [A-2 > V, V-9 > H, E-7 > D]Rev D-4F | Ac-FVEKFKDAHKDYFAKFWD-NH₂ | 417 |
| [A-2 > V, V-9 > H, D-11 > E]Rev D-4F | Ac-FVEKFKEAHKEYFAKFWD-NH₂ | 418 |
| [A-2 > V, V-9 > H, D-18 > E]Rev D-4F | Ac-FVEKFKEAHKDYFAKFWE-NH₂ | 419 |
| [A-8 > H]Rev-4F | Ac-FAEKFKEHVKDYFAKFWD-NH₂ | 420 |
| [A-8 > H, D-E switched]Rev-4F | Ac-FADKFKDHVKEYFAKFWE-NH₂ | 421 |
| [A-8 > H, D > E]Rev-4F | Ac-FAEKFKEHVKEYFAKFWE-NH₂ | 422 |
| [A-8 > H, E > D]Rev-4F | Ac-FADKFKDHVKDYFAKFWD-NH₂ | 423 |
| [A-8 > H, E-3 > D]Rev-4F | Ac-FADKFKEHVKDYFAKFWD-NH₂ | 424 |
| [A-8 > H, E-7 > D]Rev-4F | Ac-FAEKFKDHVKDYFAKFWD-NH₂ | 425 |
| [A-8 > H, D-11 > E]Rev-4F | Ac-FAEKFKEHVKEYFAKFWD-NH₂ | 426 |
| [A-8 > H, D-18 > E]Rev-4F | Ac-FAEKFKEHVKDYFAKFWE-NH₂ | 427 |
| [A-8 > F, F-13 > H]Rev-4F | Ac-FAEKFKEFVKDYHAKFWD-NH₂ | 428 |
| [A-8 > F, F-13 > H, D-E switched]Rev- | Ac-FADKFKDFVKEYHAKFWE-NH₂ | 429 |
| 4F | | |
| [A-8 > F, F-13 > H, E-3 > D]Rev-4F | Ac-FADKFKEFVKDYHAKFWD-NH₂ | 430 |
| [A-8 > F, F-13 > H, E-7 > D]Rev-4F | Ac-FAEKFKDFVKDYHAKFWD-NH₂ | 431 |
| [A-8 > F, F-13 > H, E > D]Rev-4F | Ac-FADKFKDFVKDYHAKFWD-NH₂ | 432 |
| [A-8 > F, F-13 > H, D > E]Rev-4F | Ac-FAEKFKEFVKEYHAKFWE-NH₂ | 433 |
| [A-8 > F, F-13 > H, D-11 > E]Rev-4F | Ac-FAEKFKEFVKEYHAKFWD-NH₂ | 434 |
| [A-8 > F, F-13 > H, D-18 > E]Rev-4F | Ac-FAEKFKEFVKDYHAKFWE-NH₂ | 435 |
| [A-8 > F, F16 > H]Rev.-4F | Ac-FAEKFKEFVKDYFAKHWD-NH₂ | 436 |
| [A-8 > F, F16 > H, D-E switched]Rev.- | Ac-FADKFKDFVKEYFAKHWE-NH₂ | 437 |
| 4F | | |
| [A-8 > F, F16 > H, D > E]Rev.-4F | Ac-FAEKFKEFVKEYFAKHWE-NH₂ | 438 |
| [A-8 > F, F16 > H, E > D]Rev.-4F | Ac-FADKFKDFVKDYFAKHWD-NH₂ | 439 |
| [A-8 > F, F16 > H, E-3 > D]Rev.-4F | Ac-FADKFKEFVKDYFAKHWD-NH₂ | 440 |
| [A-8 > F, F16 > H, E-7 > D]Rev.-4F | Ac-FAEKFKDFVKDYFAKHWD-NH₂ | 441 |
| [A-8 > F, F16 > H, D-11 > E]Rev.-4F | Ac-FAEKFKEFVKEYFAKHWD-NH₂ | 442 |
| [A-8 > F, F16 > H, D-18 > E]Rev.-4F | Ac-FAEKFKEFVKDYFAKHWE-NH₂ | 443 |
| Examples of class A 4F and Rev 4F analogs with beta-Nph. Similarly, | | |
| alpha-Nph analogs can be designed. Similarly to the above analogs, His | | |
| can be incorporated to Nph analogs. D > E analogs, E > D analogs and | | |
| D-E switch analogs are additional possibilities similarly to the above | | |
| described analogs. | | |
| 4Nph | Ac-DWNphKANphYDKVAEKNphKEANph-NH₂ | 444 |
| [D-E switched]4Nph | Ac-EWNphKANphYEKVADKNphKDANph-NH₂ | 445 |
| [D > E]4Nph | Ac-EWNphKANphYEKVAEKNphKEANph-NH₂ | 446 |
| [E > D]4Nph | Ac-DWNphKANphYDKVADKNphKDANph-NH₂ | 447 |
| [D-1 > E]4Nph | Ac-EWNphKANphYDKVAEKNphKEANph-NH₂ | 448 |
| [D-8 > E]4Nph | Ac-DWNphKANphYEKVAEKNphKEANph-NH₂ | 449 |
| [E-12 > D]4Nph | Ac-DWNphKANphYDKVADKNphKEANph-NH₂ | 450 |
| [E-16 > D]4Nph | Ac-DWNphKANphYDKVAEKNphKDANph-NH2 | 451 |
| As described above for 4Nph, a minimum of 7 additional analogs for each | | |
| of the analogs given below. | | |
| [F-3,6, > Nph]4F | Ac-DWNphKANphYDKVAEKFKEAF-NH₂ | 452 |
| [F-14,18 > Nph]4F | Ac-DWFKAFYDKVAEKNphKEANph-NH₂ | 453 |
| [[F-3 > Nph]4F | Ac-DWNphKAFYDKVAEKFKEAF-NH₂ | 454 |
| [F-6 > Nph]4F | Ac-DWFKANphYDKVAEKFKEAF-NH₂ | 455 |
| [F-14 > Nph]4F | Ac-DWFKAFYDKVAEKNphKEAF-NH₂ | 456 |
| [F-18 > Nph]4F | Ac-DWFKAFYDKVAEKFKEANph-NH₂ | 457 |
| For each of the analog described below, a minimum of 7 additional | | |
| analogs are possible as described above by switching D-E, D > E and | | |
| E > D and single D or E analogs. | | |
| Rev-4Nph | Ac-NphAEKNphKEAVKDYNphAKNphWD-NH₂ | 458 |
| [F-3,6 > Nph]Rev 4F | Ac-NphAEKNphKEAVKDYFAKFWD-NH₂ | 459 |
| [F-13,16]Rev-4F | Ac-FAEKFKEAVKDYNphAKNphWD-NH2 | 460 |
| [F-3 > Nph]Rev-4F | Ac-NphAEKFKEAVKDYFAKFWD-NH₂ | 461 |
| [F-6 > Nph]Rev-4F | Ac-FAEKNphKEAVKDYFAKFWD-NH₂ | 462 |
| [F-13 > Nph]Rev-4F | Ac-FAEKFKEAVKDYNphAKFWD-NH₂ | 463 |
| [F-16 > Nph]Rev-4F | Ac-FAEKFKEAVKDYFAKNphWD-NH₂ | 464 |
| For the analogs described below, additional analogs are possible by | | |
| incorporating His or alpha-Nph and beta-Nph | | |
| Rev-[D > E]-4F | Ac-FAEKFKEAVKEYFAKFWE-NH₂ | 465 |
| Rev-[E > D]4F | Ac-FADKFKDAVKDYFAKFWD-NH₂ | 466 |
| Rev-R4-4F | Ac-FAERFREAVKDYFAKFWD-NH₂ | 467 |
| Rev-R6-4F | Ac-FAEKFREAVKDYFAKFWD-NH₂ | 468 |
| Rev-R10-4F | Ac-FAEKFKEAVRDYFAKFWD-NH₂ | 469 |
| Rev-R14-4F | Ac-FAEKFKEAVKDYFARFWD-NH₂ | 470 |
| Rev-[D > E]-4F | Ac-FAEKFKEAVKEYFAKFWE-NH₂ | 471 |
| Rev-[E > D]4F | Ac-FADKFKDAVKDYFAKFWD-NH₂ | 472 |
| Rev-R4-4F | Ac-FAERFREAVKDYFAKFWD-NH₂ | 473 |
| Rev-R6-4F | Ac-FAEKFREAVKDYFAKFWD-NH₂ | 474 |
| Rev-R10-4F | Ac-FAEKFKEAVRDYFAKFWD-NH₂ | 475 |
| Rev-R14-4F | Ac-FAEKFKEAVKDYFARFWD-NH₂ | 476 |
| Rev-[D > E]-4F | Ac-FAEKFKEAVKEYFAKFWE-NH₂ | 477 |
| Rev-[E > D]4F | Ac-FADKFKDAVKDYFAKFWD-NH₂ | 478 |
| Rev-R4-4F | Ac-FAERFREAVKDYFAKFWD-NH₂ | 479 |
| Rev-R6-4F | Ac-FAEKFREAVKDYFAKFWD-NH₂ | 480 |
| Rev-R10-4F | Ac-FAEKFKEAVRDYFAKFWD-NH₂ | 481 |
| Rev-R14-4F | Ac-FAEKFKEAVKDYFARFWD-NH₂ | 482 |
| Rev-R4-4F | Ac-FAERFREAVKDYFAKFWD-NH₂ | 483 |
| Rev-R6-4F | Ac-FAEKFREAVKDYFAKFWD-NH₂ | 484 |
| Rev-R10-4F | Ac-FAEKFKEAVRDYFAKFWD-NH₂ | 485 |
| Rev-R14-4F | Ac-FAEKFKEAVKDYFARFWD-NH₂ | 486 |
| Rev-[D > E]-4F | Ac-FAEKFKEAVKEYFAKFWE-NH₂ | 487 |
| Rev-[E > D]4F | Ac-FADKFKDAVKDYFAKFWD-NH₂ | 488 |
| Rev-R4-4F | Ac-FAERFREAVKDYFAKFWD-NH₂ | 489 |
| Rev-R6-4F | Ac-FAEKFREAVKDYFAKFWD-NH₂ | 490 |
| Rev-R10-4F | Ac-FAEKFKEAVRDYFAKFWD-NH₂ | 491 |
| Rev-R14-4F | Ac-FAEKFKEAVKDYFARFWD-NH₂ | 492 |
| For each of the analogs below; additional H and Nph analogs are pos- | | |
| sible using the examples described above. Each analog can yield 7 | | |
| analogs with the changes described in the examples given above. | | |
| Rev3F-2 | Ac-LFEKFAEAFKDYVAKWKD-NH₂ | 493 |
| RevR4-3F-2 | Ac-LFERFAEAFKDYVAKWKD-NH₂ | 494 |
| RevR10-3F2 | Ac-LFEKFAEAFRDYVAKWKD-NH₂ | 495 |
| RevR15-3F-2 | Ac-LFEKFAEAFKDYVARWKD-NH₂ | 496 |
| Rev R17-3F-2 | Ac-LFEKFAEAFKDYVAKWRD-NH₂ | 497 |
| Rev[D > E]3F2 | Ac-LFEKFAEAFKEYVAKWKE-NH₂ | 498 |
| Rev[E > D]3F-2 | Ac-LFDKFADAFKDYVAKWKD-NH₂ | 499 |
| Rev-[E3 > D]-3F-2 | Ac-LFDKFAEAFKDYVAKWKD-NH₂ | 500 |
| Rev-[E7 > D]-3F-2 | Ac-LFEKFADAFKDYVAKWKD-NH₂ | 501 |
| Rev[D11 > E]3F-2 | Ac-LFEKFAEAFKEYVAKWKD-NH₂ | 502 |
| Rev-[D18 > E]3F-2 | Ac-LFEKFAEAFKDYVAKWKE-NH₂ | 503 |
| Rev3F-1 | Ac-FAEKAWEFVKDYFAKLKD-NH₂ | 504 |
| RevR4-3F-1 | Ac-FAERAWEFVKDYFAKLKD-NH₂ | 505 |
| RevR10-3F-1 | Ac-FAEKAWEFVKDYFAKLKD-NH₂ | 506 |
| RevR15-3F-1 | Ac-FAEKAWEFVKDYFAKLKD-NH₂ | 507 |
| RevR17-3F-1 | Ac-FAEKAWEFVKDYFAKLRD-NH₂ | 508 |
| Rev[D > E]3F-1 | Ac-FAEKAWEFVKEYFAKLKE-NH₂ | 509 |
| Rev[E > D]3F-1 | Ac-FADKAWDFVKDYFAKLKD-NH₂ | 510 |
| Rev[E3 > D]-3F-1 | Ac-FADKAWEFVKDYFAKLKD-NH₂ | 511 |
| Rev[E7 > D]3F-1 | Ac-FAEKAWDFVKDYFAKLKD-NH₂ | 512 |
| Rev-[D11 > E]3F-1 | Ac-FAEKAWEFVKEYFAKLKD-NH₂ | 513 |
| Rev-[D18 > E]3F-1 | Ac-FAEKAWEFVKDYFAKLKE-NH₂ | 514 |
| Rev-5F | Ac-FFEKFKEFVKDYFAKLWD-NH₂ | 515 |
| Rev-[D > E]5F | Ac-FFEKFKEFVKEYFAKLWE-NH₂ | 516 |
| Rev-[E > D]5F | Ac-FFDKFKDFVKDYFAKLWD-NH₂ | 517 |
| Rev-R4-5F | Ac-FFERFKEFVKDYFAKLWD-NH₂ | 518 |
| Rev-R6-5F | Ac-FFEKFREFVKDYFAKLWD-NH₂ | 519 |
| Rev-R10-5F | Ac-FFEKFKEFVRDYFAKLWD-NH₂ | 520 |
| Rev-R15-5F | Ac-FFEKFKEFVKDYFARLWD-NH₂ | 521 |
| Rev-[E3 > D]-5F | Ac-FFDKFKEFVKDYFAKLWD-NH₂ | 522 |
| Rev-[E7 > D]5F | Ac-FFEKFKDFVKDYFAKLWD-NH₂ | 523 |
| Rev-[D11 > E]-5F | Ac-FFEKFKEFVKEYFAKLWD-NH₂ | 524 |
| Rev-[D18 > E]-5F | Ac-FFEKFKEFVKDYFAKLWE-NH₂ | 525 |
| Rev-5F-2 | Ac-FLEKFKEFVKDYFAKFWD-NH₂ | 526 |
| Rev-[D > E]-5F-2 | Ac-FLEKFKEFVKEYFAKFWE-NH₂ | 527 |
| Rev-[E > D]-5F-2 | Ac-FLDKFKEFVKDYFAKFWD-NH₂ | 528 |
| Rev-[E3 > D]-5F-2 | Ac-FLDKFKEFVKDYFAKFWD-NH₂ | 529 |
| Rev-[E7 > D]-5F-2 | Ac-FLEKFKDFVKDYFAKFWD-NH₂ | 530 |
| Rev-[D11 > E]-5F-2 | Ac-FLEKFKEFVKEYFAKFWD-NH₂ | 531 |
| Rev-[D18 > E]-5F-2 | Ac-FLEKFKEFVKDYFAKFWE-NH₂ | 532 |
| Rev-R4-5F-2 | Ac-FLERFKEFVKDYFAKFWD-NH₂ | 533 |
| Rev-R6-5F-2 | Ac-FLEKFREFVKDYFAKFWD-NH₂ | 534 |
| RevR10-5F-2 | Ac-FLEKFKEFVRDYFAKFWD-NH₂ | 535 |
| Rev-R16-5F-2 | Ac-FLEKFKEFVKDYFARFWD-NH₂ | 536 |
| Rev-6F | Ac-FFEKFKEFFKDYFAKLWD-NH₂ | 537 |
| Rev-[D > E]-6F | Ac-FFEKFKEFFKEYFAKLWE-NH₂ | 538 |
| Rev-[E > D]-6F | Ac-FFDKFKDFFKDYFAKLWD-NH₂ | 539 |
| Rev-R4-6F | Ac-FFERFKEFFKDYFAKLWD-NH₂ | 540 |
| Rev-R6-6F | Ac-FFEKFREFFKDYFAKLWD-NH₂ | 541 |
| Rev-R10-6F | Ac-FFEKFKEFFRDYFAKLWD-NH₂ | 542 |
| Rev-R14-6F | Ac-FFERFKEFFKDYFARLWD-NH₂ | 543 |
| Rev-[E3 > D]-6F | Ac-FFDKFKEFFKDYFAKLWD-NH₂ | 544 |
| Rev-[E7 > D]-6F | Ac-FFEKFKDFFKDYFAKLWD-NH₂ | 545 |
| Rev-[D11 > E]-6F | Ac-FFEKFKEFFKEYFAKLWD-NH₂ | 546 |
| Rev-[D18 > E]-6F | Ac-FFEKFKEFFKDYFAKLWE-NH₂ | 547 |
| Rev-4F | Ac-FAEKFKEAVKDYFAKFWD-NH₂ | 548 |
| Rev-[D > E]-4F | Ac-FAEKFKEAVKEYFAKFWE-NH₂ | 549 |
| Rev-[E > D]4F | Ac-FADKFKDAVKDYFAKFWD-NH₂ | 550 |
| Rev-R4-4F | Ac-FAERFREAVKDYFAKFWD-NH₂ | 551 |
| Rev-R6-4F | Ac-FAEKFREAVKDYFAKFWD-NH₂ | 552 |
| Rev-R10-4F | Ac-FAEKFKEAVRDYFAKFWD-NH₂ | 553 |
| Rev-R14-4F | Ac-FAEKFKEAVKDYFARFWD-NH₂ | 554 |
| 4F-2 | Ac-DKWKAVYDKFAEAFKEFF-NH₂ | 555 |
| [D > E]-4F-2 | Ac-EKWKAVYEKFAEAFKEFF-NH₂ | 556 |
| [E > D]-4F-2 | Ac-DKWKAVYDKFADAFKDFF-NH₂ | 557 |
| R2-4F-2 | Ac-DRWKAVYDKFAEAFKEFF-NH₂ | 558 |
| R4-4F-2 | Ac-DKWRAVYDKFAEAFKEFF-NH₂ | 559 |
| R9-4F-2 | Ac-DKWKAVYDRFAEAFKEFF-NH₂ | 560 |
| R14-4F-2 | Ac-DKWKAVYDKFAEAFREFF-NH₂ | 561 |
| Rev4F-2 | Ac-FFEKFAEAFKDYVAKWKD-NH₂ | 562 |
| Rev-[D > E]-4F-2 | Ac-FFEKFAEAFKEYVAKWKE-NH₂ | 563 |
| Rev-[E > D]-3F-2 | Ac-FFDKFADAFKDYVAKWKD-NH₂ | 564 |
| Rev-R4-4F-2 | Ac-FFERFAEAFKDYVAKWKD-NH₂ | 565 |
| Rev-R10-4F-2 | Ac-FFERFAEAFRDYVAKWKD-NH₂ | 566 |
| Rev-R15-4F-2 | Ac-FFEKFAEAFKDYVARWKD-NH₂ | 567 |
| Rev-R17-4F-2 | Ac-FFERFAEAFKDYVAKWRD-NH₂ | 568 |
| Rev-[E3 > D]-4F-2 | Ac-FFDKFAEAFKDYVAKWKD-NH₂ | 569 |
| Rev-[E7 > D]-4F-2 | Ac-FFEKFADAFKDYVAKWKD-NH₂ | 570 |
| Rev-[D11 > E]-4F-2 | Ac-FFERFAEAFKEYVAKWKD-NH₂ | 571 |
| Rev-[D18 > E]-4F-2 | Ac-FFERFAEAFKDYVAKWKE-NH₂ | 572 |
| Rev-7F | Ac-FFEKFKEFFKDYFAKFWD-NH₂ | 573 |
| Rev-[E > D]-7F | Ac-FFDKFKDFFKDYFAKFWD-NH₂ | 574 |
| Rev-[D > E]-7F | Ac-FFEKFKEFFKEYFAKFWE-NH₂ | 575 |
| Rev-R4-7F | Ac-FFERFKEFFKDYFAKFWD-NH₂ | 576 |
| Rev-R6-7F | Ac-FFEKFREFFKDYFAKFWD-NH₂ | 577 |
| Rev-R10-7F | Ac-FFEKFKEFFRDYFAKFWD-NH₂ | 578 |
| Rev-R14-7F | Ac-FFEKFKEFFKDYFARFWD-NH₂ | 579 |
| Rev-[E3 > D]-7F | Ac-FFDKFKEFFKDYFAKFWD-NH₂ | 580 |
| Rev-[E7 > D]7F | Ac-FFEKFKDFFKDYFAKFWD-NH₂ | 581 |
| Rev-[D11 > E]-7F | Ac-FFEKFKEFFKEYFAKFWD-NH₂ | 582 |
| Rev-[D18 > E]-7F | Ac-FFEKFKEFFKDYFAKFWE-NH₂ | 583 |

It is also noted that any of the peptides described herein can comprise non-natural amino acids in addition to or instead of the corresponding natural amino acids identified herein. Such modifications include, but are not limited to acetylation, amidation, formylation, methylation, sulfation, and the like. Illustrative non-natural amino acids include, but are not limited to Ornithine, norleucine, norvaline, N-methylvaline, 6-N-methyllysine, N-methylisoleucine, N-methylglycine, sarcosine, inosine, allo-isoleucine, isodesmolysine, 4-hydroxyproline, 3-hydroxyproline, allo-hydroxylysine, hydroxylisine, N-ethylasparagine, N-ethylglycine, 2,3-diaminopropionic acid, 2,2′-diaminopropionic acid, desmosine, 2,4-diaminobutyric acid, 2-aminopimelic acid, 3-aminoisobutyric acid, 2-aminoisobutyric acid, 2-aminoheptanoic acid, 6-aminocaproic acid, 4-aminobutyric acid, 2-aminobutyric acid, beta-alanine, 3-aminoadipic acid, 2-aminoadipic acid, and the like. In certain embodiments and one or more of the “natural” amino acids of the peptides described herein, can be substituted with the corresponding non-natural amino acid (e.g., as describe above).

In certain embodiments, this invention contemplates particularly the use of modified lysines. Such modifications include, but are not limited to, biotin modification of epsilon lysines and/or methylation of the epsilon lysines. Illustrative peptide comprising epsilon methylated lysines include, but are not limited to: Ac-D-W-F-K(eCH₃)₂-A-F-Y-D-K(eCH₃)₂-V-A-E-K(eCH₃)₂-F-K(eCH₃)-2-E-A-F-NH(CH₃)₂ (SEQ ID NO:584) and: Ac-DWFK(eCH₃)₂AFYDK(eCH₃)₂VAEK(eCH₃)₂FK(eCH₃)₂EAF-NH(CH₃) (SEQ ID NO:585). Other modified amino acids include but are not limited to ornithine analogs and homoaminoalanine analogs (instead of (CH₂)₄—NH₂ for Lys it can be —(CH₂)₂—NH₂ for Haa and —(CH₂)₃—NH₂ for Orn] and the like. It is noted that these modifications are illustrative and not intended to be limiting. Illustrative 4F analogues that possess modified amino acids are shown in Table 6.

TABLE 6
Illustrative 4F analogs that comprise modified amino acids.

| | |
|---|---|
| | SEQ |
| Peptide | ID NO |

εN-Dimethyl-Lys derivative of 4F (εN-Dime):

| | |
|---|---|
| Ac-D-W-F-K(εN-Dime)-A-F-Y-D-K(εN-Dime)-V-A-E-K(εN-Dime)-F- | 586 |
| K(εN-Dime)-E-A-F-NH₂ | |
| Ac-D-W-F-K-(εN-Dime)-A-F-Y-D-K(εN-Dime)-V-A-E-K(εN-Dime)-F- | 587 |
| K((εN-Dime)-E-A-F-NH-Me | |
| Ac-D-W-F-K-(εN-Dime)-A-F-Y-D-K(εN-Dime)-V-A-E-K(εN-Dime)-F- | 588 |
| K(εN-Dime)-E-A-F-N-(Me)₂ | |

εN-Diethyl-Lys derivatives of 4F (εN-Diet)

| | |
|---|---|
| Ac-D-W-F-K(εN-Diet)-A-F-Y-D-K(εN-Diet)-V-A-E-K(εN-Diet)-F- | 589 |
| K(εN-Diet)-E-A-F-NH₂ | |
| Ac-D-W-F-K(εN-Diet)-A-F-Y-D-K(εN-Diet)-V-A-E-K(εN-Diet)-F- | 590 |
| K(εN-Diet)-E-A-F-NH-Et | |
| Ac-D-W-F-K(εN-Diet)-A-F-Y-D-K(εN-Diet)-V-A-E-K(εN-Diet)-F- | 591 |
| K(εN-Diet)-E-A-F-NH-(Et)₂ | |

εN-Monomethyl-Lys derivative of 4F (εN-Me)

| | |
|---|---|
| Ac-D-W-F-K(εN-Me)-A-F-Y-D-K(εN-Me)-V-A-E-K(εN-Me)-F- | 592 |
| K(εN-Me)-E-A-F-NH₂ | |
| Ac-D-W-F-K(εN-Me)-A-F-Y-D-K(εN-Me)-V-A-E-K(εN-Me)-F- | 593 |
| K(εN-Me)-E-A-F-NH-Me | |
| Ac-D-W-F-K(εN-Me)-A-F-Y-D-K(εN-Me)-V-A-E-K(εN-Me)-F- | 594 |
| K(εN-Me)-E-A-F-N-(Me)₂ | |

εN-ethylLys derivative of 4F (εN-Et)

| | |
|---|---|
| Ac-D-W-F-K(εN-Et)-A-F-Y-D-K(εN-Et)-V-A-E-K(εN-Et)-F- | 595 |
| K(εN-Et)-E-A-F-NH₂ | |
| Ac-D-W-F-K(εN-Et)-A-F-Y-D-K(εN-Et)-V-A-E-K(εN-Et)-F- | 596 |
| K(εN-Et)-E-A-F-NH-Et | |
| Ac-D-W-F-K(εN-Et)-A-F-Y-D-K(εN-Et)-V-A-E-K(εN-Et)-F- | 597 |
| K(εN-Et)-E-A-F-NH-(Et)₂ | |

HomoLys analogs of 4F (hK) (—CH₂)_{5—}NH₂:

| | |
|---|---|
| Ac-D-W-F-hK-A-F-Y-D-hK-V-A-E-hK-F-hK-E-A-F-NH₂ | 598 |
| Ac-D-W-F-hK(εN-Dime)-A-F-Y-D-hK(εN-Dime)-V-A-E-hK(εN- | 599 |
| Dime)-F-hK(εN-Dime)-E-A-F-NH₂ | |
| Ac-D-W-F-hK(εN-Dime)-A-F-Y-D-hK(εN-Dime)-V-A-E-hK(εN- | 600 |
| Dime)-F-hK(εN-Dime)-E-A-F-N-(Me)₂ | |
| Ac-D-W-F-hK(εN-Dime)-A-F-Y-D-hK(εN-Dime)-V-A-E-hK(εN- | 601 |
| Dime)-F-hK(εN-Dime)-E-A-F-NH-Me | |
| Ac-D-W-F-hK(εN-Diet)-A-F-Y-D-hK(εN-Diet)-V-A-E-hK(εN-Diet)-F- | 602 |
| hK(εN-Diet)-E-A-F-NH-Et | |
| Ac-D-W-F-hK(εN-Me)-A-F-Y-D-hK(εN-Me)-V-A-E-hK(εN-Me)-F- | 603 |
| hK(εN-Me)-E-A-F-NH₂ | |
| Ac-D-W-F-hK(εN-Me)-A-F-Y-D-hK(εN-Me)-V-A-E-hK(εN-Me)-F- | 604 |
| hK(εN-Me)-E-A-F-NH-Me | |
| Ac-D-W-F-hK(εN-Me)-A-F-Y-D-hK(εN-Me)-V-A-E-hK(εN-Me)-F- | 605 |
| hK(εN-Me)-E-A-F-N-(Me)₂ | |
| Ac-D-W-F-hK(εN-Et)-A-F-Y-D-hK(εN-Et)-V-A-E-hK(εN-Et)-F- | 606 |
| hK(εN-Et)-E-A-F-NH₂ | |
| Ac-D-W-F-hK(εN-Et)-A-F-Y-D-hK(εN-Et)-V-A-E-hK(εN-Et)-F- | 607 |
| hK(εN-Et)-E-A-F-NH-Et | |
| Ac-D-W-F-hK(εN-Et)-A-F-Y-D-hK(εN-Et)-V-A-E-hK(εN-Et)-F- | 608 |
| hK(εN-Et)-E-A-F-NH-(Et)₂ | |

4F analogs in which K is replaced O (O = Ornithine, —(CH₂)₃—NH₂):

| | |
|---|---|
| Ac-D-W-F-O-A-F-Y-D-O-V-A-E-O-F-O-E-A-F-NH₂ | 609 |
| Ac-D-W-F-O(δN-Dime)-A-F-Y-D-O(δN-Dime)-V-A-E-O(δN-Dime)- | 610 |
| F-O(δN-Dime)-E-A-F-NH₂ | |
| Ac-D-W-F-O(δN-Dime)-A-F-Y-D-)(δN-Dime)-V-A-E-O(δN-Dime)-F- | 611 |
| O(δN-Dime)-E-A-F-N-(Me)₂ | |
| Ac-D-W-F-O(δN-Dime)-A-F-Y-D-O(δN-Dime)-V-A-E-O(δN-Dime)-F- | 612 |
| O(δN-Dime)-E-A-F-NH-Me | |
| Ac-D-W-F-O(δN-Diet)-A-F-Y-D-O(δN-Diet)-V-A-E-O(δN-Diet)-F- | 613 |
| O(δN-Diet)-E-A-F-NH-Et | |
| Ac-D-W-F-O(δN-Me)-A-F-Y-D-O(δN-Me)-V-A-E-O(δN-Me)-F- | 614 |
| O(δN-Me)-E-A-F-NH₂ | |
| Ac-D-W-F-O(δN-Me)-A-F-Y-D-O(δN-Me)-V-A-E-O(δN-Me)-F- | 615 |
| O(δN-Me)-E-A-F-NH-Me | |
| Ac-D-W-F-O(δN-Me)-A-F-Y-D-O(δN-Me)-V-A-E-O(δN-Me)-F- | 616 |
| O(δN-Me)-E-A-F-N-(Me)₂ | |
| Ac-D-W-F-O(δN-Et)-A-F-Y-D-O(δN-Et)-V-A-E-O(δN-EO-F- | 617 |
| O(δN-Et)-E-A-F-NH₂ | |
| Ac-D-W-F-O(δN-Et)-A-F-Y-D-O(δN-Et)-V-A-E-O(δN-Et)-F- | 618 |
| O(δN-Et)-E-A-F-NH-Et | |
| Ac-D-W-F-O(δN-Et)-A-F-Y-D-O(δN-Et)-V-A-E-OdεN-Et)-F- | 619 |
| O(δN-Et)-E-A-F-NH-(ET)₂ | |

The peptides and modifications shown above are intended to be illustrative and not limiting.

D) Smaller Peptides.

It was also a surprising discovery that certain small peptides consisting of a minimum of three amino acids preferentially (but not necessarily) with one or more of the amino acids being the D-stereoisomer of the amino acid, and possessing hydrophobic domains to permit lipid protein interactions, and hydrophilic domains to permit a degree of water solubility also possess significant anti-inflammatory properties and are useful in treating one or more of the pathologies described herein. The “small peptides” typically range in length from 2 amino acids to about 15 amino acids, more preferably from about 3 amino acids to about 10 or 11 amino acids, and most preferably from about 4 to about 8 or 10 amino acids. In various embodiments the peptides are typically characterized by having hydrophobic terminal amino acids or terminal amino acids rendered hydrophobic by the attachment of one or more hydrophobic “protecting” groups. Various “small peptides” are described in copending applications U.S. Ser. No. 10/649,378, filed Aug. 26, 2003, and in U.S. Ser. No. 10/913,800, filed on Aug. 6, 2004, and in PCT Application PCT/US2004/026288.

In certain embodiments, the peptides can be characterized by Formula XXV, below:

X¹-X²-X³ₙ-X⁴ XXV

where, n is 0 or 1, X¹ is a hydrophobic amino acid and/or bears a hydrophobic protecting group, X⁴ is a hydrophobic amino acid and/or bears a hydrophobic protecting group; and when n is 0 X² is an acidic or a basic amino acid; when n is 1: X² and X³ are independently an acidic amino acid, a basic amino acid, an aliphatic amino acid, or an aromatic amino acid such that when X² is an acidic amino acid; X³ is a basic amino acid, an aliphatic amino acid, or an aromatic amino acid; when X² is a basic amino acid; X³ is an acidic amino acid, an aliphatic amino acid, or an aromatic amino acid; and when X² is an aliphatic or aromatic amino acid, X³ is an acidic amino acid, or a basic amino acid.

Longer peptides (e.g., up to 10, 11, or 15 amino acids) are also contemplated within the scope of this invention. Typically where the shorter peptides (e.g., peptides according to Formula XXV) are characterized by an acidic, basic, aliphatic, or aromatic amino acid, the longer peptides are characterized by acidic, basic, aliphatic, or aromatic domains comprising two or more amino acids of that type.

1) Functional Properties of Active Small Peptides.

It was a surprising finding of this invention that a number of physical properties predict the ability of small peptides (e.g., less than 10 amino acids, preferably less than 8 amino acids, more preferably from about 3 to about 5 or 6 amino acids) of this invention to render HDL more anti-inflammatory and to mitigate atherosclerosis and/or other pathologies characterized by an inflammatory response in a mammal. The physical properties include high solubility in ethyl acetate (e.g., greater than about 4 mg/mL), and solubility in aqueous buffer at pH 7.0. Upon contacting phospholipids such as 1,2-Dimyristoyl-sn-glycero-3-phosphocholine (DMPC), in an aqueous environment, the particularly effective small peptides induce or participate in the formation of particles with a diameter of approximately 7.5 nm (±0.1 nm), and/or induce or participate in the formation of stacked bilayers with a bilayer dimension on the order of 3.4 to 4.1 nm with spacing between the bilayers in the stack of approximately 2 nm, and/or also induce or participate in the formation of vesicular structures of approximately 38 nm). In certain preferred embodiments, the small peptides have a molecular weight of less than about 900 Da.

Thus, in certain embodiments, this invention contemplates small peptides that ameliorate one or more symptoms of an indication/pathology described herein, e.g., an inflammatory condition, where the peptide(s): ranges in length from about 3 to about 8 amino acids, preferably from about 3 to about 6, or 7 amino acids, and more preferably from about 3 to about 5 amino acids; are soluble in ethyl acetate at a concentration greater than about 4 mg/mL; are soluble in aqueous buffer at pH 7.0; when contacted with a phospholipid in an aqueous environment, form particles with a diameter of approximately 7.5 nm and/or form stacked bilayers with a bilayer dimension on the order of 3.4 to 4.1 nm with spacing between the bilayers in the stack of approximately 2 nm; have a molecular weight less than about 900 daltons; convert pro-inflammatory HDL to anti-inflammatory HDL or make anti-inflammatory HDL more anti-inflammatory. In certain embodiments the peptides include, but are not limited to peptides having the amino acid sequence Lys-Arg-Asp-Ser (SEQ ID NO:620), especially in which Lys-Arg-Asp and Ser are all L amino acids. In certain embodiments, these small peptides protect a phospholipid against oxidation by an oxidizing agent. In certain embodiments the compositions and methods described herein exclude the amino acid sequence Lys-Arg-Asp-Ser (SEQ ID NO:620), especially in which Lys-Arg-Asp and Ser are all L amino acids.

While these small peptides need not be so limited, in certain embodiments, these small peptides can include the small peptides described below.

2) Tripeptides.

It was discovered that certain tripeptides (3 amino acid peptides) can be synthesized that show desirable properties as described herein (e.g., the ability to convert pro-inflammatory HDL to anti-inflammatory HDL, the ability to decrease LDL-induced monocyte chemotactic activity generated by artery wall cells. In certain embodiments, the peptides are characterized by Formula XXV, wherein N is zero, shown below as Formula XXVI:

X′-X²-X⁴ XXVI

where the end amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, nicotinyl-, etc., and the C-terminus blocked with (tBu)-OtBu, etc.). In certain embodiments, the X² amino acid is either acidic (e.g., aspartic acid, glutamic acid, etc.) or basic (e.g., histidine, arginine, lysine, etc.). The peptide can be all L-amino acids or include one or more or all D-amino acids.

Certain tripeptides of this invention include, but are not limited to the peptides shown in Table 7.

TABLE 7
Examples of certain preferred tripeptides bearing
hydrophobic blocking groups and acidic,
basic, or histidine central amino acids.

| X¹ | X² | X³ | X⁴ |
|---|---|---|---|
| Boc-Lys(εBoc) | Arg | | Ser(tBu)-OtBu |
| Boc-Lys(εBoc) | Arg | | Thr(tBu)-OtBu |
| Boc-Trp | Arg | | Ile-OtBu |
| Boc-Trp | Arg | | Leu-OtBu |
| Boc-Phe | Arg | | Ile-OtBu |
| Boc-Phe | Arg | | Leu-OtBu |
| Boc-Lys(εBoc) | Glu | | Ser(tBu)-OtBu |
| Boc-Lys(εBoc) | Glu | | Thr(tBu)-OtBu |
| Boc-Lys(εBoc) | Asp | | Ser(tBu)-OtBu |
| Boc-Lys(εBoc) | Asp | | Thr(tBu)-OtBu |
| Boc-Lys(εBoc) | Arg | | Ser(tBu)-OtBu |
| Boc-Lys(εBoc) | Arg | | Thr(tBu)-OtBu |
| Boc-Leu | Glu | | Ser(tBu)-OtBu |
| Boc-Leu | Glu | | Thr(tBu)-OtBu |
| Fmoc-Trp | Arg | | Ser(tBu)-OtBu |
| Fmoc-Trp | Asp | | Ser(tBu)-OtBu |
| Fmoc-Trp | Glu | | Ser(tBu)-OtBu |
| Fmoc-Trp | Arg | | Ser(tBu)-OtBu |
| Boc-Lys(εBoc) | Glu | | Leu-OtBu |
| Fmoc-Leu | Arg | | Ser(tBu)-OtBu |
| Fmoc-Leu | Asp | | Ser(tBu)-OtBu |
| Fmoc-Leu | Glu | | Ser(tBu)-OtBu |
| Fmoc-Leu | Arg | | Ser(tBu)-OtBu |
| Fmoc-Leu | Arg | | Thr(tBu)-OtBu |
| Boc-Glu | Asp | | Tyr(tBu)-OtBu |
| Fmoc-Lys(εFmoc) | Arg | | Ser(tBu)-OtBu |
| Fmoc-Trp | Arg | | Ile-OtBu |
| Fmoc-Trp | Arg | | Leu-OtBu |
| Fmoc-Phe | Arg | | Ile-OtBu |
| Fmoc-Phe | Arg | | Leu-OtBu |
| Boc-Trp | Arg | | Phe-OtBu |
| Boc-Trp | Arg | | Tyr-OtBu |
| Fmoc-Trp | Arg | | Phe-OtBu |
| Fmoc-Trp | Arg | | Tyr-OtBu |
| Boc-Orn(δBoc) | Arg | | Ser(tBu)-OtBu |
| Nicotinyl Lys(εBoc) | Arg | | Ser(tBu)-OtBu |
| Nicotinyl Lys(εBoc) | Arg | | Thr(tBu)-OtBu |
| Fmoc-Leu | Asp | | Thr(tBu)-OtBu |
| Fmoc-Leu | Glu | | Thr(tBu)-OtBu |
| Fmoc-Leu | Arg | | Thr(tBu)-OtBu |
| Fmoc-norLeu | Arg | | Ser(tBu)-OtBu |
| Fmoc-norLeu | Asp | | Ser(tBu)-OtBu |
| Fmoc-norLeu | Glu | | Ser(tBu)-OtBu |
| Fmoc-Lys(εBoc) | Arg | | Ser(tBu)-OtBu |
| Fmoc-Lys(εBoc) | Arg | | Thr(tBu)-OtBu |
| Fmoc-Lys(εBoc) | Glu | | Ser(tBu)-OtBu |
| Fmoc-Lys(εBoc) | Glu | | Thr(tBu)-OtBu |
| Fmoc-Lys(εBoc) | Asp | | Ser(tBu)-OtBu |
| Fmoc-Lys(εBoc) | Asp | | Thr(tBu)-OtBu |
| Fmoc-Lys(εBoc) | Glu | | Leu-OtBu |
| Fmoc-Lys(εBoc) | Arg | | Leu-OtBu |
| Fmoc-Lys(εFmoc) | Arg | | Thr(tBu)-OtBu |
| Fmoc-Lys(εFmoc) | Glu | | Ser(tBu)-OtBu |
| Fmoc-Lys(εFmoc) | Glu | | Thr(tBu)-OtBu |
| Fmoc-Lys(εFmoc) | Asp | | Ser(tBu)-OtBu |
| Fmoc-Lys(εFmoc) | Asp | | Thr(tBu)-OtBu |
| Fmoc-Lys(εFmoc) | Arg | | Ser(tBu)-OtBu |
| Fmoc-Lys(εFmoc)) | Glu | | Leu-OtBu |
| Boc-Lys(εFmoc) | Asp | | Ser(tBu)-OtBu |
| Boc-Lys(εFmoc) | Asp | | Thr(tBu)-OtBu |
| Boc-Lys(εFmoc) | Arg | | Thr(tBu)-OtBu |
| Boc-Lys(εFmoc) | Glu | | Leu-OtBu |
| Boc-Orn(δFmoc) | Glu | | Ser(tBu)-OtBu |
| Boc-Orn(δFmoc) | Asp | | Ser(tBu)-OtBu |
| Boc-Orn(δFmoc) | Asp | | Thr(tBu)-OtBu |
| Boc-Orn(δFmoc) | Arg | | Thr(tBu)-OtBu |
| Boc-Orn(δFmoc) | Glu | | Thr(tBu)-OtBu |
| Fmoc-Trp | Asp | | Ile-OtBu |
| Fmoc-Trp | Arg | | Ile-OtBu |
| Fmoc-Trp | Glu | | Ile-OtBu |
| Fmoc-Trp | Asp | | Leu-OtBu |
| Fmoc-Trp | Glu | | Leu-OtBu |
| Fmoc-Phe | Asp | | Ile-OtBu |
| Fmoc-Phe | Asp | | Leu-OtBu |
| Fmoc-Phe | Glu | | Leu-OtBu |
| Fmoc-Trp | Arg | | Phe-OtBu |
| Fmoc-Trp | Glu | | Phe-OtBu |
| Fmoc-Trp | Asp | | Phe-OtBu |
| Fmoc-Trp | Asp | | Tyr-OtBu |
| Fmoc-Trp | Arg | | Tyr-OtBu |
| Fmoc-Trp | Glu | | Tyr-OtBu |
| Fmoc-Trp | Arg | | Thr(tBu)-OtBu |
| Fmoc-Trp | Asp | | Thr(tBu)-OtBu |
| Fmoc-Trp | Glu | | Thr(tBu)-OtBu |
| Boc-Phe | Arg | | norLeu-OtBu |
| Boc-Phe | Glu | | norLeu-OtBu |
| Fmoc-Phe | Asp | | norLeu-OtBu |
| Boc-Glu | His | | Tyr(tBu)-OtBu |
| Boc-Leu | His | | Ser(tBu)-OtBu |
| Boc-Leu | His | | Thr(tBu)-OtBu |
| Boc-Lys(εBoc) | His | | Ser(tBu)-OtBu |
| Boc-Lys(εBoc) | His | | Thr(tBu)-OtBu |
| Boc-Lys(εBoc) | His | | Leu-OtBu |
| Boc-Lys(εFmoc) | His | | Ser(tBu)-OtBu |
| Boc-Lys(εFmoc) | His | | Thr(tBu)-OtBu |
| Boc-Lys(εFmoc) | His | | Leu-OtBu |
| Boc-Orn(δBoc) | His | | Ser(tBu)-OtBu |
| Boc-Orn(δFmoc) | His | | Thr(tBu)-OtBu |
| Boc-Phe | His | | Ile-OtBu |
| Boc-Phe | His | | Leu-OtBu |
| Boc-Phe | His | | norLeu-OtBu |
| Boc-Phe | Lys | | Leu-OtBu |
| Boc-Trp | His | | Ile-OtBu |
| Boc-Trp | His | | Leu-OtBu |
| Boc-Trp | His | | Phe-OtBu |
| Boc-Trp | His | | Tyr-OtBu |
| Boc-Phe | Lys | | Leu-OtBu |
| Fmoc-Lys(εFmoc) | His | | Ser(tBu)-OtBu |
| Fmoc-Lys(εFmoc) | His | | Thr(tBu)-OtBu |
| Fmoc-Lys(εFmoc) | His | | Leu-OtBu |
| Fmoc-Leu | His | | Ser(tBu)-OtBu |
| Fmoc-Leu | His | | Thr(tBu)-OtBu |
| Fmoc-Lys(εBoc) | His | | Ser(tBu)-OtBu |
| Fmoc-Lys(εBoc) | His | | Thr(tBu)-OtBu |
| Fmoc-Lys(εBoc) | His | | Leu-OtBu |
| Fmoc-Lys(εFmoc) | His | | Ser(tBu)-OtBu |
| Fmoc-Lys(εFmoc) | His | | Thr(tBu)-OtBu |
| Fmoc-norLeu | His | | Ser(tBu)-OtBu |
| Fmoc-Phe | His | | Ile-OtBu |
| Fmoc-Phe | His | | Leu-OtBu |
| Fmoc-Phe | His | | norLeu-OtBu |
| Fmoc-Trp | His | | Ser(tBu)-OtBu |
| Fmoc-Trp | His | | Ile-OtBu |
| Fmoc-Trp | His | | Leu-OtBu |
| Fmoc-Trp | His | | Phe-OtBu |
| Fmoc-Trp | His | | Tyr-OtBu |
| Fmoc-Trp | His | | Thr(tBu)-OtBu |
| Nicotinyl Lys(εBoc) | His | | Ser(tBu)-OtBu |
| Nicotinyl Lys(εBoc) | His | | Thr(tBu)-OtBu |

While the peptides of Table 7 are illustrated with particular protecting groups, it is noted that any of these groups may be eliminated and/or substituted with other protecting groups as described herein.

3) Small Peptides with Central Acidic and Basic Amino Acids.

In certain embodiments, the peptides of this invention range from four amino acids to about ten amino acids. The terminal amino acids are typically hydrophobic either because of a hydrophobic side chain or because the terminal amino acids bear one or more hydrophobic protecting groups end amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, Nicotinyl-, etc., and the C-terminus blocked with (tBu)-OtBu, etc.). Typically, the central portion of the peptide comprises a basic amino acid and an acidic amino acid (e.g., in a 4 mer) or a basic domain and/or an acidic domain in a longer molecule.

These four-mers can be represented by Formula XXV in which X¹ and X⁴ are hydrophobic and/or bear hydrophobic protecting group(s) as described herein and X² is acidic while X³ is basic or X² is basic while X³ is acidic. The peptide can be all L-amino acids or include one or more or all D-amino acids.

Certain preferred of this invention include, but are not limited to the peptides shown in Table 8.

TABLE 8
Illustrative examples of small peptides with
central acidic and basic amino acids.

| | | | | SEQ ID |
|---|---|---|---|---|
| X¹ | X² | X³ | X⁴ | NO |
| Boc-Lys(εBoc) | Arg | Asp | Ser(tBu)-OtBu | 620 |
| Boc-Lys(εBoc) | Arg | Asp | Thr(tBu)-OtBu | 621 |
| Boc-Trp | Arg | Asp | Ile-OtBu | 622 |
| Boc-Trp | Arg | Asp | Leu-OtBu | 623 |
| Boc-Phe | Arg | Asp | Leu-OtBu | 624 |
| Boc-Phe | Arg | Asp | Ile-OtBu | 625 |
| Boc-Phe | Arg | Asp | norLeu-OtBu | 626 |
| Boc-Phe | Arg | Glu | norLeu-OtBu | 627 |
| Boc-Phe | Arg | Glu | Ile-OtBu | 628 |
| Boc-Phe | Asp | Arg | Ile-OtBu | 629 |
| Boc-Phe | Glu | Arg | Ile-OtBu | 630 |
| Boc-Phe | Asp | Arg | Leu-OtBu | 631 |
| Boc-Phe | Arg | Glu | Leu-OtBu | 632 |
| Boc-Phe | Glu | Arg | Leu-OtBu | 633 |
| Boc-Phe | Asp | Arg | norLeu-OtBu | 634 |
| Boc-Phe | Glu | Arg | norLeu-OtBu | 635 |
| Boc-Lys(εBoc) | Glu | Arg | Ser(tBu)-OtBu | 636 |
| Boc-Lys(εBoc) | Glu | Arg | Thr(tBu)-OtBu | 637 |
| Boc-Lys(εBoc) | Asp | Arg | Ser(tBu)-OtBu | 638 |
| Boc-Lys(εBoc) | Asp | Arg | Thr(tBu)-OtBu | 639 |
| Boc-Lys(εBoc) | Arg | Glu | Ser(tBu)-OtBu | 640 |
| Boc-Lys(εBoc) | Arg | Glu | Thr(tBu)-OtBu | 641 |
| Boc-Leu | Glu | Arg | Ser(tBu)-OtBu | 642 |
| Boc-Leu | Glu | Arg | Thr(tBu)-OtBu | 643 |
| Fmoc-Trp | Arg | Asp | Ser(tBu)-OtBu | 644 |
| Fmoc-Trp | Asp | Arg | Ser(tBu)-OtBu | 645 |
| Fmoc-Trp | Glu | Arg | Ser(tBu)-OtBu | 646 |
| Fmoc-Trp | Arg | Glu | Ser(tBu)-OtBu | 647 |
| Boc-Lys(εBoc) | Glu | Arg | Leu-OtBu | 648 |
| Fmoc-Leu | Arg | Asp | Ser(tBu)-OtBu | 649 |
| Fmoc-Leu | Asp | Arg | Ser(tBu)-OtBu | 650 |
| Fmoc-Leu | Glu | Arg | Ser(tBu)-OtBu | 651 |
| Fmoc-Leu | Arg | Glu | Ser(tBu)-OtBu | 652 |
| Fmoc-Leu | Arg | Asp | Thr(tBu)-OtBu | 653 |
| Boc-Glu | Asp | Arg | Tyr(tBu)-OtBu | 654 |
| Fmoc-Lys(εFmoc) | Arg | Asp | Ser(tBu)-OtBu | 655 |
| Fmoc-Trp | Arg | Asp | Ile-OtBu | 656 |
| Fmoc-Trp | Arg | Asp | Leu-OtBu | 657 |
| Fmoc-Phe | Arg | Asp | Ile-OtBu | 658 |
| Fmoc-Phe | Arg | Asp | Leu-OtBu | 659 |
| Boc-Trp | Arg | Asp | Phe-OtBu | 660 |
| Boc-Trp | Arg | Asp | Tyr-OtBu | 661 |
| Fmoc-Trp | Arg | Asp | Phe-OtBu | 662 |
| Fmoc-Trp | Arg | Asp | Tyr-OtBu | 663 |
| Boc-Orn(δBoc) | Arg | Glu | Ser(tBu)-OtBu | 664 |
| Nicotinyl Lys(εBoc) | Arg | Asp | Ser(tBu)-OtBu | 665 |
| Nicotinyl Lys(εBoc) | Arg | Asp | Thr(tBu)-OtBu | 666 |
| Fmoc-Leu | Asp | Arg | Thr(tBu)-OtBu | 667 |
| Fmoc-Leu | Glu | Arg | Thr(tBu)-OtBu | 668 |
| Fmoc-Leu | Arg | Glu | Thr(tBu)-OtBu | 669 |
| Fmoc-norLeu | Arg | Asp | Ser(tBu)-OtBu | 670 |
| Fmoc-norLeu | Asp | Arg | Ser(tBu)-OtBu | 671 |
| Fmoc-norLeu | Glu | Arg | Ser(tBu)-OtBu | 672 |
| Fmoc-norLeu | Arg | Glu | Ser(tBu)-OtBu | 673 |
| Fmoc-Lys(εBoc) | Arg | Asp | Ser(tBu)-OtBu | 674 |
| Fmoc-Lys(εBoc) | Arg | Asp | Thr(tBu)-OtBu | 675 |
| Fmoc-Lys(εBoc) | Glu | Arg | Ser(tBu)-OtBu | 676 |
| Fmoc-Lys(εBoc) | Glu | Arg | Thr(tBu)-OtBu | 677 |
| Fmoc-Lys(εBoc) | Asp | Arg | Ser(tBu)-OtBu | 678 |
| Fmoc-Lys(εBoc) | Asp | Arg | Thr(tBu)-OtBu | 679 |
| Fmoc-Lys(εBoc) | Arg | Glu | Ser(tBu)-OtBu | 680 |
| Fmoc-Lys(εBoc) | Arg | Glu | Thr(tBu)-OtBu | 681 |
| Fmoc-Lys(εBoc) | Glu | Arg | Leu-OtBu | 682 |
| Fmoc-Lys(εBoc) | Arg | Glu | Leu-OtBu | 683 |
| Fmoc-Lys(εFmoc) | Arg | Asp | Thr(tBu)-OtBu | 684 |
| Fmoc-Lys(εFmoc) | Glu | Arg | Ser(tBu)-OtBu | 685 |
| Fmoc-Lys(εFmoc) | Glu | Arg | Thr(tBu)-OtBu | 686 |
| Fmoc-Lys(εFmoc) | Asp | Arg | Ser(tBu)-OtBu | 687 |
| Fmoc-Lys(εFmoc) | Asp | Arg | Thr(tBu)-OtBu | 688 |
| Fmoc-Lys(εFmoc) | Arg | Glu | Ser(tBu)-OtBu | 689 |
| Fmoc-Lys(εFmoc) | Arg | Glu | Thr(tBu)-OtBu | 690 |
| Fmoc-Lys(εFmoc)) | Glu | Arg | Leu-OtBu | 691 |
| Boc-Lys(εFmoc) | Arg | Asp | Ser(tBu)-OtBu | 692 |
| Boc-Lys(εFmoc) | Arg | Asp | Thr(tBu)-OtBu | 693 |
| Boc-Lys(εFmoc) | Glu | Arg | Ser(tBu)-OtBu | 694 |
| Boc-Lys(εFmoc) | Glu | Arg | Thr(tBu)-OtBu | 695 |
| Boc-Lys(εFmoc) | Asp | Arg | Ser(tBu)-OtBu | 696 |
| Boc-Lys(εFmoc) | Asp | Arg | Thr(tBu)-OtBu | 697 |
| Boc-Lys(εFmoc) | Arg | Glu | Ser(tBu)-OtBu | 698 |
| Boc-Lys(εFmoc) | Arg | Glu | Thr(tBu)-OtBu | 699 |
| Boc-Lys(εFmoc) | Glu | Arg | Leu-OtBu | 700 |
| Boc-Orn(δFmoc) | Arg | Glu | Ser(tBu)-OtBu | 701 |
| Boc-Orn(δFmoc) | Glu | Arg | Ser(tBu)-OtBu | 702 |
| Boc-Orn(δFmoc) | Arg | Asp | Ser(tBu)-OtBu | 703 |
| Boc-Orn(δFmoc) | Asp | Arg | Ser(tBu)-OtBu | 704 |
| Boc-Orn(δFmoc) | Asp | Arg | Thr(tBu)-OtBu | 705 |
| Boc-Orn(δFmoc) | Arg | Asp | Thr(tBu)-OtBu | 706 |
| Boc-Orn(δFmoc) | Glu | Arg | Thr(tBu)-OtBu | 707 |
| Boc-Orn(δFmoc) | Arg | Glu | Thr(tBu)-OtBu | 708 |
| Fmoc-Trp | Asp | Arg | Ile-OtBu | 709 |
| Fmoc-Trp | Arg | Glu | Ile-OtBu | 710 |
| Fmoc-Trp | Glu | Arg | Ile-OtBu | 711 |
| Fmoc-Trp | Asp | Arg | Leu-OtBu | 712 |
| Fmoc-Trp | Arg | Glu | Leu-OtBu | 713 |
| Fmoc-Trp | Glu | Arg | Leu-OtBu | 714 |
| Fmoc-Phe | Asp | Arg | Ile-OtBu | 715 |
| Fmoc-Phe | Arg | Glu | Ile-OtBu | 716 |
| Fmoc-Phe | Glu | Arg | Ile-OtBu | 717 |
| Fmoc-Phe | Asp | Arg | Leu-OtBu | 718 |
| Fmoc-Phe | Arg | Glu | Leu-OtBu | 719 |
| Fmoc-Phe | Glu | Arg | Leu-OtBu | 720 |
| Fmoc-Trp | Arg | Asp | Phe-OtBu | 721 |
| Fmoc-Trp | Arg | Glu | Phe-OtBu | 722 |
| Fmoc-Trp | Glu | Arg | Phe-OtBu | 723 |
| Fmoc-Trp | Asp | Arg | Tyr-OtBu | 724 |
| Fmoc-Trp | Arg | Glu | Tyr-OtBu | 725 |
| Fmoc-Trp | Glu | Arg | Tyr-OtBu | 726 |
| Fmoc-Trp | Arg | Asp | Thr(tBu)-OtBu | 727 |
| Fmoc-Trp | Asp | Arg | Thr(tBu)-OtBu | 728 |
| Fmoc-Trp | Arg | Glu | Thr(tBu)-OtBu | 729 |
| Fmoc-Trp | Glu | Arg | Thr(tBu)-OtBu | 730 |
| Fmoc-Phe | Arg | Asp | norLeu-OtBu | 731 |
| Fmoc-Phe | Arg | Glu | norLeu-OtBu | 732 |
| Boc-Phe | Lys | Asp | Leu-OtBu | 733 |
| Boc-Phe | Asp | Lys | Leu-OtBu | 734 |
| Boc-Phe | Lys | Glu | Leu-OtBu | 735 |
| Boc-Phe | Glu | Lys | Leu-OtBu | 736 |
| Boc-Phe | Lys | Asp | Ile-OtBu | 737 |
| Boc-Phe | Asp | Lys | Ile-OtBu | 738 |
| Boc-Phe | Lys | Glu | Ile-OtBu | 739 |
| Boc-Phe | Glu | Lys | Ile-OtBu | 740 |
| Boc-Phe | Lys | Asp | norLeu-OtBu | 741 |
| Boc-Phe | Asp | Lys | norLeu-OtBu | 742 |
| Boc-Phe | Lys | Glu | norLeu-OtBu | 743 |
| Boc-Phe | Glu | Lys | norLeu-OtBu | 744 |
| Boc-Phe | His | Asp | Leu-OtBu | 745 |
| Boc-Phe | Asp | His | Leu-OtBu | 746 |
| Boc-Phe | His | Glu | Leu-OtBu | 747 |
| Boc-Phe | Glu | His | Leu-OtBu | 748 |
| Boc-Phe | His | Asp | Ile-OtBu | 749 |
| Boc-Phe | Asp | His | Ile-OtBu | 750 |
| Boc-Phe | His | Glu | Ile-OtBu | 751 |
| Boc-Phe | Glu | His | Ile-OtBu | 752 |
| Boc-Phe | His | Asp | norLeu-OtBu | 753 |
| Boc-Phe | Asp | His | norLeu-OtBu | 754 |
| Boc-Phe | His | Glu | norLeu-OtBu | 755 |
| Boc-Phe | Glu | His | norLeu-OtBu | 756 |
| Boc-Lys(εBoc) | Lys | Asp | Ser(tBu)-OtBu | 757 |
| Boc-Lys(εBoc) | Asp | Lys | Ser(tBu)-OtBu | 758 |
| Boc-Lys(εBoc) | Lys | Glu | Ser(tBu)-OtBu | 759 |
| Boc-Lys(εBoc) | Glu | Lys | Ser(tBu)-OtBu | 760 |
| Boc-Lys(εBoc) | His | Asp | Ser(tBu)-OtBu | 761 |
| Boc-Lys(εBoc) | Asp | His | Ser(tBu)-OtBu | 762 |
| Boc-Lys(εBoc) | His | Glu | Ser(tBu)-OtBu | 763 |
| Boc-Lys(εBoc) | Glu | His | Ser(tBu)-OtBu | 764 |

While the peptides of Table 8 are illustrated with particular protecting groups, it is noted that these groups may be substituted with other protecting groups as described herein and/or one or more of the shown protecting group can be eliminated.

4) Small Peptides Having Either an Acidic or Basic Amino Acid in the center together with a central aliphatic amino acid.

In certain embodiments, the peptides of this invention range from four amino acids to about ten amino acids. The terminal amino acids are typically hydrophobic either because of a hydrophobic side chain or because the terminal amino acids bear one or more hydrophobic protecting groups. End amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, Nicotinyl-, etc., and the C-terminus blocked with (tBu)-OtBu, etc.). Typically, the central portion of the peptide comprises a basic or acidic amino acid and an aliphatic amino acid (e.g., in a 4 mer) or a basic domain or an acidic domain and an aliphatic domain in a longer molecule.

These four-mers can be represented by Formula XXV in which X¹ and X⁴ are hydrophobic and/or bear hydrophobic protecting group(s) as described herein and X² is acidic or basic while X³ is aliphatic or X² is aliphatic while X³ is acidic or basic. The peptide can be all L-amino acids or include one, or more, or all D-amino acids.

Certain preferred peptides of this invention include, but are not limited to the peptides shown in Table 9.

TABLE 9
Examples of certain preferred peptides having
either an acidic or basic amino acid in the
center together with a central aliphatic
amino acid.

| | | | | SEQ ID |
|---|---|---|---|---|
| X¹ | X² | X³ | X⁴ | NO |
| Fmoc-Lys(εBoc) | Leu | Arg | Ser(tBu)-OtBu | 765 |
| Fmoc-Lys(εBoc) | Arg | Leu | Ser(tBu)-OtBu | 766 |
| Fmoc-Lys(εBoc) | Leu | Arg | Thr(tBu)-OtBu | 767 |
| Fmoc-Lys(εBoc) | Arg | Leu | Thr(tBu)-OtBu | 768 |
| Fmoc-Lys(εBoc) | Glu | Leu | Ser(tBu)-OtBu | 769 |
| Fmoc-Lys(εBoc) | Leu | Glu | Ser(tBu)-OtBu | 770 |
| Fmoc-Lys(εBoc) | Glu | Leu | Thr(tBu)-OtBu | 771 |
| Fmoc-Lys(εFmoc) | Leu | Arg | Ser(tBu)-OtBu | 772 |
| Fmoc-Lys(εFmoc) | Leu | Arg | Thr(tBu)-OtBu | 773 |
| Fmoc-Lys(εFmoc) | Glu | Leu | Ser(tBu)-OtBu | 774 |
| Fmoc-Lys(εFmoc) | Glu | Leu | Thr(tBu)-OtBu | 775 |
| Boc-Lys(εFmoc) | Glu | Ile | Thr(tBu)-OtBu | 776 |
| Boc-Lys(εFmoc) | Leu | Arg | Ser(tBu)-OtBu | 777 |
| Boc-Lys(εFmoc) | Leu | Arg | Thr(tBu)-OtBu | 778 |
| Boc-Lys(εFmoc) | Glu | Leu | Ser(tBu)-OtBu | 779 |
| Boc-Lys(εFmoc) | Glu | Leu | Thr(tBu)-OtBu | 780 |
| Boc-Lys(εBoc) | Leu | Arg | Ser(tBu)-OtBu | 781 |
| Boc-Lys(εBoc) | Arg | Phe | Thr(tBu)-OtBu | 782 |
| Boc-Lys(εBoc) | Leu | Arg | Thr(tBu)-OtBu | 783 |
| Boc-Lys(εBoc) | Glu | lle | Thr(tBu) | 784 |
| Boc-Lys(εBoc) | Glu | Val | Thr(tBu) | 785 |
| Boc-Lys(εBoc) | Glu | Ala | Thr(tBu) | 786 |
| Boc-Lys(εBoc) | Glu | Gly | Thr(tBu) | 787 |
| Boc--Lys(εBoc) | Glu | Leu | Ser(tBu)-OtBu | 788 |
| Boc-Lys(εBoc) | Glu | Leu | Thr(tBu)-OtBu | 789 |

While the peptides of Table 9 are illustrated with particular protecting groups, it is noted that these groups may be substituted with other protecting groups as described herein and/or one or more of the shown protecting group can be eliminated.

5) Small Peptides Having Either an Acidic or Basic Amino Acid in the Center Together with a Central Aromatic Amino Acid.

In certain embodiments, the “small” peptides of this invention range from four amino acids to about ten amino acids. The terminal amino acids are typically hydrophobic either because of a hydrophobic side chain or because the terminal amino acids bear one or more hydrophobic protecting groups end amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, Nicotinyl-, etc., and the C-terminus blocked with (tBu)-OtBu, etc.). Typically, the central portion of the peptide comprises a basic or acidic amino acid and an aromatic amino acid (e.g., in a 4 mer) or a basic domain or an acidic domain and an aromatic domain in a longer molecule.

These four-mers can be represented by Formula XXV in which X¹ and X⁴ are hydrophobic and/or bear hydrophobic protecting group(s) as described herein and X² is acidic or basic while X³ is aromatic or X² is aromatic while X³ is acidic or basic. The peptide can be all L-amino acids or include one, or more, or all D-amino acids. Five-mers can be represented by a minor modification of Formula XXV in which X⁵ is inserted as shown in Table 10 and in which X⁵ is typically an aromatic amino acid, e.g.,

X¹-X²-X³ₙ-X⁵ₚ-X⁴ XXVII

where X¹, X², X³, and X⁴ are as described above, p is 0 or 1 and X⁵ is typically an aromatic amino acid.

Certain preferred peptides of this invention include, but are not limited to the peptides shown in Table 10.

TABLE 10
Examples of certain preferred peptides having
either an acidic or basic amino acid in the
center together with a central aromatic amino
acid.

| | | | | | SEQ |
|---|---|---|---|---|---|
| | | | | | ID |
| X¹ | X² | X³ | X⁵ | X⁴ | NO |
| Fmoc-Lys(εBoc) | Arg | Trp | | Tyr(tBu)-OtBu | 790 |
| Fmoc-Lys(εBoc) | Trp | Arg | | Tyr(tBu)-OtBu | 791 |
| Fmoc-Lys(εBoc) | Arg | Tyr | | Trp-OtBu | 792 |
| Fmoc-Lys(εBoc) | Tyr | Arg | | Trp-OtBu | 793 |
| Fmoc-Lys(εBoc) | Arg | Tyr | Trp | Thr(tBu)-OtBu | 794 |
| Fmoc-Lys(εBoc) | Arg | Tyr | | Thr(tBu)-OtBu | 795 |
| Fmoc-Lys(εBoc) | Arg | Trp | | Thr(tBu)-OtBu | 796 |
| Fmoc-Lys(εFmoc) | Arg | Trp | | Tyr(tBu)-OtBu | 797 |
| Fmoc-Lys(εFmoc) | Arg | Tyr | | Trp-OtBu | 798 |
| Fmoc-Lys(εFmoc) | Arg | Tyr | Trp | Thr(tBu)-OtBu | 799 |
| Fmoc-Lys(εFmoc) | Arg | Tyr | | Thr(tBu)-OtBu | 800 |
| Fmoc-Lys(εFmoc) | Arg | Trp | | Thr(tBu)-OtBu | 801 |
| Boc-Lys(εFmoc) | Arg | Trp | | Tyr(tBu)-OtBu | 802 |
| Boc-Lys(εFmoc) | Arg | Tyr | | Trp-OtBu | 803 |
| Boc-Lys(εFmoc) | Arg | Tyr | Trp | Thr(tBu)-OtBu | 804 |
| Boc-Lys(εFmoc) | Arg | Tyr | | Thr(tBu)-OtBu | 805 |
| Boc-Lys(εFmoc) | Arg | Trp | | Thr(tBu)-OtBu | 806 |
| Boc-Glu | Lys(εFmoc) | Arg | | Tyr(tBu)-OtBu | 807 |
| Boc-Lys(εBoc) | Arg | Trp | | Tyr(tBu)-OtBu | 808 |
| Boc-Lys(εBoc) | Arg | Tyr | | Trp-OtBu | 809 |
| Boc-Lys(εBoc) | Arg | Tyr | Trp | Thr(tBu)-OtBu | 810 |
| Boc-Lys(εBoc) | Arg | Tyr | | Thr(tBu)-OtBu | 811 |
| Boc-Lys(εBoc) | Arg | Phe | | Thr(tBu)-OtBu | 812 |
| Boc-Lys(εBoc) | Arg | Trp | | Thr(tBu)-OtBu | 813 |

While the peptides of Table 10 are illustrated with particular protecting groups, it is noted that these groups may be substituted with other protecting groups as described herein and/or one or more of the shown protecting groups can be eliminated.

6) Small Peptides Having Aromatic Amino Acids or Aromatic Amino Acids Separated by Histidine(s) at the Center.

In certain embodiments, the peptides of this invention are characterized by π electrons that are exposed in the center of the molecule which allow hydration of the particle and that allow the peptide particles to trap pro-inflammatory oxidized lipids such as fatty acid hydroperoxides and phospholipids that contain an oxidation product of arachidonic acid at the sn-2 position.

In certain embodiments, these peptides consist of a minimum of 4 amino acids and a maximum of about 10 amino acids, preferentially (but not necessarily) with one or more of the amino acids being the D-sterioisomer of the amino acid, with the end amino acids being hydrophobic either because of a hydrophobic side chain or because the terminal amino acid(s) bear one or more hydrophobic blocking group(s), (e.g., an N-terminus blocked with Boc-, Fmoc-, Nicotinyl-, and the like, and a C-terminus blocked with (tBu)-OtBu groups and the like). Instead of having an acidic or basic amino acid in the center, these peptides generally have an aromatic amino acid at the center or have aromatic amino acids separated by histidine in the center of the peptide.

Certain preferred peptides of this invention include, but are not limited to the peptides shown in Table 11.

TABLE 11
Examples of peptides having aromatic amino acids in the
center or aromatic amino acids or aromatic domains separated
by one or more histidines.

| X¹ | X² | X³ | X⁴ | X⁵ | SEQ ID NO |
|---|---|---|---|---|---|
| Boc-Lys(εBoc) | Phe | Trp | Phe | Ser(tBu)-OtBu | 814 |
| Boc-Lys(εBoc) | Phe | Trp | Phe | Thr(tBu)-OtBu | 815 |
| Boc-Lys(εBoc) | Phe | Tyr | Phe | Ser(tBu)-OtBu | 816 |
| Boc-Lys(εBoc) | Phe | Tyr | Phe | Thr(tBu)-OtBu | 817 |
| Boc-Lys(εBoc) | Phe | His | Phe | Ser(tBu)-OtBu | 818 |
| Boc-Lys(εBoc) | Phe | His | Phe | Thr(tBu)-OtBu | 819 |
| Boc-Lys(εBoc) | Val | Phe | Phe-Tyr | Ser(tBu)-OtBu | 820 |
| Nicotinyl-Lys(εBoc) | Phe | Trp | Phe | Ser(tBu)-OtBu | 821 |
| Nicotinyl-Lys(εBoc) | Phe | Trp | Phe | Thr(tBu)-OtBu | 822 |
| Nicotinyl-Lys(εBoc) | Phe | Tyr | Phe | Ser(tBu)-OtBu | 823 |
| Nicotinyl-Lys(εBoc) | Phe | Tyr | Phe | Thr(tBu)-OtBu | 824 |
| Nicotinyl-Lys(εBoc) | Phe | His | Phe | Ser(tBu)-OtBu | 825 |
| Nicotinyl-Lys(εBoc) | Phe | His | Phe | Thr(tBu)-OtBu | 826 |
| Boc-Leu | Phe | Trp | Phe | Thr(tBu)-OtBu | 827 |
| Boc-Leu | Phe | Trp | Phe | Ser(tBu)-OtBu | 828 |

While the peptides of Table 11 are illustrated with particular protecting groups, it is noted that these groups may be substituted with other protecting groups as described herein and/or one or more of the shown protecting group can be eliminated.

7) Summary of Tripeptides and Tetrapeptides.

For the sake of clarity, a number of tripeptides and tetrapeptides of this invention are generally summarized below in Table 12.

TABLE 12
General structure of certain peptides of this invention.

| X¹ | X² | X³ | | X⁴ |
|---|---|---|---|---|
| hydrophobic side chain | Acidic or | — | | hydrophobic side |
| or hydrophobic | Basic | | | chain or |
| protecting group(s) | | | | hydrophobic |
| | | | | protecting group(s) |
| hydrophobic side chain | Basic | Acidic | | hydrophobic side |
| or hydrophobic | | | | chain or |
| protecting group(s) | | | | hydrophobic |
| | | | | protecting group(s) |
| hydrophobic side chain | Acidic | Basic | | hydrophobic side |
| or hydrophobic | | | | chain or |
| protecting group(s) | | | | hydrophobic |
| | | | | protecting group(s) |
| hydrophobic side chain | Acidic or | Aliphatic | | hydrophobic side |
| or hydrophobic | Basic | | | chain or |
| protecting group(s) | | | | hydrophobic |
| | | | | protecting group(s) |
| hydrophobic side chain | Aliphatic | Acidic or | | hydrophobic side |
| or hydrophobic | | Basic | | chain or |
| protecting group(s) | | | | hydrophobic |
| | | | | protecting group(s) |
| hydrophobic side chain | Acidic or | Aromatic | | hydrophobic side |
| or hydrophobic | Basic | | | chain or |
| protecting group(s) | | | | hydrophobic |
| | | | | protecting group(s) |
| hydrophobic side chain | Aromatic | Acidic or | | hydrophobic side |
| or hydrophobic | | Basic | | chain or |
| protecting group(s) | | | | hydrophobic |
| | | | | protecting group(s) |
| hydrophobic side chain | Aromatic | His | Aromatic | hydrophobic side |
| or hydrophobic | | | | chain or |
| protecting group(s) | | | | hydrophobic |
| | | | | protecting group(s) |

Where longer peptides are desired, X² and X³ can represent domains (e.g., regions of two or more amino acids of the specified type) rather than individual amino acids. Table 12 is intended to be illustrative and not limiting. Using the teaching provided herein, other suitable peptides can readily be identified.

8) Paired Amino Acids and Dipeptides.

In certain embodiments, this invention pertains to the discovery that certain pairs of amino acids, administered in conjunction with each other or linked to form a dipeptide have one or more of the properties described herein. Thus, without being bound to a particular theory, it is believed that when the pairs of amino acids are administered in conjunction with each other, as described herein, they are capable participating in or inducing the formation of micelles in vivo.

Similar to the other small peptides described herein, it is believed that the pairs of peptides will associate in vivo, and demonstrate physical properties including high solubility in ethyl acetate (e.g., greater than about 4 mg/mL), solubility in aqueous buffer at pH 7.0. Upon contacting phospholipids such as 1,2-Dimyristoyl-sn-glycero-3-phosphocholine (DMPC), in an aqueous environment, it is believed the pairs of amino acids induce or participate in the formation of particles with a diameter of approximately 7.5 nm (±0.1 nm), and/or induce or participate in the formation of stacked bilayers with a bilayer dimension on the order of 3.4 to 4.1 nm with spacing between the bilayers in the stack of approximately 2 nm, and/or also induce or participate in the formation of vesicular structures of approximately 38 nm).

Moreover, it is further believed that the pairs of amino acids can display one or more of the following physiologically relevant properties:
1. They convert pro-inflammatory HDL to anti-inflammatory HDL or make anti-inflammatory HDL more anti-inflammatory;
2. They decrease LDL-induced monocyte chemotactic activity generated by artery wall cells;
3. They stimulate the formation and cycling of pre-β HDL;
4. They raise HDL cholesterol; and/or
5. They increase HDL paraoxonase activity.

The pairs of amino acids can be administered as separate amino acids (administered sequentially or simultaneously, e.g., in a combined formulation) or they can be covalently coupled directly or through a linker (e.g., a PEG linker, a carbon linker, a branched linker, a straight chain linker, a heterocyclic linker, a linker formed of derivatized lipid, etc.). In certain embodiments, the pairs of amino acids are covalently linked through a peptide bond to form a dipeptide. In various embodiments while the dipeptides will typically comprise two amino acids each bearing an attached protecting group, this invention also contemplates dipeptides wherein only one of the amino acids bears one or more protecting groups.

The pairs of amino acids typically comprise amino acids where each amino acid is attached to at least one protecting group (e.g., a hydrophobic protecting group as described herein). The amino acids can be in the D or the L form. In certain embodiments, where the amino acids comprising the pairs are not attached to each other, each amino acid bears two protecting groups (e.g., such as molecules 1 and 2 in Table 13).

TABLE 13
Illustrative amino acid pairs of this invention.

| | Amino Acid Pair/dipeptide |
|---|---|
| 1. | Boc-Arg-OtBu* |
| 2. | Boc-Glu-OtBu* |
| 3. | Boc-Phe-Arg-OtBu** |
| 4. | Boc-Glu-Leu-OtBu** |
| 5. | Boc-Arg-Glu-OtBu*** |

*This would typically be administered in conjunction with a second amino acid.
**In certain embodiments, these dipeptides would be administered in conjunction with each other.
***In certain embodiments, this peptide would be administered either alone or in combination with one of the other peptides described herein..

Suitable pairs of amino acids can readily be identified by providing the pair of protected amino acids and/or a dipeptide and then screening the pair of amino acids/dipeptide for one or more of the physical and/or physiological properties described above. In certain embodiments, this invention excludes pairs of amino acids and/or dipeptides comprising aspartic acid and phenylalanine. In certain embodiments, this invention excludes pairs of amino acids and/or dipeptides in which one amino acid is (−)-N-[(trans-4-isopropylcyclohexane)carbonyl]-D-phenylalanine (nateglinide).

In certain embodiments, the amino acids comprising the pair are independently selected from the group consisting of an acidic amino acid (e.g., aspartic acid, glutamic acid, etc.), a basic amino acid (e.g., lysine, arginine, histidine, etc.), and a non-polar amino acid (e.g., alanine, valine, leucine, isoleucine, proline, phenylalanine, tryptophan, methionine, etc.). In certain embodiments, where the first amino acid is acidic or basic, the second amino acid is non-polar and where the second amino acid is acidic or basic, the first amino acid is non-polar. In certain embodiments, where the first amino acid is acidic, the second amino acid is basic, and vice versa. (see, e.g., Table 14).

Similar combinations can be obtained by administering pairs of dipeptides. Thus, for example in certain embodiments, molecules 3 and 4 in Table 13 would be administered in conjunction with each other.

TABLE 14
Certain generalized amino acid pairs/dipeptides.

| | First Amino acid | Second Amino acid |
|---|---|---|
| 1. | Acidic | Basic |
| 2. | Basic | Acidic |
| 3. | Acidic | Non-polar |
| 4. | Non-polar | Acidic |
| 5. | Basic | Non-polar |
| 6. | Non-polar | Basic |

It is noted that these amino acid pairs/dipeptides are intended to be illustrative and not limiting. Using the teaching provided herein other suitable amino acid pairs/dipeptides can readily be determined.

In certain embodiments, however, dipeptides and/or amino acid pairs comprising L-Glu-L-Trp, e.g., as described in U.S. Pat. No. 5,807,830 and/or any other peptides disclosed in this patent, are expressly excluded from the methods and/or formulations described herein.

E) Apo-J (G* Peptides).

It was also a discovery of this invention that peptides that mimicking the amphipathic helical domains of apo J are capable of mitigating one or more symptoms of atherosclerosis and/or other pathologies described herein. Apolipoprotein J possesses a wide nonpolar face termed globular protein-like, or G* amphipathic helical domains. The class G amphipathic helix is found in globular proteins, and thus, the name class G. This class of amphipathic helix is characterized by a random distribution of positively charged and negatively charged residues on the polar face with a narrow nonpolar face. Because of the narrow nonpolar face this class does not readily associate with phospholipids. The G* of amphipathic helix possesses similar, but not identical, characteristics to the G amphipathic helix. Similar to the class G amphipathic helix, the G* class peptides possesses a random distribution of positively and negatively charged residues on the polar face. However, in contrast to the class G amphipathic helix which has a narrow nonpolar face, this class has a wide nonpolar face that allows this class to readily bind phospholipid and the class is termed G* to differentiate it from the G class of amphipathic helix.

A number of suitable G* amphipathic peptides are described in copending applications U.S. Ser. No. 10/120,508, filed Apr. 5, 2002, U.S. Ser. No. 10/520,207, filed Apr. 1, 2003, and PCT Application PCT/US03/09988, filed Apr. 1, 2003. In addition, a variety of suitable peptides of this invention that are related to G* amphipathic helical domains of apo J are illustrated in Table 15.

TABLE 15
Certain peptides for use in this invention related
to G* amphipathic helical domains of apo J.

| Amino Acid Sequence | SEQ ID NO |
|---|---|
| LLEQLNEQFNWVSRLANLTQGE | 829 |
| LLEQLNEQFNWVSRLANL | 830 |
| NELQEMSNQGSKYVNKEIQNAVNGV | 831 |
| IQNAVNGVKQIKTLIEKTNEE | 832 |
| RKTLLSNLEEAKKKKEDALNETRESETKLKEL | 833 |
| PGVCNETMMALWEECK | 834 |
| PCLKQTCMKFYARVCR | 835 |
| ECKPCLKQTCMKFYARVCR | 836 |
| LVGRQLEEFL | 837 |
| MNGDRIDSLLEN | 838 |
| QQTHMLDVMQD | 839 |
| FSRASSIIDELFQD | 840 |
| PFLEMIHEAQQAMDI | 841 |
| PTEFIREGDDD | 842 |
| RMKDQCDKCREILSV | 843 |
| PSQAKLRRELDESLQVAERLTRKYNELLKSYQ | 844 |
| LLEQLNEQFNWVSRLANLTEGE | 845 |
| DQYYLRVTTVA | 846 |
| PSGVTEVVVKLFDS | 847 |
| PKFMETVAEKALQEYRKKHRE | 848 |

The peptides of this invention, however, are not limited to G* variants of apo J. Generally speaking G* domains from essentially any other protein preferably apo proteins are also suitable. The particular suitability of such proteins can readily be determined using assays for protective activity (e.g., protecting LDL from oxidation, and the like), e.g., as illustrated herein in the Examples. Some particularly preferred proteins include G* amphipathic helical domains or variants thereof (e.g., conservative substitutions, and the like) of proteins including, but not limited to apo AI, apo AIV, apo E, apo CII, apo CIII, and the like.

Certain preferred peptides for related to G* amphipathic helical domains related to apoproteins other than apo J are illustrated in Table 16.

TABLE 16
Certain peptides for use in this invention
related to G* amphipathic helical domains
related to apoproteins other than apo J.

| | SEQ ID |
|---|---|
| Amino Acid Sequence | NO |
| WDRVKDLATVYVDVLKDSGRDYVSQF | 849 |
| (Related to the 8 to 33 region of apo AI) | |
| VATVMWDYFSQLSNNAKEAVEHLQK | 850 |
| (Related to the 7 to 31 region of apo AIV) | |
| RWELALGRFWDYLRWVQTLSEQVQEEL | 851 |
| (Related to the 25 to 51 region of apo E) | |
| LSSQVTQELRALMDETMKELKELKAYKSELEEQLT | 852 |
| (Related to the 52 to 83 region of apo E) | |
| ARLSKELQAAQARLGADMEDVCGRLV | 853 |
| (Related to the 91 to 116 region of apo E) | |
| VRLASHLRKLRKRLLRDADDLQKRLA | 854 |
| (Related to the 135 to 160 region of apo E) | |
| PLVEDMQRQWAGLVEKVQA | 855 |
| (267 to 285 of apo E. 27) | |
| MSTYTGIFTDQVLSVLK | 856 |
| (Related to the 60 to 76 region of apo CII) | |
| LLSFMQGYMKHATKTAKDALSS | 857 |
| (Related to the 8 to 29 region of apo CIII) | |

Additional illustrative G* peptides are shown in Table 17.

TABLE 17
Additional illustrative G* peptides.

| | SEQ ID |
|---|---|
| Peptide | NO |
| Ac-Lys-Trp-Ile-Tyr-His-Leu-Thr-Glu-Gly- | 858 |
| Ser-Thr-Asp-Leu-Arg-Thr-Glu-Gly-NH₂ | |
| Ac-Lys-Trp-Phe-Tyr-His-Leu-Thr-Glu-Gly- | 859 |
| Ser-Thr-Asp-Leu-Arg-Thr-Glu-Gly-NH₂ | |
| Ac-Lys-Trp-Leu-Tyr-His-Leu-Thr-Glu-Gly- | 860 |
| Ser-Thr-Asp-Leu-Arg-Thr-Glu-Gly-NH₂ | |
| Ac-Lys-Trp-Val-Tyr-His-Leu-Thr-Glu-Gly- | 861 |
| Ser-Thr-Asp-Leu-Arg-Thr-Glu-Gly-NH₂ | |
| Ac-Lys-Tyr-Ile-Trp-His-Leu-Thr-Glu-Gly- | 862 |
| Ser-Thr-Asp-Leu-Arg-Thr-Glu-Gly-NH₂ | |
| Ac-Lys-Trp-Ile-Tyr-His-Phe-Thr-Glu-Gly- | 863 |
| Ser-Thr-Asp-Leu-Arg-Thr-Glu-Gly-NH₂ | |
| Ac-Lys-Trp-Phe-Tyr-His-Ile-Thr-Glu-Gly- | 864 |
| Ser-Thr-Asp-Leu-Arg-Thr-Glu-Gly-NH₂ | |
| Ac-Lys-Trp-Leu-Tyr-His-Val-Thr-Glu-Gly- | 865 |
| Ser-Thr-Asp-Leu-Arg-Thr-Glu-Gly-NH₂ | |
| Ac-Lys-Trp-Val-Tyr-His-Tyr-Thr-Glu-Gly- | 866 |
| Ser-Thr-Asp-Leu-Arg-Thr-Glu-Gly-NH₂ | |
| Ac-Lys-Tyr-Ile-Trp-His-Phe-Thr-Glu-Gly- | 867 |
| Ser-Thr-Asp-Leu-Arg-Thr-Glu-Gly-NH₂ | |
| Ac-Lys-Tyr-Ile-Trp-His-Ile-Thr-Glu-Gly- | 868 |
| Ser-Thr-Asp-Leu-Arg-Thr-Glu-Gly-NH₂ | |
| Ac-Lys-Tyr-Ile-Trp-His-Val-Thr-Glu-Gly- | 869 |
| Ser-Thr-Asp-Leu-Arg-Thr-Glu-Gly-NH₂ | |
| Ac-Lys-Tyr-Ile-Trp-His-Tyr-Thr-Glu-Gly- | 870 |
| Ser-Thr-Asp-Leu-Arg-Thr-Glu-Gly-NH₂ | |
| Ac-Lys-Phe-Ile-Trp-His-Leu-Thr-Glu-Gly- | 871 |
| Ser-Thr-Asp-Leu-Arg-Thr-Glu-Gly-NH₂ | |
| Ac-Lys-Leu-Ile-Trp-His-Leu-Thr-Glu-Gly- | 872 |
| Ser-Thr-Asp-Leu-Arg-Thr-Glu-Gly-NH₂ | |
| Ac-Lys-Ile-Ile-Trp-His-Leu-Thr-Glu-Gly- | 873 |
| Ser-Thr-Asp-Leu-Arg-Thr-Glu-Gly-NH₂ | |
| Ac-Lys-Tyr-Ile-Trp-Phe-Leu-Thr-Glu-Gly- | 874 |
| Ser-Thr-Asp-Leu-Arg-Thr-Glu-Gly-NH₂ | |
| Ac-Lys-Trp-Ile-Tyr-Phe-Leu-Thr-Glu-Gly- | 875 |
| Ser-Thr-Asp-Leu-Arg-Thr-Glu-Gly-NH₂ | |
| Ac-Lys-Trp-Ile-Tyr-Leu-Leu-Thr-Glu-Gly- | 876 |
| Ser-Thr-Asp-Leu-Arg-Thr-Glu-Gly-NH₂ | |
| Ac-Lys-Trp-Ile-Tyr-His-Phe-Thr-Glu-Gly- | 877 |
| Ser-Thr-Asp-Leu-Arg-Thr-Glu-Gly-NH₂ | |
| Ac-Lys-Trp-Ile-Tyr-His-Tyr-Thr-Glu-Gly- | 878 |
| Ser-Thr-Asp-Leu-Arg-Thr-Glu-Gly-NH₂ | |
| Ac-Lys-Trp-Ile-Tyr-His-Ile-Thr-Glu-Gly- | 879 |
| Ser-Thr-Asp-Leu-Arg-Thr-Glu-Gly-NH₂ | |
| Ac-Lys-Trp-Ile-Tyr-His-Leu-Ser-Glu-Gly- | 880 |
| Ser-Thr-Asp-Leu-Arg-Thr-Glu-Gly-NH₂ | |
| Ac-Lys-Trp-Ile-Tyr-His-Leu-Thr-Asp-Gly- | 881 |
| Ser-Thr-Asp-Leu-Arg-Thr-Glu-Gly-NH₂ | |
| Ac-Lys-Trp-Ile-Tyr-His-Leu-Thr-Glu-Gly- | 882 |
| Thr-Ser-Asp-Leu-Arg-Thr-Glu-Gly-NH₂ | |
| Ac-Lys-Trp-Ile-Tyr-His-Leu-Thr-Glu-Gly- | 883 |
| Ser-Thr-Glu-Leu-Arg-Thr-Glu-Gly-NH₂ | |
| Ac-Lys-Trp-Ile-Tyr-His-Leu-Thr-Glu-Gly- | 884 |
| Ser-Thr-Asp-Phe-Arg-Thr-Glu-Gly-NH₂ | |
| Ac-Lys-Trp-Ile-Tyr-His-Leu-Thr-Glu-Gly- | 885 |
| Ser-Thr-Asp-Tyr-Arg-Thr-Glu-Gly-NH₂ | |
| Ac-Lys-Trp-Ile-Tyr-His-Leu-Thr-Glu-Gly- | 886 |
| Ser-Thr-Asp-Ile-Arg-Thr-Glu-Gly-NH₂ | |
| Ac-Lys-Trp-Ile-Tyr-His-Leu-Thr-Glu-Gly- | 887 |
| Ser-Thr-Asp-Val-Arg-Thr-Glu-Gly-NH₂ | |
| Ac-Lys-Trp-Ile-Tyr-His-Leu-Thr-Glu-Gly- | 888 |
| Ser-Thr-Asp-Leu-Lys-Thr-Glu-Gly-NH₂ | |
| Ac-Lys-Trp-Ile-Tyr-His-Leu-Thr-Glu-Gly- | 889 |
| Ser-Thr-Asp-Leu-Arg-Ser-Glu-Gly-NH₂ | |
| Ac-Lys-Trp-Ile-Tyr-His-Leu-Thr-Glu-Gly- | 890 |
| Ser-Thr-Asp-Leu-Arg-Thr-Asp-Gly-NH₂ | |
| Ac-Lys-Trp-Ile-Tyr-His-Leu-Thr-Glu-Gly- | 891 |
| Ser-Thr-Asp-Ile-Lys-Thr-Glu-Gly-NH₂ | |
| Ac-Lys-Trp-Ile-Tyr-His-Leu-Thr-Glu-Gly- | 892 |
| Ser-Thr-Asp-Ile-Arg-Ser-Glu-Gly-NH₂ | |
| Ac-Lys-Trp-Ile-Tyr-His-Leu-Thr-Glu-Gly- | 893 |
| Ser-Thr-Asp-Ile-Lys-Ser-Glu-Gly-NH₂ | |
| Ac-Lys-Trp-Ile-Tyr-His-Leu-Thr-Glu-Gly- | 894 |
| Ser-Thr-Asp-Ile-Lys-Ser-Asp-Gly-NH₂ | |
| Ac-Arg-Trp-Ile-Tyr-His-Leu-Thr-Glu-Gly- | 895 |
| Ser-Thr-Asp-Leu-Arg-Thr-Glu-Gly-NH₂ | |
| Ac-Arg-Tyr-Ile-Trp-His-Leu-Thr-Glu-Gly- | 896 |
| Ser-Thr-Asp-Ile-Arg-Thr-Glu-Gly-NH₂ | |
| Ac-Arg-Trp-Ile-Tyr-His-Leu-Thr-Glu-Gly- | 897 |
| Ser-Thr-Asp-Ile-Arg-Thr-Asp-Gly-NH₂ | |
| Ac-Arg-Trp-Ile-Phe-His-Leu-Thr-Glu-Gly- | 898 |
| Ser-Thr-Asp-Ile-Arg-Thr-Glu-Gly-NH₂ | |
| Ac-Arg-Trp-Ile-Tyr-His-Leu-Thr-Glu-Gly- | 899 |
| Ser-Thr-Asp-Leu-Lys-Thr-Glu-Gly-NH₂ | |
| Ac-Arg-Trp-Ile-Tyr-His-Leu-Thr-Asp-Gly- | 900 |
| Ser-Thr-Asp-Ile-Arg-Thr-Glu-Gly-NH₂ | |
| Ac-Arg-Trp-Ile-Tyr-His-Leu-Thr-Asp-Gly- | 901 |
| Ser-Thr-Asp-Leu-Arg-Thr-Glu-Gly-NH₂ | |
| Ac-Arg-Trp-Ile-Tyr-Phe-Leu-Thr-Glu-Gly- | 902 |
| Ser-Thr-Asp-Ile-Arg-Thr-Glu-Gly-NH₂ | |
| Ac-Arg-Trp-Ile-Tyr-Phe-Leu-Thr-Glu-Gly- | 903 |
| Ser-Thr-Asp-Leu-Arg-Thr-Glu-Gly-NH₂ | |
| Ac-Lys-Trp-Phe-Tyr-His-Leu-Thr-Glu-Gly- | 904 |
| Ser-Thr-Asp-Phe-Arg-Thr-Glu-Gly-NH₂ | |
| Ac-Arg-Trp-Phe-Tyr-His-Leu-Thr-Glu-Gly- | 905 |
| Ser-Thr-Asp-Leu-Arg-Thr-Glu-Gly-NH₂ | |
| Ac-Lys-Trp-Ile-Phe-His-Leu-Thr-Glu-Gly- | 906 |
| Ser-Thr-Asp-Ile-Arg-Thr-Asp-Gly-NH₂ | |
| Ac-Arg-Trp-Ile-Tyr-His-Leu-Thr-Glu-Gly- | 907 |
| Ser-Thr-Asp-Ile-Arg-Thr-Asp-Gly-NH₂ | |
| Ac-Arg-Trp-Ile-Tyr-His-Leu-Thr-Glu-Gly- | 908 |
| Ser-Thr-Asp-Leu-Arg-Thr-Asp-Gly-NH₂ | |
| Ac-Lys-Trp-Ile-Tyr-His-Leu-Thr-Glu-Gly- | 909 |
| Ser-Thr-Asp-Ile-Lys-Thr-Glu-Gly-NH₂ | |
| Ac-Lys-Trp-Ile-Tyr-His-Leu-Thr-Glu-Gly- | 910 |
| Ser-Thr-Asp-Ile-Lys-Thr-Asp-Gly-NH₂ | |
| Ac-Lys-Trp-Ile-Tyr-His-Leu-Thr-Glu-Gly- | 911 |
| Ser-Thr-Asp-Phe-Lys-Thr-Glu-Gly-NH₂ | |
| Ac-Lys-Trp-Ile-Tyr-His-Leu-Thr-Glu-Gly- | 912 |
| Ser-Thr-Asp-Tyr-Lys-Thr-Glu-Gly-NH₂ | |
| Ac-Lys-Trp-Ile-Tyr-His-Leu-Thr-Glu-Gly- | 913 |
| Ser-Thr-Asp-Ile-Arg-Thr-Glu-Gly-NH₂ | |
| Ac-Lys-Trp-Phe-Tyr-His-Phe-Thr-Glu-Gly- | 914 |
| Ser-Thr-Asp-Leu-Arg-Thr-Glu-Gly-NH₂ | |
| Ac-Arg-Trp-Phe-Tyr-His-Phe-Thr-Glu-Gly- | 915 |
| Ser-Thr-Asp-Leu-Arg-Thr-Glu-Gly-NH₂ | |
| Ac-Lys-Trp-Phe-Tyr-His-Phe-Thr-Glu-Gly- | 916 |
| Ser-Thr-Asp-Phe-Arg-Thr-Glu-Gly-NH₂ | |
| Ac-Lys-Trp-Phe-Tyr-His-Phe-Thr-Asp-Gly- | 917 |
| Ser-Thr-Asp-Ile-Arg-Thr-Glu-Gly-NH₂ | |
| Ac-Arg-Trp-Phe-Tyr-His-Phe-Thr-Glu-Gly- | 918 |
| Ser-Thr-Asp-Leu-Arg-Thr-Glu-Gly-NH₂ | |
| Ac-Arg-Trp-Phe-Tyr-His-Phe-Thr-Glu-Gly- | 919 |
| Ser-Thr-Asp-Phe-Arg-Thr-Glu-Gly-NH₂ | |
| Ac-Arg-Trp-Phe-Tyr-His-Phe-Thr-Glu-Gly- | 920 |
| Ser-Thr-Asp-Phe-Arg-Thr-Asp-Gly-NH₂ | |
| Ac-Glu-Lys-Cys-Val-Glu-Glu-Phe-Lys-Ser- | 921 |
| Leu-Thr-Ser-Cys-Leu-Asp-Ser-Lys-Ala- | |
| Phe-NH₂ | |
| Ac-Asp-Lys-Cys-Val-Glu-Glu-Phe-Lys-Ser- | 922 |
| Leu-Thr-Ser-Cys-Leu-Asp-Ser-Lys-Ala- | |
| Phe-NH₂ | |
| Ac-Glu-Lys-Cys-Val-Asp-Glu-Phe-Lys-Ser- | 923 |
| Leu-Thr-Ser-Cys-Leu-Asp-Ser-Lys-Ala- | |
| Phe-NH₂ | |
| Ac-Glu-Lys-Cys-Val-Glu-Asp-Phe-Lys-Ser- | 924 |
| Leu-Thr-Ser-Cys-Leu-Asp-Ser-Lys-Ala- | |
| Phe-NH₂ | |
| Ac-Glu-Arg-Cys-Val-Glu-Glu-Phe-Lys-Ser- | 925 |
| Leu-Thr-Ser-Cys-Leu-Asp-Ser-Lys-Ala- | |
| Phe-NH₂ | |
| Ac-Asp-Lys-Cys-Val-Asp-Asp-Phe-Lys-Ser- | 926 |
| Leu-Thr-Ser-Cys-Leu-Asp-Ser-Lys-Ala- | |
| Phe-NH₂ | |
| Ac-Asp-Arg-Cys-Val-Glu-Glu-Phe-Lys-Ser- | 927 |
| Leu-Thr-Ser-Cys-Leu-Asp-Ser-Lys-Ala- | |
| Phe-NH₂ | |
| Ac-Glu-Arg-Cys-Val-Asp-Asp-Phe-Lys-Ser- | 928 |
| Leu-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 929 |
| Phe-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 930 |
| Ile-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 931 |
| Val-Thr-Ser-Cys-Leu-Asp-Ser-Lys-Ala- | |
| Phe-NH₂ | |
| Ac-Glu-Arg-Cys-Val-Glu-Glu-Phe-Lys-Ser- | 932 |
| Tyr-Thr-Ser-Cys-Leu-Asp-Ser-Lys-Ala- | |
| Phe-NH₂ | |
| Ac-Glu-Arg-Cys-Val-Glu-Glu-Phe-Lys-Ser- | 933 |
| Phe-Thr-Ser-Cys-Leu-Asp-Ser-Lys-Ala- | |
| Phe-NH₂ | |
| Ac-Glu-Arg-Cys-Val-Glu-Glu-Phe-Lys-Ser- | 934 |
| Ile-Thr-Ser-Cys-Leu-Asp-Ser-Lys-Ala- | |
| Phe-NH₂ | |
| Ac-Glu-Arg-Cys-Val-Glu-Glu-Phe-Lys-Ser- | 935 |
| Val-Thr-Ser-Cys-Leu-Asp-Ser-Lys-Ala- | |
| Phe-NH₂ | |
| Ac-Glu-Arg-Cys-Val-Glu-Glu-Phe-Lys-Ser- | 936 |
| Tyr-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 937 |
| Phe-Thr-Thr-Cys-Leu-Asp-Ser-Lys-Ala- | |
| Phe-NH₂ | |
| Ac-Glu-Lys-Cys-Val-Glu-Glu-Phe-Lys-Ser- | 938 |
| Ile-Ser-Ser-Cys-Leu-Asp-Ser-Lys-Ala- | |
| Phe-NH₂ | |
| Ac-Glu-Lys-Cys-Val-Glu-Glu-Phe-Lys-Ser- | 939 |
| Val-Ser-Thr-Cys-Leu-Asp-Ser-Lys-Ala- | |
| Phe-NH₂ | |
| Ac-Glu-Lys-Cys-Val-Glu-Glu-Phe-Lys-Ser- | 940 |
| Tyr-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 941 |
| Phe-Thr-Thr-Cys-Leu-Asp-Ser-Lys-Ala- | |
| Phe-NH₂ | |
| Ac-Glu-Lys-Cys-Val-Glu-Glu-Phe-Lys-Ser- | 942 |
| Phe-Ser-Ser-Cys-Leu-Asp-Ser-Lys-Ala- | |
| Phe-NH₂ | |
| Ac-Glu-Lys-Cys-Val-Glu-Glu-Phe-Lys-Ser- | 943 |
| Phe-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 944 |
| Phe-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 945 |
| Phe-Thr-Ser-Cys-Leu-Asp-Ser-Lys-Ala- | |
| Phe-NH₂ | |
| Ac-Glu-Lys-Cys-Val-Glu-Glu-Phe-Lys-Ser- | 946 |
| Phe-Thr-Ser-Cys-Phe-Asp-Ser-Lys-Ala- | |
| Phe-NH₂ | |
| Ac-Glu-Lys-Cys-Val-Glu-Glu-Phe-Lys-Ser- | 947 |
| Phe-Thr-Ser-Cys-Phe-Glu-Ser-Lys-Ala- | |
| Phe-NH₂ | |
| Ac-Glu-Lys-Cys-Val-Glu-Glu-Phe-Lys-Ser- | 948 |
| Phe-Thr-Ser-Cys-Leu-Glu-Ser-Lys-Ala- | |
| Phe-NH₂ | |
| Ac-Glu-Lys-Cys-Val-Glu-Glu-Phe-Lys-Ser- | 949 |
| Phe-Thr-Ser-Cys-Ile-Asp-Ser-Lys-Ala- | |
| Phe-NH₂ | |
| Ac-Glu-Lys-Cys-Val-Glu-Glu-Leu-Lys-Ser- | 950 |
| Phe-Thr-Ser-Cys-Phe-Asp-Ser-Lys-Ala- | |
| Phe-NH₂ | |
| Ac-Asp-Lys-Cys-Val-Glu-Glu-Phe-Lys-Ser- | 951 |
| Phe-Thr-Ser-Cys-Phe-Asp-Ser-Lys-Ala- | |
| Phe-NH₂ | |
| Ac-Asp-Lys-Cys-Val-Glu-Glu-Phe-Lys-Ser- | 952 |
| Phe-Thr-Ser-Cys-Phe-Glu-Ser-Lys-Ala- | |
| Phe-NH₂ | |
| Ac-Glu-Arg-Cys-Val-Glu-Glu-Phe-Lys-Ser- | 953 |
| Phe-Thr-Ser-Cys-Phe-Asp-Ser-Lys-Ala- | |
| Phe-NH₂ | |
| Ac-Glu-Lys-Cys-Phe-Glu-Glu-Phe-Lys-Ser- | 954 |
| Phe-Thr-Ser-Cys-Phe-Asp-Ser-Lys-Ala- | |
| Phe-NH₂ | |
| Ac-Glu-Lys-Cys-Phe-Glu-Glu-Phe-Lys-Ser- | 955 |
| Phe-Thr-Ser-Cys-Phe-Glu-Ser-Lys-Ala- | |
| Phe-NH₂ | |
| Ac-Glu-Lys-Cys-Val-Glu-Glu-Phe-Lys-Ser- | 956 |
| Phe-Ser-Ser-Cys-Phe-Glu-Ser-Lys-Ala- | |
| Phe-NH₂ | |
| Ac-Glu-Lys-Cys-Val-Glu-Glu-Phe-Lys-Ser- | 957 |
| Phe-Gln-Ser-Cys-Phe-Asp-Ser-Lys-Ala- | |
| Phe-NH₂ | |
| Ac-Glu-Lys-Cys-Phe-Glu-Glu-Phe-Lys-Ser- | 958 |
| Phe-Gln-Ser-Cys-Phe-Asp-Ser-Lys-Ala- | |
| Phe-NH₂ | |
| Ac-Glu-Lys-Cys-Val-Glu-Glu-Phe-Lys-Gln- | 959 |
| Phe-Thr-Ser-Cys-Phe-Asp-Ser-Lys-Ala- | |
| Phe-NH₂ | |
| Ac-Glu-Lys-Cys-Val-Glu-Glu-Phe-Lys-Gln- | 960 |
| Leu-Thr-Ser-Cys-Leu-Asp-Ser-Lys-Ala- | |
| Phe-NH₂ | |
| Ac-Glu-Lys-Cys-Phe-Glu-Glu-Phe-Lys-Ser- | 961 |
| Phe-Gln-Ser-Cys-Leu-Asp-Ser-Lys-Ala- | |
| Phe-NH₂ | |
| Ac-Glu-Lys-Cys-Val-Glu-Glu-Phe-Lys-Gln- | 962 |
| Phe-Thr-Ser-Cys-Phe-Asp-Ser-Lys-Ala- | |
| Phe-NH₂ | |
| Ac-Glu-Lys-Cys-Val-Glu-Glu-Phe-Lys-Ser- | 963 |
| Phe-Thr-Ser-Cys-Phe-Glu-Ser-Lys-Ala- | |
| Phe-NH₂ | |
| Ac-Glu-Arg-Cys-Phe-Glu-Glu-Phe-Lys-Ser- | 964 |
| Phe-Thr-Ser-Cys-Phe-Asp-Ser-Lys-Ala- | |
| Phe-NH₂ | |
| Ac-Asp-Lys-Cys-Phe-Glu-Glu-Phe-Lys-Ser- | 965 |
| Phe-Thr-Ser-Cys-Phe-Asp-Ser-Lys-Ala- | |
| Phe-NH₂ | |
| Ac-Glu-Arg-Cys-Val-Glu-Glu-Phe-Lys-Ser- | 966 |
| Leu-Thr-Ser-Cys-Leu-Glu-Ser-Lys-Ala- | |
| Phe-NH₂ | |
| Ac-Glu-Lys-Cys-Val-Glu-Glu-Phe-Lys-Ser- | 967 |
| Leu-Thr-Ser-Cys-Leu-Asp-Ser-Lys-Phe- | |
| Phe-NH₂ | |
| Ac-Glu-Lys-Cys-Phe-Glu-Glu-Phe-Lys-Ser- | 968 |
| Phe-Thr-Ser-Cys-Phe-Asp-Ser-Lys-Phe- | |
| Phe-NH₂ | |
| Ac-Asp-Lys-Cys-Phe-Glu-Glu-Phe-Lys-Ser- | 969 |
| Phe-Thr-Ser-Cys-Leu-Asp-Ser-Lys-Phe- | |
| Phe-NH₂ | |
| Ac-Asp-Lys-Cys-Phe-Glu-Glu-Phe-Lys-Ser- | 970 |
| Phe-Thr-Ser-Cys-Leu-Glu-Ser-Lys-Phe- | |
| Phe-NH₂ | |
| Ac-Asp-Lys-Cys-Phe-Glu-Glu-Leu-Lys-Ser- | 971 |
| Phe-Thr-Ser-Cys-Leu-Asp-Ser-Lys-Phe- | |
| Phe-NH₂ | |
| Ac-Glu-Arg-Cys-Phe-Glu-Glu-Phe-Lys-Ser- | 972 |
| Phe-Thr-Ser-Cys-Leu-Asp-Ser-Lys-Phe- | |
| Phe-NH₂ | |
| Ac-Glu-Lys-Ala-Val-Glu-Glu-Phe-Lys-Ser- | 973 |
| Phe-Thr-Ser-Cys-Leu-Asp-Ser-Lys-Ala- | |
| Phe-NH₂ | |
| Ac-Asp-Lys-Ala-Val-Glu-Glu-Phe-Lys-Ser- | 974 |
| Phe-Thr-Ser-Cys-Leu-Asp-Ser-Lys-Phe- | |
| Phe-NH₂ | |
| Ac-Glu-Lys-Ala-Val-Glu-Glu-Phe-Lys-Ser- | 975 |
| Phe-Thr-Ser-Ala-Leu-Asp-Ser-Lys-Ala- | |
| Phe-NH₂ | |
| Ac-Asp-Lys-Ala-Val-Glu-Glu-Phe-Lys-Ser- | 976 |
| Phe-Thr-Ser-Ala-Leu-Asp-Ser-Lys-Ala- | |
| Phe-NH₂ | |
| Ac-Asp-Arg-Ala-Phe-Glu-Glu-Phe-Lys-Ser- | 977 |
| Phe-Thr-Ser-Cys-Leu-Asp-Ser-Lys-Phe- | |
| Phe-NH₂ | |
| Ac-Asp-Arg-Ala-Phe-Glu-Glu-Phe-Lys-Ser- | 978 |
| Phe-Thr-Ser-Ala-Leu-Asp-Ser-Lys-Phe- | |
| Phe-NH₂ | |
| Ac-Asp-Lys-Cys-Phe-Glu-Glu-Phe-Lys-Ser- | 979 |
| Phe-Thr-Ser-Cys-Phe-Glu-Ser-Lys-Phe- | |
| Phe-NH₂ | |
| Ac-Glu-Lys-Cys-Tyr-Glu-Glu-Phe-Lys-Ser- | 980 |
| Phe-Thr-Ser-Cys-Leu-Asp-Ser-Lys-Phe- | |
| Phe-NH₂ | |
| Ac-Asp-Lys-Cys-Trp-Glu-Glu-Phe-Lys-Ser- | 981 |
| Phe-Thr-Ser-Cys-Leu-Asp-Ser-Lys-Phe- | |
| Phe-NH₂ | |
| Ac-Glu-Lys-Cys-Phe-Glu-Glu-Phe-Lys-Ser- | 982 |
| Tyr-Thr-Ser-Cys-Leu-Asp-Ser-Lys-Phe- | |
| Phe-NH₂ | |
| Ac-Glu-Lys-Cys-Phe-Glu-Glu-Phe-Lys-Ser- | 983 |
| Trp-Thr-Ser-Cys-Leu-Asp-Ser-Lys-Phe- | |
| Phe-NH₂ | |
| Ac-Glu-Lys-Cys-Val-Glu-Glu-Phe-Lys-Ser- | 984 |
| Trp-Thr-Ser-Cys-Leu-Asp-Ser-Lys-Ala- | |
| Phe-NH₂ | |
| Ac-Asp-Lys-Cys-Phe-Glu-Glu-Phe-Lys-Ser- | 985 |
| Trp-Thr-Ser-Cys-Leu-Asp-Ser-Lys-Ala- | |
| Phe-NH₂ | |

Other suitable peptides include, but are not limited to the peptides of Table 18.

TABLE 18
Illustrative peptides having an
improved hydrophobic phase.

| | | SEQ ID |
|---|---|---|
| Name | Sequence | NO |
| V2W3A5F1017- | Ac-Asp-Val-Trp-Lys-Ala-Ala- | 986 |
| D-4F | Tyr-Asp-Lys-Phe-Ala-Glu-Lys- | |
| | Phe-Lys-Glu-Phe-Phe-NH2 | |
| V2W3F10-D-4F | Ac-Asp-Val-Trp-Lys-Ala-Phe- | 987 |
| | Tyr-Asp-Lys-Phe-Ala-Glu-Lys- | |
| | Phe-Lys-Glu-Ala-Phe-NH2 | |
| W3-D-4F | Ac-Asp-Phe-Trp-Lys-Ala-Phe- | 988 |
| | Tyr-Asp-Lys-Val-Ala-Glu-Lys- | |
| | Phe-Lys-Glu-Ala-Phe-NH2 | |

The peptides described here (V2W3A5F10,17-D-4F; V2W3F10-D-4F; W3-D-4F) may be more potent than the original D-4F.

Still other suitable peptides include, but are not limited to: P¹-Dimethyltyrosine-D-Arg-Phe-Lys-P² (SEQ ID NO:989) and P¹-Dimethyltyrosine-Arg-Glu-Leu-P² where P1 and P2 are protecting groups as described herein. In certain embodiments, these peptides include, but are not limited to BocDimethyltyrosine-D-Arg-Phe-Lys(OtBu) and BocDimethyltyrosine-Arg-Glu-Leu(OtBu).

In certain embodiments, the peptides of this invention include peptides comprising or consisting of the amino acid sequence LAEYHAK (SEQ ID NO:990) comprising at least one D amino acid and/or at least one or two terminal protecting groups. In certain embodiments, this invention includes a peptide that ameliorates one or more symptoms of an inflammatory condition, wherein the peptide: ranges in length from about 3 to about 10 amino acids; comprises an amino acid sequence where the sequence comprises acidic or basic amino acids alternating with aromatic or hydrophobic amino acids; comprises hydrophobic terminal amino acids or terminal amino acids bearing a hydrophobic protecting group. In certain embodiments, the peptide is not the sequence LAEYHAK (SEQ ID NO:991) comprising all L amino acids; where the peptide converts pro-inflammatory HDL to anti-inflammatory HDL and/or makes anti-inflammatory HDL more anti-inflammatory.

It is also noted that the peptides listed in the Tables herein are not fully inclusive. Using the teaching provided herein, other suitable peptides can routinely be produced (e.g., by conservative or semi-conservative substitutions (e.g., D replaced by E), extensions, deletions, and the like). Thus, for example, one embodiment utilizes truncations of any one or more of peptides identified by SEQ ID Nos:829-857.

Longer peptides are also suitable. Such longer peptides may entirely form a class G or G* amphipathic helix, or the G amphipathic helix (helices) can form one or more domains of the peptide. In addition, this invention contemplates multimeric versions of the peptides. Thus, for example, the peptides illustrated in the tables herein can be coupled together (directly or through a linker (e.g., a carbon linker, or one or more amino acids) with one or more intervening amino acids). Suitable linkers include, but are not limited to Proline (-Pro-), Gly₄Ser₃ (SEQ ID NO: 992), and the like. Thus, one illustrative multimeric peptide according to this invention is (D-J336)-P-(D-J336) (i.e. Ac-L-L-E-Q-L-N-E-Q-F-N-W-V-S-R-L-A-N-L-T-Q-G-E-P-L-L-E-Q-L-N-E-Q-F-N-W-V-S-R-L-A-N-L-T-Q-G-E-NH₂, SEQ ID NO: 993).

This invention also contemplates the use of “hybrid” peptides comprising a one or more G or G* amphipathic helical domains and one or more class A amphipathic helices. Suitable class A amphipathic helical peptides are described in PCT publication WO 02/15923. Thus, by way of illustration, one such “hybrid” peptide is (D-J336)-Pro-(4F) (i.e. Ac-L-L-E-Q-L-N-E-Q-F-N-W-V-S-R-L-A-N-L-T-Q-G-E-P-D-W-F-K-A-F-Y-D-K-V-A-E-K-F-K-E-A-F-NH₂, SEQ ID NO:994), and the like.

Using the teaching provided herein, one of skill can routinely modify the illustrated amphipathic helical peptides to produce other suitable apo J variants and/or amphipathic G and/or A helical peptides of this invention. For example, routine conservative or semi-conservative substitutions (e.g., E for D) can be made of the existing amino acids. The effect of various substitutions on lipid affinity of the resulting peptide can be predicted using the computational method described by Palgunachari et al. (1996) Arteriosclerosis, Thrombosis, & Vascular Biology 16: 328-338. The peptides can be lengthened or shortened as long as the class helix structure(s) are preserved. In addition, substitutions can be made to render the resulting peptide more similar to peptide(s) endogenously produced by the subject species. An example of another class A helical peptide that can be used with the inventions described herein is the peptide D-R-L-K-A-F-Y-D-K-V-A-W-K-L-K-E-A-F (SEQ ID NO:995) which was reported to have membrane-binding properties (Mozsolits et al. (2004) Eur. Biophys. J., 33: 98-108).

While, in preferred embodiments, the peptides of this invention utilize naturally-occurring amino acids or D forms of naturally occurring amino acids, substitutions with non-naturally occurring amino acids (e.g., methionine sulfoxide, methionine methylsulfonium, norleucine, episilon-aminocaproic acid, 4-aminobutanoic acid, tetrahydroisoquinoline-3-carboxylic acid, 8-aminocaprylic acid, 4-aminobutyric acid, Lys(N(epsilon)-trifluoroacetyl), α-aminoisobutyric acid, and the like) are also contemplated.

New peptides can be designed and/or evaluated using computational methods. Computer programs to identify and classify amphipathic helical domains are well known to those of skill in the art and many have been described by Jones et al., (1992) J. Lipid Res. 33: 287-296). Such programs include, but are not limited to the helical wheel program (WHEEL or WHEEL/SNORKEL), helical net program (HELNET, HELNET/SNORKEL, HELNET/Angle), program for addition of helical wheels (COMBO or COMBO/SNORKEL), program for addition of helical nets (COMNET, COMNET/SNORKEL, COMBO/SELECT, COMBO/NET), consensus wheel program (CONSENSUS, CONSENSUS/SNORKEL), and the like.

F) Blocking Groups and D Residues.

While the various peptides and/or amino acid pairs described herein may be shown with no protecting groups, in certain embodiments (e.g., for oral administration), they can bear one, two, three, four, or more protecting groups. The protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g., an acetyl group protecting the amino terminus and/or an amide group protecting the carboxyl terminus. One example of such a “dual protected peptide is Ac-L-L-E-Q-L-N-E-Q-F-N-W-V-S-R-L-A-N-L-T-Q-G-E-NH₂ (SEQ ID NO:829 with blocking groups), either or both of these protecting groups can be eliminated and/or substituted with another protecting group as described herein.

Without being bound by a particular theory, it was a discovery of this invention that blockage, particularly of the amino and/or carboxyl termini of the subject peptides of this invention greatly improves oral delivery and significantly increases serum half-life. It was also a surprising discovery, however, that in certain embodiments, particular when used in conjunction with the salicylanilides (e.g., niclosamide) and other delivery agents described herein, any or all of the protecting groups can be omitted and the peptides are still orally administrable. Nevertheless, in certain embodiments the peptides, even when formulated with and/or administered in conjunction with a salicylanilide or other delivery agent as described herein bears one or more protecting groups (e.g., terminal protecting groups).

A wide number of protecting groups are suitable for this purpose. Such groups include, but are not limited to acetyl, amide, and alkyl groups with acetyl and alkyl groups being particularly preferred for N-terminal protection and amide groups being preferred for carboxyl terminal protection. In certain particularly preferred embodiments, the protecting groups include, but are not limited to alkyl chains as in fatty acids, propeonyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g., groups having the formula: CH₃—(CH₂)ₙ—CO— where n ranges from about 1 to about 20, preferably from about 1 to about 16 or 18, more preferably from about 3 to about 13, and most preferably from about 3 to about 10.

In certain particularly preferred embodiments, the protecting groups include, but are not limited to alkyl chains as in fatty acids, propeonyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g., groups having the formula: CH₃—(CH₂)ₙ—CO— where n ranges from about 3 to about 20, preferably from about 3 to about 16, more preferably from about 3 to about 13, and most preferably from about 3 to about 10.

Other protecting groups include, but are not limited to Fmoc, t-butoxycarbonyl (t-BOC), 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br-Z), Benzyloxymethyl (Bom), cyclohexyloxy (cHxO),t-butoxymethyl (Bum), t-butoxy (tBuO), t-Butyl (tBu), Acetyl (Ac), and Trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.). In one preferred embodiment, for example, acetylation is accomplished during the synthesis when the peptide is on the resin using acetic anhydride. Amide protection can be achieved by the selection of a proper resin for the synthesis. During the synthesis of the peptides described herein in the examples, rink amide resin was used. After the completion of the synthesis, the semipermanent protecting groups on acidic bifunctional amino acids such as Asp and Glu and basic amino acid Lys, hydroxyl of Tyr are all simultaneously removed. The peptides released from such a resin using acidic treatment comes out with the n-terminal protected as acetyl and the carboxyl protected as NH, and with the simultaneous removal of all of the other protecting groups.

In certain particularly preferred embodiments, the peptides comprise one or more D-form (dextro rather than levo) amino acids as described herein. In certain embodiments at least two enantiomeric amino acids, more preferably at least 4 enantiomeric amino acids and most preferably at least 8 or 10 enantiomeric amino acids are “D” form amino acids. In certain embodiments every other, or even every amino acid (e.g., every enantiomeric amino acid) of the peptides described herein is a D-form amino acid.

In certain embodiments at least 50% of the enantiomeric amino acids are “D” form, more preferably at least 80% of the enantiomeric amino acids are “D” form, and most preferably at least 90% or even all of the enantiomeric amino acids are “D” form amino acids.

G) Peptide Mimetics.

In addition to the peptides described herein, it is believed that the salicylanilides (e.g., niclosamide) and other delivery agents described herein are also useful to improve in vivo activity of orally delivered peptide mimetics. Peptide analogs are commonly used in the pharmaceutical industry as non-peptide drugs with properties analogous to those of the template peptide. These types of non-peptide compound are termed “peptide mimetics” or “peptidomimetics” (Fauchere (1986) Adv. Drug Res. 15: 29; Veber and Freidinger (1985) TINS p. 392; and Evans et al. (1987) J. Med. Chem. 30: 1229) and are usually developed with the aid of computerized molecular modeling. Peptide mimetics that are structurally similar to therapeutically useful peptides may be used to produce an equivalent therapeutic or prophylactic effect.

Generally, peptidomimetics are structurally similar to a paradigm polypeptide (e.g., SEQ ID NO:5 shown in Table I), but have one or more peptide linkages optionally replaced by a linkage selected from the group consisting of: —CH₂NH—, —CH₂S—, —CH₂—CHᵣ, —CH═CH— (cis and trans), —COCH₂—, —CH(OH)CH₂—, —CH₂SO—, etc. by methods known in the art and further described in the following references: Spatola (1983) p. 267 in Chemistry and Biochemistry of Amino Acids, Peptides, and Proteins, B. Weinstein, eds., Marcel Dekker, New York,; Spatola (1983) Vega Data 1(3) Peptide Backbone Modifications. (general review); Morley (1980) Trends Pharm Sci pp. 463-468 (general review); Hudson et al. (1979) Int J Pept Prot Res 14:177-185 (—CH₂NH—, CH₂CH₂—); Spatola et al. (1986) Life Sci 38:1243-1249 (—CH₂—S); Hann, (1982) J Chem Soc Perkin Trans I 307-314 (—CH—CH—, cis and trans); Almquist et al. (1980) J Med. Chem. 23:1392-1398 (—COCH₂—); Jennings-White et al. (1982) Tetrahedron Lett. 23:2533 (—COCH₂—); Szelke et al., European Appln. EP 45665 (1982) CA: 97:39405 (1982) (—CH(OH)CH2—); Holladay et al. (1983) Tetrahedron Lett 24:4401-4404 (—C(OH)CH₂—); and Hruby (1982) Life Sci., 31:189-199 (—CH₂—S—)).

One particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), reduced antigenicity, and others.

In addition, circularly permutations of the peptides described herein or constrained peptides (including cyclized peptides) comprising a consensus sequence or a substantially identical consensus sequence variation may be generated by methods known in the art (Rizo and Gierasch (1992) Ann. Rev. Biochem. 61: 387); for example, by adding internal cysteine residues capable of forming intramolecular disulfide bridges which cyclize the peptide.

### V. Pharmaceutical Formulations.

A) Pharmaceutical Formulations.

In order to carry out the methods of the invention, one or more therapeutic peptides, mimetics, etc., described herein are reacted with a salicylanilide (e.g., niclosamide or niclosamide analogue) to form a complex (e.g., a peptide-salicylanilide complex) which can easily be administered to a mammal, e.g., to an subject diagnosed as having one or more symptoms of atherosclerosis, or as being at risk for atherosclerosis and or the various other pathologies described herein.

In various embodiments the “active agent(s)”, therapeutic peptides, mimetics, or small organic molecules described herein, are formulated in combination with one or more of the salicylanilides (e.g., niclosamide or niclosamide analogue) or one of the other delivery agents described herein to form a complex. The active agent(s)-salicylanilide complex can be administered in the “native” form or, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents and/or salicylanilides (e.g., various moieties comprising the complex) can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 00/059863 which is incorporated herein by reference. Similarly, acid salts of therapeutic peptides, mimetics, and small organic molecules can be prepared from the free base using conventional methodology, that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric, acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The active agents identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., atherosclerosis and/or symptoms thereof). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

In various embodiments, the complexes of this invention can be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., active peptide and salicylanilide) and the resulting composition is compressed. Where necessary, the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments the excipients (carriers) are sterile and generally free of undesirable matter. These compositions can be sterilized by conventional, well-known sterilization techniques. For various oral dosage form excipients such as tablets and capsules sterility is not required. The USP/NF standard is usually sufficient.

In therapeutic applications, the compositions of this invention are administered, e.g., orally administered, to a patient suffering from one or more symptoms of the one or more pathologies described herein, or at risk for one or more of the pathologies described herein in an amount sufficient to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of active agent(s) can vary widely, and will be selected primarily based on activity of the active ingredient(s), body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic regimen in a particular subject or group of subjects.

In certain embodiments, the active agents of this invention are administered orally (e.g., via a tablet, capsule, caplet, gel cap, etc.). It was a surprising discovery that therapeutic peptides when formulated as a complex with one or more salicylanilides, e.g., as described herein, can be orally administered and achieve therapeutically effective levels, particularly. It was particularly surprising that when so administered, the therapeutic peptide can be an L-form peptide and need not bear protecting groups. The complexation of therapeutic peptide with a salicylanilide is not limited to unprotected L-form peptides. To the contrary, the use salicylanilides and/or other delivery agent(s) with L-form peptides bearing one or more protecting groups, D-form peptides, and D-form peptides bearing one or more protecting groups is also contemplated.

In certain embodiments the active agents (and/or complexes) of this invention are administered as an injectable in accordance with standard methods well known to those of skill in the art. In other preferred embodiments, the agents/complexes, can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other formulations for topical drug delivery include, but are not limited to, ointments and creams. Ointments are semisolid preparations that are typically based on petrolatum or other petroleum derivatives. Creams containing the selected active agent/complex are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

As indicated above, various buccal, and sublingual formulations are also contemplated.

The use of salicylanilide/peptide complexes as described herein need not be limited to oral delivery. In certain embodiments the use of such delivery vehicles is also contemplated in formulations intended for transdermal delivery, injectable delivery, surgical implantation, nasal delivery, rectal delivery, and the like.

In another embodiment, the complexes described herein can be provided as a “concentrate”, e.g., in a storage container (e.g., in a premature volume) ready for dilution, or in a soluble capsule ready for addition to a volume of water. In certain embodiments the salicylanilide and the therapeutic agent are provided separately for later complexation.

The foregoing formulations and administration methods are intended to be illustrative and not limiting. It will be appreciated that, using the teaching provided herein, other suitable formulations and modes of administration can be readily devised.

B) Lipid-Based Formulations.

In certain embodiments, the peptide/salicylanilide complexes are administered in conjunction with one or more lipids. The lipids can be formulated as an excipient to protect and/or enhance transport/uptake of the active agents (e.g., peptides) or they can be administered separately.

Without being bound by a particular theory, it was discovered of this invention that administration (e.g., oral administration) of certain phospholipids can significantly increase HDL/LDL ratios. In addition, it is believed that certain medium-length phospholipids are transported by a process different than that involved in general lipid transport. Thus, co-administration of certain medium-length phospholipids with the active agents of this invention confer a number of advantages: They protect the active agents from digestion or hydrolysis, they improve uptake, and they improve HDL/LDL ratios.

The lipids can be formed into liposomes that encapsulate the active agents of this invention and/or they can be complexed/admixed with the active agents and/or they can be covalently coupled to the active agents. Methods of making liposomes and encapsulating reagents are well known to those of skill in the art (see, e.g., Martin and Papahadjopoulos (1982) J. Biol. Chem., 257: 286-288; Papahadjopoulos et al. (1991) Proc. Natl. Acad. Sci. USA, 88: 11460-11464; Huang et al. (1992) Cancer Res., 52:6774-6781; Lasic et al. (1992) FEBS Lett., 312: 255-258., and the like).

Preferred phospholipids for use in these methods have fatty acids ranging from about 4 carbons to about 24 carbons in the sn-1 and sn-2 positions. In certain preferred embodiments, the fatty acids are saturated. In other preferred embodiments, the fatty acids can be unsaturated. Various preferred fatty acids are illustrated in Table 19.

TABLE 19
Preferred fatty acids in the sn-1 and/or sn-2 position of the preferred
phospholipids for administration of active agents described herein.

| Carbon No. | Common Name | IUPAC Name |
|---|---|---|
| 3:0 | Propionoyl | Trianoic |
| 4:0 | Butanoyl | Tetranoic |
| 5:0 | Pentanoyl | Pentanoic |
| 6:0 | Caproyl | Hexanoic |
| 7:0 | Heptanoyl | Heptanoic |
| 8:0 | Capryloyl | Octanoic |
| 9:0 | Nonanoyl | Nonanoic |
| 10:0 | Capryl | Decanoic |
| 11:0 | Undcanoyl | Undecanoic |
| 12:0 | Lauroyl | Dodecanoic |
| 13:0 | Tridecanoyl | Tridecanoic |
| 14:0 | Myristoyl | Tetradecanoic |
| 15:0 | Pentadecanoyl | Pentadecanoic |
| 16:0 | Palmitoyl | Hexadecanoic |
| 17:0 | Heptadecanoyl | Heptadecanoic |
| 18:0 | Stearoyl | Octadecanoic |
| 19:0 | Nonadecanoyl | Nonadecanoic |
| 20:0 | Arachidoyl | Eicosanoic |
| 21:0 | Heniecosanoyl | Heniecosanoic |
| 22:0 | Behenoyl | Docosanoic |
| 23:0 | Trucisanoyl | Trocosanoic |
| 24:0 | Lignoceroyl | Tetracosanoic |
| 14:1 | Myristoleoyl (9-cis) | |
| 14:1 | Myristelaidoyl (9-trans) | |
| 16:1 | Palmitoleoyl (9-cis) | |
| 16:1 | Palmitelaidoyl (9-trans) | |

The fatty acids in these positions can be the same or different. Particularly preferred phospholipids have phosphorylcholine at the sn-3 position.

### VI. Additional Pharmacologically Active Agents.

A) Combined Active Agents

In various embodiments, the use of combinations of two or more active agents described is contemplated in the treatment of the various pathologies/indications described herein. The use of combinations of active agents can alter pharmacological activity, bioavailability, and the like.

By way of illustration, it is noted that D-4F and L-4F rapidly associates with pre-beta HDL and HDL and then are rapidly cleared from the circulation (it is essentially non-detectable 6 hours after an oral dose), while D-[113-122]apoJ slowly associates with pre-beta HDL and to a lesser extent with HDL but remains associated with these HDL fractions for at least 36 hours. FREL associates with HDL and only HDL but remains 15 detectable in HDL for much longer than D-4F (i.e., it is detectable in HDL 48 hours after a single oral dose in mice). In certain embodiments this invention thus contemplates combinations of, for example, these three peptides to reduce the amount to reduce production expense, and/or to optimize dosage regimen, therapeutic profile, and the like. In certain embodiments combinations of the active agents described herein can be simply coadministered and/or added together to form a single pharmaceutical formulation. Tn certain embodiments the various active agent(s) can be complexed together (e.g., via hydrogen bonding) to form active agent complexes that are more effective than the parent agents.

B) Use with Additional Pharmacologically Active Materials.

Additional pharmacologically active materials (i.e., drugs) can be delivered in conjunction with one or more of the active agents described herein. In certain embodiments, such agents include, but are not limited to agents that reduce the risk of atherosclerotic events and/or complications thereof. Such agents include, but are not limited to beta blockers, beta blockers and thiazide diuretic combinations, statins, aspirin, ace inhibitors, ace receptor inhibitors (ARBs), and the like.

It was discovered that, adding a low dosage active agent (e.g., of D-4F) (1 μg/ml) to the drinking water of apoE null mice for 24 hours did not significantly improve HDL function (see, e.g., related application U.S. Ser. No. 10/423,830, filed on Apr. 25, 2003, which is incorporated herein by reference). In addition, adding 0.05 mg/ml of atorvastatin or pravastatin alone to the drinking water of the apoE null mice for 24 hours did not improve HDL function. However, when D-4F1 μg/ml was added to the drinking water together with 0.05 mg/ml of atorvastatin or pravastatin there was a significant improvement in HDL function). Indeed the pro-inflammatory apoE null HDL became as anti-inflammatory as 350 μg/ml of normal human HDL (h, HDL see, e.g., related application U.S. Ser. No. 10/423,830).

Thus, doses of D-4F alone, or statins alone, which by themselves had no effect on HDL function when given together acted synergistically. When D-4F and a statin were given together to apo E null mice, their pro-inflammatory HDL at 50 μg/ml of HDL-cholesterol became as effective as normal human HDL at 350 μg/ml of HDL-cholesterol in preventing the inflammatory response induced by the action of HPODE oxidizing PAPC in cocultures of human artery wall cells.

Thus, in certain embodiments this invention provides methods for enhancing the activity of statins. The methods generally involve administering one or more of the active agents described herein, as described herein in conjunction with one or more statins. The active agents achieve synergistic action between the statin and the agent(s) to ameliorate one or more symptoms of atherosclerosis. In this context statins can be administered at significantly lower dosages thereby avoiding various harmful side effects (e.g., muscle wasting) associated with high dosage statin use and/or the anti-inflammatory properties of statins at any given dose are significantly enhanced.

Suitable statins include, but are not limited to pravastatin (Pravachol/Bristol-Myers Squibb), simvastatin (Zocor/Merck), lovastatin (Mevacor/Merck), and the like.

In various embodiments the active agent(s) described herein are administered in conjunction with one or more beta blockers. Suitable beta blockers include, but are not limited to cardioselective (selective beta 1 blockers), e.g., acebutolol (Sectral™), atenolol (Tenormin™), betaxolol (Kerlone™), bisoprolol (Zebeta™), metoprolol (Lopressor™), and the like. Suitable non-selective blockers (block beta 1 and beta 2 equally) include, but are not limited to carteolol (Cartrol™), nadolol (Corgard™), penbutolol (Levatol™), pindolol (Visken™), propranolol (Inderal™), timolol (Blockadren™), labetalol (Normodyne™, Trandate™), and the like.

Suitable beta blocker thiazide diuretic combinations include, but are not limited to Lopressor HCT, ZIAC, Tenoretic, Corzide, Timolide, Inderal LA 40/25, Inderide, Normozide, and the like.

Suitable ace inhibitors include, but are not limited to captopril (e.g., Capoten™ by Squibb), benazepril (e.g., Lotensin™ by Novartis), enalapril (e.g., Vasotec™ by Merck), fosinopril (e.g., Monopril™ by Bristol-Myers), lisinopril (e.g., Prinivil™ by Merck or Zestril™ by Astra-Zeneca), quinapril (e.g., Accupril™ by Parke-Davis), ramipril (e.g., Altace™ by Hoechst Marion Roussel, King Pharmaceuticals), imidapril, perindopril erbumine (e.g., Aceon™ by Rhone-Polenc Rorer), trandolapril (e.g., Mavik™ by Knoll Pharmaceutical), and the like. Suitable ARBS (Ace Receptor Blockers) include but are not limited to losartan (e.g., Cozaar™ by Merck), irbesartan (e.g., Avapro™ by Sanofi), candesartan (e.g., Atacand™ by Astra Merck), valsartan (e.g., Diovan™ by Novartis), and the like.

In various embodiments, one or more agents described herein are administered with one or more of the drugs identified below.

Thus, in certain embodiments one or more active agents are administered in conjunction with cholesteryl ester transfer protein (CETP) inhibitors (e.g., torcetrapib, ITT-705. CP-529414) and/or acyl-CoA:cholesterol O-acyltransferase (ACAT) inhibitors (e.g., Avasimibe (CI-1011), CP 113818, F-1394, and the like), and/or immunomodulators (e.g., FTY720 (sphingosine-1-phosphate receptor agonist), Thalomid (thalidomide), Imuran (azathioprine), Copaxone (glatiramer acetate), Certican® (everolimus), Neoral®(cyclosporine), and the like), and/or dipeptidyl-peptidase-4 (DPP4) inhibitors (e.g., 2-Pyrrolidinecarbonitrile, 1-[[[2-[(5-cyano-2-pyridinyl)amino]ethyl]amino]acetyl], see also U.S. Patent Publication 2005-0070530), and/or calcium channel blockers (e.g., Adalat, Adalat CC, Calan, Calan SR, Cardene, Cardizem, Cardizem CD, Cardizem SR, Dilacor-XR, DynaCirc, Isoptin, Isoptin SR, Nimotop, Norvasc, Plendil, Procardia, Procardia XL, Vascor, Verelan), and/or peroxisome proliferator-activated receptor (PPAR) agonists for, e.g., α, γ; δ receptors (e.g., Azelaoyl PAF, 2-Bromohexadecanoic acid, Ciglitizone, Clofibrate, 15-Deoxy-δ^{12,14}-prostaglandin J₂, Fenofibrate, Fmoc-Leu-OH, GW1929, GW7647, 8(S)-Hydroxy-(5Z,9E,11Z,14Z)-eicosatetraenoic acid (8(S)-HETE), Leukotriene B₄, LY-171,883 (Tomelukast), Prostaglandin A₂, Prostaglandin J₂, Tetradecylthioacetic acid (TTA), Troglitazone (CS-045), WY-14643 (Pirinixic acid)), and the like.

In certain embodiments one or more of the active agents are administered in conjunction with fibrates (e.g., clofibrate (atromid), gemfibrozil (lopid), fenofibrate (tricor), etc.), bile acid sequestrants (e.g., cholestyramine, colestipol, etc.), cholesterol absorption blockers (e.g., ezetimibe (Zetia), etc.), Vytorin ((ezetimibe/simvastatin combination), and/or steroids, warfarin, and/or aspirin, and/or Bcr-Abl inhibitors/antagonists (e.g., Gleevec (Imatinib Mesylate), AMN₁₀₇, STI571 (CGP57148B), ON 012380, PLX225, and the like), and/or renin angiotensin pathway blockers (e.g., Losartan (Cozaar®), Valsartan (Diovan®), Irbesartan (Avapro®), Candesartan (Atacand®), and the like), and/or angiotensin II receptor antagonists (e.g., losartan (Cozaar), valsartan (Diovan), irbesartan (Avapro), candesartan (Atacand) and telmisartan (Micardis), etc.), and/or PKC inhibitors (e.g., Calphostin C, Chelerythrine chloride, Chelerythrine.chloride, Copper bis-3,5-diisopropylsalicylate, Ebselen, EGF Recepior (human) (651-658) (N-Myristoylated), Go 6976, H-7 dihydrochloride, 1-O-Hexadecyl-2-O-methyl-rac-glycerol, Hexadecyl-phosphocholine (C_{16:0}); Miltefosine, Hypericin, Melittin (natural), Melittin (synthetic), ML-7 hydrochloride, ML-9 hydrochloride, Palmitoyl-DL-carnitine.hydrochloride, Protein Kinase C (19-31), Protein Kinase C (19-36), Quercetin.dihydrate, Quercetin.dihydrate, D-erythro-Sphingosine (isolated), D-erythro-Sphingosine (synthetic), Sphingosine, N,N-dimethyl, D-erythro-Sphingosine, Dihydro-, D-erythro-Sphingosine, N,N-Dimethyl-, D-erythro-Sphingosine chloride, N,N,N-Trimethyl-, Staurosporine, Bisindolylmaleimide I, G-6203, and the like).

In certain embodiments, one or more of the active agents are administered in conjunction with ApoAI, Apo A-I derivatives and/or agonists (e.g., ApoAI milano, see, e.g., U.S. Patent Publications 20050004082, 20040224011, 20040198662, 20040181034, 20040122091, 20040082548, 20040029807, 20030149094, 20030125559, 20030109442, 20030065195, 20030008827, and 20020071862, and U.S. Pat. Nos. 6,831,105, 6,790,953, 6,773,719, 6,713,507, 6,703,422, 6,699,910, 6,680,203, 6,673,780, 6,646,170, 6,617,134, 6,559,284, 6,506,879, 6,506,799, 6,459,003, 6,423,830, 6,410,802, 6,376,464, 6,367,479, 6,329,341, 6,287,590, 6,090,921, 5,990,081, and the like), renin inhibitors (e.g., SPP630 and SPP635, SPP100, Aliskiren, and the like), and/or MR antagonist (e.g., spironolactone, aldosterone glucuronide, and the like), and/or aldosterone synthase inhibitors, and/or alpha-adrenergic antagonists (e.g., Aldomet® (Methyldopa), Cardura® (Doxazosin), Catapres®; Catapres-TTS®; Duraclon™ (Clonidine), Dibenzyline® (Phenoxybenzamine), Hylorel® (Guanadrel), Hytrin® (Terazosin), Minipress® (Prazosin), Tenex® (Guanfacine), Guanabenz, Phentolamine, Reserpine, and the like), and/or liver X receptor (LXR) agonists (e.g., T0901317, GW3965, ATI-829, acetyl-podocarpic dimer (APD), and the like), and/or farnesoid X receptor (FXR) agonists (e.g., GW4064, 6alpha-ethyl-chenodeoxycholic acid (6-ECDCA), T0901317, and the like), and/or plasminogen activator-1 (PAI-1) inhibitors (see, e.g., oxime-based PAI-1 inhibitors, see also U.S. Pat. No. 5,639,726, and the like), and/or low molecular weight heparin, and/or AGE inhibitors/breakers (e.g., Benfotiamine, aminoguanidine, pyridoxamine, Tenilsetam, Pimagedine, and the like) and/or ADP receptor blockers (e.g., Clopidigrel, AZD6140, and the like), and/or ABCA1 agonists, and/or scavenger receptor B1 agonists, and/or Adiponectic receptor agonist or adiponectin inducers, and/or stearoyl-CoA Desaturase I (SCD1) inhibitors, and/or Cholesterol synthesis inhibitors (non-statins), and/or Diacylglycerol Acyltransferase I (DGAT1) inhibitors, and/or Acetyl CoA Carboxylase 2 inhibitors, and/or LP-PLA2 inhibitors, and/or GLP-1, and/or glucokinase activator, and/or CB-1 agonists, and/or anti-thrombotic/coagulants, and/or Factor Xa inhibitors, and/or GPIIb/IIIa inhibitors, and/or Factor VIIa inhibitors, and/or Tissue factor inhibitors, and/or anti-inflammatory drugs, and/or Probucol and derivatives (e.g., AGI-1067, etc.), and/or CCR2 antagonists, and/or CX3CR1 antagonists, and/or IL-1 antagonists, and/or nitrates and NO donors, and/or phosphodiesterase inhibitors, and the like.

C) Administration.

Typically the peptide-salicylanilide complex(s) described herein will be administered to a mammal (e.g., a human) in need thereof. Such a mammal will typically include a mammal (e.g., a human) having or at risk for one or more of the pathologies described herein.

The complex(es) can be administered, as described herein, according to any of a number of standard methods including, but not limited to injection, suppository, nasal spray, time-release implant, transdermal patch, and the like. In one particularly preferred embodiment, the complex(es) are administered orally (e.g., as a syrup, capsule, or tablet).

The methods involve the administration of a complex comprising a single active agent (e.g. peptide) or a complex comprising a plurality of peptides, or a plurality of complexes to provide a collection of complexes comprising multiple active agents. The complex(es) can be provided as monomers (e.g., in separate or combined formulations), or in dimeric, oligomeric or polymeric forms. In certain embodiments, the multimeric forms may comprise associated monomers (e.g., ionically or hydrophobically linked) while certain other multimeric forms comprise covalently linked monomers (directly linked or through a linker).

While the invention is described with respect to use in humans, it is also suitable for animal, e.g., veterinary use. Thus certain preferred organisms include, but are not limited to humans, non-human primates, canines, equines, felines, porcines, ungulates, largomorphs, and the like.

The methods of this invention are not limited to humans or non-human animals showing one or more symptom(s) of the pathologies described herein, but are also useful in a prophylactic context. Thus, the complexes of this invention can be administered to organisms to prevent the onset/development of one or more symptoms of the pathologies described herein (e.g., atherosclerosis, stroke, etc.). Particularly preferred subjects in this context are subjects showing one or more risk factors for the pathology. Thus, for example, in the case of atheroklerosis, risk factors include family history, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.

### VII. Kits for the Treatment of One or More Indications.

In another embodiment this invention provides kits for amelioration of one or more symptoms of atherosclerosis or for the prophylactic treatment of a subject (human or animal) at risk for atherosclerosis and/or the treatment or prophylaxis of one or more of the conditions described herein. The kits preferably comprise a container containing one or more of the complexes described herein. The complex(es) can be provided in a unit dosage formulation (e.g., suppository, tablet, caplet, patch, etc.) and/or may be optionally combined with one or more pharmaceutically acceptable excipients.

The kit can, optionally, further comprise one or more other agents used in the treatment of the condition/pathology of interest. Such agents include, but are not limited to, beta blockers, vasodilators, aspirin, statins, ace inhibitors or ace receptor inhibitors (ARBs) and the like, e.g., as described above.

In addition, the kits optionally include labeling and/or instructional materials providing directions (i.e., protocols) for the practice of the methods or use of the “therapeutics” or “prophylactics” of this invention. Preferred instructional materials describe the use of one or more active agent(s) of this invention to mitigate one or more symptoms of atherosclerosis (or other pathologies described herein) and/or to prevent the onset or increase of one or more of such symptoms in an individual at risk for atherosclerosis (or other pathologies described herein). The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to Internet sites that provide such instructional materials.

### VIII. Indications.

The complexes comprising the active agents (e.g., peptides, small organic molecules, amino acid pairs, etc.) described herein are effective for mitigating one or more symptoms and/or reducing the rate of onset and/or severity of one or more indications described herein. In particular, the active agents (e.g., peptides, small organic molecules, amino acid pairs, etc.) described herein are effective for mitigating one or more symptoms of atherosclerosis. Without being bound to a particular theory, it is believed that the peptides bind the “seeding molecules” required for the formation of pro-inflammatory oxidized phospholipids such as Ox-PAPC, POVPC, PGPC, and PEIPC.

In addition, since many inflammatory conditions and/or other pathologies are mediated at least in part by oxidized lipids, we believe that the complexes comprising the peptides of this invention are effective in ameliorating conditions that are characterized by the formation of biologically active oxidized lipids. In addition, there are a number of other conditions for which the active agents described herein appear to be efficacious.

A number of pathologies for which the active agents described herein appear to be a palliative and/or a preventative are shown in Table 20.

TABLE 20
Summary of conditions in which the active agents (e.g., D-
4F) have been shown to be or are believed to be effective.

atherosclerosis/symptoms/consequences thereof

plaque formation
lesion formation
myocardial infarction
stroke

congestive heart failure
vascular function:

arteriole function
arteriolar disease

associated with aging
associated with Alzheimer's disease
associated with chronic kidney disease
associated with hypertension
associated with multi-infarct dementia
associated with subarachnoid hemorrhage

peripheral vascular disease

pulmonary disease:

chronic obstructive pulmonary disease (COPD),
emphysema
asthma
idiopathic pulmonary fibrosis
Pulmonary fibrosis
adult respiratory distress syndrome

osteoporosis
Paget's disease
coronary calcification
autoimmune:

rheumatoid arthritis
polyarteritis nodosa
polymyalgia rheumatica
lupus erythematosus
multiple sclerosis
Wegener's granulomatosis
central nervous system vasculitis (CNSV)
Sjögren's syndrome
Scleroderma
polymyositis.

AIDS inflammatory response
infections:

bacterial
fungal
viral
parasitic
influenza

avian flu

viral pneumonia
endotoxic shock syndrome
sepsis
sepsis syndrome
(clinical syndrome where it appears that the patient is septic
but no organisms are recovered from the blood)

trauma/wound:

organ transplant
transplant atherosclerosis
transplant rejection
corneal ulcer
chronic/non-healing wound
ulcerative colitis
reperfusion injury (prevent and/or treat)
ischemic reperfusion injury (prevent and/or treat)
spinal cord injuries (mitigating effects)

cancers

myeloma/multiple myeloma
ovarian cancer
breast cancer
colon cancer
bone cancer

osteoarthritis
inflammatory bowel disease
allergic rhinitis
cachexia
diabetes
Alzheimer's disease
implanted prosthesis
biofilm formation
Crohns' disease
dermatitis, acute and chronic

eczema
psoriasis
contact dermatitis
scleroderma

diabetes and related conditions

Type I Diabetes
Type II Diabetes
Juvenile Onset Diabetes
Prevention of the onset of diabetes
Diabetic Nephropathy
Diabetic Neuropathy
Diabetic Retinopathy

erectile dysfunction
macular degeneration
multiple sclerosis
nephropathy
neuropathy
Parkinson's Disease
peripheral Vascular Disease
meningitis
Specific biological activities:

increase Heme Oxygenase 1
increase extracellular superoxide dismutase
prevent endothelial sloughing
prevent the association of myeloperoxidase with ApoA-I
prevent the nitrosylation of tyrosine in ApoA-I
render HDL anti-inflammatory
improve vasoreactivity
increase the formation of pre-beta HDL
promote reverse cholesterol transport
promote reverse cholesterol transport from macrophages
synergize the action of statins

It is noted that the conditions listed in Table 20 are intended to be illustrative and not limiting.

### EXAMPLES

The following examples are offered to illustrate, but not to limit the claimed invention.

### Example 1

### Niclosamide Enhances Uptake/Bioavailability of Orally Administered Peptides

We previously reported that the amino acid sequence D-W-F-K-A-F-Y-D-K-V-A-E-KF-K-E-A-F(SEQ-ID-NO:5) bearing at least one protecting group (see, e.g., U.S. Pat. No. 6,933,279) when synthesized from all L-amino acids (L-4F) and administered orally to mice was rapidly degraded and did not significantly alter the protective capacity of HDL to inhibit LDL-induced monocyte chemotactic activity in cultures of human artery wall cells (Navab et al. (2002) Circulation 105: 290-292).

It was a surprising finding of this invention that administering L-4F with niclosamide orally to mice resulted in significant improvement in the ability of HDL from these mice to inhibit LDL-induced monocyte chemotactic activity. In contrast orally administering either agent alone was ineffective or significantly less effective.

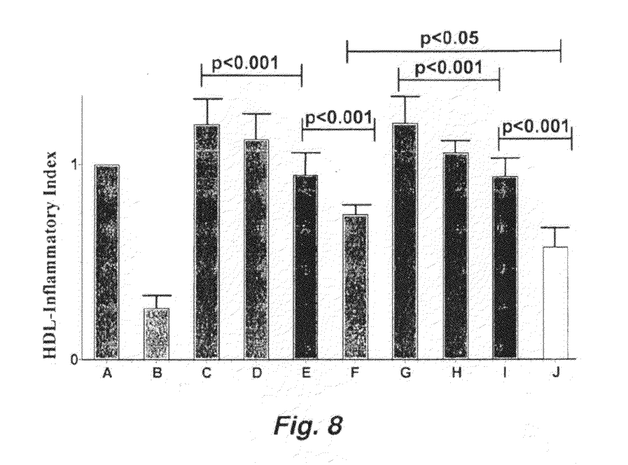

As shown in FIG. 8, the combination of oral Niclosamide and L-4F was potent in a mouse model of atherosclerosis. 11-month-old female apoE null mice were fasted during the day. At night the mice were provided chow containing or not containing additions. In the first experiment the mice were given chow alone (C) or chow supplemented with 8.0 micrograms of Niclosamide (2′,5-Dichloro-4′-nitrosalicylanilide; Niclosamide, Sigma catalog number N-3510 Page 1711 2006-2007 catalog Empirical Formula (Hill Notation): C₁₃H₈C₁₂N₂O₄ Formula Weight: 327.12, CAS Number: 50-65-7 Batch 105K0666 EC 200-056-8) per gram of chow (D) or chow supplemented with 2.0 micrograms of L-4F (free base) per gram of chow (E), or chow supplemented with 8.0 micrograms of Niclosamide together with 2.0 micrograms of L-4F (free base) per gram of chow (F). The mice were only given one gram of chow per mouse (n=8 mice per group) so that they would consume all of the chow. In the morning after the chow was consumed the mice were bled and their plasma was sucrose cryopreserved and fractionated by FPLC and the HDL-containing fractions were tested for their ability to inhibit monocyte chemotactic activity induced by a standard control human LDL (A) in cultures of human aortic endothelial cells. The mouse HDL (C-J) was also compared to a standard human HDL (B) that was added at the same concentrations as the mouse HDL. The resulting monocyte chemotactic activity was normalized to the standard control LDL added alone (A). The results are plotted as the HDL-inflammatory index, which is the result of dividing the monocyte chemotactic activity measured for each condition by the monocyte chemotactic activity obtained by the standard control LDL added alone, which was normalized to 1.0 as described previously (Navab et al. (2004) J Lipid Res, 45: 993-1007).

A second experiment was performed as described for the first experiment with 8 mice in each group except that the additions to the chow were different. Chow alone in the second experiment (G) was compared to chow supplemented with 100 micrograms of Niclosamide per gram of chow (H), or supplemented with 10 micrograms of L-4F (free base) per gram of mouse chow (I), or supplemented with 10 micrograms of L-4F (free base) together with 100 micrograms of Niclosamide per gram of chow (J). As in the first experiment the mice were only given one gram of chow per mouse so that they would consume all of the chow. In the morning this second group of mice were bled and their HDL tested in the human artery wall cell culture together with the HDL from the first experiment.

The data indicate that addition of either 2 (E) or 10 (I) micrograms of L-4F to the chow slightly but significantly improved the HDL-inflammatory index and the difference between these two doses in the absence of Niclosamide was not significant confirming our previous report (Navab et al. (2002) Circulation, 105: 290-292). As shown in FIGS. 8 (D) and (H), administering Niclosamide by itself was ineffective. Surprisingly the oral combination of Niclosamide with L-4F in each case resulted in dramatic statistically significant improvement in the HDL-inflammatory index. The use of 10 micrograms of L-4F together with 100 micrograms of Niclosamide (J) was significantly better than 2 micrograms of L-4F together with 8 micrograms of Niclosamide (F).

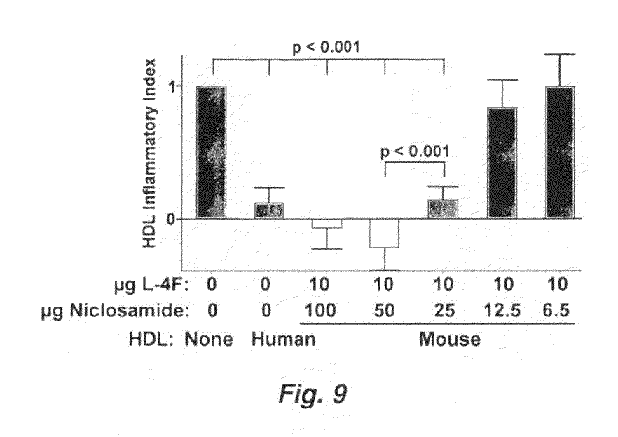

As shown in FIG. 9, administration of Niclosamide as an oral bolus by gastric gavage (stomach tube) immediately followed by administration of L-4F as an oral bolus by stomach tube rendered apoE null mouse HDL anti-inflammatory. Ten mg of Niclosamide was placed in a glass-glass homogenizer with mortar and round bottom pestle (Kontes Dounce Tissue grinder, K885300-0015 available from Fisher, VWR) and 200 μL of ethanol was added. The Niclosamide ethanol mixture was homogenized using 2-3 strokes and distilled water was added and the mixture further homogenized using 5-10 strokes and the volume was adjusted to 10 mL with distilled water. Serial dilutions of this mixture were made using distilled water to give the micrograms of Niclosamide shown on the x-axis, which were contained in 100 μL. L-4F (free base) was diluted with water to give 10 μg per 100 μL of water. One hundred microliters of the Niclosamide solution was given by stomach tube to each mouse in each group of twelve-month-old non-fasting female apoE null mice (n=4 per group) and immediately followed by 100 μL containing 10 μg of L-4F (free base) in water. The mice were fasted and after 7 hours they were bled and their plasma was sucrose cryopreserved. The plasma was fractionated by FPLC and the HDL-containing fractions were tested for their ability to inhibit the induction of monocyte chemotactic activity by a standard control human LDL, which was added to cultures of human aortic endothelial cells. The standard control human LDL was also added by itself or with a standard control human HDL. The values obtained by the standard control human LDL alone were normalized to 1.0. The values obtained after the addition of the standard control HDL or the mouse HDL were compared to the values obtained by the standard control LDL alone to give the HDL Inflammatory Index.

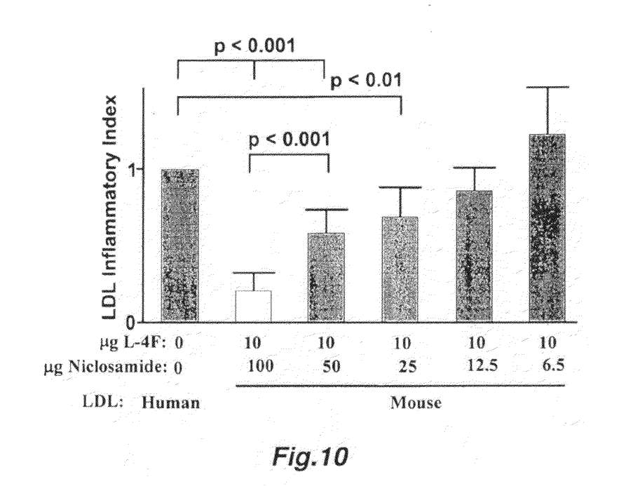

FIG. 10 shows that Administration of Niclosamide as an oral bolus by stomach tube immediately followed by administration of L-4F as an oral bolus by stomach tube significantly reduced the ability of apoE null mouse LDL to induce monocyte chemotactic activity in cultures of human aortic endothelial cells. The LDL fractions from the mice described in FIG. 9 were tested for their ability to induce monocyte chemotactic activity in cultures of human aortic endothelial cells and compared to a standard control human LDL whose values were normalized to 1.0 for the LDL-inflammatory index.

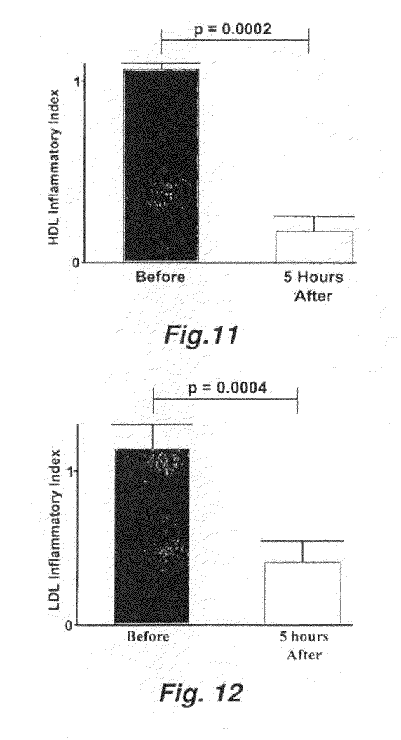

FIG. 11 shows that oral administration of niclosamide (5.0 mg/kg body weight) immediately followed by oral administration of L-4F (0.5 mg/kg/body weight) renders monkey HDL anti-inflammatory. One hundred mg of niclosamide was placed in a glass-glass homogenizer with mortar and round bottom pestle (Kontes Dounce Tissue grinder, K885300-0015 available from Fisher, VWR) and 200 μL of ethanol was added. The Niclosamide ethanol mixture was homogenized using 2-3 strokes and distilled water was added and the mixture further homogenized using 5-10 strokes and the volume was adjusted to 10 mL with distilled water. The niclosamide mixture was again mixed immediately before the dose was removed as the Niclosamide tends to settle out. Each of 4 monkeys (2 Female and 2 Male) were given 5.0 mg/kg body weight of Niclosamide contained in 2.5 mL of the mixture by stomach tube. L-4F (free base) was added to 10 mL of distilled water in the glass-glass homogenizer and homogenized using 5-10 strokes. Immediately after administration of the Niclosamide mixture each monkey was given 0.5 mg/kg body weight of L-4F (free base) contained in 2.5 mL water by stomach tube. Blood was obtained 5 hours later and the plasma was separated by FPLC and the lipoproteins tested as described in FIG. 8 for the HDL-inflammatory index and FIG. 10 for the LDL-inflammatory index. The data shown are the Mean±S.D. for the HDL Inflammatory Index for monkey HDL before and 5 hours after treatment (the data for the standard control human LDL alone and the standard control human LDL plus the standard control human HDL are not shown in the figure).

Oral administration of niclosamide (5.0 mg/kg body weight) immediately followed by oral administration of L-4F (0.5 mg/kg/body weight) significantly reduced the ability of monkey LDL to induce monocyte chemotactic activity in cultures of human aortic endothelial cells (see, e.g., FIG. 12). The LDL fractions from the monkey plasma described in FIG. 11 were tested as described in FIG. 10.

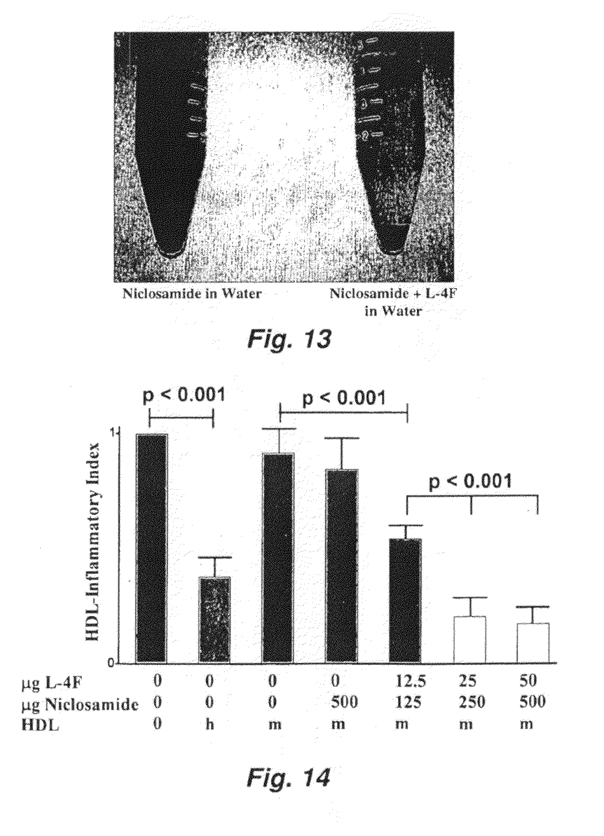

Niclosamide is relatively insoluble in aqueous solutions even when added in ethanol and homogenized. It was a surprising finding of this invention that L-4F solubilized niclosamide in aqueous solution as shown in FIG. 13. Niclosamide at 10 mg per mL was added to water, or to water containing 1.0 mg/mL L-4F (free base) and was homogenized in a glass-glass homogenizer. The solutions were stored at 4° C. for ten days and photographed (see FIG. 13).

The solutions of Niclosamide with or without L-4F shown above in FIG. 13 were serially diluted and given by gastric gavage (stomach tube) to fasting seven month old female apoE null mice in a volume of 100 μL per mouse (n=8 per group). Blood was collected 6 hrs following treatment while the mice were still fasting and the plasma was separated by FPLC and the HDL fractions were tested as described in FIG. 8 and the data are shown in FIG. 14.

The micrograms of L-4F and/or niclosamides are shown on the X-axis. Six hours after administration the mice were bled and the ability of mouse HDL (m) or human HDL (h) to inhibit LDL-induced monocyte chemotactic activity in cultures of human aortic endothelial cells was determined and plotted as the HDL-inflammatory index as described for FIG. 8.

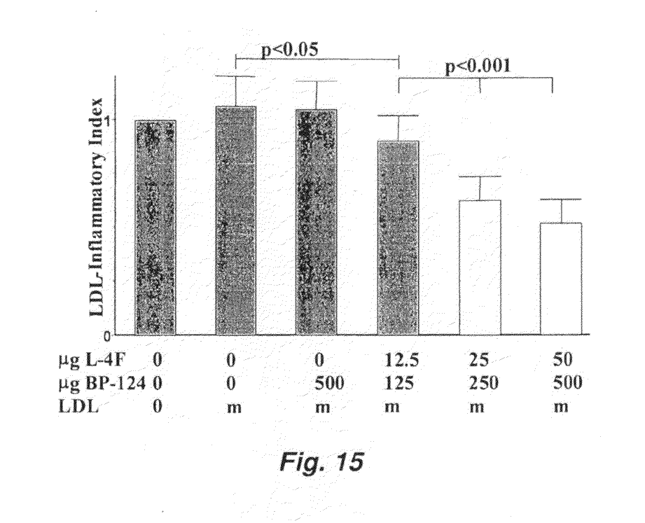

As shown in FIG. 15, administration of the L-4F together with the solubilized niclosamide resulted in a significant reduction in the ability of mouse LDL to induce monocyte chemotactic activity in cultures of human aortic endothelial cells.

The data in FIGS. 14 and 15 demonstrate the remarkable, novel, and unexpected findings that the peptide L-4F solubilizes niclosamide and results in a therapeutic combination that renders HDL anti-inflammatory and significantly reduces the inflammatory properties of LDL in a mouse model of atherosclerosis.

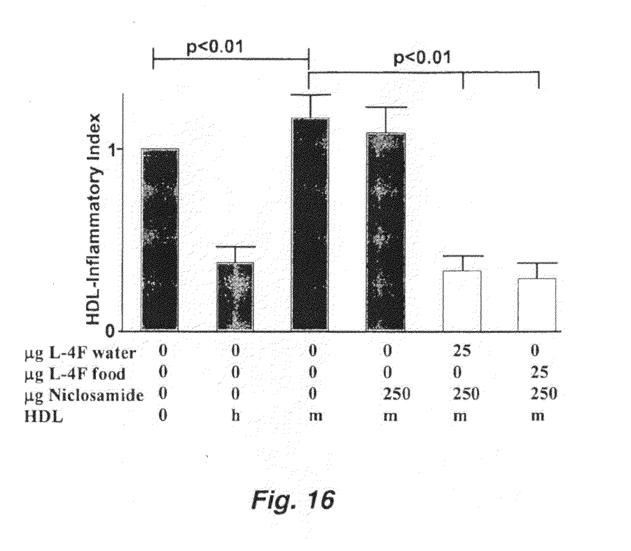

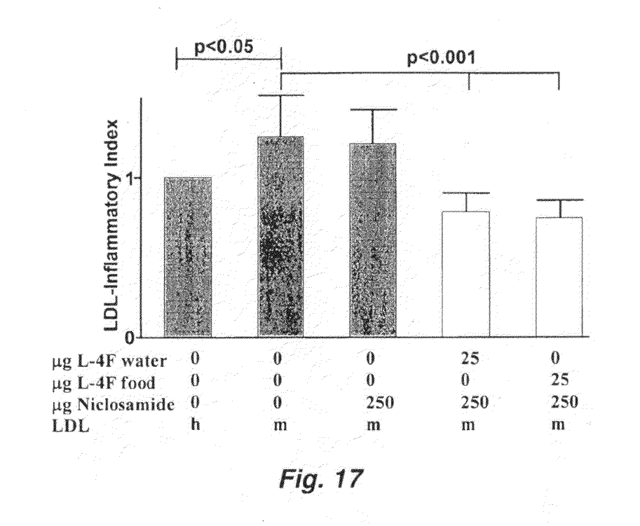

It was also a surprising finding of this invention that administration of Niclosamide in mouse chow greatly enhanced the ability of L-4F to render HDL anti-inflammatory and to decrease the ability of LDL to induce monocyte chemotactic activity in cultures of human aortic endothelial cells even when the L-4F was administered in the drinking water (see, e.g., FIGS. 16 and 17).

L-4F was previously thought to be ineffective in rendering HDL anti-inflammatory and ineffective in reducing the ability of LDL to induce monocyte chemotactic activity in cultures of human aortic endothelial cells if the peptide was given orally (see, e.g., Navab et al. (2002) Circulation, 105: 290-292). The data in FIGS. 8-17 demonstrate the surprising and unexpected finding that if L-4F is given orally with niclosamide it is highly effective in rendering HDL anti-inflammatory and highly effective in reducing the inflammatory properties of LDL. This invention also demonstrates the surprising and unexpected finding that L-4F solubilizes niclosamide.

### Example 2

### Salicylanilides Combined with L-4F Enhance Formation of Pre-Beta HDL

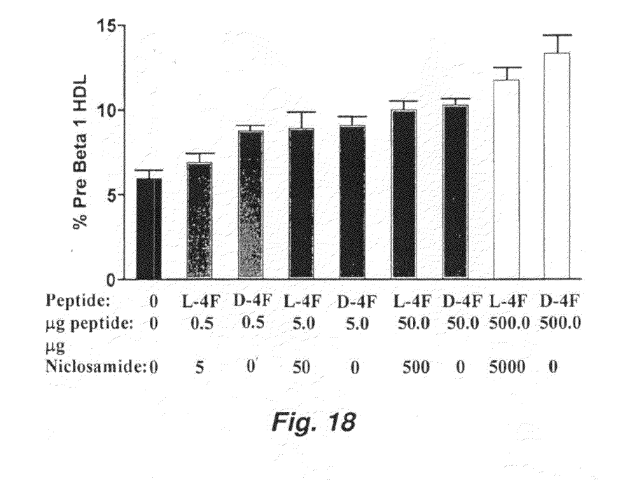

Niclosamide plus L-4F causes the formation of pre-β HDL in apoE null mice after oral administration (see, e.g., FIG. 18). D-4F (free base) was dissolved in 0.1% Tween20 in ammonium bicarbonate buffer (ABCT) pH 7.0. L-4F (free base) plus niclosamide were dissolved in ABCT in a ratio of 1:10 (L-4F:Niclosamide; wt:wt). ABCT alone or ABCT containing the micrograms of L-4F or D-4F with or without the micrograms of niclosamide shown in FIG. 18 on the X-axis were administered in 100 μL by stomach tube to 8 month old female apoE null mice that were fasted overnight (n=8 per group). Thirty to forty minutes later the mice were bled and the percent of apolipoprotein A-I contained in pre-β-1 HDL was determined in triplicate 2-dimensional gels by scanning. The data shown are the Mean±S.D.

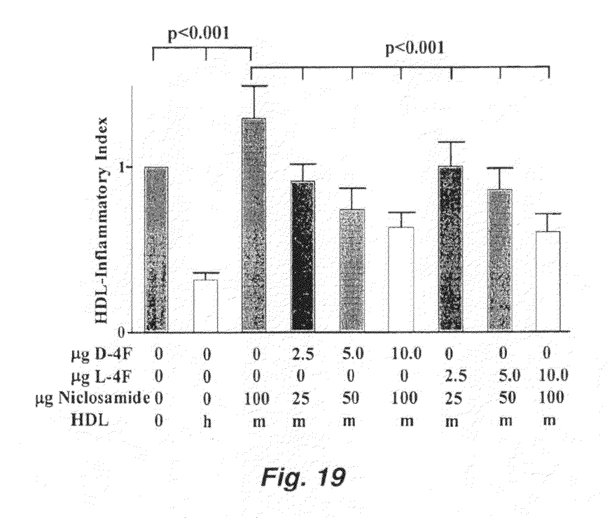

It was also a surprising discovery that oral co-administration of niclosamide and L-4F improved the inflammatory properties of apoE null mouse HDL (as measured in a cell-based assay) to a degree similar to that seen when niclosamide was administered with D-4F (see, e.g., FIG. 19).

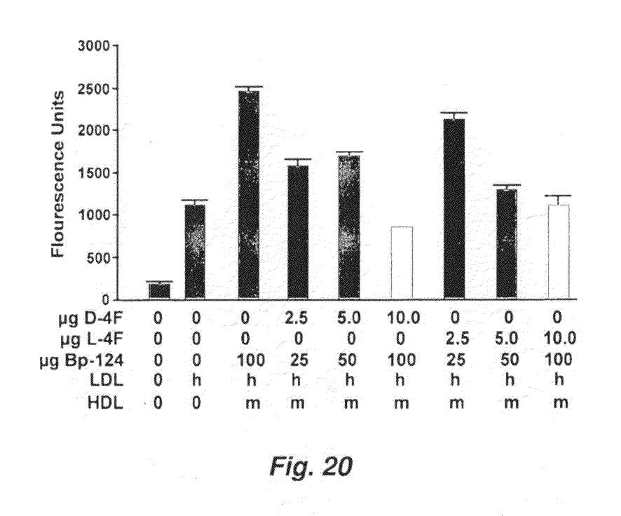

Similar results were obtained when the inflammatory properties of HDL were measured by a cell-free assay (see, e.g., FIG. 20).

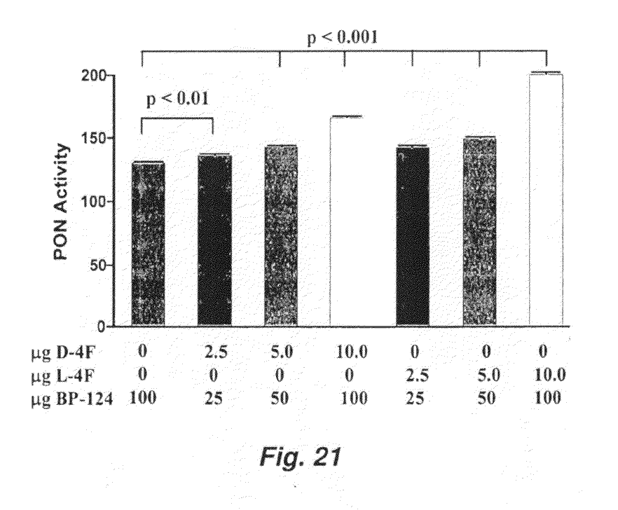

It was also a surprising discovery that when niclosamide and L-4F were co-administered orally to apoE null mice the increase in paraoxonase activity was similar to that seen when niclosamide was co-administered with D-4F (see, e.g. FIG. 21).

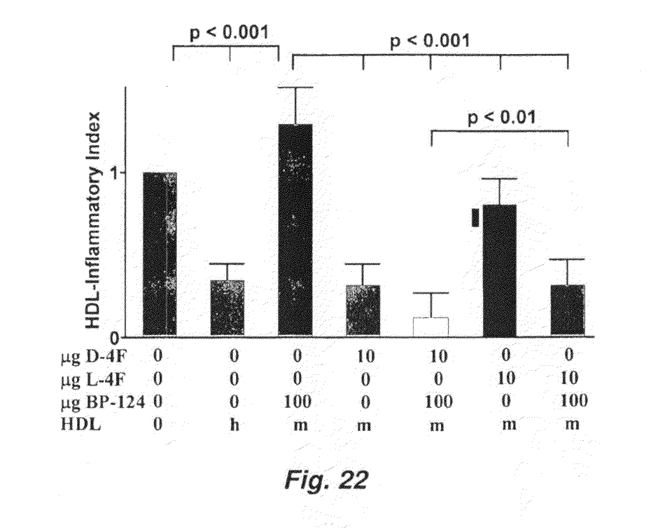

Oral co-administration of niclosamide with either D-4F or L-4F enhanced the ability of both peptides to improve HDL inflammatory properties in apoE null mice. In the absence of niclosamide, however, D-4F was able to render apoE null mouse HDL anti-inflammatory to a degree comparable to normal human HDL while L-4F was only able to achieve this degree of efficacy when co-administered with niclosamide (see, e.g., FIG. 22).

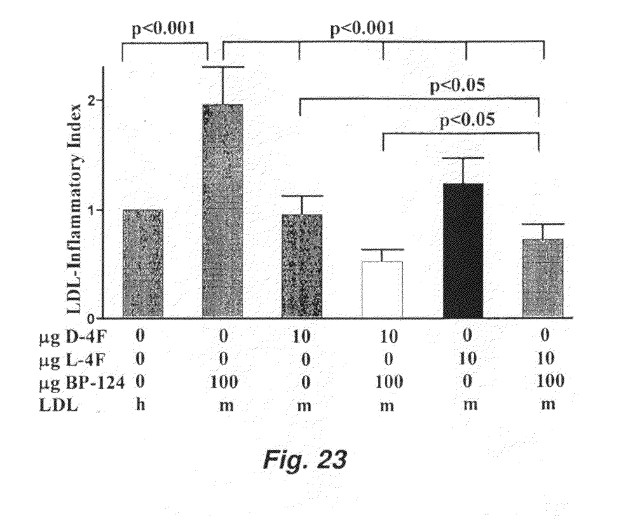

As shown in FIG. 23 the inflammatory properties of LDL from apoE null mice were reduced by the co-administration orally of niclosamide and L-4F.

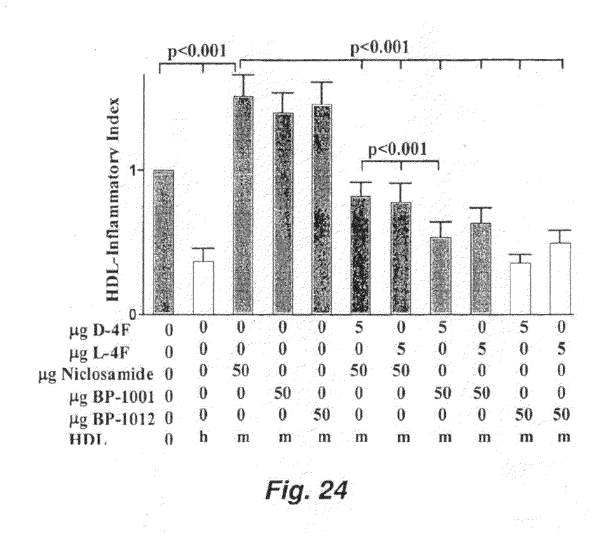

It was a surprising discovery of this invention that some of the salicylanilides described in FIGS. 24-26 were even more potent than niclosamide in rendering apoE null mouse HDL anti-inflammatory when administered orally together with either D-4F or L-4F. As shown in FIG. 24 neither niclosamide nor the new salicylanilides were anti-inflammatory when administered without the peptides.

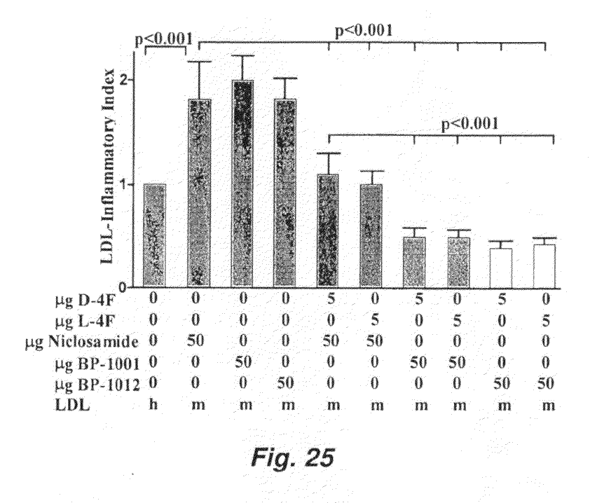

As shown in FIG. 25 the new salicylanilides (BP-1001 and BP-1012) were also more potent in reducing the inflammatory properties of LDL than niclosamide when co-administered with D-4F or L-4F.

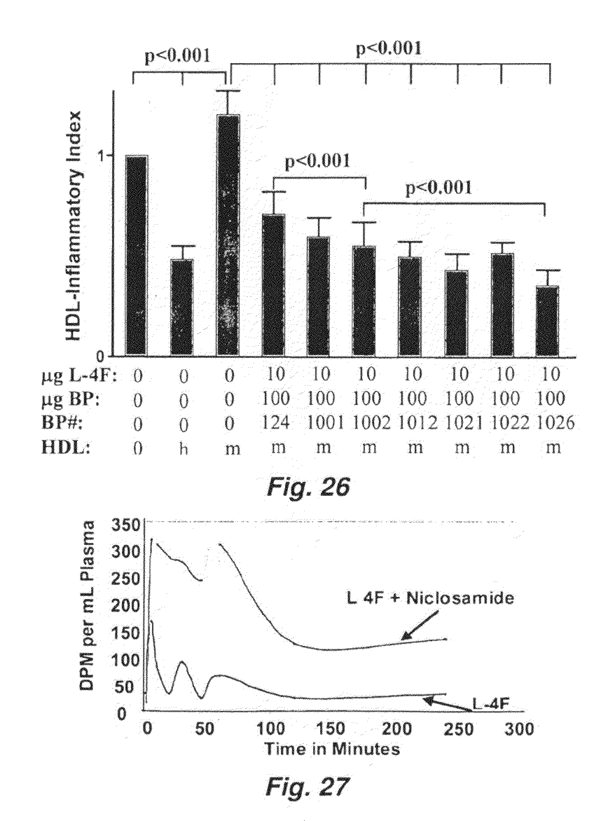

As shown in FIG. 26, other salicylanilides were similar to niclosamide (BP-124) in bioactivity while still others were more potent.

### Example 3

### Niclosamide Increases L-4F Absorption in ApoE Null Mice

L-4F absorption was determined with and without niclosamide (BP-124) using ¹⁴C-L-4F. Fasted female apoE null mice 6-months of age (n=4 per group) were administered by stomach tube L-4F (21,000 dpm containing 10 micrograms of L-4F per mouse) with or without 100 micrograms of niclosamide in 200 μL 0.1% Tween20 in ammonium bicarbonate at pH 7.0. Fasting was continued and the mice were bled at the time points shown on the X-axis in FIG. 27 and the dpm per mL plasma determined. The area under the curve (AUC) in FIG. 27 for the mice receiving L-4F+niclosamide was 4.4 times greater than the AUC for the mice receiving L-4F without niclosamide.

The data indicate that one of the mechanisms by which niclosamide enhances the in vivo bioactivity of L-4F is by increasing the absorption of L-4F.

The foregoing data (Examples 1, 2, and 3) show that the combination of niclosamide or other salicylanilides with L-4F, and presumably other therapeutic peptides, appears to have great potential for oral therapy. Based on these data it is believed that the use of niclosamide or other salicylanilides with other peptides or proteins will make new oral therapeutics possible.

The data in FIG. 27 indicate that without niclosamide administration of ¹⁴C-L-4F by stomach tube resulted in low plasma levels that lasted no more than 5 minutes. In contrast, when ¹⁴C-L-4F was administered with niclosamide a Cₘₐₓ of approximately 150 nanograms/mL was achieved which persisted for more than an hour and at a lower level for up to four hours.

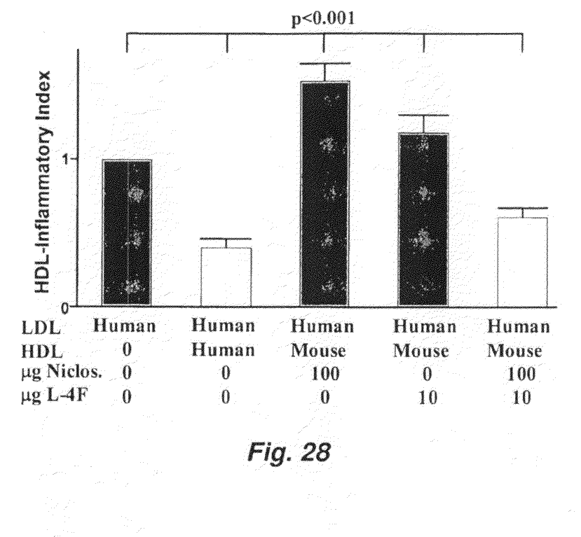

The data in FIG. 28 demonstrate that the ¹⁴C-L-4F used in FIG. 28 was biologically active when given with niclosamide. Fasted apoE null mice 5-months of age (n=4 per group) were administered by stomach tube ¹⁴C-L-4F (21,000 dpm containing 10 μg of L-4F per mouse) with or without 100 μg of niclosamide (Niclos.) in 200 μl. Fasting was continued and the mice were bled 5 hours later and the HDL inflammatory index determined in cultures of human aortic endothelial cells as described in FIG. 8. Briefly, To determine the HDL-inflammatory index lipoproteins were added to human aortic endothelial cell cultures as described previously (Navab et al., (2005) Circulation Research 97: 524-532). A normal control human LDL was added to each well in triplicate at a final concentration of 100 μg/mL of LDL-cholesterol. A normal human HDL was added to three wells containing human LDL at a final concentration of 50 μg/mL HDL-cholesterol as a positive control. HDL from the mice at a final concentration of 50 μg/mL HDL-cholesterol was added in triplicate to other wells containing human LDL. After 8 hours of culture the supernatants were removed and monocyte chemotactic activity was determined as previously described (Navab et al. (2001) J. Lipid Res., 42: 1308-1317; Danciger et al. (2004) J. Immunol. Meth., 288: 123-124). The values obtained from wells containing the human LDL but no HDL were normalized to 1.0. The values obtained from wells containing the human LDL with either human or mouse HDL were divided by the values obtained from wells with human LDL without added HDL to give the HDL-inflammatory index as previously described (Ansell et al. (2003) Circulation 108: 2751-2756). The data in FIG. 28 demonstrate that the ¹⁴C-L-4F used in the experiments described in FIG. 27 was biologically active.

### Example 4

### Niclosamide Plus L-4F Administered Orally (but not L-4F Alone) Reduces Lesions in Mouse Models Of Atherosclerosis

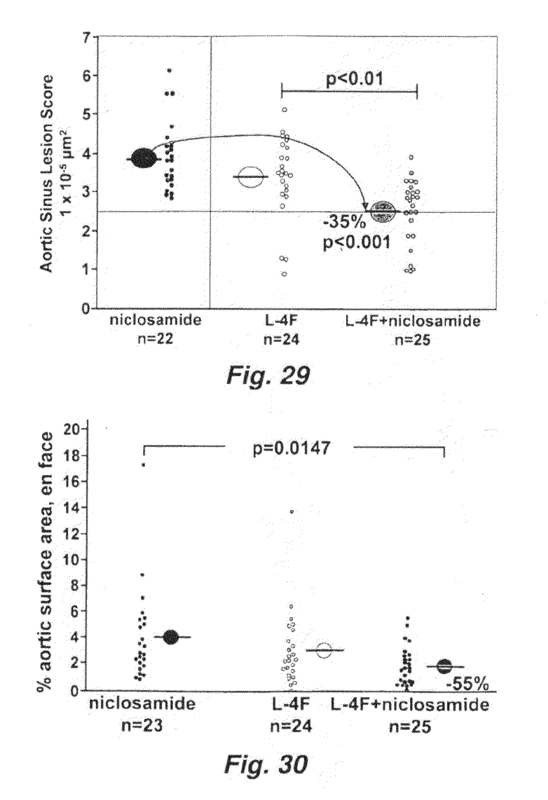

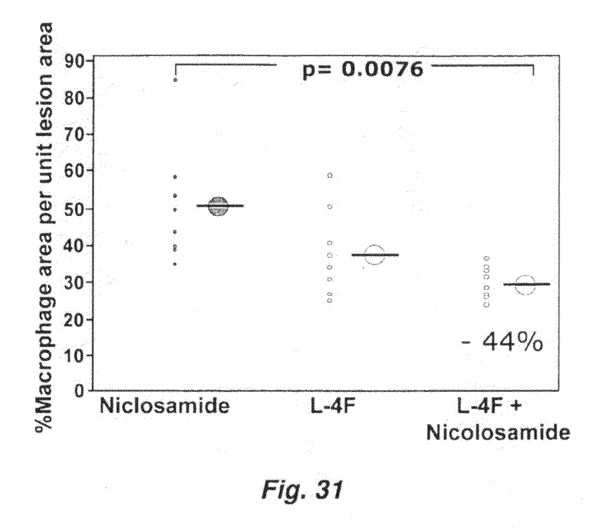

In another experiment, seventeen week old female apoE null mice were divided into three groups: Group I received niclosamide 250 μg/mouse/day in rodent chow. Group II received L-4F at 25 μg/mouse/day in rodent chow. Group III received niclosamide at 250 μg/mouse/day together with L-4F 25 μg/mouse/day in rodent chow. All three groups received pravastatin 50 μg/mouse/day in drinking water. After 14 weeks the mice were sacrificed and aortic sinus lesion area was determined. As shown in FIGS. 29-31 oral administration of L-4F together with niclosamide but not without niclosamide significantly inhibits atherosclerosis in apoE Null mice.

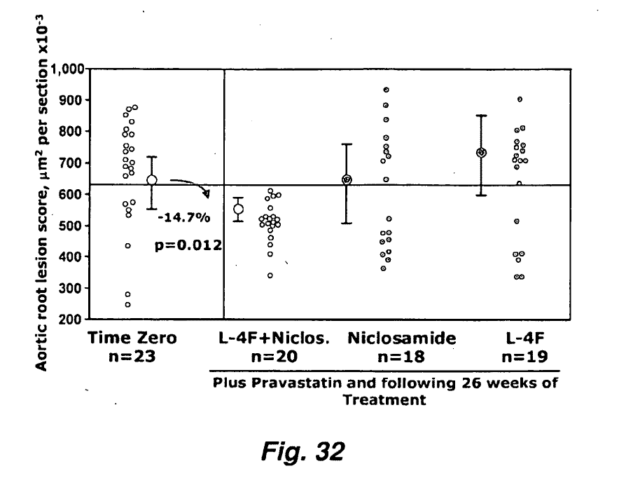

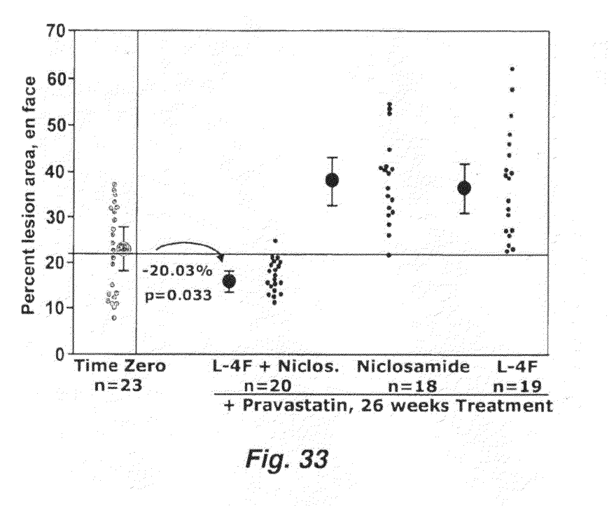

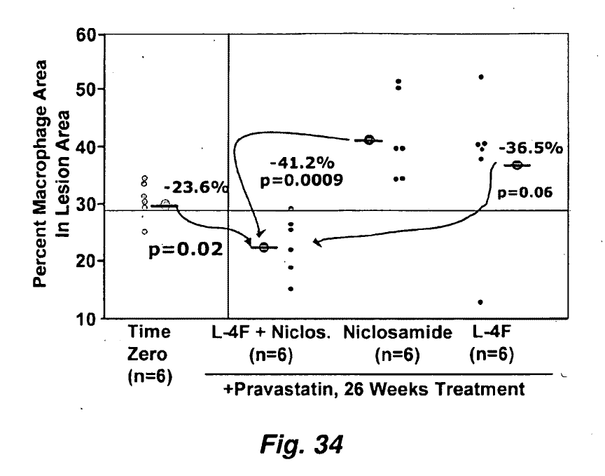

In still another experiments, nine and half months-old female apoE null mice were divided into four groups: Group I was sacrificed to establish base line lesion area (Time Zero). Group II received niclosamide at 2 mg/mouse/day in rodent chow. Group III received L-4F at 200 μg/mouse/day in rodent chow. Group IV received niclosamide (Niclos.) at 2 mg/mouse/day together with L-4F 200 μg/mouse/day in rodent chow. Groups II-IV received pravastatin 50 μg/mouse/day in drinking water. After 26 weeks the mice were sacrificed and aortic sinus lesion area was determined. The data in FIGS. 32-34 demonstrate that the combination of L-4F plus niclosamide caused lesion regression in old apoE null mice. In contrast, neither niclosamide nor L-4F without niclosamide significantly reduced lesions.

L-4F forms a class A amphipathic helix. The sequence comprising residues 113-122 in apolipoprotein J (apoJ) comprises a potential G* helix. Administration of this peptide synthesized from all D-amino acids, D[113-122]apoJ, dramatically improved HDL inflammatory properties and reduced atherosclerosis in apoE null mice (Navab et al. (2005) Arterioscler. Thromb. Vasc. Biol. 25: 1932-1937).

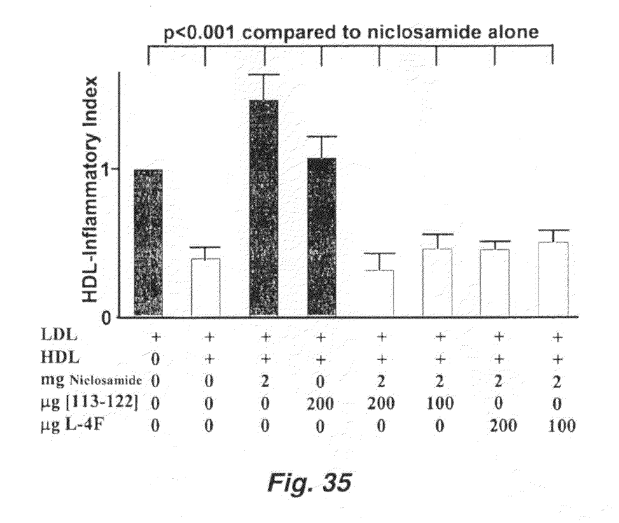

To determine whether niclosamide could improve activity of the L-form of apoJ, ten month old apoE null mice (n=4 per group) were administered by stomach tube 2 mg of niclosamide or 200 μg of L-[113-122]apoJ or 2 mg of niclosamide together with 100 or 200 μg of L-[113-122]apoJ or were administered 2 mg of niclosamide together with 100 or 200 μg of L-4F. Eight hours later the mice were bled and the HDL inflammatory index was determined in cultures of human aortic endothelial cells as described in FIG. 8. As shown in FIG. 35 oral administration of the same peptide but synthesized from all L-amino acids and administered with niclosamide rendered apoE null mouse HDL anti-inflammatory to the same degree as normal human HDL, but when the peptide was administered orally without niclosamide this was not the case.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 5

### Niclosamide Interacts with L-4F to Form a Complex that is Resistant to the Action of Trypsin Allowing L-4F to be Absorbed after Oral Administration in a Biologically Active Form

It was a surprising discovery of this invention that niclosamide forms a complex with L-4F that can be isolated by simple physical-chemical means. As shown in FIG. 36 after incubating L-4F and niclosamide at conditions similar to that encountered in the stomach a L-4F-niclosamide complex formed that was easily isolated by differential centrifugation. As shown in FIG. 37 oral administration of this L-4F-niclosamide complex was highly effective in rendering HDL from apoE null mice anti-inflammatory. In contrast, administering L-4F orally without niclosamide was not effective (see 12KS for L-4F without niclosamide in FIG. 37).

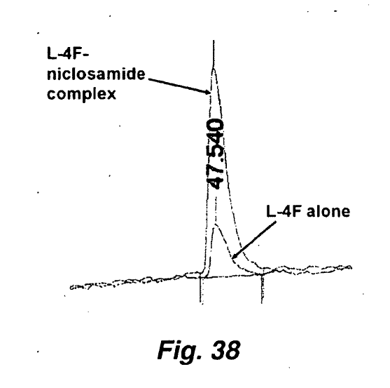

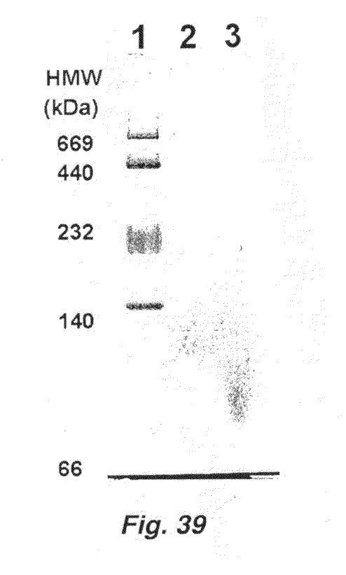

It was also a surprising discovery of this invention that the complex formed by L-4F and niclosamide is resistant to trypsin degradation compared to L-4F that was not complexed to niclosamide (FIG. 38). The area under the curve for the persistence in the plasma of radioactive L-4F when orally administered with niclosamide was 4.4-fold greater than was the case for administration of the radioactive L-4F without niclosamide (FIG. 27). It is interesting that the area under the curve for L-4 remaining after trypsin treatment in FIG. 39 when the L-4F was complexed with niclosamide was about 4-fold greater than was the case when L-4F was not complexed with niclosamide and was treated with trypsin. These data suggest that one of the mechanisms by which the L-4F-niclosamide complex results in greater bioactivity after oral administration is because the complex formed protects L-4F against the action of digestive enzymes in the gastrointestinal tract.

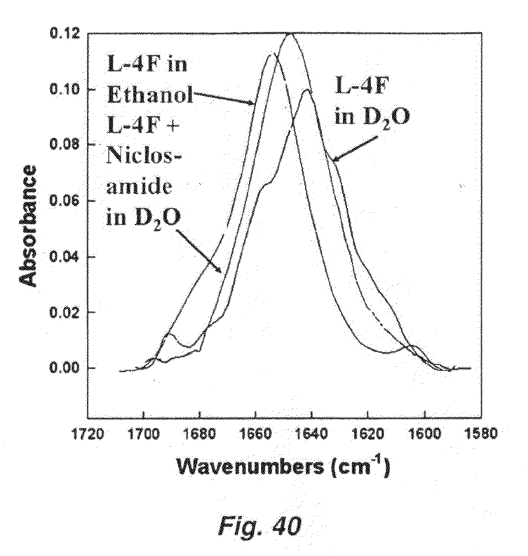

In an aqueous environment L-4F which has a molecular weight of 2310 daltons self-associates and forms micelles which have a molecular weight of >100 kDa (see lane 2 in FIG. 39). When complexed to niclosamide the micelles formed by L-4F self-association in an aqueous environment are much smaller (see lane 3 in FIG. 39). As demonstrated in FIG. 40 the L-4F-niclosamide complex results in preservation of the helical structure of L-4F and a minimum formation of beta sheet aggregates. This favorable conformational change induced in L-4F by complexing with niclosamide likely both protects the peptide from the action of proteolytic enzymes in the digestive tract and also promotes greater absorption and bioactivity.

1. A method of enhancing the in vivo activity of a therapeutic peptide orally administered to a mammal, said method comprising reacting the peptide with a salicylanilide and/or with the parent acid or amine of the salicylanilide and/or with acetyl salicylic acid or a derivative of acetyl salicylic to form a complex with said peptide whereby the peptide complex shows enhanced in vivo activity as compared to the untreated peptide.

2. The method of claim 1, wherein said reacting is under acidic conditions.

3. The method of claim 1, wherein said reacting is at a pH ranging from about pH 1 to about pH 7.

4. The method of claim 3, wherein said reacting is at a temperature ranging from 30° C. to about 60° C.

5. The method of claim 3, wherein said reacting is at a temperature of about 37° C.

6. The method of claim 1, wherein said reacting is at room temperature.

7. The method of claim 1, wherein said salicylanilide is niclosamide or a niclosamide analogue.

8. The method of claim 1, wherein said niclosamide or niclosamide analogue is selected from the group consisting of 2′5-dichloro-4′-nitrosalicylanilide, 5-chloro-salicyl-(2-chloro-4-nitro) anilide 2-aminoethanol salt, 5-chloro-salicyl-(2-chloro-4-nitro) anilide piperazine salt, and 5-chloro-salicyl-(2-chloro-4-nitro) anilide monohydrate.

9. The method of claim 1, wherein said niclosamide analogue is a compound in one or more of FIGS. 2, 3, 4, 5, 6, 7, and/or Table 1.

10. The method of claim 1, wherein said parent acid and/or said parent amine is an acid or amine in Table 1.

11. The method of claim 1, wherein said peptide ranges in length from 3 amino acids to 300 amino acids.

12. The method of claim 1, wherein said peptide forms an amphipathic helix.

13. The method of claim 1, wherein said peptide is selected from the group consisting of ApoJ, ApoA-I, ApoA-I milano, and 18A.

14. The method of claim 1, wherein said peptide comprises a class A amphipathic helix.

15. The method of claim 1, wherein said peptide consists of all “L” amino acids.

16. (canceled)

17. The method of claim 1, wherein said peptide consists of all “D” amino acids.

18. The method of claim 1, wherein said peptide is a D or L peptide whose sequence is shown in any of Tables 2-11 and/or SEQ ID Nos:1-995.

19. The method of claim 18, wherein said peptide consists of all L amino acids.

20. The method of claim 18, wherein said peptide comprises a protecting group at the amino or carboxyl terminus.

21. The method of claim 18, wherein said peptide comprises a first protecting group coupled to the amino terminus and a second protecting group coupled to the carboxyl terminus.

22. The method of claim 21, wherein said protecting group is a protecting group selected from the group consisting of acetyl, amide, and 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl-Z),2-bromobenzyloxycarbonyl (2-Br—Z), Benzyloxymethyl (Bom), t-butoxycarbonyl (Boc), cyclohexyloxy (cHxO),t-butoxymethyl (Bum), t-butoxy (tBuO), t-Butyl (tBu), Acetyl (Ac), and Trifluoroacetyl (TFA).

23. The method of claim 21, wherein said first protecting group is a protecting group selected from the group consisting of acetyl, propeonyl, and a 3 to 20 carbon alkyl.

24. The method of claim 23, wherein said second protecting group is an amide.

25. The method of claim 1, wherein said peptide is a D or L peptide comprising the amino acid sequence DWFKAFYDKVAEKFKEAF (SEQ ID NO:5) or the amino acid sequence FAEKFKEAVKDYFAKFWD (SEQ ID NO:104).

26. The method of claim 25, wherein said peptide comprises a carboxyl terminal protecting group and an amino terminal protecting group.

27. The method of claim 26, wherein:
said peptide comprises a protecting group coupled to the carboxyl terminus and said carboxyl terminal protecting group is an amide; and
said peptide comprises a protecting group coupled to the amino terminus and said amino terminal protecting group is an acetyl.

28. The method of claim 27, wherein said salicylanilide is niclosamide or a niclosamide analogue.

29. (canceled)

30. A method of preparing an orally deliverable therapeutic peptide, said method comprising synthesizing said peptide with one or more amino acids that are acetylated at the epsilon position of the amino acid with a salicylanilide and/or with the parent acid or amine of the salicylanilide and/or with acetyl salicylic acid or a derivative of acetyl salicylic to form an adduct with said peptide whereby the peptide adduct shows enhanced in vivo activity as compared to the untreated peptide.

31. The method of claim 30, wherein said peptide is acylated at one or more lysines.

32. A composition comprising a modified peptide having the structure of a complex formed by reacting a therapeutically active peptide with a salicylanilide and/or with the parent acid or amine of the salicylanilide and/or with acetyl salicylic acid or a derivative of acetyl salicylic to form a complex with said peptide whereby the modified peptide shows enhanced resistance to proteolysis and/or enhanced in vivo activity as compared to the untreated peptide.

33-39. (canceled)

40. The composition of claim 32, wherein said niclosamide analogue is a compound in one or more of FIG. 2, 3, 4, 5, 6, or 7, and/or Table 1.

41. The composition of claim 32, wherein said parent acid and/or said parent amine is an acid or amine in Table 1.

42. The composition of claim 32, wherein said peptide ranges in length from 3 amino acids to about 300 amino acids.

43. The composition of claim 32, wherein said peptide comprises an amphipathic helix.

44. The composition of claim 32, wherein said peptide is selected from the group consisting of ApoJ, ApoA-I, ApoA-I milano, and 18A.

45-48. (canceled)

49. The composition of claim 32, wherein said peptide is a D or L peptide whose sequence is shown in any of Tables 2-11 and/or SEQ ID Nos:1-995.

50-51. (canceled)

52. The composition of claim 49, wherein said peptide comprises a first protecting group coupled to the amino terminus and a second protecting group coupled to the carboxyl terminus.

53. The composition of claim 52, wherein said protecting group is a protecting group selected from the group consisting of acetyl, amide, and 3 to 20 carbon alkyl groups, Fmoc, Tboc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z),2-bromobenzyloxycarbonyl (2-Br—Z), Benzyloxymethyl (Bom), t-butoxycarbonyl (Boc), cyclohexyloxy (cHxO),t-butoxymethyl (Bum), t-butoxy (tBuO), t-Butyl (tBu), Acetyl (Ac), and Trifluoroacetyl (TFA).

54. The composition of claim 52, wherein said first protecting group is a protecting group selected from the group consisting of acetyl, propeonyl, and a 3 to 20 carbon alkyl.

55. The composition of claim 54, wherein said second protecting group is an amide.

56-60. (canceled)

61. The composition of claim 32, wherein said peptide is combined with a pharmaceutically acceptable excipient.

62. The composition of claim 61, wherein said excipient is suitable for administration by a route selected from the group consisting of oral administration, nasal administration, rectal administration, intraperitoneal injection, intravascular injection, subcutaneous injection, transcutaneous administration, inhalation administration, and intramuscular injection.

63. The composition of claim 62, wherein said composition is formulated as a unit dosage formulation.

64. An orally deliverable therapeutic peptide said peptide comprising:
a therapeutic peptide comprising one or more amino acids that are acetylated at the epsilon position of the amino acid with the a salicylanilide and/or with the parent acid or amine of the salicylanilide and/or with acetyl salicylic acid or a derivative of acetyl salicylic to form an adduct with said peptide whereby the peptide adduct shows enhanced in vivo activity as compared to the untreated peptide.

65-68. (canceled)

69. The peptide of claim 64, wherein said peptide comprises an amphipathic helix.

70. The peptide of claim 64, wherein said peptide is selected from the group consisting of ApoJ, ApoA-I, ApoA-I milano, and 18A.

71. (canceled)

72. A method of mitigating one or more symptoms of a pathology characterized by an inflammatory response in a mammal, said method comprising:
orally administering to said mammal a modified amphipathic helical peptide that mitigates one or more symptoms of atherosclerosis or other pathology characterized by an inflammatory response, whereby said oral delivery provides in vivo activity of said peptide to mitigate one or more symptoms of said pathology, and where said modified peptide has the structure of a complex formed by reacting a therapeutically active peptide with a salicylanilide and/or with the parent acid or amine of the salicylanilide and/or with acetyl salicylic acid or a derivative of acetyl salicylic to form a complex with said peptide whereby the peptide-salicylanilide complex shows enhanced resistance to proteolysis and/or enhanced in vivo activity as compared to the untreated peptide.

73-78. (canceled)

79. The method of claim 72, wherein said niclosamide analogue is a compound in FIG. 2, 3, 4, 5, 6, 7, and/or Table 1.

80. The method of claim 72, wherein said parent acid and/or said parent amine is an acid or amine in Table 1.

81. The method of claim 72, wherein said peptide ranges in length from 3 amino acids to 300 amino acids.

82. The method of claim 72, wherein said peptide is selected from the group consisting of ApoJ, ApoA-I, ApoA-I milano, and 18A.

83-86. (canceled)

87. The method of claim 72, wherein said peptide is a D or L peptide whose sequence is shown in any of Tables 2-11 and/or SEQ ID Nos:1-995.

88-101. (canceled)

102. The method of claim 72, wherein said pathology is selected from the group consisting of atherosclerosis, rheumatoid arthritis, lupus erythematous, polyarteritis nodosa, osteoporosis, Alzheimer's disease, multiple sclerosis, chronic obstructive pulmonary disease, asthma, diabetes, chronic renal disease, and a viral illnesses.

103-104. (canceled)


SEQUENCE LISTING (995 sequences)

SEQ ID NO: 1 - protein, 18 aa, Artificial Sequence
  Synthetic peptide with optional protecting groups.
DWLKAFYDKVAEKLKEAF

SEQ ID NO: 2 - protein, 18 aa, Artificial Sequence
  Synthetic peptide with optional protecting groups.
DWLKAFYDKVAEKLKEAF

SEQ ID NO: 3 - protein, 18 aa, Artificial Sequence
  Synthetic peptide with optional protecting groups.
DWFKAFYDKVAEKLKEAF

SEQ ID NO: 4 - protein, 18 aa, Artificial Sequence
  Synthetic peptide with optional protecting groups.
DWLKAFYDKVAEKFKEAF

SEQ ID NO: 5 - protein, 18 aa, Artificial Sequence
  Synthetic peptide with optional protecting groups.
DWFKAFYDKVAEKFKEAF

SEQ ID NO: 6 - protein, 18 aa, Artificial Sequence
  Synthetic peptide with optional protecting groups.
DWLKAFYDKVFEKFKEFF

SEQ ID NO: 7 - protein, 18 aa, Artificial Sequence
  Synthetic peptide with optional protecting groups.
DWLKAFYDKFFEKFKEFF

SEQ ID NO: 8 - protein, 18 aa, Artificial Sequence
  Synthetic peptide with optional protecting groups.
DWFKAFYDKFFEKFKEFF

SEQ ID NO: 9 - protein, 18 aa, Artificial Sequence
  Synthetic peptide with optional protecting groups.
DWLKAFYDKVAEKLKEFF

SEQ ID NO: 10 - protein, 18 aa, Artificial Sequence
  Synthetic peptide with optional protecting groups.
DWLKAFYDKVFEKFKEAF

SEQ ID NO: 11 - protein, 18 aa, Artificial Sequence
  Synthetic peptide with optional protecting groups.
DWLKAFYDKVFEKLKEFF

SEQ ID NO: 12 - protein, 18 aa, Artificial Sequence
  Synthetic peptide with optional protecting groups.
DWLKAFYDKVAEKFKEFF

SEQ ID NO: 13 - protein, 18 aa, Artificial Sequence
  Synthetic peptide with optional protecting groups.
DWLKAFYDKVFEKFKEFF

SEQ ID NO: 14 - protein, 18 aa, Artificial Sequence
  Synthetic peptide with optional protecting groups.
EWLKLFYEKVLEKFKEAF

SEQ ID NO: 15 - protein, 18 aa, Artificial Sequence
  Synthetic peptide with optional protecting groups.
EWLKAFYDKVAEKFKEAF

SEQ ID NO: 16 - protein, 18 aa, Artificial Sequence
  Synthetic peptide with optional protecting groups.
EWLKAFYDKVAEKLKEFF

SEQ ID NO: 17 - protein, 18 aa, Artificial Sequence
  Synthetic peptide with optional protecting groups.
EWLKAFYDKVFEKFKEAF

SEQ ID NO: 18 - protein, 18 aa, Artificial Sequence
  Synthetic peptide with optional protecting groups.
EWLKAFYDKVFEKLKEFF

SEQ ID NO: 19 - protein, 18 aa, Artificial Sequence
  Synthetic peptide with optional protecting groups.
EWLKAFYDKVAEKFKEFF

SEQ ID NO: 20 - protein, 18 aa, Artificial Sequence
  Synthetic peptide with optional protecting groups.
EWLKAFYDKVFEKFKEFF

SEQ ID NO: 21 - protein, 14 aa, Artificial Sequence
  Synthetic peptide with optional protecting groups.
AFYDKVAEKLKEAF

SEQ ID NO: 22 - protein, 14 aa, Artificial Sequence
  Synthetic peptide with optional protecting groups.
AFYDKVAEKFKEAF

SEQ ID NO: 23 - protein, 14 aa, Artificial Sequence
  Synthetic peptide with optional protecting groups.
AFYDKVAEKFKEAF

SEQ ID NO: 24 - protein, 14 aa, Artificial Sequence
  Synthetic peptide with optional protecting groups.
AFYDKFFEKFKEFF

SEQ ID NO: 25 - protein, 14 aa, Artificial Sequence
  Synthetic peptide with optional protecting groups.
AFYDKFFEKFKEFF

SEQ ID NO: 26 - protein, 14 aa, Artificial Sequence
  Synthetic peptide with optional protecting groups.
AFYDKVAEKFKEAF

SEQ ID NO: 27 - protein, 14 aa, Artificial Sequence
  Synthetic peptide with optional protecting groups.
AFYDKVAEKLKEFF

SEQ ID NO: 28 - protein, 14 aa, Artificial Sequence
  Synthetic peptide with optional protecting groups.
AFYDKVFEKFKEAF

SEQ ID NO: 29 - protein, 14 aa, Artificial Sequence
  Synthetic peptide with optional protecting groups.
AFYDKVFEKLKEFF

SEQ ID NO: 30 - protein, 14 aa, Artificial Sequence
  Synthetic peptide with optional protecting groups.
AFYDKVAEKFKEFF

SEQ ID NO: 31 - protein, 14 aa, Artificial Sequence
  Synthetic peptide with optional protecting groups.
KAFYDKVFEKFKEF

SEQ ID NO: 32 - protein, 14 aa, Artificial Sequence
  Synthetic peptide with optional protecting groups.
LFYEKVLEKFKEAF

SEQ ID NO: 33 - protein, 14 aa, Artificial Sequence
  Synthetic peptide with optional protecting groups.
AFYDKVAEKFKEAF

SEQ ID NO: 34 - protein, 14 aa, Artificial Sequence
  Synthetic peptide with optional protecting groups.
AFYDKVAEKLKEFF

SEQ ID NO: 35 - protein, 14 aa, Artificial Sequence
  Synthetic peptide with optional protecting groups.
AFYDKVFEKFKEAF

SEQ ID NO: 36 - protein, 14 aa, Artificial Sequence
  Synthetic peptide with optional protecting groups.
AFYDKVFEKLKEFF

SEQ ID NO: 37 - protein, 14 aa, Artificial Sequence
  Synthetic peptide with optional protecting groups.
AFYDKVAEKFKEFF

SEQ ID NO: 38 - protein, 14 aa, Artificial Sequence
  Synthetic peptide with optional protecting groups.
AFYDKVFEKFKEFF

SEQ ID NO: 39 - protein, 18 aa, Artificial Sequence
  Synthetic peptide with optional protecting groups.
DWLKALYDKVAEKLKEAL

SEQ ID NO: 40 - protein, 18 aa, Artificial Sequence
  Synthetic peptide with optional protecting groups.
DWFKAFYEKVAEKLKEFF

SEQ ID NO: 41 - protein, 18 aa, Artificial Sequence
  Synthetic peptide with optional protecting groups.
DWFKAFYEKFFEKFKEFF

SEQ ID NO: 42 - protein, 18 aa, Artificial Sequence
  Synthetic peptide with optional protecting groups.
EWLKALYEKVAEKLKEAL

SEQ ID NO: 43 - protein, 18 aa, Artificial Sequence
  Synthetic peptide with optional protecting groups.
EWLKAFYEKVAEKLKEAF

SEQ ID NO: 44 - protein, 18 aa, Artificial Sequence
  Synthetic peptide with optional protecting groups.
EWFKAFYEKVAEKLKEFF

SEQ ID NO: 45 - protein, 18 aa, Artificial Sequence
  Synthetic peptide with optional protecting groups.
EWLKAFYEKVFEKFKEFF

SEQ ID NO: 46 - protein, 18 aa, Artificial Sequence
  Synthetic peptide with optional protecting groups.
EWLKAFYEKFFEKFKEFF

SEQ ID NO: 47 - protein, 18 aa, Artificial Sequence
  Synthetic peptide with optional protecting groups.
EWFKAFYEKFFEKFKEFF

SEQ ID NO: 48 - protein, 18 aa, Artificial Sequence
  Synthetic peptide with optional protecting groups.
DFLKAWYDKVAEKLKEAW

SEQ ID NO: 49 - protein, 18 aa, Artificial Sequence
  Synthetic peptide with optional protecting groups.
EFLKAWYEKVAEKLKEAW

SEQ ID NO: 50 - protein, 18 aa, Artificial Sequence
  Synthetic peptide with optional protecting groups.
DFWKAWYDKVAEKLKEWW

SEQ ID NO: 51 - protein, 18 aa, Artificial Sequence
  Synthetic peptide with optional protecting groups.
EFWKAWYEKVAEKLKEWW

SEQ ID NO: 52 - protein, 18 aa, Artificial Sequence
  Synthetic peptide with optional protecting groups.
DKLKAFYDKVFEWAKEAF

SEQ ID NO: 53 - protein, 18 aa, Artificial Sequence
  Synthetic peptide with optional protecting groups.
DKWKAVYDKFAEAFKEFL

SEQ ID NO: 54 - protein, 18 aa, Artificial Sequence
  Synthetic peptide with optional protecting groups.
EKLKAFYEKVFEWAKEAF

SEQ ID NO: 55 - protein, 18 aa, Artificial Sequence
  Synthetic peptide with optional protecting groups.
EKWKAVYEKFAEAFKEFL

SEQ ID NO: 56 - protein, 18 aa, Artificial Sequence
  Synthetic peptide with optional protecting groups.
DWLKAFVDKFAEKFKEAY

SEQ ID NO: 57 - protein, 18 aa, Artificial Sequence
  Synthetic peptide with optional protecting groups.
EKWKAVYEKFAEAFKEFL

SEQ ID NO: 58 - protein, 18 aa, Artificial Sequence
  Synthetic peptide with optional protecting groups.
DWLKAFVYDKVFKLKEFF

SEQ ID NO: 59 - protein, 18 aa, Artificial Sequence
  Synthetic peptide with optional protecting groups.
EWLKAFVYEKVFKLKEFF

SEQ ID NO: 60 - protein, 18 aa, Artificial Sequence
  Synthetic peptide with optional protecting groups.
DWLRAFYDKVAEKLKEAF

SEQ ID NO: 61 - protein, 18 aa, Artificial Sequence
  Synthetic peptide with optional protecting groups.
EWLRAFYEKVAEKLKEAF

SEQ ID NO: 62 - protein, 18 aa, Artificial Sequence
  Synthetic peptide with optional protecting groups.
DWLKAFYDRVAEKLKEAF

SEQ ID NO: 63 - protein, 18 aa, Artificial Sequence
  Synthetic peptide with optional protecting groups.
EWLKAFYERVAEKLKEAF

SEQ ID NO: 64 - protein, 18 aa, Artificial Sequence
  Synthetic peptide with optional protecting groups.
DWLKAFYDKVAERLKEAF

SEQ ID NO: 65 - protein, 18 aa, Artificial Sequence
  Synthetic peptide with optional protecting groups.
EWLKAFYEKVAERLKEAF

SEQ ID NO: 66 - protein, 18 aa, Artificial Sequence
  Synthetic peptide with optional protecting groups.
DWLKAFYDKVAEKLREAF

SEQ ID NO: 67 - protein, 18 aa, Artificial Sequence
  Synthetic peptide with optional protecting groups.
EWLKAFYEKVAEKLREAF

SEQ ID NO: 68 - protein, 18 aa, Artificial Sequence
  Synthetic peptide with optional protecting groups.
DWLKAFYDRVAERLKEAF

SEQ ID NO: 69 - protein, 18 aa, Artificial Sequence
  Synthetic peptide with optional protecting groups.
EWLKAFYERVAERLKEAF

SEQ ID NO: 70 - protein, 18 aa, Artificial Sequence
  Synthetic peptide with optional protecting groups.
DWLRAFYDKVAEKLREAF

SEQ ID NO: 71 - protein, 18 aa, Artificial Sequence
  Synthetic peptide with optional protecting groups.
EWLRAFYEKVAEKLREAF

SEQ ID NO: 72 - protein, 18 aa, Artificial Sequence
  Synthetic peptide with optional protecting groups.
DWLRAFYDRVAEKLKEAF

SEQ ID NO: 73 - protein, 18 aa, Artificial Sequence
  Synthetic peptide with optional protecting groups.
EWLRAFYERVAEKLKEAF

SEQ ID NO: 74 - protein, 18 aa, Artificial Sequence
  Synthetic peptide with optional protecting groups.
DWLKAFYDKVAERLREAF

SEQ ID NO: 75 - protein, 18 aa, Artificial Sequence
  Synthetic peptide with optional protecting groups.
EWLKAFYEKVAERLREAF

SEQ ID NO: 76 - protein, 18 aa, Artificial Sequence
  Synthetic peptide with optional protecting groups.
DWLRAFYDKVAERLKEAF

SEQ ID NO: 77 - protein, 18 aa, Artificial Sequence
  Synthetic peptide with optional protecting groups.
EWLRAFYEKVAERLKEAF

SEQ ID NO: 78 - protein, 37 aa, Artificial Sequence
  Synthetic peptide with optional protecting groups.
DWLKAFYDKVAEKLKEAFPDWLKAFYDKVAEKLKEAF

SEQ ID NO: 79 - protein, 37 aa, Artificial Sequence
  Synthetic peptide with optional protecting groups.
DWLKAFYDKVAEKLKEFFPDWLKAFYDKVAEKLKEFF

SEQ ID NO: 80 - protein, 37 aa, Artificial Sequence
  Synthetic peptide with optional protecting groups.
DWFKAFYDKVAEKLKEAFPDWFKAFYDKVAEKLKEAF

SEQ ID NO: 81 - protein, 37 aa, Artificial Sequence
  Synthetic peptide with optional protecting groups.
DKLKAFYDKVFEWAKEAFPDKLKAFYDKVFEWLKEAF

SEQ ID NO: 82 - protein, 37 aa, Artificial Sequence
  Synthetic peptide with optional protecting groups.
DKWKAVYDKFAEAFKEFLPDKWKAVYDKFAEAFKEFL

SEQ ID NO: 83 - protein, 37 aa, Artificial Sequence
  Synthetic peptide with optional protecting groups.
DWFKAFYDKVAEKFKEAFPDWFKAFYDKVAEKFKEAF

SEQ ID NO: 84 - protein, 37 aa, Artificial Sequence
  Synthetic peptide with optional protecting groups.
DWLKAFVYDKVFKLKEFFPDWLKAFVYDKVFKLKEFF

SEQ ID NO: 85 - protein, 37 aa, Artificial Sequence
  Synthetic peptide with optional protecting groups.
DWLKAFYDKFAEKFKEFFPDWLKAFYDKFAEKFKEFF

SEQ ID NO: 86 - protein, 18 aa, Artificial Sequence
  Synthetic peptide with optional protecting groups.
EWFKAFYEKVAEKFKEAF

SEQ ID NO: 87 - protein, 14 aa, Artificial Sequence
  Synthetic peptide with optional protecting groups.
DWFKAFYDKVAEKF

SEQ ID NO: 88 - protein, 14 aa, Artificial Sequence
  Synthetic peptide with optional protecting groups.
FKAFYDKVAEKFKE

SEQ ID NO: 89 - protein, 14 aa, Artificial Sequence
  Synthetic peptide with optional protecting groups.
FKAFYEKVAEKFKE

SEQ ID NO: 90 - protein, 17 aa, Artificial Sequence
  Synthetic peptide with optional protecting groups.
NMAFKAFYDKVAEKFKE

SEQ ID NO: 91 - protein, 17 aa, Artificial Sequence
  Synthetic peptide with optional protecting groups.
NMAFKAFYEKVAEKFKE

SEQ ID NO: 92 - protein, 21 aa, Artificial Sequence
  Synthetic peptide with optional protecting groups.
NMADWFKAFYDKVAEKFKEAF

SEQ ID NO: 93 - protein, 21 aa, Artificial Sequence
  Synthetic peptide with optional protecting groups.
NMAEWFKAFYEKVAEKFKEAF

SEQ ID NO: 94 - protein, 17 aa, Artificial Sequence
  Synthetic peptide with optional protecting groups.
NMAAFYDKVAEKFKEAF

SEQ ID NO: 95 - protein, 17 aa, Artificial Sequence
  Synthetic peptide with optional protecting groups.
NMADWFKAFYDKVAEKF

SEQ ID NO: 96 - protein, 18 aa, Artificial Sequence
  Synthetic peptide with optional protecting groups.
DWLKAFYDKVFEKFKEFF

SEQ ID NO: 97 - protein, 18 aa, Artificial Sequence
  Synthetic peptide with optional protecting groups.
EWLKAFYEKVFEKFKEFF

SEQ ID NO: 98 - protein, 14 aa, Artificial Sequence
  Synthetic peptide with optional protecting groups.
AFYDKVFEKFKEFF

SEQ ID NO: 99 - protein, 14 aa, Artificial Sequence
  Synthetic peptide with optional protecting groups.
AFYEKVFEKFKEFF

SEQ ID NO: 100 - protein, 14 aa, Artificial Sequence
  Synthetic peptide with optional protecting groups.
DWLKAFYDKVFEKF

SEQ ID NO: 101 - protein, 14 aa, Artificial Sequence
  Synthetic peptide with optional protecting groups.
EWLKAFYEKVFEKF

SEQ ID NO: 102 - protein, 14 aa, Artificial Sequence
  Synthetic peptide with optional protecting groups.
LKAFYDKVFEKFKE

SEQ ID NO: 103 - protein, 14 aa, Artificial Sequence
  Synthetic peptide with optional protecting groups.
LKAFYEKVFEKFKE

SEQ ID NO: 104 - protein, 18 aa, Artificial Sequence
  Synthetic peptide with optional protecting groups.
FAEKFKEAVKDYFAKFWD

SEQ ID NO: 105 - protein, 18 aa, Artificial Sequence
  Synthetic peptide with optional protecting groups.
DKWKAVYDKFAEAFKEFL

SEQ ID NO: 106 - protein, 18 aa, Artificial Sequence
  Synthetic peptide with optional protecting groups.
DKLKAFYDKVFEWAKEAF

SEQ ID NO: 107 - protein, 18 aa, Artificial Sequence
  Synthetic peptide with optional protecting groups.
EWFKAFYEKVADKFKDAF

SEQ ID NO: 108 - protein, 18 aa, Artificial Sequence
  Synthetic peptide with optional protecting groups.
EWFKAFYDKVADKFKEAF

SEQ ID NO: 109 - protein, 18 aa, Artificial Sequence
  Synthetic peptide with optional protecting groups.
DWFKAFYEKVADKFKEAF

SEQ ID NO: 110 - protein, 18 aa, Artificial Sequence
  Synthetic peptide with optional protecting groups.
DWFKAFYEKVAEKFKDAF

SEQ ID NO: 111 - protein, 18 aa, Artificial Sequence
  Synthetic peptide with optional protecting groups.
DFWKAFYDKVAEKFKEAF

SEQ ID NO: 112 - protein, 18 aa, Artificial Sequence
  Synthetic peptide with optional protecting groups.
EFWKAFYEKVADKFKDAF

SEQ ID NO: 113 - protein, 18 aa, Artificial Sequence
  Synthetic peptide with optional protecting groups.
EFWKAFYDKVADKFKEAF

SEQ ID NO: 114 - protein, 18 aa, Artificial Sequence
  Synthetic peptide with optional protecting groups.
DFWKAFYEKVADKFKEAF

SEQ ID NO: 115 - protein, 18 aa, Artificial Sequence
  Synthetic peptide with optional protecting groups.
DFWKAFYEKVAEKFKDAF

SEQ ID NO: 116 - protein, 18 aa, Artificial Sequence
  Synthetic peptide with optional protecting groups.
DWFKAYFDKVAEKFKEAF

SEQ ID NO: 117 - protein, 18 aa, Artificial Sequence
  Synthetic peptide with optional protecting groups.
EWFKAYFEKVADKFKDAF

SEQ ID NO: 118 - protein, 18 aa, Artificial Sequence
  Synthetic peptide with optional protecting groups.
EWFKAYFDKVADKFKEAF

SEQ ID NO: 119 - protein, 18 aa, Artificial Sequence
  Synthetic peptide with optional protecting groups.
DWFKAYFEKVADKFKEAF

SEQ ID NO: 120 - protein, 18 aa, Artificial Sequence
  Synthetic peptide with optional protecting groups.
DWFKAYFEKVAEKFKDAF

SEQ ID NO: 121 - protein, 18 aa, Artificial Sequence
  Synthetic peptide with optional protecting groups.
DWFKAFVDKYAEKFKEAF

SEQ ID NO: 122 - protein, 18 aa, Artificial Sequence
  Synthetic peptide with optional protecting groups.
EWFKAFVEKYADKFKDAF

SEQ ID NO: 123 - protein, 18 aa, Artificial Sequence
  Synthetic peptide with optional protecting groups.
EWFKAFVDKYADKFKEAF

SEQ ID NO: 124 - protein, 18 aa, Artificial Sequence
  Synthetic peptide with optional protecting groups.
DWFKAFVEKYADKFKEAF

SEQ ID NO: 125 - protein, 18 aa, Artificial Sequence
  Synthetic peptide with optional protecting groups.
DWFKAFVEKYAEKFKDAF

SEQ ID NO: 126 - protein, 18 aa, Artificial Sequence
  Synthetic peptide with optional protecting groups.
DWFKAFYDKAVEKFKEAF

SEQ ID NO: 127 - protein, 18 aa, Artificial Sequence
  Synthetic peptide with optional protecting groups.
EWFKAFYEKAVDKFKDAF

SEQ ID NO: 128 - protein, 18 aa, Artificial Sequence
  Synthetic peptide with optional protecting groups.
EWFKAFYDKAVDKFKEAF

SEQ ID NO: 129 - protein, 18 aa, Artificial Sequence
  Synthetic peptide with optional protecting groups.
DWFKAFYEKAVDKFKEAF

SEQ ID NO: 130 - protein, 18 aa, Artificial Sequence
  Synthetic peptide with optional protecting groups.
DWFKAFYEKAVEKFKDAF

SEQ ID NO: 131 - protein, 18 aa, Artificial Sequence
  Synthetic peptide with optional protecting groups.
DWFKAFYDKVFEKAKEAF

SEQ ID NO: 132 - protein, 18 aa, Artificial Sequence
  Synthetic peptide with optional protecting groups.
EWFKAFYEKVFDKAKDAF

SEQ ID NO: 133 - protein, 18 aa, Artificial Sequence
  Synthetic peptide with optional protecting groups.
EWFKAFYDKVFDKAKEAF

SEQ ID NO: 134 - protein, 18 aa, Artificial Sequence
  Synthetic peptide with optional protecting groups.
DWFKAFYEKVFDKAKEAF

SEQ ID NO: 135 - protein, 18 aa, Artificial Sequence
  Synthetic peptide with optional protecting groups.
DWFKAFYEKVFEKAKDAF

SEQ ID NO: 136 - protein, 18 aa, Artificial Sequence
  Synthetic peptide with optional protecting groups.
DWFKAFYDKVAEKAKEFF

SEQ ID NO: 137 - protein, 18 aa, Artificial Sequence
  Synthetic peptide with optional protecting groups.
EWFKAFYEKVADKAKDFF

SEQ ID NO: 138 - protein, 18 aa, Artificial Sequence
  Synthetic peptide with optional protecting groups.
EWFKAFYDKVADKAKEFF

SEQ ID NO: 139 - protein, 18 aa, Artificial Sequence
  Synthetic peptide with optional protecting groups.
DWFKAFYEKVADKAKEFF

SEQ ID NO: 140 - protein, 18 aa, Artificial Sequence
  Synthetic peptide with optional protecting groups.
DWFKAFYEKVAEKAKDFF

SEQ ID NO: 141 - protein, 18 aa, Artificial Sequence
  Synthetic peptide with optional protecting groups.
DWFKAFYDKVAEKFKEFA

SEQ ID NO: 142 - protein, 18 aa, Artificial Sequence
  Synthetic peptide with optional protecting groups.
EWFKAFYEKVADKFKDFA

SEQ ID NO: 143 - protein, 18 aa, Artificial Sequence
  Synthetic peptide with optional protecting groups.
EWFKAFYDKVADKFKEFA

SEQ ID NO: 144 - protein, 18 aa, Artificial Sequence
  Synthetic peptide with optional protecting groups.
DWFKAFYEKVADKFKEFA

SEQ ID NO: 145 - protein, 18 aa, Artificial Sequence
  Synthetic peptide with optional protecting groups.
DWFKAFYEKVAEKFKDFA

SEQ ID NO: 146 - protein, 18 aa, Artificial Sequence
  Synthetic peptide with optional protecting groups.
DAFKAFYDKVAEKFKEWF

SEQ ID NO: 147 - protein, 18 aa, Artificial Sequence
  Synthetic peptide with optional protecting groups.
EAFKAFYEKVADKFKDWF

SEQ ID NO: 148 - protein, 18 aa, Artificial Sequence
  Synthetic peptide with optional protecting groups.
EAFKAFYDKVADKFKEWF

SEQ ID NO: 149 - protein, 18 aa, Artificial Sequence
  Synthetic peptide with optional protecting groups.
DAFKAFYEKVADKFKEWF

SEQ ID NO: 150 - protein, 18 aa, Artificial Sequence
  Synthetic peptide with optional protecting groups.
DAFKAFYEKVAEKFKDWF

SEQ ID NO: 151 - protein, 18 aa, Artificial Sequence
  Synthetic peptide with optional protecting groups.
DAFKAFYDKVWEKFKEAF

SEQ ID NO: 152 - protein, 18 aa, Artificial Sequence
  Synthetic peptide with optional protecting groups.
EAFKAFYEKVWDKFKDAF

SEQ ID NO: 153 - protein, 18 aa, Artificial Sequence
  Synthetic peptide with optional protecting groups.
EAFKAFYDKVWDKFKEAF

SEQ ID NO: 154 - protein, 18 aa, Artificial Sequence
  Synthetic peptide with optional protecting groups.
DAFKAFYEKVWDKFKEAF

SEQ ID NO: 155 - protein, 18 aa, Artificial Sequence
  Synthetic peptide with optional protecting groups.
DAFKAFYEKVWEKFKDAF

SEQ ID NO: 156 - protein, 18 aa, Artificial Sequence
  Synthetic peptide with optional protecting groups.
DYFKAFWDKVAEKFKEAF

SEQ ID NO: 157 - protein, 18 aa, Artificial Sequence
  Synthetic peptide with optional protecting groups.
EYFKAFWEKVADKFKDAF

SEQ ID NO: 158 - protein, 18 aa, Artificial Sequence
  Synthetic peptide with optional protecting groups.
EYFKAFWDKVADKFKEAF

SEQ ID NO: 159 - protein, 18 aa, Artificial Sequence
  Synthetic peptide with optional protecting groups.
DYFKAFWEKVADKFKEAF

SEQ ID NO: 160 - protein, 18 aa, Artificial Sequence
  Synthetic peptide with optional protecting groups.
DYFKAFWEKVAEKFKDAF

SEQ ID NO: 161 - protein, 18 aa, Artificial Sequence
  Synthetic peptide with optional protecting groups.
DWAKAFYDKVAEKFKEFF

SEQ ID NO: 162 - protein, 18 aa, Artificial Sequence
  Synthetic peptide with optional protecting groups.
EWAKAFYEKVADKFKDFF

SEQ ID NO: 163 - protein, 18 aa, Artificial Sequence
  Synthetic peptide with optional protecting groups.
EWAKAFYDKVADKFKEFF

SEQ ID NO: 164 - protein, 18 aa, Artificial Sequence
  Synthetic peptide with optional protecting groups.
DWAKAFYEKVADKFKEFF

SEQ ID NO: 165 - protein, 18 aa, Artificial Sequence
  Synthetic peptide with optional protecting groups.
DWAKAFYEKVAEKFKDFF

SEQ ID NO: 166 - protein, 18 aa, Artificial Sequence
  Synthetic peptide with optional protecting groups.
DWFKAAYDKVAEKFKEFF

SEQ ID NO: 167 - protein, 18 aa, Artificial Sequence
  Synthetic peptide with optional protecting groups.
EWFKAAYEKVADKFKDFF

SEQ ID NO: 168 - protein, 18 aa, Artificial Sequence
  Synthetic peptide with optional protecting groups.
EWFKAAYDKVADKFKEFF

SEQ ID NO: 169 - protein, 18 aa, Artificial Sequence
  Synthetic peptide with optional protecting groups.
DWFKAAYEKVADKFKEFF

SEQ ID NO: 170 - protein, 18 aa, Artificial Sequence
  Synthetic peptide with optional protecting groups.
DWFKAAYEKVAEKFKDFF

SEQ ID NO: 171 - protein, 18 aa, Artificial Sequence
  Synthetic peptide with optional protecting groups.
DWFKAFADKVAEKFKEYF

SEQ ID NO: 172 - protein, 18 aa, Artificial Sequence
  Synthetic peptide with optional protecting groups.
EWFKAFAEKVADKFKDYF

SEQ ID NO: 173 - protein, 18 aa, Artificial Sequence
  Synthetic peptide with optional protecting groups.
EWFKAFADKVADKFKEYF

SEQ ID NO: 174 - protein, 18 aa, Artificial Sequence
  Synthetic peptide with optional protecting groups.
DWFKAFAEKVADKFKEYF

SEQ ID NO: 175 - protein, 18 aa, Artificial Sequence
  Synthetic peptide with optional protecting groups.
DWFKAFAEKVAEKFKDYF

SEQ ID NO: 176 - protein, 18 aa, Artificial Sequence
  Synthetic peptide with optional protecting groups.
DWFKAFYDKAAEKFKEVF

SEQ ID NO: 177 - protein, 18 aa, Artificial Sequence
  Synthetic peptide with optional protecting groups.
EWFKAFYEKAADKFKDVF

SEQ ID NO: 178 - protein, 18 aa, Artificial Sequence
  Synthetic peptide with optional protecting groups.
EWFKAFYDKAADKFKEVF

SEQ ID NO: 179 - protein, 18 aa, Artificial Sequence
  Synthetic peptide with optional protecting groups.
DWFKAFYEKAADKFKEVF

SEQ ID NO: 180 - protein, 18 aa, Artificial Sequence
  Synthetic peptide with optional protecting groups.
DWFKAFYEKAAEKFKDVF

SEQ ID NO: 181 - protein, 18 aa, Artificial Sequence
  Synthetic peptide with optional protecting groups.
DWYKAFFDKVAEKFKEAF

SEQ ID NO: 182 - protein, 18 aa, Artificial Sequence
  Synthetic peptide with optional protecting groups.
EWYKAFFEKVADKFKDAF

SEQ ID NO: 183 - protein, 18 aa, Artificial Sequence
  Synthetic peptide with optional protecting groups.
EWYKAFFDKVADKFKEAF

SEQ ID NO: 184 - protein, 18 aa, Artificial Sequence
  Synthetic peptide with optional protecting groups.
DWYKAFFEKVADKFKEAF

SEQ ID NO: 185 - protein, 18 aa, Artificial Sequence
  Synthetic peptide with optional protecting groups.
DWYKAFFEKVAEKFKDAF

SEQ ID NO: 186 - protein, 18 aa, Artificial Sequence
  Synthetic peptide with optional protecting groups.
DWVKAFYDKFAEKFKEAF

SEQ ID NO: 187 - protein, 18 aa, Artificial Sequence
  Synthetic peptide with optional protecting groups.
EWVKAFYEKFADKFKDAF

SEQ ID NO: 188 - protein, 18 aa, Artificial Sequence
  Synthetic peptide with optional protecting groups.
EWVKAFYDKFADKFKEAF

SEQ ID NO: 189 - protein, 18 aa, Artificial Sequence
  Synthetic peptide with optional protecting groups.
DWVKAFYEKFADKFKEAF

SEQ ID NO: 190 - protein, 18 aa, Artificial Sequence
  Synthetic peptide with optional protecting groups.
DWVKAFYEKFAEKFKDAF

SEQ ID NO: 191 - protein, 18 aa, Artificial Sequence
  Synthetic peptide with optional protecting groups.
DWFKAFFDKVAEKYKEAF

SEQ ID NO: 192 - protein, 18 aa, Artificial Sequence
  Synthetic peptide with optional protecting groups.
EWFKAFFEKVADKYKDAF

SEQ ID NO: 193 - protein, 18 aa, Artificial Sequence
  Synthetic peptide with optional protecting groups.
EWFKAFFDKVADKYKEAF

SEQ ID NO: 194 - protein, 18 aa, Artificial Sequence
  Synthetic peptide with optional protecting groups.
DWFKAFFEKVADKYKEAF

SEQ ID NO: 195 - protein, 18 aa, Artificial Sequence
  Synthetic peptide with optional protecting groups.
DWFKAFFEKVADKYKEAF

SEQ ID NO: 196 - protein, 18 aa, Artificial Sequence
  Synthetic peptide with optional protecting groups.
DWFKAFFDKVAEKFKEAY

SEQ ID NO: 197 - protein, 18 aa, Artificial Sequence
  Synthetic peptide with optional protecting groups.
EWFKAFFEKVADKFKDAY

SEQ ID NO: 198 - protein, 18 aa, Artificial Sequence
  Synthetic peptide with optional protecting groups.
EWFKAFFDKVADKFKEAY

SEQ ID NO: 199 - protein, 18 aa, Artificial Sequence
  Synthetic peptide with optional protecting groups.
DWFKAFFEKVADKFKEAY

SEQ ID NO: 200 - protein, 18 aa, Artificial Sequence
  Synthetic peptide with optional protecting groups.
DWFKAFFEKVAEKFKDAY

SEQ ID NO: 201 - protein, 18 aa, Artificial Sequence
  Synthetic peptide with optional protecting groups.
DWFKAFYDKFAEKFKEAV

SEQ ID NO: 202 - protein, 18 aa, Artificial Sequence
  Synthetic peptide with optional protecting groups.
EWFKAFYEKFADKFKDAV

SEQ ID NO: 203 - protein, 18 aa, Artificial Sequence
  Synthetic peptide with optional protecting groups.
EWFKAFYDKFADKFKEAV

SEQ ID NO: 204 - protein, 18 aa, Artificial Sequence
  Synthetic peptide with optional protecting groups.
DWFKAFYEKFADKFKEAV

SEQ ID NO: 205 - protein, 18 aa, Artificial Sequence
  Synthetic peptide with optional protecting groups.
DWFKAFYEKFAEKFKDAV

SEQ ID NO: 206 - protein, 18 aa, Artificial Sequence
  Synthetic peptide with optional protecting groups.
DKFKAFYDKVAEKFWEAF

SEQ ID NO: 207 - protein, 18 aa, Artificial Sequence
  Synthetic peptide with optional protecting groups.
EKFKAFYEKVADKFWDAF

SEQ ID NO: 208 - protein, 18 aa, Artificial Sequence
  Synthetic peptide with optional protecting groups.
EKFKAFYDKVADKFWEAF

SEQ ID NO: 209 - protein, 18 aa, Artificial Sequence
  Synthetic peptide with optional protecting groups.
DKFKAFYEKVADKFWEAF

SEQ ID NO: 210 - protein, 18 aa, Artificial Sequence
  Synthetic peptide with optional protecting groups.
DKFKAFYEKVAEKFWDAF

SEQ ID NO: 211 - protein, 18 aa, Artificial Sequence
  Synthetic peptide with optional protecting groups.
DKWKAFYDKVAEKFFEAF

SEQ ID NO: 212 - protein, 18 aa, Artificial Sequence
  Synthetic peptide with optional protecting groups.
EKWKAFYEKVADKFFDAF

SEQ ID NO: 213 - protein, 18 aa, Artificial Sequence
  Synthetic peptide with optional protecting groups.
EKWKAFYDKVADKFFEAF

SEQ ID NO: 214 - protein, 18 aa, Artificial Sequence
  Synthetic peptide with optional protecting groups.
DKWKAFYEKVADKFFEAF

SEQ ID NO: 215 - protein, 18 aa, Artificial Sequence
  Synthetic peptide with optional protecting groups.
DKWKAFYEKVAEKFFDAF

SEQ ID NO: 216 - protein, 18 aa, Artificial Sequence
  Synthetic peptide with optional protecting groups.
DKFKAFYDKWAEVFKEAF

SEQ ID NO: 217 - protein, 18 aa, Artificial Sequence
  Synthetic peptide with optional protecting groups.
EKFKAFYEKWADVFKDAF

SEQ ID NO: 218 - protein, 18 aa, Artificial Sequence
  Synthetic peptide with optional protecting groups.
EKFKAFYDKWADVFKEAF

SEQ ID NO: 219 - protein, 18 aa, Artificial Sequence
  Synthetic peptide with optional protecting groups.
DKFKAFYEKWADVFKEAF

SEQ ID NO: 220 - protein, 18 aa, Artificial Sequence
  Synthetic peptide with optional protecting groups.
DKFKAFYEKWAEVFKDAF

SEQ ID NO: 221 - protein, 18 aa, Artificial Sequence
  Synthetic peptide with optional protecting groups.
DKFKAFYDKVAEFWKEAF

SEQ ID NO: 222 - protein, 18 aa, Artificial Sequence
  Synthetic peptide with optional protecting groups.
EKFKAFYEKVADFWKDAF

SEQ ID NO: 223 - protein, 18 aa, Artificial Sequence
  Synthetic peptide with optional protecting groups.
EKFKAFYDKVADFWKEAF

SEQ ID NO: 224 - protein, 18 aa, Artificial Sequence
  Synthetic peptide with optional protecting groups.
DKFKAFYEKVADFWKEAF

SEQ ID NO: 225 - protein, 18 aa, Artificial Sequence
  Synthetic peptide with optional protecting groups.
DKFKAFYEKVAEFWKDAF

SEQ ID NO: 226 - protein, 18 aa, Artificial Sequence
  Synthetic peptide with optional protecting groups.
FAEKFKEAVKDYFAKFWD

SEQ ID NO: 227 - protein, 18 aa, Artificial Sequence
  Synthetic peptide with optional protecting groups.
FADKFKDAVKEYFAKFWE

SEQ ID NO: 228 - protein, 18 aa, Artificial Sequence
  Synthetic peptide with optional protecting groups.
FADKFKEAVKDYFAKFWE

SEQ ID NO: 229 - protein, 18 aa, Artificial Sequence
  Synthetic peptide with optional protecting groups.
FAEKFKDAVKEYFAKFWD

SEQ ID NO: 230 - protein, 18 aa, Artificial Sequence
  Synthetic peptide with optional protecting groups.
FAEKFKDAVKDYFAKFWE

SEQ ID NO: 231 - protein, 18 aa, Artificial Sequence
  Synthetic peptide with optional protecting groups.
FWEKFKEAVKDYFAKFAD

SEQ ID NO: 232 - protein, 18 aa, Artificial Sequence
  Synthetic peptide with optional protecting groups.
FWDKFKDAVKEYFAKFAE

SEQ ID NO: 233 - protein, 18 aa, Artificial Sequence
  Synthetic peptide with optional protecting groups.
FADKFKEAVKDYFAKFWE

SEQ ID NO: 234 - protein, 18 aa, Artificial Sequence
  Synthetic peptide with optional protecting groups.
FAEKFKDAVKEYFAKFWD

SEQ ID NO: 235 - protein, 18 aa, Artificial Sequence
  Synthetic peptide with optional protecting groups.
FAEKFKDAVKDYFAKFWE

SEQ ID NO: 236 - protein, 18 aa, Artificial Sequence
  Synthetic peptide with optional protecting groups.
FFEKFKEAVKDYFAKAWD

SEQ ID NO: 237 - protein, 18 aa, Artificial Sequence
  Synthetic peptide with optional protecting groups.
FFDKFKDAVKEYFAKAWE

SEQ ID NO: 238 - protein, 18 aa, Artificial Sequence
  Synthetic peptide with optional protecting groups.
FFDKFKEAVKDYFAKAWE

SEQ ID NO: 239 - protein, 18 aa, Artificial Sequence
  Synthetic peptide with optional protecting groups.
FFEKFKDAVKEYFAKAWD

SEQ ID NO: 240 - protein, 18 aa, Artificial Sequence
  Synthetic peptide with optional protecting groups.
FFEKFKDAVKDYFAKAWE

SEQ ID NO: 241 - protein, 18 aa, Artificial Sequence
  Synthetic peptide with optional protecting groups.
FAEKAKEFVKDYFAKFWD

SEQ ID NO: 242 - protein, 18 aa, Artificial Sequence
  Synthetic peptide with optional protecting groups.
FADKAKDFVKEYFAKFWE

SEQ ID NO: 243 - protein, 18 aa, Artificial Sequence
  Synthetic peptide with optional protecting groups.
FADKAKEFVKDYFAKFWE

SEQ ID NO: 244 - protein, 18 aa, Artificial Sequence
  Synthetic peptide with optional protecting groups.
FAEKAKDFVKEYFAKFWD

SEQ ID NO: 245 - protein, 18 aa, Artificial Sequence
  Synthetic peptide with optional protecting groups.
FAEKAKDFVKDYFAKFWE

SEQ ID NO: 246 - protein, 18 aa, Artificial Sequence
  Synthetic peptide with optional protecting groups.
FAEKFKEVAKDYFAKFWD

SEQ ID NO: 247 - protein, 18 aa, Artificial Sequence
  Synthetic peptide with optional protecting groups.
FADKFKDVAKEYFAKFWE

SEQ ID NO: 248 - protein, 18 aa, Artificial Sequence
  Synthetic peptide with optional protecting groups.
FADKFKEVAKDYFAKFWE

SEQ ID NO: 249 - protein, 18 aa, Artificial Sequence
  Synthetic peptide with optional protecting groups.
FAEKFKDVAKEYFAKFWD

SEQ ID NO: 250 - protein, 18 aa, Artificial Sequence
  Synthetic peptide with optional protecting groups.
FAEKFKDVAKDYFAKFWE

SEQ ID NO: 251 - protein, 18 aa, Artificial Sequence
  Synthetic peptide with optional protecting groups.
FAEKFKEAYKDVFAKFWD

SEQ ID NO: 252 - protein, 18 aa, Artificial Sequence
  Synthetic peptide with optional protecting groups.
FADKFKDAYKEVFAKFWE

SEQ ID NO: 253 - protein, 18 aa, Artificial Sequence
  Synthetic peptide with optional protecting groups.
FADKFKEAYKDVFAKFWE

SEQ ID NO: 254 - protein, 18 aa, Artificial Sequence
  Synthetic peptide with optional protecting groups.
FAEKFKDAYKEVFAKFWD

SEQ ID NO: 255 - protein, 18 aa, Artificial Sequence
  Synthetic peptide with optional protecting groups.
FAEKFKDAYKDVFAKFWE

SEQ ID NO: 256 - protein, 18 aa, Artificial Sequence
  Synthetic peptide with optional protecting groups.
FAEKFKEAVKDFYAKFWD

SEQ ID NO: 257 - protein, 18 aa, Artificial Sequence
  Synthetic peptide with optional protecting groups.
FADKFKDAVKEFYAKFWE

SEQ ID NO: 258 - protein, 18 aa, Artificial Sequence
  Synthetic peptide with optional protecting groups.
FADKFKEAVKDFYAKFWE

SEQ ID NO: 259 - protein, 18 aa, Artificial Sequence
  Synthetic peptide with optional protecting groups.
FAEKFKDAVKEFYAKFWD

SEQ ID NO: 260 - protein, 18 aa, Artificial Sequence
  Synthetic peptide with optional protecting groups.
FAEKFKDAVKDFYAKFWE

SEQ ID NO: 261 - protein, 18 aa, Artificial Sequence
  Synthetic peptide with optional protecting groups.
FAEKFWEAVKDYFAKFKD

SEQ ID NO: 262 - protein, 18 aa, Artificial Sequence
  Synthetic peptide with optional protecting groups.
FADKFWDAVKEYFAKFKE

SEQ ID NO: 263 - protein, 18 aa, Artificial Sequence
  Synthetic peptide with optional protecting groups.
FADKFWEAVKDYFAKFKE

SEQ ID NO: 264 - protein, 18 aa, Artificial Sequence
  Synthetic peptide with optional protecting groups.
FAEKFWDAVKEYFAKFKD

SEQ ID NO: 265 - protein, 18 aa, Artificial Sequence
  Synthetic peptide with optional protecting groups.
FAEKFWDAVKDYFAKFKE

SEQ ID NO: 266 - protein, 18 aa, Artificial Sequence
  Synthetic peptide with optional protecting groups.
AFEKFKEAVKDYFAKFWD

SEQ ID NO: 267 - protein, 18 aa, Artificial Sequence
  Synthetic peptide with optional protecting groups.
AFDKFKDAVKEYFAKFWE

SEQ ID NO: 268 - protein, 18 aa, Artificial Sequence
  Synthetic peptide with optional protecting groups.
AFDKFKEAVKDYFAKFWE

SEQ ID NO: 269 - protein, 18 aa, Artificial Sequence
  Synthetic peptide with optional protecting groups.
AFEKFKDAVKEYFAKFWD

SEQ ID NO: 270 - protein, 18 aa, Artificial Sequence
  Synthetic peptide with optional protecting groups.
AFEKFKDAVKDYFAKFWE

SEQ ID NO: 271 - protein, 18 aa, Artificial Sequence
  Synthetic peptide with optional protecting groups.
VAEKFKEAFKDYFAKFWD

SEQ ID NO: 272 - protein, 18 aa, Artificial Sequence
  Synthetic peptide with optional protecting groups.
VADKFKDAFKEYFAKFWE

SEQ ID NO: 273 - protein, 18 aa, Artificial Sequence
  Synthetic peptide with optional protecting groups.
VADKFKEAFKDYFAKFWE

SEQ ID NO: 274 - protein, 18 aa, Artificial Sequence
  Synthetic peptide with optional protecting groups.
VAEKFKDAFKEYFAKFWD

SEQ ID NO: 275 - protein, 18 aa, Artificial Sequence
  Synthetic peptide with optional protecting groups.
VAEKFKDAFKDYFAKFWE

SEQ ID NO: 276 - protein, 18 aa, Artificial Sequence
  Synthetic peptide with optional protecting groups.
YAEKFKEAVKDFFAKFWD

SEQ ID NO: 277 - protein, 18 aa, Artificial Sequence
  Synthetic peptide with optional protecting groups.
YADKFKDAVKEFFAKFWE

SEQ ID NO: 278 - protein, 18 aa, Artificial Sequence
  Synthetic peptide with optional protecting groups.
YADKFKEAVKDFFAKFWE

SEQ ID NO: 279 - protein, 18 aa, Artificial Sequence
  Synthetic peptide with optional protecting groups.
YAEKFKDAVKEFFAKFWD

SEQ ID NO: 280 - protein, 18 aa, Artificial Sequence
  Synthetic peptide with optional protecting groups.
YAEKFKDAVKDFFAKFWE

SEQ ID NO: 281 - protein, 18 aa, Artificial Sequence
  Synthetic peptide with optional protecting groups.
AAEKFKEFVKDYFAKFWD

SEQ ID NO: 282 - protein, 18 aa, Artificial Sequence
  Synthetic peptide with optional protecting groups.
AADKFKDFVKEYFAKFWE

SEQ ID NO: 283 - protein, 18 aa, Artificial Sequence
  Synthetic peptide with optional protecting groups.
AADKFKEFVKDYFAKFWE

SEQ ID NO: 284 - protein, 18 aa, Artificial Sequence
  Synthetic peptide with optional protecting groups.
AAEKFKDFVKEYFAKFWD

SEQ ID NO: 285 - protein, 18 aa, Artificial Sequence
  Synthetic peptide with optional protecting groups.
AAEKFKDFVKDYFAKFWE

SEQ ID NO: 286 - protein, 18 aa, Artificial Sequence
  Synthetic peptide with optional protecting groups.
FFEKAKEAVKDYFAKFWD

SEQ ID NO: 287 - protein, 18 aa, Artificial Sequence
  Synthetic peptide with optional protecting groups.
FFDKAKDAVKEYFAKFWE

SEQ ID NO: 288 - protein, 18 aa, Artificial Sequence
  Synthetic peptide with optional protecting groups.
FFDKAKEAVKDYFAKFWE

SEQ ID NO: 289 - protein, 18 aa, Artificial Sequence
  Synthetic peptide with optional protecting groups.
FFEKAKDAVKEYFAKFWD

SEQ ID NO: 290 - protein, 18 aa, Artificial Sequence
  Synthetic peptide with optional protecting groups.
FFEKAKDAVKDYFAKFWE

SEQ ID NO: 291 - protein, 18 aa, Artificial Sequence
  Synthetic peptide with optional protecting groups.
FYEKFKEAVKDAFAKFWD

SEQ ID NO: 292 - protein, 18 aa, Artificial Sequence
  Synthetic peptide with optional protecting groups.
FYDKFKDAVKEAFAKFWE

SEQ ID NO: 293 - protein, 18 aa, Artificial Sequence
  Synthetic peptide with optional protecting groups.
FYDKFKEAVKDAFAKFWE

SEQ ID NO: 294 - protein, 18 aa, Artificial Sequence
  Synthetic peptide with optional protecting groups.
FYEKFKDAVKEAFAKFWD

SEQ ID NO: 295 - protein, 18 aa, Artificial Sequence
  Synthetic peptide with optional protecting groups.
FYEKFKDAVKDAFAKFWE

SEQ ID NO: 296 - protein, 18 aa, Artificial Sequence
  Synthetic peptide with optional protecting groups.
FVEKFKEAAKDYFAKFWD

SEQ ID NO: 297 - protein, 18 aa, Artificial Sequence
  Synthetic peptide with optional protecting groups.
FVDKFKDAAKEYFAKFWE

SEQ ID NO: 298 - protein, 18 aa, Artificial Sequence
  Synthetic peptide with optional protecting groups.
FVDKFKEAAKDYFAKFWE

SEQ ID NO: 299 - protein, 18 aa, Artificial Sequence
  Synthetic peptide with optional protecting groups.
FVEKFKDAAKEYFAKFWD

SEQ ID NO: 300 - protein, 18 aa, Artificial Sequence
  Synthetic peptide with optional protecting groups.
FVEKFKDAAKDYFAKFWE

SEQ ID NO: 301 - protein, 18 aa, Artificial Sequence
  Synthetic peptide with optional protecting groups.
FAEKYKEAVKDFFAKFWD

SEQ ID NO: 302 - protein, 18 aa, Artificial Sequence
  Synthetic peptide with optional protecting groups.
FADKYKDAVKEFFAKFWE

SEQ ID NO: 303 - protein, 18 aa, Artificial Sequence
  Synthetic peptide with optional protecting groups.
FADKYKEAVKDFFAKFWE

SEQ ID NO: 304 - protein, 18 aa, Artificial Sequence
  Synthetic peptide with optional protecting groups.
FAEKYKDAVKEFFAKFWD

SEQ ID NO: 305 - protein, 18 aa, Artificial Sequence
  Synthetic peptide with optional protecting groups.
FAEKYKDAVKDFFAKFWE

SEQ ID NO: 306 - protein, 18 aa, Artificial Sequence
  Synthetic peptide with optional protecting groups.
FAEKVKEAFKDYFAKFWD

SEQ ID NO: 307 - protein, 18 aa, Artificial Sequence
  Synthetic peptide with optional protecting groups.
FADKVKDAFKEYFAKFWE

SEQ ID NO: 308 - protein, 18 aa, Artificial Sequence
  Synthetic peptide with optional protecting groups.
FADKVKEAFKDYFAKFWE

SEQ ID NO: 309 - protein, 18 aa, Artificial Sequence
  Synthetic peptide with optional protecting groups.
FAEKVKDAFKEYFAKFWD

SEQ ID NO: 310 - protein, 18 aa, Artificial Sequence
  Synthetic peptide with optional protecting groups.
FAEKVKDAFKDYFAKFWE

SEQ ID NO: 311 - protein, 18 aa, Artificial Sequence
  Synthetic peptide with optional protecting groups.
FAEKFKEYVKDAFAKFWD

SEQ ID NO: 312 - protein, 18 aa, Artificial Sequence
  Synthetic peptide with optional protecting groups.
FADKFKDYVKEAFAKFWE

SEQ ID NO: 313 - protein, 18 aa, Artificial Sequence
  Synthetic peptide with optional protecting groups.
FADKFKEYVKDAFAKFWE

SEQ ID NO: 314 - protein, 18 aa, Artificial Sequence
  Synthetic peptide with optional protecting groups.
FAEKFKDYVKEAFAKFWD

SEQ ID NO: 315 - protein, 18 aa, Artificial Sequence
  Synthetic peptide with optional protecting groups.
FAEKFKDYVKDAFAKFWE

SEQ ID NO: 316 - protein, 18 aa, Artificial Sequence
  Synthetic peptide with optional protecting groups.
FAEKFKEAFKDYVAKFWD

SEQ ID NO: 317 - protein, 18 aa, Artificial Sequence
  Synthetic peptide with optional protecting groups.
FADKFKDAFKEYVAKFWE

SEQ ID NO: 318 - protein, 18 aa, Artificial Sequence
  Synthetic peptide with optional protecting groups.
FADKFKEAFKDYVAKFWE

SEQ ID NO: 319 - protein, 18 aa, Artificial Sequence
  Synthetic peptide with optional protecting groups.
FAEKFKDAFKEYVAKFWD

SEQ ID NO: 320 - protein, 18 aa, Artificial Sequence
  Synthetic peptide with optional protecting groups.
FAEKFKDAFKDYVAKFWE

SEQ ID NO: 321 - protein, 18 aa, Artificial Sequence
  Synthetic peptide with optional protecting groups.
FAEKFKEAFKDYFAKVWD

SEQ ID NO: 322 - protein, 18 aa, Artificial Sequence
  Synthetic peptide with optional protecting groups.
FADKFKDAFKEYFAKVWE

SEQ ID NO: 323 - protein, 18 aa, Artificial Sequence
  Synthetic peptide with optional protecting groups.
FADKFKEAFKDYFAKVWE

SEQ ID NO: 324 - protein, 18 aa, Artificial Sequence
  Synthetic peptide with optional protecting groups.
FAEKFKDAFKEYFAKVWD

SEQ ID NO: 325 - protein, 18 aa, Artificial Sequence
  Synthetic peptide with optional protecting groups.
FAEKFKDAFKDYFAKVWE

SEQ ID NO: 326 - protein, 18 aa, Artificial Sequence
  Synthetic peptide with optional protecting groups.
FAEKFKEAVKDFFAKYWD

SEQ ID NO: 327 - protein, 18 aa, Artificial Sequence
  Synthetic peptide with optional protecting groups.
FADKFKDAVKEFFAKYWE

SEQ ID NO: 328 - protein, 18 aa, Artificial Sequence
  Synthetic peptide with optional protecting groups.
FADKFKEAVKDFFAKYWE

SEQ ID NO: 329 - protein, 18 aa, Artificial Sequence
  Synthetic peptide with optional protecting groups.
FAEKFKDAVKEFFAKYWD

SEQ ID NO: 330 - protein, 18 aa, Artificial Sequence
  Synthetic peptide with optional protecting groups.
FAEKFKDAVKDFFAKYWE

SEQ ID NO: 331 - protein, 18 aa, Artificial Sequence
  Synthetic peptide with optional protecting groups.
WAEKFFEAVKDYFAKFKD

SEQ ID NO: 332 - protein, 18 aa, Artificial Sequence
  Synthetic peptide with optional protecting groups.
WADKFFDAVKEYFAKFKE

SEQ ID NO: 333 - protein, 18 aa, Artificial Sequence
  Synthetic peptide with optional protecting groups.
WADKFFEAVKDYFAKFKE

SEQ ID NO: 334 - protein, 18 aa, Artificial Sequence
  Synthetic peptide with optional protecting groups.
WAEKFFDAVKEYFAKFKD

SEQ ID NO: 335 - protein, 18 aa, Artificial Sequence
  Synthetic peptide with optional protecting groups.
WAEKFFDAVKDYFAKFKE

SEQ ID NO: 336 - protein, 18 aa, Artificial Sequence
  Synthetic peptide with optional protecting groups.
FAEKWFEAVKDYFAKFKD

SEQ ID NO: 337 - protein, 18 aa, Artificial Sequence
  Synthetic peptide with optional protecting groups.
FADKWFDAVKEYFAKFKE

SEQ ID NO: 338 - protein, 18 aa, Artificial Sequence
  Synthetic peptide with optional protecting groups.
FADKWFEAVKDYFAKFKE

SEQ ID NO: 339 - protein, 18 aa, Artificial Sequence
  Synthetic peptide with optional protecting groups.
FAEKWFDAVKEYFAKFKD

SEQ ID NO: 340 - protein, 18 aa, Artificial Sequence
  Synthetic peptide with optional protecting groups.
FAEKWFDAVKDYFAKFKE

SEQ ID NO: 341 - protein, 18 aa, Artificial Sequence
  Synthetic peptide with optional protecting groups.
FAEKFVEAWKDYFAKFKD

SEQ ID NO: 342 - protein, 18 aa, Artificial Sequence
  Synthetic peptide with optional protecting groups.
FADKFVDAWKEYFAKFKE

SEQ ID NO: 343 - protein, 18 aa, Artificial Sequence
  Synthetic peptide with optional protecting groups.
FADKFVEAWKDYFAKFKE

SEQ ID NO: 344 - protein, 18 aa, Artificial Sequence
  Synthetic peptide with optional protecting groups.
FAEKFVDAWKEYFAKFKD

SEQ ID NO: 345 - protein, 18 aa, Artificial Sequence
  Synthetic peptide with optional protecting groups.
FAEKFVDAWKDYFAKFKE

SEQ ID NO: 346 - protein, 18 aa, Artificial Sequence
  Synthetic peptide with optional protecting groups.
FYEKFAEAVKDWFAKFKD

SEQ ID NO: 347 - protein, 18 aa, Artificial Sequence
  Synthetic peptide with optional protecting groups.
FYDKFADAVKEWFAKFKE

SEQ ID NO: 348 - protein, 18 aa, Artificial Sequence
  Synthetic peptide with optional protecting groups.
FYDKFAEAVKDWFAKFKE

SEQ ID NO: 349 - protein, 18 aa, Artificial Sequence
  Synthetic peptide with optional protecting groups.
FYEKFADAVKEWFAKFKD

SEQ ID NO: 350 - protein, 18 aa, Artificial Sequence
  Synthetic peptide with optional protecting groups.
FYEKFADAVKDWFAKFKE

SEQ ID NO: 351 - protein, 18 aa, Artificial Sequence
  Synthetic peptide with optional protecting groups.
XXXXXXXXXXXXXXXXXX

SEQ ID NO: 352 - protein, 18 aa, Artificial Sequence
  Synthetic peptide with optional protecting groups.
DWFKHFYDKVAEKFKEAF

SEQ ID NO: 353 - protein, 18 aa, Artificial Sequence
  Synthetic peptide with optional protecting groups.
EWFKHFYEKVADKFKDAF

SEQ ID NO: 354 - protein, 18 aa, Artificial Sequence
  Synthetic peptide with optional protecting groups.
EWFKHFYDKVAEKFKEAF

SEQ ID NO: 355 - protein, 18 aa, Artificial Sequence
  Synthetic peptide with optional protecting groups.
DWFKHFYEKVAEKFKEAF

SEQ ID NO: 356 - protein, 18 aa, Artificial Sequence
  Synthetic peptide with optional protecting groups.
DWFKHFYDKVADKFKEAF

SEQ ID NO: 357 - protein, 18 aa, Artificial Sequence
  Synthetic peptide with optional protecting groups.
DWFKHFYDKVAEKFKDAF

SEQ ID NO: 358 - protein, 18 aa, Artificial Sequence
  Synthetic peptide with optional protecting groups.
DWHKFFYDKVAEKFKEAF

SEQ ID NO: 359 - protein, 18 aa, Artificial Sequence
  Synthetic peptide with optional protecting groups.
EWHKFFYEKVADKFKDAF

SEQ ID NO: 360 - protein, 18 aa, Artificial Sequence
  Synthetic peptide with optional protecting groups.
EWHKFFYDKVAEKFKEAF

SEQ ID NO: 361 - protein, 18 aa, Artificial Sequence
  Synthetic peptide with optional protecting groups.
DWHKFFYEKVAEKFKEAF

SEQ ID NO: 362 - protein, 18 aa, Artificial Sequence
  Synthetic peptide with optional protecting groups.
DWHKFFYDKVADKFKEAF

SEQ ID NO: 363 - protein, 18 aa, Artificial Sequence
  Synthetic peptide with optional protecting groups.
DWHKFFYDKVAEKFKDAF

SEQ ID NO: 364 - protein, 18 aa, Artificial Sequence
  Synthetic peptide with optional protecting groups.
DWFKFHYDKVAEKFKEAF

SEQ ID NO: 365 - protein, 18 aa, Artificial Sequence
  Synthetic peptide with optional protecting groups.
EWFKFHYEKVADKFKDAF

SEQ ID NO: 366 - protein, 18 aa, Artificial Sequence
  Synthetic peptide with optional protecting groups.
EWFKFHYDKVAEKFKEAF

SEQ ID NO: 367 - protein, 18 aa, Artificial Sequence
  Synthetic peptide with optional protecting groups.
DWFKFHYEKVAEKFKEAF

SEQ ID NO: 368 - protein, 18 aa, Artificial Sequence
  Synthetic peptide with optional protecting groups.
DWFKFHYDKVADKFKEAF

SEQ ID NO: 369 - protein, 18 aa, Artificial Sequence
  Synthetic peptide with optional protecting groups.
DWFKFHYDKVAEKFKDAF

SEQ ID NO: 370 - protein, 18 aa, Artificial Sequence
  Synthetic peptide with optional protecting groups.
DWFKVFYDKHAEKFKEAF

SEQ ID NO: 371 - protein, 18 aa, Artificial Sequence
  Synthetic peptide with optional protecting groups.
EWFKVFYEKHADKFKDAF

SEQ ID NO: 372 - protein, 18 aa, Artificial Sequence
  Synthetic peptide with optional protecting groups.
EWFKVFYDKHAEKFKEAF

SEQ ID NO: 373 - protein, 18 aa, Artificial Sequence
  Synthetic peptide with optional protecting groups.
DWFKVFYEKHAEKFKEAF

SEQ ID NO: 374 - protein, 18 aa, Artificial Sequence
  Synthetic peptide with optional protecting groups.
DWFKVFYDKHADKFKEAF

SEQ ID NO: 375 - protein, 18 aa, Artificial Sequence
  Synthetic peptide with optional protecting groups.
DWFKVFYDKHAEKFKDAF

SEQ ID NO: 376 - protein, 18 aa, Artificial Sequence
  Synthetic peptide with optional protecting groups.
DWFKAFYDKVAEKFKEHF

SEQ ID NO: 377 - protein, 18 aa, Artificial Sequence
  Synthetic peptide with optional protecting groups.
EWFKAFYEKVADKFKDHF

SEQ ID NO: 378 - protein, 18 aa, Artificial Sequence
  Synthetic peptide with optional protecting groups.
EWFKAFYDKVAEKFKEHF

SEQ ID NO: 379 - protein, 18 aa, Artificial Sequence
  Synthetic peptide with optional protecting groups.
DWFKAFYEKVAEKFKEHF

SEQ ID NO: 380 - protein, 18 aa, Artificial Sequence
  Synthetic peptide with optional protecting groups.
DWFKAFYDKVADKFKEHF

SEQ ID NO: 381 - protein, 18 aa, Artificial Sequence
  Synthetic peptide with optional protecting groups.
DWFKAFYDKVAEKFKDHF

SEQ ID NO: 382 - protein, 18 aa, Artificial Sequence
  Synthetic peptide with optional protecting groups.
DWFKAFYDKVAEKFKEFH

SEQ ID NO: 383 - protein, 18 aa, Artificial Sequence
  Synthetic peptide with optional protecting groups.
EWFKAFYEKVADKFKDFH

SEQ ID NO: 384 - protein, 18 aa, Artificial Sequence
  Synthetic peptide with optional protecting groups.
EWFKAFYDKVAEKFKEFH

SEQ ID NO: 385 - protein, 18 aa, Artificial Sequence
  Synthetic peptide with optional protecting groups.
DWFKAFYDKVAEKFKEFH

SEQ ID NO: 386 - protein, 18 aa, Artificial Sequence
  Synthetic peptide with optional protecting groups.
DWFKAFYEKVAEKFKEFH

SEQ ID NO: 387 - protein, 18 aa, Artificial Sequence
  Synthetic peptide with optional protecting groups.
DWFKAFYDKVAEKFKEFH

SEQ ID NO: 388 - protein, 18 aa, Artificial Sequence
  Synthetic peptide with optional protecting groups.
DWFKAFYDKVAEKFKDFH

SEQ ID NO: 389 - protein, 18 aa, Artificial Sequence
  Synthetic peptide with optional protecting groups.
FAEKFKEAVKDYFAKFWD

SEQ ID NO: 390 - protein, 18 aa, Artificial Sequence
  Synthetic peptide with optional protecting groups.
FHEKFKEAVKDYFAKFWD

SEQ ID NO: 391 - protein, 18 aa, Artificial Sequence
  Synthetic peptide with optional protecting groups.
FHEKFKEAVKEYFAKFWE

SEQ ID NO: 392 - protein, 18 aa, Artificial Sequence
  Synthetic peptide with optional protecting groups.
FHDKFKDAVKDYFAKFWD

SEQ ID NO: 393 - protein, 18 aa, Artificial Sequence
  Synthetic peptide with optional protecting groups.
FHDKFKDAVKEYFAKFWE

SEQ ID NO: 394 - protein, 18 aa, Artificial Sequence
  Synthetic peptide with optional protecting groups.
FHDKFKEAVKDYFAKFWD

SEQ ID NO: 395 - protein, 18 aa, Artificial Sequence
  Synthetic peptide with optional protecting groups.
FHEKFKDAVKDYFAKFWD

SEQ ID NO: 396 - protein, 18 aa, Artificial Sequence
  Synthetic peptide with optional protecting groups.
FHEKFKEAVKEYFAKFWD

SEQ ID NO: 397 - protein, 18 aa, Artificial Sequence
  Synthetic peptide with optional protecting groups.
FHEKFKEAVKDYFAKFWE

SEQ ID NO: 398 - protein, 18 aa, Artificial Sequence
  Synthetic peptide with optional protecting groups.
HFEKFKEAVKDYFAKFWD

SEQ ID NO: 399 - protein, 18 aa, Artificial Sequence
  Synthetic peptide with optional protecting groups.
HFDKFKDAVKEYFAKFWE

SEQ ID NO: 400 - protein, 18 aa, Artificial Sequence
  Synthetic peptide with optional protecting groups.
HFEKFKEAVKEYFAKFWE

SEQ ID NO: 401 - protein, 18 aa, Artificial Sequence
  Synthetic peptide with optional protecting groups.
HFDKFKEAVKDYFAKFWD

SEQ ID NO: 402 - protein, 18 aa, Artificial Sequence
  Synthetic peptide with optional protecting groups.
HFEKFKDAVKDYFAKFWD

SEQ ID NO: 403 - protein, 18 aa, Artificial Sequence
  Synthetic peptide with optional protecting groups.
HFEKFKEAVKEYFAKFWD

SEQ ID NO: 404 - protein, 18 aa, Artificial Sequence
  Synthetic peptide with optional protecting groups.
HFEKFKEAVKDYFAKFWE

SEQ ID NO: 405 - protein, 18 aa, Artificial Sequence
  Synthetic peptide with optional protecting groups.
FFEKHKEAVKDYFAKFWD

SEQ ID NO: 406 - protein, 18 aa, Artificial Sequence
  Synthetic peptide with optional protecting groups.
FFDKHKDAVKEYFAKFWE

SEQ ID NO: 407 - protein, 18 aa, Artificial Sequence
  Synthetic peptide with optional protecting groups.
FFEKHKEAVKEYFAKFWE

SEQ ID NO: 408 - protein, 18 aa, Artificial Sequence
  Synthetic peptide with optional protecting groups.
FFDKHKDAVKDYFAKFWD

SEQ ID NO: 409 - protein, 18 aa, Artificial Sequence
  Synthetic peptide with optional protecting groups.
FFDKHKEAVKDYFAKFWD

SEQ ID NO: 410 - protein, 18 aa, Artificial Sequence
  Synthetic peptide with optional protecting groups.
FFEKHKEAVKEYFAKFWD

SEQ ID NO: 411 - protein, 18 aa, Artificial Sequence
  Synthetic peptide with optional protecting groups.
FFEKHKEAVKDYFAKFWE

SEQ ID NO: 412 - protein, 18 aa, Artificial Sequence
  Synthetic peptide with optional protecting groups.
FVEKFKEAHKDYFAKFWD

SEQ ID NO: 413 - protein, 18 aa, Artificial Sequence
  Synthetic peptide with optional protecting groups.
FVDKFKDAHKEYFAKFWE

SEQ ID NO: 414 - protein, 18 aa, Artificial Sequence
  Synthetic peptide with optional protecting groups.
FVEKFKEAHKEYFAKFWE

SEQ ID NO: 415 - protein, 18 aa, Artificial Sequence
  Synthetic peptide with optional protecting groups.
FVDKFKDAHKDYFAKFWD

SEQ ID NO: 416 - protein, 18 aa, Artificial Sequence
  Synthetic peptide with optional protecting groups.
FVDKFKEAHKDYFAKFWD

SEQ ID NO: 417 - protein, 18 aa, Artificial Sequence
  Synthetic peptide with optional protecting groups.
FVEKFKDAHKDYFAKFWD

SEQ ID NO: 418 - protein, 18 aa, Artificial Sequence
  Synthetic peptide with optional protecting groups.
FVEKFKEAHKEYFAKFWD

SEQ ID NO: 419 - protein, 18 aa, Artificial Sequence
  Synthetic peptide with optional protecting groups.
FVEKFKEAHKDYFAKFWE

SEQ ID NO: 420 - protein, 18 aa, Artificial Sequence
  Synthetic peptide with optional protecting groups.
FAEKFKEHVKDYFAKFWD

SEQ ID NO: 421 - protein, 18 aa, Artificial Sequence
  Synthetic peptide with optional protecting groups.
FADKFKDHVKEYFAKFWE

SEQ ID NO: 422 - protein, 18 aa, Artificial Sequence
  Synthetic peptide with optional protecting groups.
FAEKFKEHVKEYFAKFWE

SEQ ID NO: 423 - protein, 18 aa, Artificial Sequence
  Synthetic peptide with optional protecting groups.
FADKFKDHVKDYFAKFWD

SEQ ID NO: 424 - protein, 18 aa, Artificial Sequence
  Synthetic peptide with optional protecting groups.
FADKFKEHVKDYFAKFWD

SEQ ID NO: 425 - protein, 18 aa, Artificial Sequence
  Synthetic peptide with optional protecting groups.
FAEKFKDHVKDYFAKFWD

SEQ ID NO: 426 - protein, 18 aa, Artificial Sequence
  Synthetic peptide with optional protecting groups.
FAEKFKEHVKEYFAKFWD

SEQ ID NO: 427 - protein, 18 aa, Artificial Sequence
  Synthetic peptide with optional protecting groups.
FAEKFKEHVKDYFAKFWE

SEQ ID NO: 428 - protein, 18 aa, Artificial Sequence
  Synthetic peptide with optional protecting groups.
FAEKFKEFVKDYHAKFWD

SEQ ID NO: 429 - protein, 18 aa, Artificial Sequence
  Synthetic peptide with optional protecting groups.
FADKFKDFVKEYHAKFWE

SEQ ID NO: 430 - protein, 18 aa, Artificial Sequence
  Synthetic peptide with optional protecting groups.
FADKFKEFVKDYHAKFWD

SEQ ID NO: 431 - protein, 18 aa, Artificial Sequence
  Synthetic peptide with optional protecting groups.
FAEKFKDFVKDYHAKFWD

SEQ ID NO: 432 - protein, 18 aa, Artificial Sequence
  Synthetic peptide with optional protecting groups.
FADKFKDFVKDYHAKFWD

SEQ ID NO: 433 - protein, 18 aa, Artificial Sequence
  Synthetic peptide with optional protecting groups.
FAEKFKEFVKEYHAKFWE

SEQ ID NO: 434 - protein, 18 aa, Artificial Sequence
  Synthetic peptide with optional protecting groups.
FAEKFKEFVKEYHAKFWD

SEQ ID NO: 435 - protein, 18 aa, Artificial Sequence
  Synthetic peptide with optional protecting groups.
FAEKFKEFVKDYHAKFWE

SEQ ID NO: 436 - protein, 18 aa, Artificial Sequence
  Synthetic peptide with optional protecting groups.
FAEKFKEFVKDYFAKHWD

SEQ ID NO: 437 - protein, 18 aa, Artificial Sequence
  Synthetic peptide with optional protecting groups.
FADKFKDFVKEYFAKHWE

SEQ ID NO: 438 - protein, 18 aa, Artificial Sequence
  Synthetic peptide with optional protecting groups.
FAEKFKEFVKEYFAKHWE

SEQ ID NO: 439 - protein, 18 aa, Artificial Sequence
  Synthetic peptide with optional protecting groups.
FADKFKDFVKDYFAKHWD

SEQ ID NO: 440 - protein, 18 aa, Artificial Sequence
  Synthetic peptide with optional protecting groups.
FADKFKEFVKDYFAKHWD

SEQ ID NO: 441 - protein, 18 aa, Artificial Sequence
  Synthetic peptide with optional protecting groups.
FAEKFKDFVKDYFAKHWD

SEQ ID NO: 442 - protein, 18 aa, Artificial Sequence
  Synthetic peptide with optional protecting groups.
FAEKFKEFVKEYFAKHWD

SEQ ID NO: 443 - protein, 18 aa, Artificial Sequence
  Synthetic peptide with optional protecting groups.
FAEKFKEFVKDYFAKHWE

SEQ ID NO: 444 - protein, 18 aa, Artificial Sequence
  Synthetic peptide with optional protecting groups.
DWXKAXYDKVAEKXKEAX

SEQ ID NO: 445 - protein, 18 aa, Artificial Sequence
  Synthetic peptide with optional protecting groups.
EWXKAXYEKVADKXKDAX

SEQ ID NO: 446 - protein, 18 aa, Artificial Sequence
  Synthetic peptide with optional protecting groups.
EWXKAXYEKVAEKXKEAX

SEQ ID NO: 447 - protein, 18 aa, Artificial Sequence
  Synthetic peptide with optional protecting groups.
DWXKAXYDKVADKXKDAX

SEQ ID NO: 448 - protein, 18 aa, Artificial Sequence
  Synthetic peptide with optional protecting groups.
EWXKAXYDKVAEKXKEAX

SEQ ID NO: 449 - protein, 18 aa, Artificial Sequence
  Synthetic peptide with optional protecting groups.
DWXKAXYEKVAEKXKEAX

SEQ ID NO: 450 - protein, 18 aa, Artificial Sequence
  Synthetic peptide with optional protecting groups.
DWXKAXYDKVADKXKEAX

SEQ ID NO: 451 - protein, 18 aa, Artificial Sequence
  Synthetic peptide with optional protecting groups.
DWXKAXYDKVAEKXKDAX

SEQ ID NO: 452 - protein, 18 aa, Artificial Sequence
  Synthetic peptide with optional protecting groups.
DWXKAXYDKVAEKFKEAF

SEQ ID NO: 453 - protein, 18 aa, Artificial Sequence
  Synthetic peptide with optional protecting groups.
DWFKAFYDKVAEKXKEAX

SEQ ID NO: 454 - protein, 18 aa, Artificial Sequence
  Synthetic peptide with optional protecting groups.
DWXKAFYDKVAEKFKEAF

SEQ ID NO: 455 - protein, 18 aa, Artificial Sequence
  Synthetic peptide with optional protecting groups.
DWFKAXYDKVAEKFKEAF

SEQ ID NO: 456 - protein, 18 aa, Artificial Sequence
  Synthetic peptide with optional protecting groups.
DWFKAFYDKVAEKXKEAF

SEQ ID NO: 457 - protein, 18 aa, Artificial Sequence
  Synthetic peptide with optional protecting groups.
DWFKAFYDKVAEKFKEAX

SEQ ID NO: 458 - protein, 18 aa, Artificial Sequence
  Synthetic peptide with optional protecting groups.
XAEKXKEAVKDYXAKXWD

SEQ ID NO: 459 - protein, 18 aa, Artificial Sequence
  Synthetic peptide with optional protecting groups.
XAEKXKEAVKDYFAKFWD

SEQ ID NO: 460 - protein, 18 aa, Artificial Sequence
  Synthetic peptide with optional protecting groups.
FAEKFKEAVKDYXAKXWD

SEQ ID NO: 461 - protein, 18 aa, Artificial Sequence
  Synthetic peptide with optional protecting groups.
XAEKFKEAVKDYFAKFWD

SEQ ID NO: 462 - protein, 18 aa, Artificial Sequence
  Synthetic peptide with optional protecting groups.
FAEKXKEAVKDYFAKFWD

SEQ ID NO: 463 - protein, 18 aa, Artificial Sequence
  Synthetic peptide with optional protecting groups.
FAEKFKEAVKDYXAKFWD

SEQ ID NO: 464 - protein, 18 aa, Artificial Sequence
  Synthetic peptide with optional protecting groups.
FAEKFKEAVKDYFAKXWD

SEQ ID NO: 465 - protein, 18 aa, Artificial Sequence
  Synthetic peptide with optional protecting groups.
FAEKFKEAVKEYFAKFWE

SEQ ID NO: 466 - protein, 18 aa, Artificial Sequence
  Synthetic peptide with optional protecting groups.
FADKFKDAVKDYFAKFWD

SEQ ID NO: 467 - protein, 18 aa, Artificial Sequence
  Synthetic peptide with optional protecting groups.
FAERFREAVKDYFAKFWD

SEQ ID NO: 468 - protein, 18 aa, Artificial Sequence
  Synthetic peptide with optional protecting groups.
FAEKFREAVKDYFAKFWD

SEQ ID NO: 469 - protein, 18 aa, Artificial Sequence
  Synthetic peptide with optional protecting groups.
FAEKFKEAVRDYFAKFWD

SEQ ID NO: 470 - protein, 18 aa, Artificial Sequence
  Synthetic peptide with optional protecting groups.
FAEKFKEAVKDYFARFWD

SEQ ID NO: 471 - protein, 18 aa, Artificial Sequence
  Synthetic peptide with optional protecting groups.
FAEKFKEAVKEYFAKFWE

SEQ ID NO: 472 - protein, 18 aa, Artificial Sequence
  Synthetic peptide with optional protecting groups.
FADKFKDAVKDYFAKFWD

SEQ ID NO: 473 - protein, 18 aa, Artificial Sequence
  Synthetic peptide with optional protecting groups.
FAERFREAVKDYFAKFWD

SEQ ID NO: 474 - protein, 18 aa, Artificial Sequence
  Synthetic peptide with optional protecting groups.
FAEKFREAVKDYFAKFWD

SEQ ID NO: 475 - protein, 18 aa, Artificial Sequence
  Synthetic peptide with optional protecting groups.
FAEKFKEAVRDYFAKFWD

SEQ ID NO: 476 - protein, 18 aa, Artificial Sequence
  Synthetic peptide with optional protecting groups.
FAEKFKEAVKDYFARFWD

SEQ ID NO: 477 - protein, 18 aa, Artificial Sequence
  Synthetic peptide with optional protecting groups.
FAEKFKEAVKEYFAKFWE

SEQ ID NO: 478 - protein, 18 aa, Artificial Sequence
  Synthetic peptide with optional protecting groups.
FADKFKDAVKDYFAKFWD

SEQ ID NO: 479 - protein, 18 aa, Artificial Sequence
  Synthetic peptide with optional protecting groups.
FAERFREAVKDYFAKFWD

SEQ ID NO: 480 - protein, 18 aa, Artificial Sequence
  Synthetic peptide with optional protecting groups.
FAEKFREAVKDYFAKFWD

SEQ ID NO: 481 - protein, 18 aa, Artificial Sequence
  Synthetic peptide with optional protecting groups.
FAEKFKEAVRDYFAKFWD

SEQ ID NO: 482 - protein, 18 aa, Artificial Sequence
  Synthetic peptide with optional protecting groups.
FAEKFKEAVKDYFARFWD

SEQ ID NO: 483 - protein, 18 aa, Artificial Sequence
  Synthetic peptide with optional protecting groups.
FAERFREAVKDYFAKFWD

SEQ ID NO: 484 - protein, 18 aa, Artificial Sequence
  Synthetic peptide with optional protecting groups.
FAEKFREAVKDYFAKFWD

SEQ ID NO: 485 - protein, 18 aa, Artificial Sequence
  Synthetic peptide with optional protecting groups.
FAEKFKEAVRDYFAKFWD

SEQ ID NO: 486 - protein, 18 aa, Artificial Sequence
  Synthetic peptide with optional protecting groups.
FAEKFKEAVKDYFARFWD

SEQ ID NO: 487 - protein, 18 aa, Artificial Sequence
  Synthetic peptide with optional protecting groups.
FAEKFKEAVKEYFAKFWE

SEQ ID NO: 488 - protein, 18 aa, Artificial Sequence
  Synthetic peptide with optional protecting groups.
FADKFKDAVKDYFAKFWD

SEQ ID NO: 489 - protein, 18 aa, Artificial Sequence
  Synthetic peptide with optional protecting groups.
FAERFREAVKDYFAKFWD

SEQ ID NO: 490 - protein, 18 aa, Artificial Sequence
  Synthetic peptide with optional protecting groups.
FAEKFREAVKDYFAKFWD

SEQ ID NO: 491 - protein, 18 aa, Artificial Sequence
  Synthetic peptide with optional protecting groups.
FAEKFKEAVRDYFAKFWD

SEQ ID NO: 492 - protein, 18 aa, Artificial Sequence
  Synthetic peptide with optional protecting groups.
FAEKFKEAVKDYFARFWD

SEQ ID NO: 493 - protein, 18 aa, Artificial Sequence
  Synthetic peptide with optional protecting groups.
LFEKFAEAFKDYVAKWKD

SEQ ID NO: 494 - protein, 18 aa, Artificial Sequence
  Synthetic peptide with optional protecting groups.
LFERFAEAFKDYVAKWKD

SEQ ID NO: 495 - protein, 18 aa, Artificial Sequence
  Synthetic peptide with optional protecting groups.
LFEKFAEAFRDYVAKWKD

SEQ ID NO: 496 - protein, 18 aa, Artificial Sequence
  Synthetic peptide with optional protecting groups.
LFEKFAEAFKDYVARWKD

SEQ ID NO: 497 - protein, 18 aa, Artificial Sequence
  Synthetic peptide with optional protecting groups.
LFEKFAEAFKDYVAKWRD

SEQ ID NO: 498 - protein, 18 aa, Artificial Sequence
  Synthetic peptide with optional protecting groups.
LFEKFAEAFKEYVAKWKE

SEQ ID NO: 499 - protein, 18 aa, Artificial Sequence
  Synthetic peptide with optional protecting groups.
LFDKFADAFKDYVAKWKD

SEQ ID NO: 500 - protein, 18 aa, Artificial Sequence
  Synthetic peptide with optional protecting groups.
LFDKFAEAFKDYVAKWKD

SEQ ID NO: 501 - protein, 18 aa, Artificial Sequence
  Synthetic peptide with optional protecting groups.
LFEKFADAFKDYVAKWKD

SEQ ID NO: 502 - protein, 18 aa, Artificial Sequence
  Synthetic peptide with optional protecting groups.
LFEKFAEAFKEYVAKWKD

SEQ ID NO: 503 - protein, 18 aa, Artificial Sequence
  Synthetic peptide with optional protecting groups.
LFEKFAEAFKDYVAKWKE

SEQ ID NO: 504 - protein, 18 aa, Artificial Sequence
  Synthetic peptide with optional protecting groups.
FAEKAWEFVKDYFAKLKD

SEQ ID NO: 505 - protein, 18 aa, Artificial Sequence
  Synthetic peptide with optional protecting groups.
FAERAWEFVKDYFAKLKD

SEQ ID NO: 506 - protein, 18 aa, Artificial Sequence
  Synthetic peptide with optional protecting groups.
FAEKAWEFVKDYFAKLKD

SEQ ID NO: 507 - protein, 18 aa, Artificial Sequence
  Synthetic peptide with optional protecting groups.
FAEKAWEFVKDYFAKLKD

SEQ ID NO: 508 - protein, 18 aa, Artificial Sequence
  Synthetic peptide with optional protecting groups.
FAEKAWEFVKDYFAKLRD

SEQ ID NO: 509 - protein, 18 aa, Artificial Sequence
  Synthetic peptide with optional protecting groups.
FAEKAWEFVKEYFAKLKE

SEQ ID NO: 510 - protein, 18 aa, Artificial Sequence
  Synthetic peptide with optional protecting groups.
FADKAWDFVKDYFAKLKD

SEQ ID NO: 511 - protein, 18 aa, Artificial Sequence
  Synthetic peptide with optional protecting groups.
FADKAWEFVKDYFAKLKD

SEQ ID NO: 512 - protein, 18 aa, Artificial Sequence
  Synthetic peptide with optional protecting groups.
FAEKAWDFVKDYFAKLKD

SEQ ID NO: 513 - protein, 18 aa, Artificial Sequence
  Synthetic peptide with optional protecting groups.
FAEKAWEFVKEYFAKLKD

SEQ ID NO: 514 - protein, 18 aa, Artificial Sequence
  Synthetic peptide with optional protecting groups.
FAEKAWEFVKDYFAKLKE

SEQ ID NO: 515 - protein, 18 aa, Artificial Sequence
  Synthetic peptide with optional protecting groups.
FFEKFKEFVKDYFAKLWD

SEQ ID NO: 516 - protein, 18 aa, Artificial Sequence
  Synthetic peptide with optional protecting groups.
FFEKFKEFVKEYFAKLWE

SEQ ID NO: 517 - protein, 18 aa, Artificial Sequence
  Synthetic peptide with optional protecting groups.
FFDKFKDFVKDYFAKLWD

SEQ ID NO: 518 - protein, 18 aa, Artificial Sequence
  Synthetic peptide with optional protecting groups.
FFERFKEFVKDYFAKLWD

SEQ ID NO: 519 - protein, 18 aa, Artificial Sequence
  Synthetic peptide with optional protecting groups.
FFEKFREFVKDYFAKLWD

SEQ ID NO: 520 - protein, 18 aa, Artificial Sequence
  Synthetic peptide with optional protecting groups.
FFEKFKEFVRDYFAKLWD

SEQ ID NO: 521 - protein, 18 aa, Artificial Sequence
  Synthetic peptide with optional protecting groups.
FFEKFKEFVKDYFARLWD

SEQ ID NO: 522 - protein, 18 aa, Artificial Sequence
  Synthetic peptide with optional protecting groups.
FFDKFKEFVKDYFAKLWD

SEQ ID NO: 523 - protein, 18 aa, Artificial Sequence
  Synthetic peptide with optional protecting groups.
FFEKFKDFVKDYFAKLWD

SEQ ID NO: 524 - protein, 18 aa, Artificial Sequence
  Synthetic peptide with optional protecting groups.
FFEKFKEFVKEYFAKLWD

SEQ ID NO: 525 - protein, 18 aa, Artificial Sequence
  Synthetic peptide with optional protecting groups.
FFEKFKEFVKDYFAKLWE

SEQ ID NO: 526 - protein, 18 aa, Artificial Sequence
  Synthetic peptide with optional protecting groups.
FLEKFKEFVKDYFAKFWD

SEQ ID NO: 527 - protein, 18 aa, Artificial Sequence
  Synthetic peptide with optional protecting groups.
FLEKFKEFVKEYFAKFWE

SEQ ID NO: 528 - protein, 18 aa, Artificial Sequence
  Synthetic peptide with optional protecting groups.
FLDKFKEFVKDYFAKFWD

SEQ ID NO: 529 - protein, 18 aa, Artificial Sequence
  Synthetic peptide with optional protecting groups.
FLDKFKEFVKDYFAKFWD

SEQ ID NO: 530 - protein, 18 aa, Artificial Sequence
  Synthetic peptide with optional protecting groups.
FLEKFKDFVKDYFAKFWD

SEQ ID NO: 531 - protein, 18 aa, Artificial Sequence
  Synthetic peptide with optional protecting groups.
FLEKFKEFVKEYFAKFWD

SEQ ID NO: 532 - protein, 18 aa, Artificial Sequence
  Synthetic peptide with optional protecting groups.
FLEKFKEFVKDYFAKFWE

SEQ ID NO: 533 - protein, 18 aa, Artificial Sequence
  Synthetic peptide with optional protecting groups.
FLERFKEFVKDYFAKFWD

SEQ ID NO: 534 - protein, 18 aa, Artificial Sequence
  Synthetic peptide with optional protecting groups.
FLEKFREFVKDYFAKFWD

SEQ ID NO: 535 - protein, 18 aa, Artificial Sequence
  Synthetic peptide with optional protecting groups.
FLEKFKEFVRDYFAKFWD

SEQ ID NO: 536 - protein, 18 aa, Artificial Sequence
  Synthetic peptide with optional protecting groups.
FLEKFKEFVKDYFARFWD

SEQ ID NO: 537 - protein, 18 aa, Artificial Sequence
  Synthetic peptide with optional protecting groups.
FFEKFKEFFKDYFAKLWD

SEQ ID NO: 538 - protein, 18 aa, Artificial Sequence
  Synthetic peptide with optional protecting groups.
FFEKFKEFFKEYFAKLWE

SEQ ID NO: 539 - protein, 18 aa, Artificial Sequence
  Synthetic peptide with optional protecting groups.
FFDKFKDFFKDYFAKLWD

SEQ ID NO: 540 - protein, 18 aa, Artificial Sequence
  Synthetic peptide with optional protecting groups.
FFERFKEFFKDYFAKLWD

SEQ ID NO: 541 - protein, 18 aa, Artificial Sequence
  Synthetic peptide with optional protecting groups.
FFEKFREFFKDYFAKLWD

SEQ ID NO: 542 - protein, 18 aa, Artificial Sequence
  Synthetic peptide with optional protecting groups.
FFEKFKEFFRDYFAKLWD

SEQ ID NO: 543 - protein, 18 aa, Artificial Sequence
  Synthetic peptide with optional protecting groups.
FFERFKEFFKDYFARLWD

SEQ ID NO: 544 - protein, 18 aa, Artificial Sequence
  Synthetic peptide with optional protecting groups.
FFDKFKEFFKDYFAKLWD

SEQ ID NO: 545 - protein, 18 aa, Artificial Sequence
  Synthetic peptide with optional protecting groups.
FFEKFKDFFKDYFAKLWD

SEQ ID NO: 546 - protein, 18 aa, Artificial Sequence
  Synthetic peptide with optional protecting groups.
FFEKFKEFFKEYFAKLWD

SEQ ID NO: 547 - protein, 18 aa, Artificial Sequence
  Synthetic peptide with optional protecting groups.
FFEKFKEFFKDYFAKLWE

SEQ ID NO: 548 - protein, 18 aa, Artificial Sequence
  Synthetic peptide with optional protecting groups.
FAEKFKEAVKDYFAKFWD

SEQ ID NO: 549 - protein, 18 aa, Artificial Sequence
  Synthetic peptide with optional protecting groups.
FAEKFKEAVKEYFAKFWE

SEQ ID NO: 550 - protein, 18 aa, Artificial Sequence
  Synthetic peptide with optional protecting groups.
FADKFKDAVKDYFAKFWD

SEQ ID NO: 551 - protein, 18 aa, Artificial Sequence
  Synthetic peptide with optional protecting groups.
FAERFREAVKDYFAKFWD

SEQ ID NO: 552 - protein, 18 aa, Artificial Sequence
  Synthetic peptide with optional protecting groups.
FAEKFREAVKDYFAKFWD

SEQ ID NO: 553 - protein, 18 aa, Artificial Sequence
  Synthetic peptide with optional protecting groups.
FAEKFKEAVRDYFAKFWD

SEQ ID NO: 554 - protein, 18 aa, Artificial Sequence
  Synthetic peptide with optional protecting groups.
FAEKFKEAVKDYFARFWD

SEQ ID NO: 555 - protein, 18 aa, Artificial Sequence
  Synthetic peptide with optional protecting groups.
DKWKAVYDKFAEAFKEFF

SEQ ID NO: 556 - protein, 18 aa, Artificial Sequence
  Synthetic peptide with optional protecting groups.
EKWKAVYEKFAEAFKEFF

SEQ ID NO: 557 - protein, 18 aa, Artificial Sequence
  Synthetic peptide with optional protecting groups.
DKWKAVYDKFADAFKDFF

SEQ ID NO: 558 - protein, 18 aa, Artificial Sequence
  Synthetic peptide with optional protecting groups.
DRWKAVYDKFAEAFKEFF

SEQ ID NO: 559 - protein, 18 aa, Artificial Sequence
  Synthetic peptide with optional protecting groups.
DKWRAVYDKFAEAFKEFF

SEQ ID NO: 560 - protein, 18 aa, Artificial Sequence
  Synthetic peptide with optional protecting groups.
DKWKAVYDRFAEAFKEFF

SEQ ID NO: 561 - protein, 18 aa, Artificial Sequence
  Synthetic peptide with optional protecting groups.
DKWKAVYDKFAEAFREFF

SEQ ID NO: 562 - protein, 18 aa, Artificial Sequence
  Synthetic peptide with optional protecting groups.
FFEKFAEAFKDYVAKWKD

SEQ ID NO: 563 - protein, 18 aa, Artificial Sequence
  Synthetic peptide with optional protecting groups.
FFEKFAEAFKEYVAKWKE

SEQ ID NO: 564 - protein, 18 aa, Artificial Sequence
  Synthetic peptide with optional protecting groups.
FFDKFADAFKDYVAKWKD

SEQ ID NO: 565 - protein, 18 aa, Artificial Sequence
  Synthetic peptide with optional protecting groups.
FFERFAEAFKDYVAKWKD

SEQ ID NO: 566 - protein, 18 aa, Artificial Sequence
  Synthetic peptide with optional protecting groups.
FFERFAEAFRDYVAKWKD

SEQ ID NO: 567 - protein, 18 aa, Artificial Sequence
  Synthetic peptide with optional protecting groups.
FFEKFAEAFKDYVARWKD

SEQ ID NO: 568 - protein, 18 aa, Artificial Sequence
  Synthetic peptide with optional protecting groups.
FFERFAEAFKDYVAKWRD

SEQ ID NO: 569 - protein, 18 aa, Artificial Sequence
  Synthetic peptide with optional protecting groups.
FFDKFAEAFKDYVAKWKD

SEQ ID NO: 570 - protein, 18 aa, Artificial Sequence
  Synthetic peptide with optional protecting groups.
FFEKFADAFKDYVAKWKD

SEQ ID NO: 571 - protein, 18 aa, Artificial Sequence
  Synthetic peptide with optional protecting groups.
FFERFAEAFKEYVAKWKD

SEQ ID NO: 572 - protein, 18 aa, Artificial Sequence
  Synthetic peptide with optional protecting groups.
FFERFAEAFKDYVAKWKE

SEQ ID NO: 573 - protein, 18 aa, Artificial Sequence
  Synthetic peptide with optional protecting groups.
FFEKFKEFFKDYFAKFWD

SEQ ID NO: 574 - protein, 18 aa, Artificial Sequence
  Synthetic peptide with optional protecting groups.
FFDKFKDFFKDYFAKFWD

SEQ ID NO: 575 - protein, 18 aa, Artificial Sequence
  Synthetic peptide with optional protecting groups.
FFEKFKEFFKEYFAKFWE

SEQ ID NO: 576 - protein, 18 aa, Artificial Sequence
  Synthetic peptide with optional protecting groups.
FFERFKEFFKDYFAKFWD

SEQ ID NO: 577 - protein, 18 aa, Artificial Sequence
  Synthetic peptide with optional protecting groups.
FFEKFREFFKDYFAKFWD

SEQ ID NO: 578 - protein, 18 aa, Artificial Sequence
  Synthetic peptide with optional protecting groups.
FFEKFKEFFRDYFAKFWD

SEQ ID NO: 579 - protein, 18 aa, Artificial Sequence
  Synthetic peptide with optional protecting groups.
FFEKFKEFFKDYFARFWD

SEQ ID NO: 580 - protein, 18 aa, Artificial Sequence
  Synthetic peptide with optional protecting groups.
FFDKFKEFFKDYFAKFWD

SEQ ID NO: 581 - protein, 18 aa, Artificial Sequence
  Synthetic peptide with optional protecting groups.
FFEKFKDFFKDYFAKFWD

SEQ ID NO: 582 - protein, 18 aa, Artificial Sequence
  Synthetic peptide with optional protecting groups.
FFEKFKEFFKEYFAKFWD

SEQ ID NO: 583 - protein, 18 aa, Artificial Sequence
  Synthetic peptide with optional protecting groups.
FFEKFKEFFKDYFAKFWE

SEQ ID NO: 584 - protein, 18 aa, Artificial Sequence
  Synthetic peptide with optional protecting groups.
DWFKAFYDKVAEKFKEAF

SEQ ID NO: 585 - protein, 18 aa, Artificial Sequence
  Synthetic peptide with optional protecting groups.
DWFKAFYDKVAEKFKEAF

SEQ ID NO: 586 - protein, 18 aa, Artificial Sequence
  Synthetic peptide with optional protecting groups.
DWFKAFYDKVAEKFKEAF

SEQ ID NO: 587 - protein, 18 aa, Artificial Sequence
  Synthetic peptide with optional protecting groups.
DWFKAFYDKVAEKFKEAF

SEQ ID NO: 588 - protein, 18 aa, Artificial Sequence
  Synthetic peptide with optional protecting groups.
DWFKAFYDKVAEKFKEAF

SEQ ID NO: 589 - protein, 18 aa, Artificial Sequence
  Synthetic peptide with optional protecting groups.
DWFKAFYDKVAEKFKEAF

SEQ ID NO: 590 - protein, 18 aa, Artificial Sequence
  Synthetic peptide with optional protecting groups.
DWFKAFYDKVAEKFKEAF

SEQ ID NO: 591 - protein, 18 aa, Artificial Sequence
  Synthetic peptide with optional protecting groups.
DWFKAFYDKVAEKFKEAF

SEQ ID NO: 592 - protein, 18 aa, Artificial Sequence
  Synthetic peptide with optional protecting groups.
DWFKAFYDKVAEKFKEAF

SEQ ID NO: 593 - protein, 18 aa, Artificial Sequence
  Synthetic peptide with optional protecting groups.
DWFKAFYDKVAEKFKEAF

SEQ ID NO: 594 - protein, 18 aa, Artificial Sequence
  Synthetic peptide with optional protecting groups.
DWFKAFYDKVAEKFKEAF

SEQ ID NO: 595 - protein, 18 aa, Artificial Sequence
  Synthetic peptide with optional protecting groups.
DWFKAFYDKVAEKFKEAF

SEQ ID NO: 596 - protein, 18 aa, Artificial Sequence
  Synthetic peptide with optional protecting groups.
DWFKAFYDKVAEKFKEAF

SEQ ID NO: 597 - protein, 18 aa, Artificial Sequence
  Synthetic peptide with optional protecting groups.
DWFKAFYDKVAEKFKEAF

SEQ ID NO: 598 - protein, 18 aa, Artificial Sequence
  Synthetic peptide with optional protecting groups.
DWFXAFYDXVAEXFXEAF

SEQ ID NO: 599 - protein, 18 aa, Artificial Sequence
  Synthetic peptide with optional protecting groups.
DWFXAFYDXVAEXFXEAF

SEQ ID NO: 600 - protein, 18 aa, Artificial Sequence
  Synthetic peptide with optional protecting groups.
DWFXAFYDXVAEXFXEAF

SEQ ID NO: 601 - protein, 18 aa, Artificial Sequence
  Synthetic peptide with optional protecting groups.
DWFXAFYDXVAEXFXEAF

SEQ ID NO: 602 - protein, 18 aa, Artificial Sequence
  Synthetic peptide with optional protecting groups.
DWFXAFYDXVAEXFXEAF

SEQ ID NO: 603 - protein, 18 aa, Artificial Sequence
  Synthetic peptide with optional protecting groups.
DWFXAFYDXVAEXFXEAF

SEQ ID NO: 604 - protein, 18 aa, Artificial Sequence
  Synthetic peptide with optional protecting groups.
DWFXAFYDXVAEXFXEAF

SEQ ID NO: 605 - protein, 18 aa, Artificial Sequence
  Synthetic peptide with optional protecting groups.
DWFXAFYDXVAEXFXEAF

SEQ ID NO: 606 - protein, 18 aa, Artificial Sequence
  Synthetic peptide with optional protecting groups.
DWFXAFYDXVAEXFXEAF

SEQ ID NO: 607 - protein, 18 aa, Artificial Sequence
  Synthetic peptide with optional protecting groups.
DWFXAFYDXVAEXFXEAF

SEQ ID NO: 608 - protein, 18 aa, Artificial Sequence
  Synthetic peptide with optional protecting groups.
DWFXAFYDXVAEXFXEAF

SEQ ID NO: 609 - protein, 18 aa, Artificial Sequence
  Synthetic peptide with optional protecting groups.
DWFXAFYDXVAEXFXEAF

SEQ ID NO: 610 - protein, 18 aa, Artificial Sequence
  Synthetic peptide with optional protecting groups.
DWFXAFYDXVAEXFXEAF

SEQ ID NO: 611 - protein, 18 aa, Artificial Sequence
  Synthetic peptide with optional protecting groups.
DWFXAFYDXVAEXFXEAF

SEQ ID NO: 612 - protein, 18 aa, Artificial Sequence
  Synthetic peptide with optional protecting groups.
DWFXAFYDXVAEXFXEAF

SEQ ID NO: 613 - protein, 18 aa, Artificial Sequence
  Synthetic peptide with optional protecting groups.
DWFXAFYDXVAEXFXEAF

SEQ ID NO: 614 - protein, 18 aa, Artificial Sequence
  Synthetic peptide with optional protecting groups.
DWFXAFYDXVAEXFXEAF

SEQ ID NO: 615 - protein, 18 aa, Artificial Sequence
  Synthetic peptide with optional protecting groups.
DWFXAFYDXVAEXFXEAF

SEQ ID NO: 616 - protein, 18 aa, Artificial Sequence
  Synthetic peptide with optional protecting groups.
DWFXAFYDXVAEXFXEAF

SEQ ID NO: 617 - protein, 18 aa, Artificial Sequence
  Synthetic peptide with optional protecting groups.
DWFXAFYDXVAEXFXEAF

SEQ ID NO: 618 - protein, 18 aa, Artificial Sequence
  Synthetic peptide with optional protecting groups.
DWFXAFYDXVAEXFXEAF

SEQ ID NO: 619 - protein, 18 aa, Artificial Sequence
  Synthetic peptide with optional protecting groups.
DWFXAFYDXVAEXFXEAF

SEQ ID NO: 620 - protein, 4 aa, Artificial Sequence
  Synthetic peptide with optional protecting groups.
KRDS

SEQ ID NO: 621 - protein, 4 aa, Artificial Sequence
  Synthetic peptide with optional protecting groups.
KRDT

SEQ ID NO: 622 - protein, 4 aa, Artificial Sequence
  Synthetic peptide with optional protecting groups.
WRDI

SEQ ID NO: 623 - protein, 4 aa, Artificial Sequence
  Synthetic peptide with optional protecting groups.
WRDL

SEQ ID NO: 624 - protein, 4 aa, Artificial Sequence
  Synthetic peptide with optional protecting groups.
FRDL

SEQ ID NO: 625 - protein, 4 aa, Artificial Sequence
  Synthetic peptide with optional protecting groups.
FRDI

SEQ ID NO: 626 - protein, 4 aa, Artificial Sequence
  Synthetic peptide with optional protecting groups.
FRDX

SEQ ID NO: 627 - protein, 4 aa, Artificial Sequence
  Synthetic peptide with optional protecting groups.
FREX

SEQ ID NO: 628 - protein, 4 aa, Artificial Sequence
  Synthetic peptide with optional protecting groups.
FREI

SEQ ID NO: 629 - protein, 4 aa, Artificial Sequence
  Synthetic peptide with optional protecting groups.
FDRI

SEQ ID NO: 630 - protein, 4 aa, Artificial Sequence
  Synthetic peptide with optional protecting groups.
FERI

SEQ ID NO: 631 - protein, 4 aa, Artificial Sequence
  Synthetic peptide with optional protecting groups.
FDRL

SEQ ID NO: 632 - protein, 4 aa, Artificial Sequence
  Synthetic peptide with optional protecting groups.
FREL

SEQ ID NO: 633 - protein, 4 aa, Artificial Sequence
  Synthetic peptide with optional protecting groups.
FERL

SEQ ID NO: 634 - protein, 4 aa, Artificial Sequence
  Synthetic peptide with optional protecting groups.
FDRX

SEQ ID NO: 635 - protein, 4 aa, Artificial Sequence
  Synthetic peptide with optional protecting groups.
FERX

SEQ ID NO: 636 - protein, 4 aa, Artificial Sequence
  Synthetic peptide with optional protecting groups.
KERS

SEQ ID NO: 637 - protein, 4 aa, Artificial Sequence
  Synthetic peptide with optional protecting groups.
KERT

SEQ ID NO: 638 - protein, 4 aa, Artificial Sequence
  Synthetic peptide with optional protecting groups.
KDRS

SEQ ID NO: 639 - protein, 4 aa, Artificial Sequence
  Synthetic peptide with optional protecting groups.
KDRT

SEQ ID NO: 640 - protein, 4 aa, Artificial Sequence
  Synthetic peptide with optional protecting groups.
KRES

SEQ ID NO: 641 - protein, 4 aa, Artificial Sequence
  Synthetic peptide with optional protecting groups.
KRET

SEQ ID NO: 642 - protein, 4 aa, Artificial Sequence
  Synthetic peptide with optional protecting groups.
LERS

SEQ ID NO: 643 - protein, 4 aa, Artificial Sequence
  Synthetic peptide with optional protecting groups.
LERT

SEQ ID NO: 644 - protein, 4 aa, Artificial Sequence
  Synthetic peptide with optional protecting groups.
WRDS

SEQ ID NO: 645 - protein, 4 aa, Artificial Sequence
  Synthetic peptide with optional protecting groups.
WDRS

SEQ ID NO: 646 - protein, 4 aa, Artificial Sequence
  Synthetic peptide with optional protecting groups.
WERS

SEQ ID NO: 647 - protein, 4 aa, Artificial Sequence
  Synthetic peptide with optional protecting groups.
WRES

SEQ ID NO: 648 - protein, 4 aa, Artificial Sequence
  Synthetic peptide with optional protecting groups.
KERL

SEQ ID NO: 649 - protein, 4 aa, Artificial Sequence
  Synthetic peptide with optional protecting groups.
LRDS

SEQ ID NO: 650 - protein, 4 aa, Artificial Sequence
  Synthetic peptide with optional protecting groups.
LDRS

SEQ ID NO: 651 - protein, 4 aa, Artificial Sequence
  Synthetic peptide with optional protecting groups.
LERS

SEQ ID NO: 652 - protein, 4 aa, Artificial Sequence
  Synthetic peptide with optional protecting groups.
LRES

SEQ ID NO: 653 - protein, 4 aa, Artificial Sequence
  Synthetic peptide with optional protecting groups.
LRDT

SEQ ID NO: 654 - protein, 4 aa, Artificial Sequence
  Synthetic peptide with optional protecting groups.
EDRY

SEQ ID NO: 655 - protein, 4 aa, Artificial Sequence
  Synthetic peptide with optional protecting groups.
KRDS

SEQ ID NO: 656 - protein, 4 aa, Artificial Sequence
  Synthetic peptide with optional protecting groups.
WRDI

SEQ ID NO: 657 - protein, 4 aa, Artificial Sequence
  Synthetic peptide with optional protecting groups.
WRDL

SEQ ID NO: 658 - protein, 4 aa, Artificial Sequence
  Synthetic peptide with optional protecting groups.
FRDI

SEQ ID NO: 659 - protein, 4 aa, Artificial Sequence
  Synthetic peptide with optional protecting groups.
FRDL

SEQ ID NO: 660 - protein, 4 aa, Artificial Sequence
  Synthetic peptide with optional protecting groups.
WRDF

SEQ ID NO: 661 - protein, 4 aa, Artificial Sequence
  Synthetic peptide with optional protecting groups.
WRDY

SEQ ID NO: 662 - protein, 4 aa, Artificial Sequence
  Synthetic peptide with optional protecting groups.
WRDF

SEQ ID NO: 663 - protein, 4 aa, Artificial Sequence
  Synthetic peptide with optional protecting groups.
WRDY

SEQ ID NO: 664 - protein, 4 aa, Artificial Sequence
  Synthetic peptide with optional protecting groups.
XRES

SEQ ID NO: 665 - protein, 4 aa, Artificial Sequence
  Synthetic peptide with optional protecting groups.
KRDS

SEQ ID NO: 666 - protein, 4 aa, Artificial Sequence
  Synthetic peptide with optional protecting groups.
KRDT

SEQ ID NO: 667 - protein, 4 aa, Artificial Sequence
  Synthetic peptide with optional protecting groups.
LDRT

SEQ ID NO: 668 - protein, 4 aa, Artificial Sequence
  Synthetic peptide with optional protecting groups.
LERT

SEQ ID NO: 669 - protein, 4 aa, Artificial Sequence
  Synthetic peptide with optional protecting groups.
LRET

SEQ ID NO: 670 - protein, 4 aa, Artificial Sequence
  Synthetic peptide with optional protecting groups.
XRDS

SEQ ID NO: 671 - protein, 4 aa, Artificial Sequence
  Synthetic peptide with optional protecting groups.
XDRS

SEQ ID NO: 672 - protein, 4 aa, Artificial Sequence
  Synthetic peptide with optional protecting groups.
XERS

SEQ ID NO: 673 - protein, 4 aa, Artificial Sequence
  Synthetic peptide with optional protecting groups.
XRES

SEQ ID NO: 674 - protein, 4 aa, Artificial Sequence
  Synthetic peptide with optional protecting groups.
KRDS

SEQ ID NO: 675 - protein, 4 aa, Artificial Sequence
  Synthetic peptide with optional protecting groups.
KRDT

SEQ ID NO: 676 - protein, 4 aa, Artificial Sequence
  Synthetic peptide with optional protecting groups.
KERS

SEQ ID NO: 677 - protein, 4 aa, Artificial Sequence
  Synthetic peptide with optional protecting groups.
KERT

SEQ ID NO: 678 - protein, 4 aa, Artificial Sequence
  Synthetic peptide with optional protecting groups.
KDRS

SEQ ID NO: 679 - protein, 4 aa, Artificial Sequence
  Synthetic peptide with optional protecting groups.
KDRT

SEQ ID NO: 680 - protein, 4 aa, Artificial Sequence
  Synthetic peptide with optional protecting groups.
KRES

SEQ ID NO: 681 - protein, 4 aa, Artificial Sequence
  Synthetic peptide with optional protecting groups.
KRET

SEQ ID NO: 682 - protein, 4 aa, Artificial Sequence
  Synthetic peptide with optional protecting groups.
KERL

SEQ ID NO: 683 - protein, 4 aa, Artificial Sequence
  Synthetic peptide with optional protecting groups.
KREL

SEQ ID NO: 684 - protein, 4 aa, Artificial Sequence
  Synthetic peptide with optional protecting groups.
KRDT

SEQ ID NO: 685 - protein, 4 aa, Artificial Sequence
  Synthetic peptide with optional protecting groups.
KERS

SEQ ID NO: 686 - protein, 4 aa, Artificial Sequence
  Synthetic peptide with optional protecting groups.
KERT

SEQ ID NO: 687 - protein, 4 aa, Artificial Sequence
  Synthetic peptide with optional protecting groups.
KDRS

SEQ ID NO: 688 - protein, 4 aa, Artificial Sequence
  Synthetic peptide with optional protecting groups.
KDRT

SEQ ID NO: 689 - protein, 4 aa, Artificial Sequence
  Synthetic peptide with optional protecting groups.
KRES

SEQ ID NO: 690 - protein, 4 aa, Artificial Sequence
  Synthetic peptide with optional protecting groups.
KRET

SEQ ID NO: 691 - protein, 4 aa, Artificial Sequence
  Synthetic peptide with optional protecting groups.
KERL

SEQ ID NO: 692 - protein, 4 aa, Artificial Sequence
  Synthetic peptide with optional protecting groups.
KRDS

SEQ ID NO: 693 - protein, 4 aa, Artificial Sequence
  Synthetic peptide with optional protecting groups.
KRDT

SEQ ID NO: 694 - protein, 4 aa, Artificial Sequence
  Synthetic peptide with optional protecting groups.
KERS

SEQ ID NO: 695 - protein, 4 aa, Artificial Sequence
  Synthetic peptide with optional protecting groups.
KERT

SEQ ID NO: 696 - protein, 4 aa, Artificial Sequence
  Synthetic peptide with optional protecting groups.
KDRS

SEQ ID NO: 697 - protein, 4 aa, Artificial Sequence
  Synthetic peptide with optional protecting groups.
KDRT

SEQ ID NO: 698 - protein, 4 aa, Artificial Sequence
  Synthetic peptide with optional protecting groups.
KRES

SEQ ID NO: 699 - protein, 4 aa, Artificial Sequence
  Synthetic peptide with optional protecting groups.
KRET

SEQ ID NO: 700 - protein, 4 aa, Artificial Sequence
  Synthetic peptide with optional protecting groups.
KERL

SEQ ID NO: 701 - protein, 4 aa, Artificial Sequence
  Synthetic peptide with optional protecting groups.
XRES

SEQ ID NO: 702 - protein, 4 aa, Artificial Sequence
  Synthetic peptide with optional protecting groups.
XERS

SEQ ID NO: 703 - protein, 4 aa, Artificial Sequence
  Synthetic peptide with optional protecting groups.
XRDS

SEQ ID NO: 704 - protein, 4 aa, Artificial Sequence
  Synthetic peptide with optional protecting groups.
XDRS

SEQ ID NO: 705 - protein, 4 aa, Artificial Sequence
  Synthetic peptide with optional protecting groups.
XDRT

SEQ ID NO: 706 - protein, 4 aa, Artificial Sequence
  Synthetic peptide with optional protecting groups.
XRDT

SEQ ID NO: 707 - protein, 4 aa, Artificial Sequence
  Synthetic peptide with optional protecting groups.
XERT

SEQ ID NO: 708 - protein, 4 aa, Artificial Sequence
  Synthetic peptide with optional protecting groups.
XRET

SEQ ID NO: 709 - protein, 4 aa, Artificial Sequence
  Synthetic peptide with optional protecting groups.
WDRI

SEQ ID NO: 710 - protein, 4 aa, Artificial Sequence
  Synthetic peptide with optional protecting groups.
WREI

SEQ ID NO: 711 - protein, 4 aa, Artificial Sequence
  Synthetic peptide with optional protecting groups.
WERI

SEQ ID NO: 712 - protein, 4 aa, Artificial Sequence
  Synthetic peptide with optional protecting groups.
WDRL

SEQ ID NO: 713 - protein, 4 aa, Artificial Sequence
  Synthetic peptide with optional protecting groups.
WREL

SEQ ID NO: 714 - protein, 4 aa, Artificial Sequence
  Synthetic peptide with optional protecting groups.
WERL

SEQ ID NO: 715 - protein, 4 aa, Artificial Sequence
  Synthetic peptide with optional protecting groups.
FDRI

SEQ ID NO: 716 - protein, 4 aa, Artificial Sequence
  Synthetic peptide with optional protecting groups.
FREI

SEQ ID NO: 717 - protein, 4 aa, Artificial Sequence
  Synthetic peptide with optional protecting groups.
FERI

SEQ ID NO: 718 - protein, 4 aa, Artificial Sequence
  Synthetic peptide with optional protecting groups.
FDRL

SEQ ID NO: 719 - protein, 4 aa, Artificial Sequence
  Synthetic peptide with optional protecting groups.
FREL

SEQ ID NO: 720 - protein, 4 aa, Artificial Sequence
  Synthetic peptide with optional protecting groups.
FERL

SEQ ID NO: 721 - protein, 4 aa, Artificial Sequence
  Synthetic peptide with optional protecting groups.
WRDF

SEQ ID NO: 722 - protein, 4 aa, Artificial Sequence
  Synthetic peptide with optional protecting groups.
WREF

SEQ ID NO: 723 - protein, 4 aa, Artificial Sequence
  Synthetic peptide with optional protecting groups.
WERF

SEQ ID NO: 724 - protein, 4 aa, Artificial Sequence
  Synthetic peptide with optional protecting groups.
WDRY

SEQ ID NO: 725 - protein, 4 aa, Artificial Sequence
  Synthetic peptide with optional protecting groups.
WREY

SEQ ID NO: 726 - protein, 4 aa, Artificial Sequence
  Synthetic peptide with optional protecting groups.
WERY

SEQ ID NO: 727 - protein, 4 aa, Artificial Sequence
  Synthetic peptide with optional protecting groups.
WRDT

SEQ ID NO: 728 - protein, 4 aa, Artificial Sequence
  Synthetic peptide with optional protecting groups.
WDRT

SEQ ID NO: 729 - protein, 4 aa, Artificial Sequence
  Synthetic peptide with optional protecting groups.
WRET

SEQ ID NO: 730 - protein, 4 aa, Artificial Sequence
  Synthetic peptide with optional protecting groups.
WERT

SEQ ID NO: 731 - protein, 4 aa, Artificial Sequence
  Synthetic peptide with optional protecting groups.
FRDX

SEQ ID NO: 732 - protein, 4 aa, Artificial Sequence
  Synthetic peptide with optional protecting groups.
FREX

SEQ ID NO: 733 - protein, 4 aa, Artificial Sequence
  Synthetic peptide with optional protecting groups.
FKDL

SEQ ID NO: 734 - protein, 4 aa, Artificial Sequence
  Synthetic peptide with optional protecting groups.
FDKL

SEQ ID NO: 735 - protein, 4 aa, Artificial Sequence
  Synthetic peptide with optional protecting groups.
FKEL

SEQ ID NO: 736 - protein, 4 aa, Artificial Sequence
  Synthetic peptide with optional protecting groups.
FEKL

SEQ ID NO: 737 - protein, 4 aa, Artificial Sequence
  Synthetic peptide with optional protecting groups.
FKDI

SEQ ID NO: 738 - protein, 4 aa, Artificial Sequence
  Synthetic peptide with optional protecting groups.
FDKI

SEQ ID NO: 739 - protein, 4 aa, Artificial Sequence
  Synthetic peptide with optional protecting groups.
FKEI

SEQ ID NO: 740 - protein, 4 aa, Artificial Sequence
  Synthetic peptide with optional protecting groups.
FEKI

SEQ ID NO: 741 - protein, 4 aa, Artificial Sequence
  Synthetic peptide with optional protecting groups.
FKDX

SEQ ID NO: 742 - protein, 4 aa, Artificial Sequence
  Synthetic peptide with optional protecting groups.
FDKX

SEQ ID NO: 743 - protein, 4 aa, Artificial Sequence
  Synthetic peptide with optional protecting groups.
FKEX

SEQ ID NO: 744 - protein, 4 aa, Artificial Sequence
  Synthetic peptide with optional protecting groups.
FEKX

SEQ ID NO: 745 - protein, 4 aa, Artificial Sequence
  Synthetic peptide with optional protecting groups.
FHDL

SEQ ID NO: 746 - protein, 4 aa, Artificial Sequence
  Synthetic peptide with optional protecting groups.
FDHL

SEQ ID NO: 747 - protein, 4 aa, Artificial Sequence
  Synthetic peptide with optional protecting groups.
FHEL

SEQ ID NO: 748 - protein, 4 aa, Artificial Sequence
  Synthetic peptide with optional protecting groups.
FEHL

SEQ ID NO: 749 - protein, 4 aa, Artificial Sequence
  Synthetic peptide with optional protecting groups.
FHDI

SEQ ID NO: 750 - protein, 4 aa, Artificial Sequence
  Synthetic peptide with optional protecting groups.
FDHI

SEQ ID NO: 751 - protein, 4 aa, Artificial Sequence
  Synthetic peptide with optional protecting groups.
FHEI

SEQ ID NO: 752 - protein, 4 aa, Artificial Sequence
  Synthetic peptide with optional protecting groups.
FEHI

SEQ ID NO: 753 - protein, 4 aa, Artificial Sequence
  Synthetic peptide with optional protecting groups.
FHDX

SEQ ID NO: 754 - protein, 4 aa, Artificial Sequence
  Synthetic peptide with optional protecting groups.
FDHX

SEQ ID NO: 755 - protein, 4 aa, Artificial Sequence
  Synthetic peptide with optional protecting groups.
FHEX

SEQ ID NO: 756 - protein, 4 aa, Artificial Sequence
  Synthetic peptide with optional protecting groups.
FEHX

SEQ ID NO: 757 - protein, 4 aa, Artificial Sequence
  Synthetic peptide with optional protecting groups.
KKDS

SEQ ID NO: 758 - protein, 4 aa, Artificial Sequence
  Synthetic peptide with optional protecting groups.
KDKS

SEQ ID NO: 759 - protein, 4 aa, Artificial Sequence
  Synthetic peptide with optional protecting groups.
KKES

SEQ ID NO: 760 - protein, 4 aa, Artificial Sequence
  Synthetic peptide with optional protecting groups.
KEKS

SEQ ID NO: 761 - protein, 4 aa, Artificial Sequence
  Synthetic peptide with optional protecting groups.
KHDS

SEQ ID NO: 762 - protein, 4 aa, Artificial Sequence
  Synthetic peptide with optional protecting groups.
KDHS

SEQ ID NO: 763 - protein, 4 aa, Artificial Sequence
  Synthetic peptide with optional protecting groups.
KHES

SEQ ID NO: 764 - protein, 4 aa, Artificial Sequence
  Synthetic peptide with optional protecting groups.
KEHS

SEQ ID NO: 765 - protein, 4 aa, Artificial Sequence
  Synthetic peptide with optional protecting groups.
KLRS

SEQ ID NO: 766 - protein, 4 aa, Artificial Sequence
  Synthetic peptide with optional protecting groups.
KRLS

SEQ ID NO: 767 - protein, 4 aa, Artificial Sequence
  Synthetic peptide with optional protecting groups.
KLRT

SEQ ID NO: 768 - protein, 4 aa, Artificial Sequence
  Synthetic peptide with optional protecting groups.
KRLT

SEQ ID NO: 769 - protein, 4 aa, Artificial Sequence
  Synthetic peptide with optional protecting groups.
KELS

SEQ ID NO: 770 - protein, 4 aa, Artificial Sequence
  Synthetic peptide with optional protecting groups.
KLES

SEQ ID NO: 771 - protein, 4 aa, Artificial Sequence
  Synthetic peptide with optional protecting groups.
KELT

SEQ ID NO: 772 - protein, 4 aa, Artificial Sequence
  Synthetic peptide with optional protecting groups.
KLRS

SEQ ID NO: 773 - protein, 4 aa, Artificial Sequence
  Synthetic peptide with optional protecting groups.
KLRT

SEQ ID NO: 774 - protein, 4 aa, Artificial Sequence
  Synthetic peptide with optional protecting groups.
KELS

SEQ ID NO: 775 - protein, 4 aa, Artificial Sequence
  Synthetic peptide with optional protecting groups.
KELT

SEQ ID NO: 776 - protein, 4 aa, Artificial Sequence
  Synthetic peptide with optional protecting groups.
KEIT

SEQ ID NO: 777 - protein, 4 aa, Artificial Sequence
  Synthetic peptide with optional protecting groups.
KLRS

SEQ ID NO: 778 - protein, 4 aa, Artificial Sequence
  Synthetic peptide with optional protecting groups.
KLRT

SEQ ID NO: 779 - protein, 4 aa, Artificial Sequence
  Synthetic peptide with optional protecting groups.
KELS

SEQ ID NO: 780 - protein, 4 aa, Artificial Sequence
  Synthetic peptide with optional protecting groups.
KELT

SEQ ID NO: 781 - protein, 4 aa, Artificial Sequence
  Synthetic peptide with optional protecting groups.
KLRS

SEQ ID NO: 782 - protein, 4 aa, Artificial Sequence
  Synthetic peptide with optional protecting groups.
KRFT

SEQ ID NO: 783 - protein, 4 aa, Artificial Sequence
  Synthetic peptide with optional protecting groups.
KLRT

SEQ ID NO: 784 - protein, 4 aa, Artificial Sequence
  Synthetic peptide with optional protecting groups.
KEIT

SEQ ID NO: 785 - protein, 4 aa, Artificial Sequence
  Synthetic peptide with optional protecting groups.
KEVT

SEQ ID NO: 786 - protein, 4 aa, Artificial Sequence
  Synthetic peptide with optional protecting groups.
KEAT

SEQ ID NO: 787 - protein, 4 aa, Artificial Sequence
  Synthetic peptide with optional protecting groups.
KEGT

SEQ ID NO: 788 - protein, 4 aa, Artificial Sequence
  Synthetic peptide with optional protecting groups.
KELS

SEQ ID NO: 789 - protein, 4 aa, Artificial Sequence
  Synthetic peptide with optional protecting groups.
KELT

SEQ ID NO: 790 - protein, 4 aa, Artificial Sequence
  Synthetic peptide with optional protecting groups.
KRWY

SEQ ID NO: 791 - protein, 4 aa, Artificial Sequence
  Synthetic peptide with optional protecting groups.
KWRY

SEQ ID NO: 792 - protein, 4 aa, Artificial Sequence
  Synthetic peptide with optional protecting groups.
KRYW

SEQ ID NO: 793 - protein, 4 aa, Artificial Sequence
  Synthetic peptide with optional protecting groups.
KYRW

SEQ ID NO: 794 - protein, 5 aa, Artificial Sequence
  Synthetic peptide with optional protecting groups.
KRYWT

SEQ ID NO: 795 - protein, 4 aa, Artificial Sequence
  Synthetic peptide with optional protecting groups.
KRYT

SEQ ID NO: 796 - protein, 4 aa, Artificial Sequence
  Synthetic peptide with optional protecting groups.
KRWT

SEQ ID NO: 797 - protein, 4 aa, Artificial Sequence
  Synthetic peptide with optional protecting groups.
KRWY

SEQ ID NO: 798 - protein, 4 aa, Artificial Sequence
  Synthetic peptide with optional protecting groups.
KRYW

SEQ ID NO: 799 - protein, 5 aa, Artificial Sequence
  Synthetic peptide with optional protecting groups.
KRYWT

SEQ ID NO: 800 - protein, 4 aa, Artificial Sequence
  Synthetic peptide with optional protecting groups.
KRYT

SEQ ID NO: 801 - protein, 4 aa, Artificial Sequence
  Synthetic peptide with optional protecting groups.
KRWT

SEQ ID NO: 802 - protein, 4 aa, Artificial Sequence
  Synthetic peptide with optional protecting groups.
KRWY

SEQ ID NO: 803 - protein, 4 aa, Artificial Sequence
  Synthetic peptide with optional protecting groups.
KRYW

SEQ ID NO: 804 - protein, 5 aa, Artificial Sequence
  Synthetic peptide with optional protecting groups.
KRYWT

SEQ ID NO: 805 - protein, 4 aa, Artificial Sequence
  Synthetic peptide with optional protecting groups.
KRYT

SEQ ID NO: 806 - protein, 4 aa, Artificial Sequence
  Synthetic peptide with optional protecting groups.
KRWT

SEQ ID NO: 807 - protein, 4 aa, Artificial Sequence
  Synthetic peptide with optional protecting groups.
EKRY

SEQ ID NO: 808 - protein, 4 aa, Artificial Sequence
  Synthetic peptide with optional protecting groups.
KRWY

SEQ ID NO: 809 - protein, 4 aa, Artificial Sequence
  Synthetic peptide with optional protecting groups.
KRYW

SEQ ID NO: 810 - protein, 5 aa, Artificial Sequence
  Synthetic peptide with optional protecting groups.
KRYWT

SEQ ID NO: 811 - protein, 4 aa, Artificial Sequence
  Synthetic peptide with optional protecting groups.
KRYT

SEQ ID NO: 812 - protein, 4 aa, Artificial Sequence
  Synthetic peptide with optional protecting groups.
KRFT

SEQ ID NO: 813 - protein, 4 aa, Artificial Sequence
  Synthetic peptide with optional protecting groups.
KRWT

SEQ ID NO: 814 - protein, 5 aa, Artificial Sequence
  Synthetic peptide with optional protecting groups.
KFWFS

SEQ ID NO: 815 - protein, 5 aa, Artificial Sequence
  Synthetic peptide with optional protecting groups.
KFWFT

SEQ ID NO: 816 - protein, 5 aa, Artificial Sequence
  Synthetic peptide with optional protecting groups.
KFYFS

SEQ ID NO: 817 - protein, 5 aa, Artificial Sequence
  Synthetic peptide with optional protecting groups.
KFYFT

SEQ ID NO: 818 - protein, 5 aa, Artificial Sequence
  Synthetic peptide with optional protecting groups.
KFHFS

SEQ ID NO: 819 - protein, 5 aa, Artificial Sequence
  Synthetic peptide with optional protecting groups.
KFHFT

SEQ ID NO: 820 - protein, 6 aa, Artificial Sequence
  Synthetic peptide with optional protecting groups.
KVFFYS

SEQ ID NO: 821 - protein, 5 aa, Artificial Sequence
  Synthetic peptide with optional protecting groups.
KFWFS

SEQ ID NO: 822 - protein, 5 aa, Artificial Sequence
  Synthetic peptide with optional protecting groups.
KFWFT

SEQ ID NO: 823 - protein, 5 aa, Artificial Sequence
  Synthetic peptide with optional protecting groups.
KFYFS

SEQ ID NO: 824 - protein, 5 aa, Artificial Sequence
  Synthetic peptide with optional protecting groups.
KFYFT

SEQ ID NO: 825 - protein, 5 aa, Artificial Sequence
  Synthetic peptide with optional protecting groups.
KFHFS

SEQ ID NO: 826 - protein, 5 aa, Artificial Sequence
  Synthetic peptide with optional protecting groups.
KFHFT

SEQ ID NO: 827 - protein, 5 aa, Artificial Sequence
  Synthetic peptide with optional protecting groups.
LFWFT

SEQ ID NO: 828 - protein, 5 aa, Artificial Sequence
  Synthetic peptide with optional protecting groups.
LFWFS

SEQ ID NO: 829 - protein, 22 aa, Artificial Sequence
  Synthetic peptide with optional protecting groups.
LLEQLNEQFNWVSRLANLTQGE

SEQ ID NO: 830 - protein, 18 aa, Artificial Sequence
  Synthetic peptide with optional protecting groups.
LLEQLNEQFNWVSRLANL

SEQ ID NO: 831 - protein, 25 aa, Artificial Sequence
  Synthetic peptide with optional protecting groups.
NELQEMSNQGSKYVNKEIQNAVNGV

SEQ ID NO: 832 - protein, 21 aa, Artificial Sequence
  Synthetic peptide with optional protecting groups.
IQNAVNGVKQIKTLIEKTNEE

SEQ ID NO: 833 - protein, 32 aa, Artificial Sequence
  Synthetic peptide with optional protecting groups.
RKTLLSNLEEAKKKKEDALNETRESETKLKEL

SEQ ID NO: 834 - protein, 16 aa, Artificial Sequence
  Synthetic peptide with optional protecting groups.
PGVCNETMMALWEECK

SEQ ID NO: 835 - protein, 16 aa, Artificial Sequence
  Synthetic peptide with optional protecting groups.
PCLKQTCMKFYARVCR

SEQ ID NO: 836 - protein, 19 aa, Artificial Sequence
  Synthetic peptide with optional protecting groups.
ECKPCLKQTCMKFYARVCR

SEQ ID NO: 837 - protein, 10 aa, Artificial Sequence
  Synthetic peptide with optional protecting groups.
LVGRQLEEFL

SEQ ID NO: 838 - protein, 12 aa, Artificial Sequence
  Synthetic peptide with optional protecting groups.
MNGDRIDSLLEN

SEQ ID NO: 839 - protein, 11 aa, Artificial Sequence
  Synthetic peptide with optional protecting groups.
QQTHMLDVMQD

SEQ ID NO: 840 - protein, 14 aa, Artificial Sequence
  Synthetic peptide with optional protecting groups.
FSRASSIIDELFQD

SEQ ID NO: 841 - protein, 15 aa, Artificial Sequence
  Synthetic peptide with optional protecting groups.
PFLEMIHEAQQAMDI

SEQ ID NO: 842 - protein, 11 aa, Artificial Sequence
  Synthetic peptide with optional protecting groups.
PTEFIREGDDD

SEQ ID NO: 843 - protein, 15 aa, Artificial Sequence
  Synthetic peptide with optional protecting groups.
RMKDQCDKCREILSV

SEQ ID NO: 844 - protein, 32 aa, Artificial Sequence
  Synthetic peptide with optional protecting groups.
PSQAKLRRELDESLQVAERLTRKYNELLKSYQ

SEQ ID NO: 845 - protein, 22 aa, Artificial Sequence
  Synthetic peptide with optional protecting groups.
LLEQLNEQFNWVSRLANLTEGE

SEQ ID NO: 846 - protein, 11 aa, Artificial Sequence
  Synthetic peptide with optional protecting groups.
DQYYLRVTTVA

SEQ ID NO: 847 - protein, 14 aa, Artificial Sequence
  Synthetic peptide with optional protecting groups.
PSGVTEVVVKLFDS

SEQ ID NO: 848 - protein, 21 aa, Artificial Sequence
  Synthetic peptide with optional protecting groups.
PKFMETVAEKALQEYRKKHRE

SEQ ID NO: 849 - protein, 26 aa, Artificial Sequence
  Synthetic peptide with optional protecting groups.
WDRVKDLATVYVDVLKDSGRDYVSQF

SEQ ID NO: 850 - protein, 25 aa, Artificial Sequence
  Synthetic peptide with optional protecting groups.
VATVMWDYFSQLSNNAKEAVEHLQK

SEQ ID NO: 851 - protein, 27 aa, Artificial Sequence
  Synthetic peptide with optional protecting groups.
RWELALGRFWDYLRWVQTLSEQVQEEL

SEQ ID NO: 852 - protein, 35 aa, Artificial Sequence
  Synthetic peptide with optional protecting groups.
LSSQVTQELRALMDETMKELKELKAYKSELEEQLT

SEQ ID NO: 853 - protein, 26 aa, Artificial Sequence
  Synthetic peptide with optional protecting groups.
ARLSKELQAAQARLGADMEDVCGRLV

SEQ ID NO: 854 - protein, 26 aa, Artificial Sequence
  Synthetic peptide with optional protecting groups.
VRLASHLRKLRKRLLRDADDLQKRLA

SEQ ID NO: 855 - protein, 19 aa, Artificial Sequence
  Synthetic peptide with optional protecting groups.
PLVEDMQRQWAGLVEKVQA

SEQ ID NO: 856 - protein, 17 aa, Artificial Sequence
  Synthetic peptide with optional protecting groups.
MSTYTGIFTDQVLSVLK

SEQ ID NO: 857 - protein, 22 aa, Artificial Sequence
  Synthetic peptide with optional protecting groups.
LLSFMQGYMKHATKTAKDALSS

SEQ ID NO: 858 - protein, 17 aa, Artificial Sequence
  Synthetic peptide with optional protecting groups.
KWIYHLTEGSTDLRTEG

SEQ ID NO: 859 - protein, 17 aa, Artificial Sequence
  Synthetic peptide with optional protecting groups.
KWFYHLTEGSTDLRTEG

SEQ ID NO: 860 - protein, 17 aa, Artificial Sequence
  Synthetic peptide with optional protecting groups.
KWLYHLTEGSTDLRTEG

SEQ ID NO: 861 - protein, 17 aa, Artificial Sequence
  Synthetic peptide with optional protecting groups.
KWVYHLTEGSTDLRTEG

SEQ ID NO: 862 - protein, 17 aa, Artificial Sequence
  Synthetic peptide with optional protecting groups.
KYIWHLTEGSTDLRTEG

SEQ ID NO: 863 - protein, 17 aa, Artificial Sequence
  Synthetic peptide with optional protecting groups.
KWIYHFTEGSTDLRTEG

SEQ ID NO: 864 - protein, 17 aa, Artificial Sequence
  Synthetic peptide with optional protecting groups.
KWFYHITEGSTDLRTEG

SEQ ID NO: 865 - protein, 17 aa, Artificial Sequence
  Synthetic peptide with optional protecting groups.
KWLYHVTEGSTDLRTEG

SEQ ID NO: 866 - protein, 17 aa, Artificial Sequence
  Synthetic peptide with optional protecting groups.
KWVYHYTEGSTDLRTEG

SEQ ID NO: 867 - protein, 17 aa, Artificial Sequence
  Synthetic peptide with optional protecting groups.
KYIWHFTEGSTDLRTEG

SEQ ID NO: 868 - protein, 17 aa, Artificial Sequence
  Synthetic peptide with optional protecting groups.
KYIWHITEGSTDLRTEG

SEQ ID NO: 869 - protein, 17 aa, Artificial Sequence
  Synthetic peptide with optional protecting groups.
KYIWHVTEGSTDLRTEG

SEQ ID NO: 870 - protein, 17 aa, Artificial Sequence
  Synthetic peptide with optional protecting groups.
KYIWHYTEGSTDLRTEG

SEQ ID NO: 871 - protein, 17 aa, Artificial Sequence
  Synthetic peptide with optional protecting groups.
KFIWHLTEGSTDLRTEG

SEQ ID NO: 872 - protein, 17 aa, Artificial Sequence
  Synthetic peptide with optional protecting groups.
KLIWHLTEGSTDLRTEG

SEQ ID NO: 873 - protein, 17 aa, Artificial Sequence
  Synthetic peptide with optional protecting groups.
KIIWHLTEGSTDLRTEG

SEQ ID NO: 874 - protein, 17 aa, Artificial Sequence
  Synthetic peptide with optional protecting groups.
KYIWFLTEGSTDLRTEG

SEQ ID NO: 875 - protein, 17 aa, Artificial Sequence
  Synthetic peptide with optional protecting groups.
KWIYFLTEGSTDLRTEG

SEQ ID NO: 876 - protein, 17 aa, Artificial Sequence
  Synthetic peptide with optional protecting groups.
KWIYLLTEGSTDLRTEG

SEQ ID NO: 877 - protein, 17 aa, Artificial Sequence
  Synthetic peptide with optional protecting groups.
KWIYHFTEGSTDLRTEG

SEQ ID NO: 878 - protein, 17 aa, Artificial Sequence
  Synthetic peptide with optional protecting groups.
KWIYHYTEGSTDLRTEG

SEQ ID NO: 879 - protein, 17 aa, Artificial Sequence
  Synthetic peptide with optional protecting groups.
KWIYHITEGSTDLRTEG

SEQ ID NO: 880 - protein, 17 aa, Artificial Sequence
  Synthetic peptide with optional protecting groups.
KWIYHLSEGSTDLRTEG

SEQ ID NO: 881 - protein, 17 aa, Artificial Sequence
  Synthetic peptide with optional protecting groups.
KWIYHLTDGSTDLRTEG

SEQ ID NO: 882 - protein, 17 aa, Artificial Sequence
  Synthetic peptide with optional protecting groups.
KWIYHLTEGTSDLRTEG

SEQ ID NO: 883 - protein, 17 aa, Artificial Sequence
  Synthetic peptide with optional protecting groups.
KWIYHLTEGSTELRTEG

SEQ ID NO: 884 - protein, 17 aa, Artificial Sequence
  Synthetic peptide with optional protecting groups.
KWIYHLTEGSTDFRTEG

SEQ ID NO: 885 - protein, 17 aa, Artificial Sequence
  Synthetic peptide with optional protecting groups.
KWIYHLTEGSTDYRTEG

SEQ ID NO: 886 - protein, 17 aa, Artificial Sequence
  Synthetic peptide with optional protecting groups.
KWIYHLTEGSTDIRTEG

SEQ ID NO: 887 - protein, 17 aa, Artificial Sequence
  Synthetic peptide with optional protecting groups.
KWIYHLTEGSTDVRTEG

SEQ ID NO: 888 - protein, 17 aa, Artificial Sequence
  Synthetic peptide with optional protecting groups.
KWIYHLTEGSTDLKTEG

SEQ ID NO: 889 - protein, 17 aa, Artificial Sequence
  Synthetic peptide with optional protecting groups.
KWIYHLTEGSTDLRSEG

SEQ ID NO: 890 - protein, 17 aa, Artificial Sequence
  Synthetic peptide with optional protecting groups.
KWIYHLTEGSTDLRTDG

SEQ ID NO: 891 - protein, 17 aa, Artificial Sequence
  Synthetic peptide with optional protecting groups.
KWIYHLTEGSTDIKTEG

SEQ ID NO: 892 - protein, 17 aa, Artificial Sequence
  Synthetic peptide with optional protecting groups.
KWIYHLTEGSTDIRSEG

SEQ ID NO: 893 - protein, 17 aa, Artificial Sequence
  Synthetic peptide with optional protecting groups.
KWIYHLTEGSTDIKSEG

SEQ ID NO: 894 - protein, 17 aa, Artificial Sequence
  Synthetic peptide with optional protecting groups.
KWIYHLTEGSTDIKSDG

SEQ ID NO: 895 - protein, 17 aa, Artificial Sequence
  Synthetic peptide with optional protecting groups.
RWIYHLTEGSTDLRTEG

SEQ ID NO: 896 - protein, 17 aa, Artificial Sequence
  Synthetic peptide with optional protecting groups.
RYIWHLTEGSTDIRTEG

SEQ ID NO: 897 - protein, 17 aa, Artificial Sequence
  Synthetic peptide with optional protecting groups.
RWIYHLTEGSTDIRTDG

SEQ ID NO: 898 - protein, 17 aa, Artificial Sequence
  Synthetic peptide with optional protecting groups.
RWIFHLTEGSTDIRTEG

SEQ ID NO: 899 - protein, 17 aa, Artificial Sequence
  Synthetic peptide with optional protecting groups.
RWIYHLTEGSTDLKTEG

SEQ ID NO: 900 - protein, 17 aa, Artificial Sequence
  Synthetic peptide with optional protecting groups.
RWIYHLTDGSTDIRTEG

SEQ ID NO: 901 - protein, 17 aa, Artificial Sequence
  Synthetic peptide with optional protecting groups.
RWIYHLTDGSTDLRTEG

SEQ ID NO: 902 - protein, 17 aa, Artificial Sequence
  Synthetic peptide with optional protecting groups.
RWIYFLTEGSTDIRTEG

SEQ ID NO: 903 - protein, 17 aa, Artificial Sequence
  Synthetic peptide with optional protecting groups.
RWIYFLTEGSTDLRTEG

SEQ ID NO: 904 - protein, 17 aa, Artificial Sequence
  Synthetic peptide with optional protecting groups.
KWFYHLTEGSTDFRTEG

SEQ ID NO: 905 - protein, 17 aa, Artificial Sequence
  Synthetic peptide with optional protecting groups.
RWFYHLTEGSTDLRTEG

SEQ ID NO: 906 - protein, 17 aa, Artificial Sequence
  Synthetic peptide with optional protecting groups.
KWIFHLTEGSTDIRTDG

SEQ ID NO: 907 - protein, 17 aa, Artificial Sequence
  Synthetic peptide with optional protecting groups.
RWIYHLTEGSTDIRTDG

SEQ ID NO: 908 - protein, 17 aa, Artificial Sequence
  Synthetic peptide with optional protecting groups.
RWIYHLTEGSTDLRTDG

SEQ ID NO: 909 - protein, 17 aa, Artificial Sequence
  Synthetic peptide with optional protecting groups.
KWIYHLTEGSTDIKTEG

SEQ ID NO: 910 - protein, 17 aa, Artificial Sequence
  Synthetic peptide with optional protecting groups.
KWIYHLTEGSTDIKTDG

SEQ ID NO: 911 - protein, 17 aa, Artificial Sequence
  Synthetic peptide with optional protecting groups.
KWIYHLTEGSTDFKTEG

SEQ ID NO: 912 - protein, 17 aa, Artificial Sequence
  Synthetic peptide with optional protecting groups.
KWIYHLTEGSTDYKTEG

SEQ ID NO: 913 - protein, 17 aa, Artificial Sequence
  Synthetic peptide with optional protecting groups.
KWIYHLTEGSTDIRTEG

SEQ ID NO: 914 - protein, 17 aa, Artificial Sequence
  Synthetic peptide with optional protecting groups.
KWFYHFTEGSTDLRTEG

SEQ ID NO: 915 - protein, 17 aa, Artificial Sequence
  Synthetic peptide with optional protecting groups.
RWFYHFTEGSTDLRTEG

SEQ ID NO: 916 - protein, 17 aa, Artificial Sequence
  Synthetic peptide with optional protecting groups.
KWFYHFTEGSTDFRTEG

SEQ ID NO: 917 - protein, 17 aa, Artificial Sequence
  Synthetic peptide with optional protecting groups.
KWFYHFTDGSTDIRTEG

SEQ ID NO: 918 - protein, 17 aa, Artificial Sequence
  Synthetic peptide with optional protecting groups.
RWFYHFTEGSTDLRTEG

SEQ ID NO: 919 - protein, 17 aa, Artificial Sequence
  Synthetic peptide with optional protecting groups.
RWFYHFTEGSTDFRTEG

SEQ ID NO: 920 - protein, 17 aa, Artificial Sequence
  Synthetic peptide with optional protecting groups.
RWFYHFTEGSTDFRTDG

SEQ ID NO: 921 - protein, 19 aa, Artificial Sequence
  Synthetic peptide with optional protecting groups.
EKCVEEFKSLTSCLDSKAF

SEQ ID NO: 922 - protein, 19 aa, Artificial Sequence
  Synthetic peptide with optional protecting groups.
DKCVEEFKSLTSCLDSKAF

SEQ ID NO: 923 - protein, 19 aa, Artificial Sequence
  Synthetic peptide with optional protecting groups.
EKCVDEFKSLTSCLDSKAF

SEQ ID NO: 924 - protein, 19 aa, Artificial Sequence
  Synthetic peptide with optional protecting groups.
EKCVEDFKSLTSCLDSKAF

SEQ ID NO: 925 - protein, 19 aa, Artificial Sequence
  Synthetic peptide with optional protecting groups.
ERCVEEFKSLTSCLDSKAF

SEQ ID NO: 926 - protein, 19 aa, Artificial Sequence
  Synthetic peptide with optional protecting groups.
DKCVDDFKSLTSCLDSKAF

SEQ ID NO: 927 - protein, 19 aa, Artificial Sequence
  Synthetic peptide with optional protecting groups.
DRCVEEFKSLTSCLDSKAF

SEQ ID NO: 928 - protein, 19 aa, Artificial Sequence
  Synthetic peptide with optional protecting groups.
ERCVDDFKSLTSCLDSKAF

SEQ ID NO: 929 - protein, 19 aa, Artificial Sequence
  Synthetic peptide with optional protecting groups.
EKCVEEFKSFTSCLDSKAF

SEQ ID NO: 930 - protein, 19 aa, Artificial Sequence
  Synthetic peptide with optional protecting groups.
EKCVEEFKSITSCLDSKAF

SEQ ID NO: 931 - protein, 19 aa, Artificial Sequence
  Synthetic peptide with optional protecting groups.
EKCVEEFKSVTSCLDSKAF

SEQ ID NO: 932 - protein, 19 aa, Artificial Sequence
  Synthetic peptide with optional protecting groups.
ERCVEEFKSYTSCLDSKAF

SEQ ID NO: 933 - protein, 19 aa, Artificial Sequence
  Synthetic peptide with optional protecting groups.
ERCVEEFKSFTSCLDSKAF

SEQ ID NO: 934 - protein, 19 aa, Artificial Sequence
  Synthetic peptide with optional protecting groups.
ERCVEEFKSITSCLDSKAF

SEQ ID NO: 935 - protein, 19 aa, Artificial Sequence
  Synthetic peptide with optional protecting groups.
ERCVEEFKSVTSCLDSKAF

SEQ ID NO: 936 - protein, 19 aa, Artificial Sequence
  Synthetic peptide with optional protecting groups.
ERCVEEFKSYTSCLDSKAF

SEQ ID NO: 937 - protein, 19 aa, Artificial Sequence
  Synthetic peptide with optional protecting groups.
EKCVEEFKSFTTCLDSKAF

SEQ ID NO: 938 - protein, 19 aa, Artificial Sequence
  Synthetic peptide with optional protecting groups.
EKCVEEFKSISSCLDSKAF

SEQ ID NO: 939 - protein, 19 aa, Artificial Sequence
  Synthetic peptide with optional protecting groups.
EKCVEEFKSVSTCLDSKAF

SEQ ID NO: 940 - protein, 19 aa, Artificial Sequence
  Synthetic peptide with optional protecting groups.
EKCVEEFKSYTSCLDSKAF

SEQ ID NO: 941 - protein, 19 aa, Artificial Sequence
  Synthetic peptide with optional protecting groups.
EKCVEEFKSFTTCLDSKAF

SEQ ID NO: 942 - protein, 19 aa, Artificial Sequence
  Synthetic peptide with optional protecting groups.
EKCVEEFKSFSSCLDSKAF

SEQ ID NO: 943 - protein, 19 aa, Artificial Sequence
  Synthetic peptide with optional protecting groups.
EKCVEEFKSFTSCLDSKAF

SEQ ID NO: 944 - protein, 19 aa, Artificial Sequence
  Synthetic peptide with optional protecting groups.
EKCVEEFKSFTSCLDSKAF

SEQ ID NO: 945 - protein, 19 aa, Artificial Sequence
  Synthetic peptide with optional protecting groups.
EKCVEEFKSFTSCLDSKAF

SEQ ID NO: 946 - protein, 19 aa, Artificial Sequence
  Synthetic peptide with optional protecting groups.
EKCVEEFKSFTSCFDSKAF

SEQ ID NO: 947 - protein, 19 aa, Artificial Sequence
  Synthetic peptide with optional protecting groups.
EKCVEEFKSFTSCFESKAF

SEQ ID NO: 948 - protein, 19 aa, Artificial Sequence
  Synthetic peptide with optional protecting groups.
EKCVEEFKSFTSCLESKAF

SEQ ID NO: 949 - protein, 19 aa, Artificial Sequence
  Synthetic peptide with optional protecting groups.
EKCVEEFKSFTSCIDSKAF

SEQ ID NO: 950 - protein, 19 aa, Artificial Sequence
  Synthetic peptide with optional protecting groups.
EKCVEELKSFTSCFDSKAF

SEQ ID NO: 951 - protein, 19 aa, Artificial Sequence
  Synthetic peptide with optional protecting groups.
DKCVEEFKSFTSCFDSKAF

SEQ ID NO: 952 - protein, 19 aa, Artificial Sequence
  Synthetic peptide with optional protecting groups.
DKCVEEFKSFTSCFESKAF

SEQ ID NO: 953 - protein, 19 aa, Artificial Sequence
  Synthetic peptide with optional protecting groups.
ERCVEEFKSFTSCFDSKAF

SEQ ID NO: 954 - protein, 19 aa, Artificial Sequence
  Synthetic peptide with optional protecting groups.
EKCFEEFKSFTSCFDSKAF

SEQ ID NO: 955 - protein, 19 aa, Artificial Sequence
  Synthetic peptide with optional protecting groups.
EKCFEEFKSFTSCFESKAF

SEQ ID NO: 956 - protein, 19 aa, Artificial Sequence
  Synthetic peptide with optional protecting groups.
EKCVEEFKSFSSCFESKAF

SEQ ID NO: 957 - protein, 19 aa, Artificial Sequence
  Synthetic peptide with optional protecting groups.
EKCVEEFKSFQSCFDSKAF

SEQ ID NO: 958 - protein, 19 aa, Artificial Sequence
  Synthetic peptide with optional protecting groups.
EKCFEEFKSFQSCFDSKAF

SEQ ID NO: 959 - protein, 19 aa, Artificial Sequence
  Synthetic peptide with optional protecting groups.
EKCVEEFKQFTSCFDSKAF

SEQ ID NO: 960 - protein, 19 aa, Artificial Sequence
  Synthetic peptide with optional protecting groups.
EKCVEEFKQLTSCLDSKAF

SEQ ID NO: 961 - protein, 19 aa, Artificial Sequence
  Synthetic peptide with optional protecting groups.
EKCFEEFKSFQSCLDSKAF

SEQ ID NO: 962 - protein, 19 aa, Artificial Sequence
  Synthetic peptide with optional protecting groups.
EKCVEEFKQFTSCFDSKAF

SEQ ID NO: 963 - protein, 19 aa, Artificial Sequence
  Synthetic peptide with optional protecting groups.
EKCVEEFKSFTSCFESKAF

SEQ ID NO: 964 - protein, 19 aa, Artificial Sequence
  Synthetic peptide with optional protecting groups.
ERCFEEFKSFTSCFDSKAF

SEQ ID NO: 965 - protein, 19 aa, Artificial Sequence
  Synthetic peptide with optional protecting groups.
DKCFEEFKSFTSCFDSKAF

SEQ ID NO: 966 - protein, 19 aa, Artificial Sequence
  Synthetic peptide with optional protecting groups.
ERCVEEFKSLTSCLESKAF

SEQ ID NO: 967 - protein, 19 aa, Artificial Sequence
  Synthetic peptide with optional protecting groups.
EKCVEEFKSLTSCLDSKFF

SEQ ID NO: 968 - protein, 19 aa, Artificial Sequence
  Synthetic peptide with optional protecting groups.
EKCFEEFKSFTSCFDSKFF

SEQ ID NO: 969 - protein, 19 aa, Artificial Sequence
  Synthetic peptide with optional protecting groups.
DKCFEEFKSFTSCLDSKFF

SEQ ID NO: 970 - protein, 19 aa, Artificial Sequence
  Synthetic peptide with optional protecting groups.
DKCFEEFKSFTSCLESKFF

SEQ ID NO: 971 - protein, 19 aa, Artificial Sequence
  Synthetic peptide with optional protecting groups.
DKCFEELKSFTSCLDSKFF

SEQ ID NO: 972 - protein, 19 aa, Artificial Sequence
  Synthetic peptide with optional protecting groups.
ERCFEEFKSFTSCLDSKFF

SEQ ID NO: 973 - protein, 19 aa, Artificial Sequence
  Synthetic peptide with optional protecting groups.
EKAVEEFKSFTSCLDSKAF

SEQ ID NO: 974 - protein, 19 aa, Artificial Sequence
  Synthetic peptide with optional protecting groups.
DKAVEEFKSFTSCLDSKFF

SEQ ID NO: 975 - protein, 19 aa, Artificial Sequence
  Synthetic peptide with optional protecting groups.
EKAVEEFKSFTSALDSKAF

SEQ ID NO: 976 - protein, 19 aa, Artificial Sequence
  Synthetic peptide with optional protecting groups.
DKAVEEFKSFTSALDSKAF

SEQ ID NO: 977 - protein, 19 aa, Artificial Sequence
  Synthetic peptide with optional protecting groups.
DRAFEEFKSFTSCLDSKFF

SEQ ID NO: 978 - protein, 19 aa, Artificial Sequence
  Synthetic peptide with optional protecting groups.
DRAFEEFKSFTSALDSKFF

SEQ ID NO: 979 - protein, 19 aa, Artificial Sequence
  Synthetic peptide with optional protecting groups.
DKCFEEFKSFTSCFESKFF

SEQ ID NO: 980 - protein, 19 aa, Artificial Sequence
  Synthetic peptide with optional protecting groups.
EKCYEEFKSFTSCLDSKFF

SEQ ID NO: 981 - protein, 19 aa, Artificial Sequence
  Synthetic peptide with optional protecting groups.
DKCWEEFKSFTSCLDSKFF

SEQ ID NO: 982 - protein, 19 aa, Artificial Sequence
  Synthetic peptide with optional protecting groups.
EKCFEEFKSYTSCLDSKFF

SEQ ID NO: 983 - protein, 19 aa, Artificial Sequence
  Synthetic peptide with optional protecting groups.
EKCFEEFKSWTSCLDSKFF

SEQ ID NO: 984 - protein, 19 aa, Artificial Sequence
  Synthetic peptide with optional protecting groups.
EKCVEEFKSWTSCLDSKAF

SEQ ID NO: 985 - protein, 19 aa, Artificial Sequence
  Synthetic peptide with optional protecting groups.
DKCFEEFKSWTSCLDSKAF

SEQ ID NO: 986 - protein, 18 aa, Artificial Sequence
  Synthetic peptide with optional protecting groups.
DVWKAAYDKFAEKFKEFF

SEQ ID NO: 987 - protein, 18 aa, Artificial Sequence
  Synthetic peptide with optional protecting groups.
DVWKAFYDKFAEKFKEAF

SEQ ID NO: 988 - protein, 18 aa, Artificial Sequence
  Synthetic peptide with optional protecting groups.
DFWKAFYDKVAEKFKEAF

SEQ ID NO: 989 - protein, 5 aa, Artificial Sequence
  Synthetic peptide with optional protecting groups.
XDRFK

SEQ ID NO: 990 - protein, 4 aa, Artificial Sequence
  Synthetic peptide with optional protecting groups.
XREL

SEQ ID NO: 991 - protein, 7 aa, Artificial Sequence
  Synthetic peptide with optional protecting groups.
LAEYHAK

SEQ ID NO: 992 - protein, 8 aa, Artificial Sequence
  Synthetic peptide linker.
GGGGSSSS

SEQ ID NO: 993 - protein, 45 aa, Artificial Sequence [sequence omitted]

SEQ ID NO: 994 - protein, 41 aa, Artificial Sequence
  Synthetic peptide with optional protecting groups.
LLEQLNEQFNWVSRLANLTQGEPDWFKAFYDKVAEKFKEAF

SEQ ID NO: 995 - protein, 18 aa, Artificial Sequence
  Synthetic peptide with optional protecting groups.
DRLKAFYDKVAWKLKEAF